(19) United States
(12) Patent Application Publication
(10) Pub. No.: US 2025/0127790 A1
(43) Pub. Date: Apr. 24, 2025

(54) COMBINATION THERAPY USING VPS34 INHIBITORS

(71) Applicant: Deciphera Pharmaceuticals, LLC, Waltham, MA (US)

(72) Inventors: Yasmin Yu, Huddinge (SE); Martin Andersson, Huddinge (SE); Daniel L. Flynn, Waltham, MA (US); Madhumita Bogdan, Waltham, MA (US)

(21) Appl. No.: 18/683,078

(22) PCT Filed: Aug. 12, 2022
(86) PCT No.: PCT/US2022/074925

§ 371 Date: Feb. 12, 2024

Related U.S. Application Data
(60) Provisional application No. 63/317,500, filed on Mar. 7, 2022.
(60) Provisional application No. 63/232,983, filed on Aug. 13, 2021.

(51) Int. Cl. A61K 31/5377; A61K 31/7084; A61P 35/00

(52) U.S. Cl. CPC A61K 31/5377; A61K 31/7084; A61P 35/00

(57) ABSTRACT

Described herein, in part, are methods of treating cancer in patients in need thereof, comprising administering to the patient a VPS34 inhibitor and one or more additional therapeutic agents, such as a STING agonist.

### CROSS-REFERENCE

This application is a U.S. National Stage application filed under 35 U.S.C. § 371 claiming benefit to International Patent Application No. PCT/US2022/074925 filed Aug. 12, 2022, which claims priority to U.S. Provisional Application No. 63/232,983 filed Aug. 13, 2021, and U.S. Provisional Application No. 63/317,500 filed Mar. 7, 2022, the contents of each of which are incorporated herein by reference.

### REFERENCE TO A SEQUENCE LISTING XML

This application contains a Sequence Listing which has been submitted electronically in XML format. The Sequence Listing XML is incorporated herein by reference. Said XML file, created on Aug. 10, 2022, is named DCP-109WO_SL.xml and is 20,390 bytes in size.

### BACKGROUND

Autophagy is a cellular process enabling the recycling of cytoplasmic components via the formation of double-membraned vesicles termed autophagosomes. In addition to autophagy-mediated vesicular trafficking, the endosomal vesicular trafficking process also enables the sequestration and destruction/recycling of cellular components. Upon fusion with lysosomes, the cargo of the autophagosome or the late-stage endosome is degraded and released back into the cell, fueling cellular metabolism. Due to its cytoprotective role, defective autophagy has been implicated in several human pathologies including cancer. Cancer cells have been shown to upregulate autophagy in response to stress factors present in the tumor microenvironment (TME) such as hypoxia, nutrient deprivation, as well as a resistance mechanism to anti-cancer therapy. Recently it has become known that the autophagy pathway and/or the endosomal pathway can be upregulated in various cancers to degrade proteins that would otherwise trigger a productive anti-tumor immune response. In certain cases, autophagy or endosomal trafficking in tumor cells leads to immune evasion by destroying the release of proinflammatory mediators, reducing immune cell recognition via MHC-I, and limiting T and natural killer (NK) cell-mediated cell death. Thus, there is a need to block the immunosuppressive effects of autophagy and endosomal trafficking in the tumor environment as a potential therapeutic strategy to improve cancer immunotherapy.

Innate immune sensing in the tumor environment is a critical for promoting spontaneous tumor-initiated T cell priming and tumor infiltration. Transcriptional profiling analyses of melanoma patients have revealed that tumors containing infiltrating activated T cells are characterized by a type I IFN signature. Murine studies have demonstrated that type I IFN signaling plays a critical role in tumor-initiated T cell priming. These findings in humans and in mice indicate that the innate immune system is defective in non-T-cell-inflamed tumors. Thus, strategies to induce type I IFN signaling and antigen-presenting cell activation in the tumor environment to bridge the innate and adaptive immune responses may have therapeutic utility in treating cancer patients. Recent work has demonstrated that activation of the STING pathway in the innate tumor environment is required for induction of a productive CD8+ T cell response against tumor-derived antigens in vivo. STING (stimulator of interferon genes) is a transmembrane protein localized to the endoplasmic reticulum that is activated in response to binding of cyclic dinucleotides (CDNs), resulting in a downstream signaling cascade involving TBK1 kinase activation, IRF-3 phosphorylation, and production of IFN-b and other cytokines. IFN-b is the major cytokine induced in response to activating STING, either by exogenous CDNs produced by bacterial infection or through binding of a structurally distinct endogenous CDNs produced by a host cyclic GMPAMP synthetase (cGAS). These observations suggest that direct activation of the STING pathway in the tumor environment by specific agonists could be an effective therapeutic strategy to promote broad tumor-initiated T cell priming against an individual's tumor antigen repertoire.

Exogenously administered STING agonists are being developed as vaccine adjuvants, as well as direct anti-tumor therapeutic effects. In addition to the administration of exogenous STING agonists, an alternative approach that modulates and upregulates endogenous STING signaling in the tumor environment may be a more preferred method of treatment. The lipid kinase vacuolar protein sorting 34 (VPS34) is a promising target for blocking autophagy- or endosomal pathway-mediated immunosuppression. VPS34, also known as class III phosphoinositide 3-kinase (PIK3C3), regulates autophagy initiation and other vesicular trafficking processes, including playing a key role in endosomal trafficking. Therefore, there is a need for therapeutics that directly activate the STING pathway for use in the treatment of disorders such as cancer.

### SUMMARY

The present disclosure, in part, is drawn to a method of treating cancers in a patient in need thereof, comprising administering to the patient a VPS34 inhibitor. It has been found that VPS34 inhibitors stimulate the endogenous CGAS/STING pathway in cancer cells or other cells in the tumor environment. Mechanistically, it has been found that VPS34 inhibition triggers an interferon (IFN) response by upregulating the cGAS-STING pathway in various cancers. The present disclosure also provides a method for turning cold tumors (tumor that evade the immune system) into hot tumors (tumors that are recognized and destroyed by the immune system) by administration of a VPS34 inhibitor. The present disclosure is further drawn to a method of the use of VPS34 inhibitors to synergize with exogenous STING agonists in increasing STING-dependent IFN response in vitro and in vivo and further converting cold tumors into hot tumors. These findings identify VPS34 inhibitors as a therapeutic strategy to activate the endogenous cGAS-STING pathway, resulting in a proinflammatory IFN response in the tumor environment and a productive anti-tumor immune response. Further, administration of a VPS34 inhibitor to a cancer patient in need of treatment could be beneficial in combination with other immunostimulatory therapeutics, such as a STING agonist, to improve the outcome of cancer immunotherapy.

In an aspect, provided herein is a method of treating cancer in a patient in need thereof, comprising: (i) administering to the patient a therapeutically effective amount of a compound represented by Formula I:

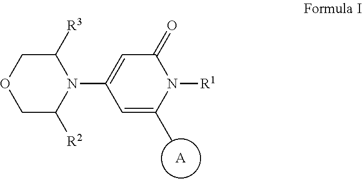

or a pharmaceutically acceptable salt, stereoisomer, or tautomer thereof, wherein: R¹, R², and R³ are independently selected from the group consisting of H, C₁-C₃haloalkyl, and C₁-C₃alkyl; A represents:

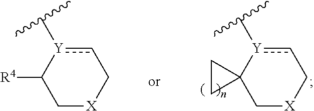

[symbol]

is a single bond or a double bond; X is selected from the group consisting of CH₂, S, SO, SO₂, NR⁵, NCOR⁵, NCOR⁹, NCOCH₂R⁹, O, and a bond; Y is selected from the group consisting of N, CH, and C, provided that, when Y is CH, [symbol] is a single bond; n is selected from 1, 2, 3 and 4; R⁴ is selected from the group consisting of H, halogen, COR⁶, C₁-C₆alkyl, C₁-C₃alkoxyC₁-C₃alkyl, C₁-C₆alkoxy, C₃-C₆cycloalkyl, C₃-C₆heterocyclyl, C₁-C₃cyanoalkyl, C₁-C₃haloalkyl, aryl, and heteroaryl, wherein said aryl and said heteroaryl are optionally substituted with one or more R⁷; R⁵ is selected from the group consisting of H, C₁-C₃fluoroalkyl, C₁-C₃alkyl, C₁-C₃alkoxyC₁-C₃alkyl, and C₃-C₆cycloalkyl; R⁶ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; each R⁷ is independently selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy and C₁-C₃alkoxy; R⁹ is selected from the group consisting of C₁-C₃alkyl, C₁-C₃alkoxy, C₃-C₆cycloalkyl, heterocyclyl, phenyl and a monocyclic heteroaryl, wherein said heterocyclyl, said phenyl and said monocyclic heteroaryl are optionally substituted with one or two R⁸; and each R⁸ is independently selected from the group consisting of halogen, C₁-C₃haloalkyl and C₁-C₃alkyl; and (ii) administering to the patient a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

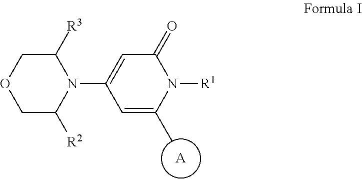

or a pharmaceutically acceptable salt, stereoisomer, or tautomer thereof, wherein: R¹, R², and R³ are independently selected from the group consisting of H, C₁-C₃haloalkyl, and C₁-C₃alkyl; A represents:

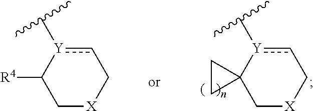

[symbol]

is a single bond or a double bond; X is selected from the group consisting of CH₂, S, SO, SO₂, NR⁵, NCOR⁵, NCOR⁹, NCOCH₂R⁹, O, and a bond; Y is selected from the group consisting of N, CH, and C, provided that, when Y is CH, [symbol] is a single bond; n is selected from 1, 2, 3 and 4; R⁴ is selected from the group consisting of H, halogen, COR⁶, C₁-C₆alkyl, C₁-C₃alkoxyC₁-C₃alkyl, C₁-C₆alkoxy, C₃-C₆cycloalkyl, C₃-C₆heterocyclyl, C₁-C₃cyanoalkyl, C₁-C₃haloalkyl, aryl, and heteroaryl, wherein said aryl and said heteroaryl are optionally substituted with one or more R⁷; R⁵ is selected from the group consisting of H, C₁-C₃fluoroalkyl, C₁-C₃alkyl, C₁-C₃alkoxyC₁-C₃alkyl, and C₃-C₆cycloalkyl; R⁶ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; each R⁷ is independently selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy and C₁-C₃alkoxy; R⁹ is selected from the group consisting of C₁-C₃alkyl, C₁-C₃alkoxy, C₃-C₆cycloalkyl, heterocyclyl, phenyl and a monocyclic heteroaryl, wherein said heterocyclyl, said phenyl and said monocyclic heteroaryl are optionally substituted with one or two R⁸; and each R⁸ is independently selected from the group consisting of halogen, C₁-C₃haloalkyl and C₁-C₃alkyl; in an amount sufficient to induce a Type I interferon response by the cell; and
(ii) a STING agonist in an amount sufficient to increase the expression level of the at least one chemokine in the cell.

In an aspect, provided herein is a method of treating cancer in a patient in need thereof, comprising administering to the patient: (i) a therapeutically effective amount of a compound represented by Formula II:

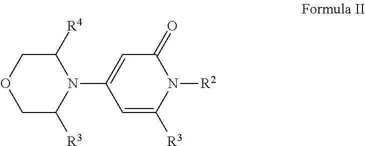

or a pharmaceutically acceptable salt, stereoisomer, or tautomer thereof, wherein: R¹ is selected from the group consisting of aryl and heteroaryl, wherein said aryl and said heteroaryl being mono- or bicyclic and each of aryl and heteroaryl is optionally substituted with one or more independent occurrences of a substituent selected from the group consisting of R⁵, R⁶, R⁷ and R⁸; each of R², R³, R⁴ is independently selected from the group consisting of H, C₁-C₃haloalkyl, and C₁-C₃alkyl; each of R⁵, R⁶, R⁷, and R⁸ is independently selected from the group consisting of halogen, C₁-C₆alkyl, C₁-C₆alkoxy, C₁-C₆haloalkyl, amino, —NHSO₂R⁹, hydroxy, phenyl, and a monocyclic heteroaryl; and R⁹ is selected from C₁-C₃haloalkyl and C₁-C₃alkyl; and (ii) a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

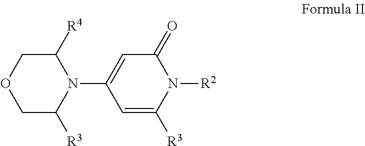

or a pharmaceutically acceptable salt, stereoisomer, or tautomer thereof, wherein: R¹ is selected from the group consisting of aryl and heteroaryl, wherein said aryl and said heteroaryl being mono- or bicyclic and each of aryl and heteroaryl is optionally substituted with one or more independent occurrences of a substituent selected from the group consisting of R⁵, R⁶, R⁷ and R⁸; each of R², R³, R⁴ is independently selected from the group consisting of H, C₁-C₃haloalkyl, and C₁-C₃alkyl; each of R⁵, R⁶, R⁷, and R⁸ is independently selected from the group consisting of halogen, C₁-C₆alkyl, C₁-C₆alkoxy, C₁-C₆haloalkyl, amino, —NHSO₂R⁹, hydroxy, phenyl, and a monocyclic heteroaryl; and R⁹ is selected from C₁-C₃haloalkyl and C₁-C₃alkyl; in an amount sufficient to induce a Type I interferon response by the cell; and (ii) a STING agonist in an amount sufficient to increase the expression level of the at least one chemokine in the cell.

In an aspect, provided herein is a method of treating cancer in a patient in need thereof, comprising administering to the patient: (i) a therapeutically effective amount of a compound represented by Formula III:

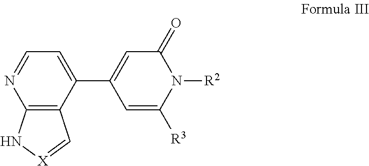

or a pharmaceutically acceptable salt, stereoisomer, or tautomer thereof, wherein: X is selected from N and CR¹; R¹ is selected from the group consisting of H, C₁-C₃alkyl, C₁-C₃haloalkyl, C₁-C₃alkoxyC₁-C₃alkyl, C₃-C₆cycloalkyl, cyano, phenyl, and monocyclic heteroaryl, wherein each of phenyl and monocyclic heteroaryl is optionally substituted with one or more substituents independently selected from the group consisting of halo, N—C₁-C₃alkylamino, N,N-diC₁-C₃alkylamino, C₃-C₆cycloalkyl, C₁-C₃alkoxyC₁-C₃alkyl, C₁-C₃haloalkyl, C₁-C₃haloalkoxy, C₁-C₃alkoxy, and C₁-C₃alkyl; R² is selected from the group consisting of H, C₁-C₃haloalkyl, and C₁-C₃alkyl; R³ is selected from the group consisting of A, phenyl, and monocyclic heteroaryl, wherein each of phenyl and monocyclic heteroaryl is optionally substituted with one or more occurrences of R⁴; each R⁴ is independently selected from the group consisting of COR⁵, halogen, C₁-C₆alkyl, C₁-C₆alkoxy, C₁-C₆haloalkoxy, amino N—C₁-C₃alkylamino, N,N-diC₁-C₃alkylamino, 1-pyrrolidinyl, 1-piperidinyl, 1-azetidinyl, NHSO₂R⁶, SO₂R⁷, hydroxy, C₃-C₆cycloalkyl, C₁-C₃alkoxyC₁-C₃alkyl, C₁-C₃cyanoalkyl and C₁-C₆haloalkyl; R⁵ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; R⁶ is selected from C₁-C₃haloalkyl and C₁-C₃alkyl; each R⁷ is independently selected from the group consisting of R⁸, C₁-C₆alkyl, N—C₁-C₃alkylamino, N,N-diC₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen; each R⁸ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁹; each R⁹ is independently selected from the group consisting of halo, N—C₁-C₃alkylamino, N,N-diC₁-C₃alkylamino, C₁-C₃alkoxyC₁-C₃alkyl, amino, C₁-C₃haloalkyl, C₁-C₃alkoxy, C₁-C₃haloalkoxy, C₃-C₆cycloalkyl and C₁-C₃alkyl; A is

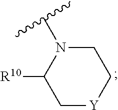

R¹⁰ is selected from the group consisting of H, halogen, COR¹¹, C₁-C₆alkyl, C₁-C₃alkoxyC₁-C₃alkyl, C₁-C₆alkoxy, C₃-C₆cycloalkyl, C₁-C₃cyanoalkyl, C₁-C₃haloalkyl, phenyl, and heteroaryl, wherein each of phenyl and heteroaryl is optionally substituted with one or more occurrences of R¹², and provided that when R¹⁰ is phenyl or heteroaryl, then X is N or CH; each R¹¹ is independently selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹³, NCOR⁷, NCOOR¹⁴, NSO₂R⁷, NCOCH₂R⁷, O, and a bond; R¹² is selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy, and C₁-C₃alkoxy; R¹³ is selected from H, C₁-C₃haloalkyl, C₁-C₃alkoxyC₁-C₃alkyl, C₁-C₃alkyl, C₃-C₆cycloalkyl; and R¹⁴ is selected from R⁸, C₁-C₆alkyl, C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen; and (ii) a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

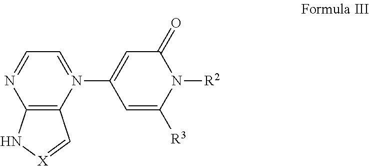

or a pharmaceutically acceptable salt, stereoisomer, or tautomer thereof, wherein: X is selected from N and CR¹; R¹ is selected from the group consisting of H, C₁-C₃alkyl, C₁-C₃haloalkyl, C₁-C₃alkoxyC₁-C₃alkyl, C₃-C₆cycloalkyl, cyano, phenyl, and monocyclic heteroaryl, wherein each of phenyl and monocyclic heteroaryl is optionally substituted with one or more substituents independently selected from the group consisting of halo, N—C₁-C₃alkylamino, N,N-diC₁-C₃alkylamino, C₃-C₆cycloalkyl, C₁-C₃alkoxyC₁-C₃alkyl, C₁-C₃haloalkyl, C₁-C₃haloalkoxy, C₁-C₃alkoxy, and C₁-C₃alkyl; R² is selected from the group consisting of H, C₁-C₃haloalkyl, and C₁-C₃alkyl; R³ is selected from the group consisting of A, phenyl, and monocyclic heteroaryl, wherein each of phenyl and monocyclic heteroaryl is optionally substituted with one or more occurrences of R⁴; each R⁴ is independently selected from the group consisting of COR⁵, halogen, C₁-C₆alkyl, C₁-C₆alkoxy, C₁-C₆haloalkoxy, amino N—C₁-C₃alkylamino, N,N-diC₁-C₃alkylamino, 1-pyrrolidinyl, 1-piperidinyl, 1-azetidinyl, NHSO₂R⁶, SO₂R⁷, hydroxy, C₃-C₆cycloalkyl, C₁-C₃alkoxyC₁-C₃alkyl, C₁-C₃cyanoalkyl and C₁-C₆haloalkyl; R⁵ is selected from the group consisting of C₁-C₃alkoxy, N₁-C₃alkylamino, N,N-diC₁-C₃alkylamino, 1-pyrrolidinyl, 1-piperidinyl, and 1-azetidinyl; R⁶ is selected from C₁-C₃haloalkyl and C₁-C₃alkyl; each R⁷ is independently selected from the group consisting of R⁸, C₁-C₆alkyl, N—C₁-C₃alkylamino, N,N-diC₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen; each R⁸ is independently selected from the group consisting of phenyl, monocyclic heteroaryl, C₃-C₆cycloalkyl, and heterocyclyl, wherein each of phenyl, monocyclic heteroaryl, C₃-C₀cycloalkyl, and heterocyclyl is optionally substituted with one or more occurrences of R⁹; each R⁹ is independently selected from the group consisting of halo, N—C₁-C₃alkylamino, N,N-diC₁-C₃alkylamino, C₁-C₃alkoxyC₁-C₃alkyl, amino, C₁-C₃haloalkyl, C₁-C₃alkoxy, C₁-C₃haloalkoxy, C₃-C₆cycloalkyl and C₁-C₃alkyl; A is

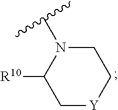

R¹⁰ is selected from the group consisting of H, halogen, COR^{DI}, C₁-C₆alkyl, C₁-C₃alkoxyC₁-C₃alkyl, C₁-C₆alkoxy, C₃-C₆cycloalkyl, C₁-C₃cyanoalkyl, C₁-C₃haloalkyl, phenyl, and heteroaryl, wherein each of phenyl and heteroaryl is optionally substituted with one or more occurrences of R¹², and provided that when R¹⁰ is phenyl or heteroaryl, then X is N or CH; each R¹¹ is independently selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹³, NCOR⁷, NCOOR¹⁴, NSO₂R⁷, NCOCH₂R⁷, O, and a bond; R¹² is selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy, and C₁-C₃alkoxy; R¹³ is selected from H, C₁-C₃haloalkyl, C₁-C₃alkoxyC₁-C₃alkyl, C₁-C₃alkyl, C₃-C₆cycloalkyl; and R¹⁴ is selected from R⁸, C₁-C₅alkyl, C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen; and in an amount sufficient to induce a Type I interferon response by the cell; and (ii) a STING agonist in an amount sufficient to increase the expression level of the at least one chemokine in the cell.

In an aspect, provided herein is a method of treating cancer in a patient in need thereof, comprising: (i) administering to the patient a therapeutically effective amount of a compound represented by Formula IV:

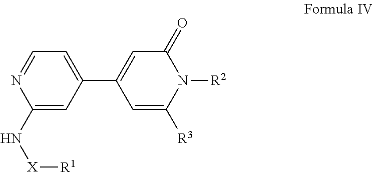

or a pharmaceutically acceptable salt, stereoisomer, or tautomer thereof, wherein: X is selected from —C(═O)— and a bond; R¹ is selected from the group consisting of H, C₁-C₃alkyl, C₁-C₃haloalkyl, C₁-C₃alkoxyC₁-C₃alkyl, C₃-C₆cycloalkyl, C₃-C₆cyclohaloalkyl, C₁-C₃alkoxy, C₁-C₃haloalkoxy, C₃-C₆cycloalkoxymethyl, N—C₁-C₃alkylamino, N,N-diC₁-C₃alkylamino, 1-pyrrolidinyl, 1-piperidinyl and 1-azetidinyl, provided that when R¹ is selected from the group consisting of C₁-C₃alkoxy, C₁-C₃haloalkoxy, N—C₁-C₃alkylamino, N,N-diC₁-C₃alkylamino, 1-pyrrolidinyl, 1-piperidinyl, and 1-azetidinyl, then X is C═O; R² is selected from the group consisting of H, C₁-C₃haloalkyl, and C₁-C₃alkyl; R³ is selected from the group consisting of A, phenyl and monocyclic heteroaryl, wherein each of phenyl and monocyclic heteroaryl is optionally substituted with one or more occurrences of a substituent independently selected from the group consisting of R⁴, R⁵, R⁶ and R⁷; each R⁴, R⁵, R⁶, and R⁷ is independently selected from the group consisting of halo, C₁-C₆alkyl, C₃-C₆cycloalkyl, C₁-C₆alkoxy, C₁-C₃haloalkoxy, N,N-diC₁-C₃alkylamino, N—C₁-C₃alkylamino, 1-azetidinyl, C₁-C₆haloalkyl, amino, NHSO₂R⁸, SO₂R⁹, and hydroxy; R⁸ is selected from C₁-C₃haloalkyl and C₁-C₃alkyl; each R⁹ is independently selected from the group consisting of R¹⁰, C₁-C₆alkyl, amino, N—C₁-C₃alkylamino, N,N-di-C₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; each R¹⁰ is independently selected from the group consisting of phenyl, monocyclic heteroaryl, C₃-C₆cycloalkyl, and heterocyclyl, wherein each phenyl, monocyclic heteroaryl, C₃-C₆cycloalkyl, and heterocyclyl is optionally substituted with one or more occurrences of R¹¹; each R¹¹ is independently selected from the group consisting of halo, C₁-C₃alkoxyC₁-C₃alkyl, amino, N—C₁-C₃alkylamino, N,N-diC₁-C₃alkylamino, C₁-C₃haloalkoxy, C₁-C₃alkoxy, C₃-C₆cycloalkyl, C₁-C₃haloalkyl, and C₁-C₃alkyl; A is:

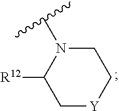

R¹² is selected from the group consisting of H, halo, COR¹³, C₁-C₆alkyl, C₁-C₃alkoxyC₁-C₃alkyl, C₁-C₆alkoxy, C₃-C₆cycloalkyl, C₁-C₃cyanoalkyl, and C₁-C₃haloalkyl; R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹⁵, NSO₂R⁹, NCOCH₂R⁹, O, and a bond; R¹⁴ is selected from the group consisting of H, C₁-C₃haloalkyl, C₁-C₃alkoxyC₁-C₃alkyl, C₁-C₃alkyl, and C₃-C₆cycloalkyl; and R¹⁵ is selected from the group consisting of R¹⁰, C₁-C₆alkyl and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and (ii) administering to the patient a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

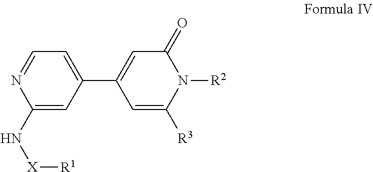

or a pharmaceutically acceptable salt, stereoisomer, or tautomer thereof, wherein: X is selected from —C(═O)— and a bond; R¹ is selected from the group consisting of H, C₁-C₃alkyl, C₁-C₃haloalkyl, C₁-C₃alkoxyC₁-C₃alkyl, C₃-C₆cycloalkyl, C₃-C₆cyclohaloalkyl, C₁-C₃alkoxy, C₁-C₃haloalkoxy, C₃-C₆cycloalkoxymethyl, N—C₁-C₃alkylamino, N,N-diC₁-C₃alkylamino, 1-pyrrolidinyl, 1-piperidinyl and 1-azetidinyl, provided that when R¹ is selected from the group consisting of C₁-C₃alkoxy, C₁-C₃haloalkoxy, N—C₁-C₃alkylamino, N,N-diC₁-C₃alkylamino, 1-pyrrolidinyl, 1-piperidinyl, and 1-azetidinyl, then X is C═O; R² is selected from the group consisting of H, C₁-C₃haloalkyl, and C₁-C₃alkyl; R³ is selected from the group consisting of A, phenyl and monocyclic heteroaryl, wherein each of phenyl and monocyclic heteroaryl is optionally substituted with one or more occurrences of a substituent independently selected from the group consisting of R⁴, R⁵, R⁶ and R⁷; each R⁴, R⁵, R⁶, and R⁷ is independently selected from the group consisting of halo, C₁-C₆alkyl, C₃-C₆cycloalkyl, C₁-C₆alkoxy, C₁-C₃haloalkoxy, N,N-diC₁-C₃alkylamino, N—C₁-C₃alkylamino, 1-azetidinyl, C₁—C haloalkyl, amino, NHSO₂R⁸, SO₂R⁹, and hydroxy; R⁸ is selected from C₁-C₃haloalkyl and C₁-C₃alkyl; each R⁹ is independently selected from the group consisting of R¹⁰, C₁-C₆alkyl, amino, N—C₁-C₃alkylamino, N,N-di-C₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; each R¹⁰ is independently selected from the group consisting of phenyl, monocyclic heteroaryl, C₃-C₆cycloalkyl, and heterocyclyl, wherein each phenyl, monocyclic heteroaryl, C₃-C₆cycloalkyl, and heterocyclyl is optionally substituted with one or more occurrences of R¹¹; each R¹¹ is independently selected from the group consisting of halo, C₁-C₃alkoxyC₁-C₃alkyl, amino, N—C₁-C₃alkylamino, N,N-diC₁-C₃alkylamino, C₁-C₃haloalkoxy, C₁-C₃alkoxy, C₃-C₆cycloalkyl, C₁-C₃haloalkyl, and C₁-C₃alkyl; A is:

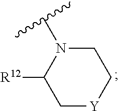

R¹² is selected from the group consisting of H, halo, COR¹³, C₁-C₆alkyl, C₁-C₃alkoxyC₁-C₃alkyl, C₁-C₆alkoxy, C₃-C₆cycloalkyl, C₁-C₃cyanoalkyl, and C₁-C₃haloalkyl; R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹⁵, NSO₂R⁹, NCOCH₂R⁹, O, and a bond; R¹⁴ is selected from the group consisting of H, C₁-C₃haloalkyl, C₁-C₃alkoxyC₁-C₃alkyl, C₁-C₃alkyl, and C₃-C₆cycloalkyl; and R¹⁵ is selected from the group consisting of R¹⁰, C₁-C₆alkyl and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and in an amount sufficient to induce a Type I interferon response by the cell; and (ii) a STING agonist in an amount sufficient to increase the expression level of the at least one chemokine in the cell.

In an aspect, provided herein is a method of treating cancer in a patient in need thereof, comprising: (i) administering to the patient a therapeutically effective amount of a compound represented by Formula V:

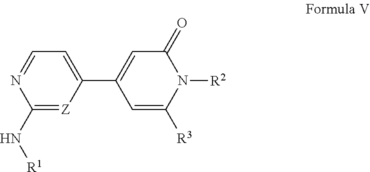

or a pharmaceutically acceptable salt, stereoisomer, or tautomer thereof, wherein: R¹ is selected from phenyl and monocyclic 5-6 membered heteroaryl, wherein each of phenyl and monocyclic 5-6 membered heteroaryl is optionally substituted with one or more substituents independently selected from the group consisting of halogen, C₁-C₆alkyl, C₃-C₄cycloalkyl, C₁-C₆alkoxy, C₁-C₆haloalkyl, C₁-C₆haloalkoxy, amino, N—C₁-C₃alkylamino and N,N-diC₁-C₃alkylamino; R² is selected from the group consisting of H, C₁-C₃haloalkyl, and C₁-C₃alkyl; R³ is selected from the group consisting of A, phenyl, and monocyclic heteroaryl, wherein each of phenyl and heteroaryl is optionally substituted with one or more occurrences of a substituent independently selected from the group consisting of R⁴, R⁵, R⁶, and R⁷; each of R⁴, R⁵, R⁶, and R⁷ is independently selected from the group consisting of halogen, C₁-C₆alkyl, C₃-C₄cycloalkyl, C₁-C₆alkoxy, C₁-C₆haloalkyl, C₁-C₆haloalkoxy, azetidine, amino, N—C₁-C₃alkylamino, N,N-diC₁-C₃alkylamino, NHSO₂R⁸, SO₂R⁹, and hydroxy; R⁸ is selected from C₁-C₃haloalkyl and C₁-C₃alkyl; each R⁹ is independently selected from the group consisting of R¹⁰, C₁-C₆alkyl, amino, N—C₁-C₃alkylamino, N,N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₆alkoxyC₁-C₃alkyl is optionally substituted with or one or more independent occurrences of halogen; each R¹⁰ is independently selected from the group consisting of phenyl, benzyl, monocyclic heteroaryl, C₃-C₆cycloalkyl, and heterocyclyl, wherein each of phenyl, benzyl, monocyclic heteroaryl, C₃-C₆cycloalkyl, and heterocyclyl is optionally substituted with one or more occurrences of R¹¹; each R¹¹ is independently selected from the group consisting of halogen, C₁-C₃haloalkyl, C₃-C₄cycloalkyl, C₁-C₃alkyl, amino, N—C₁-C₃alkylamino, N,N-diC₁-C₃alkylamino, and C₁-C₃alkoxyC₁-C₃alkyl; A is

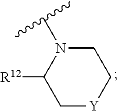

R² is selected from the group consisting of H, halogen, COR¹³, C₁-C₅alkyl, C₃-C₆cycloalkyl, C₁-C₃alkoxyC₁-C₃alkyl, C₁-C₆alkoxy, C₃-C₆cycloalkyl, C₁-C₃cyanoalkyl, and C₁-C₃haloalkyl; R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹⁵, NSO₂R⁹, NCOCH₂R⁹, O, and a bond; R¹⁴ is selected from H, C₁-C₃haloalkyl, C₁-C₃alkoxyC₁-C₃alkyl, C₁-C₃alkyl, and C₃-C₆cycloalkyl; and R¹⁵ is selected from R¹⁰, C₁-C₆alkyl, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and Z is selected from CH and N; and (ii) administering to the patient a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

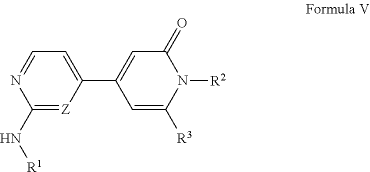

or a pharmaceutically acceptable salt, stereoisomer, or tautomer thereof, wherein: R¹ is selected from phenyl and monocyclic 5-6 membered heteroaryl, wherein each of phenyl and monocyclic 5-6 membered heteroaryl is optionally substituted with one or more substituents independently selected from the group consisting of halogen, C₁-C₆alkyl, C₃-C₄cycloalkyl, C₁-C₆alkoxy, C₁-C₆haloalkyl, C₁-C₆haloalkoxy, amino, N—C₁-C₃alkylamino and N,N-diC₁-C₃alkylamino; R² is selected from the group consisting of H, C₁-C₃haloalkyl, and C₁-C₃alkyl; R³ is selected from the group consisting of A, phenyl, and monocyclic heteroaryl, wherein each of phenyl and heteroaryl is optionally substituted with one or more occurrences of a substituent independently selected from the group consisting of R⁴, R⁵, R⁶, and R⁷; each of R⁴, R⁵, R⁶, and R⁷ is independently selected from the group consisting of halogen, C₁-C₆alkyl, C₃-C₄cycloalkyl, C₁-C₆alkoxy, C₁-C₆haloalkyl, C₁-C₆haloalkoxy, azetidine, amino, N—C₁-C₃alkylamino, N,N-diC₁-C₃alkylamino, NHSO₂R⁸, SO₂R⁹, and hydroxy; R⁸ is selected from C₁-C₃haloalkyl and C₁-C₃alkyl; each R⁹ is independently selected from the group consisting of R¹⁰, C₁-C₆alkyl, amino, N—C₁-C₃alkylamino, N,N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; each R¹⁰ is independently selected from the group consisting of phenyl, benzyl, monocyclic heteroaryl, C₃-C₆cycloalkyl, and heterocyclyl, wherein each of phenyl, benzyl, monocyclic heteroaryl, C₃-C₆cycloalkyl, and heterocyclyl is optionally substituted with one or more occurrences of R¹¹; each R¹¹ is independently selected from the group consisting of halogen, C₁-C₃haloalkyl, C₃-C₄cycloalkyl, C₁-C₃alkyl, amino, N—C₁-C₃alkylamino, N,N-diC₁-C₃alkylamino, and C₁-C₃alkoxyC₁-C₃alkyl; A is

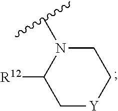

R¹² is selected from the group consisting of H, halogen, COR¹³, C₁-C₆alkyl, C₃-C₆cycloalkyl, C₁-C₃alkoxyC₁-C₃alkyl, C₁-C₆alkoxy, C₃-C₆cycloalkyl, C₁-C₃cyanoalkyl, and C₁-C₃haloalkyl; R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹⁵, NSO₂R⁹, NCOCH₂R⁹, O, and a bond; R¹⁴ is selected from H, C₁-C₃haloalkyl, C₁-C₃alkoxyC₁-C₃alkyl, C₁-C₃alkyl, and C₃-C₆cycloalkyl; and R¹⁵ is selected from R¹⁰, C₁-C₆alkyl, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and Z is selected from CH and N; and in an amount sufficient to induce a Type I interferon response by the cell; and (ii) a STING agonist in an amount sufficient to increase the expression level of the at least one chemokine in the cell.

In an aspect, provided herein is a method of treating cancer in a patient in need thereof, comprising: (i) administering to the patient a therapeutically effective amount of a compound represented by Formula VI:

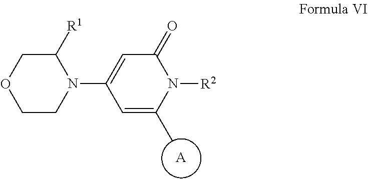

or a pharmaceutically acceptable salt, stereoisomer, or tautomer thereof, wherein: R¹ is selected from C₁-C₃alkyl and cyclopropyl; R² is selected from the group consisting of H, C₁-C₃haloalkyl, and C₁-C₃alkyl; A is selected from:

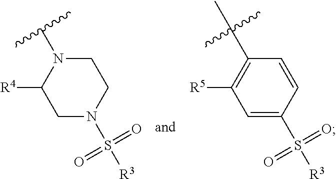

each R³ is independently selected from the group consisting of R⁶, C₁-C₆alkyl, amino N—C₁-C₃alkylamino, N,N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R⁶, and each of C₁-C₆alkyl and C₁-C₃alkoxyC₁-C₃alkyl is optionally substituted with or one or more independent occurrences of halogen; R⁴ is selected from the group consisting of C₁-C₆alkyl, C₁-C₆alkoxy, C₁-C₆haloalkyl, C₃-C₆cycloalkyl, and phenyl, wherein phenyl is optionally substituted with one or more occurrences of a substituent independently selected from the group consisting of fluoro, chloro, methyl, methoxy, dimethylamino, trifluoromethoxy, trifluoromethyl, and cyclopropyl; R⁵ is selected from the group consisting of halogen, C₁-C₆alkyl, C₁-C₆alkoxy, C₁-C₆haloalkyl, and C₃-C₆cycloalkyl; each R⁶ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁷; and each R⁷ is independently selected from the group consisting of halogen, amino, N—C₁-C₃alkylamino, N,N-diC₁-C₃alkylamino and C₁-C₃alkoxyC₁-C₃alkyl, C₁-C₃alkoxy, C₁-C₃haloalkoxy, C₃-C₆cycloalkyl, C₁-C₃haloalkyl, and C₁-C₃alkyl; and (ii) administering to the patient a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

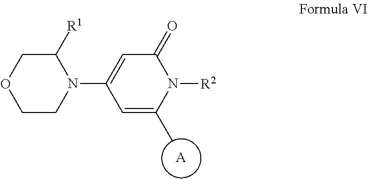

or a pharmaceutically acceptable salt, stereoisomer, or tautomer thereof, wherein: R¹ is selected from C₁-C₃alkyl and cyclopropyl; R² is selected from the group consisting of H, C₁-C₃haloalkyl, and C₁-C₃alkyl; A is selected from:

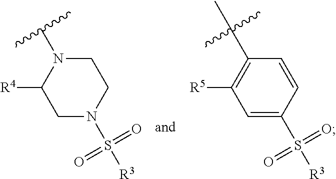

each R³ is independently selected from the group consisting of R⁶, C₁-C₆alkyl, amino N—C₁-C₃alkylamino, N, N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R⁶, and each of C₁-C₆alkyl and C₁-C₃alkoxyC₁-C₃alkyl is optionally substituted with or one or more independent occurrences of halogen; R⁴ is selected from the group consisting of C₁-C₆alkyl, C₁-C₆alkoxy, C₁-C₆haloalkyl, C₃-C₆cycloalkyl, and phenyl, wherein phenyl is optionally substituted with one or more occurrences of a substituent independently selected from the group consisting of fluoro, chloro, methyl, methoxy, dimethylamino, trifluoromethoxy, trifluoromethyl, and cyclopropyl; R⁵ is selected from the group consisting of halogen, C₁-C₆alkyl, C₁-C₆alkoxy, C₁-C₆haloalkyl, and C₃-C₆cycloalkyl; each R⁶ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁷; and each R⁷ is independently selected from the group consisting of halogen, amino, N—C₁-C₃alkylamino, N,N-diC₁-C₃alkylamino and C₁-C₃alkoxyC₁-C₃alkyl, C₁-C₃alkoxy, C₁-C₃haloalkoxy, C₃-C₆cycloalkyl, C₁-C₃haloalkyl, and C₁-C₃alkyl; in an amount sufficient to induce a Type I interferon response by the cell; and (ii) a STING agonist in an amount sufficient to increase the expression level of the at least one chemokine in the cell.

In an aspect, provided herein is a method of treating cancer in a patient in need thereof, comprising: (i) means for inducing a Type I interferon response in a cancerous cell in the patient; and (ii) administering a therapeutically effective amount of a STING agonist to the patient;

wherein the therapeutically effective amount of the STING agonist results in an increased expression level of at least one chemokine in the patient as compared to any increase in the expression level of the at least one chemokine resulting from administering the compound to the patient.

### BRIEF DESCRIPTION OF THE DRAWINGS

FIG. 1 shows that Compound 1 and Compound 2 increase mRNA expression of IFNB1, IRF1, IRF7 and IRF9 in A-498 and 786-O cells.

FIG. 2 shows the increase in phosphorylation of STAT1 with Compound 1 treatment in A-498 and 786-O cells, which is reversed by α-IFNAR2.

FIGS. 3A and 3B show the modulation of proteins within and downstream of the STING pathway by Compound 1 and 2 and the effects of siRNA knockdown of those proteins in A-498 and 786-O cells.

FIGS. 4A and 4B show that Compound 1 and Compound 2 increase mRNA expression of IFNB1, IRF7, CCL5 and CXCL10 which is reversed by siSTING and siCGAS in A-498 and 786-O cells.

FIGS. 5A and 5B show that Compound 1 and Compound 2 increase secretion of CCL5 and CXCL10 which is reversed by siSTING and siCGAS.

FIGS. 6A and 6B show that siRNA knockdown of VPS34 increases STING, pIRF3, STAT1 and pSTAT1 and knockdown of STING or CGAS reverses this effect in A-498 cells.

FIGS. 7A, 7B, 7C, and 7D show that siRNA knockdown of VPS34 increases mRNA expression of IFNB1, IRF7, CCL5 and CXCL10 and knockdown of STING or cGAS reverses this effect in A-498 cells.

FIGS. 8A, 8B, and 8C show that siRNA knockdown of VPS34 increases secretion of IFNβ, CCL5 and CXCL10 proteins and knockdown of STING or cGAS reverses this effect in A-498 cells.

FIGS. 9A and 9B show that Compound 1 and Compound 2 in combination with a STING agonist, ADU-S100, increases gene expression of IFNB1, CCL5 and CXCL10 in A-498 and 786-O cells, compared to single agent treatments.

FIGS. 10A and 10B show that Compound 1 and Compound 2 in combination with a STING agonist, ADU-S100, increases secretion of IFNβ, CCL5 and CXCL10 in A-498 and 786-O cells, compared to single agent treatments.

FIGS. 11A, 11B, and 11C shows that Compound 1 increases mRNA expression of IFNB1, CCL5 and CXCL10 in combination with the STING agonist ADU-S100 in Renca cells, which are reversed by knockdown of STING.

FIGS. 12A, 12B, and 12C show that Compound 1 and Compound 2 increases mRNA expression of IFNB1, CCL5 and CXCL10 in combination with the STING agonist ADU-S100 in B16-F10 cells.

FIGS. 13A, 13B, 13C, and 13D show that Compound 1 alone and in combination with a STING agonist, ADU-S100, triggers caspase-dependent apoptosis in Renca cells.

FIGS. 14A, 14B, and 14C show that Compound 1 and Compound 2 increase mRNA expression of IFNB1, CCL5 and CXCL10 which is reversed by siRNA knockdown of STING in Me30966 melanoma cells.

FIGS. 15A, 15B, 15C, and 15D show that Compound 1 and Compound 2 enhances the activity of endogenous STING agonist cGAMP by increasing mRNA expression and protein secretion of CCL5 and CXCL10 in CT26, 4T1 and YUMM cancer cell lines.

FIG. 16A shows that the combination of ADU-S100 with Compound 1 results in delayed degradation of activated STING in B16F10, CT26, DC2.4 and Renca cell lines. FIG. 16B shows that the combination of ADU-S100 with Compound 1 or Compound 2 results in increased interferon-sensitive response element (ISRE) and NFκB activity when compared to single agent alone in the THP-1 reporter cell line. FIG. 16C shows that the increased ISRE and NFκB activity in the THP-1 cell line is STING dependent.

FIG. 17A shows tumor growth curves from in vivo studies of Compound 1 and ADU-S100 in B16-F10 tumor-bearing mice. FIG. 17B shows Kaplan-Meier mice survival curves from in vivo studies of Compound 1 and ADU-S100 in B16-F10 tumor bearing mice.

### DETAILED DESCRIPTION

The definitions set forth in this application are intended to clarify terms used throughout this application. Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in stable compounds. It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

It has been found that VPS34 inhibitors can activate a type I IFN signaling in a through activation of the cGAS/STING pathway. In some embodiments, both pharmacological and siRNA-mediated VPS34 inhibition increases signaling of the cGAS-STING pathway. In some embodiments, an increase in signaling of the cGAS-STING pathway leads to expression and secretion of IFNβ, CCL5, and CXCL10. In some embodiments, a combination of VPS34 inhibitor and a STING agonist further induces cytokine release in both human and murine cancer cells. In other embodiments, the VPS34 inhibitor Compound 1 treatment sensitizes Renca and B16-F10 tumor-bearing mice to STING agonist treatment and significantly improved mice survival.

### Definitions

As used herein, “Compound 1” refers to a compound having the structure:

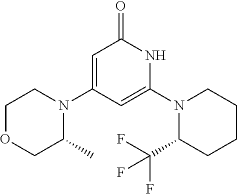 [SMILES: C[C@@H]1COCCN1c1cc(N2CCCC[C@@H]2C(F)(F)F)[nH]c(=O)c1]

and having the name 4-((R)-3-methylmorpholino)-6-((R)-2-(trifluoromethyl)piperidin-1-yl)pyridin-2(1H)-one.

As used herein, “Compound 2” refers to a compound having the structure:

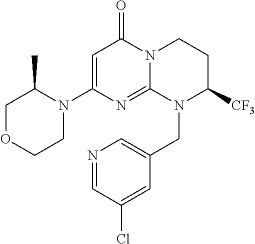 [SMILES: C[C@@H]1COCCN1c1cc(=O)n2c(n1)N(Cc1cncc(Cl)c1)[C@H](C(F)(F)F)CC2]

and having the name (S)-9-((5-chloropyridin-3-yl)methyl)-2-((R)-3-methylmorpholino)-8-(trifluoromethyl)-6,7,8,9-tetrahydro-4H-pyrimido[1,2-a]pyrimidin-4-one.

As used herein, the term “C₁-C₆alkyl” means both linear and branched chain saturated hydrocarbon groups with 1 to 6 carbon atoms. Examples of C₁-C₆alkyl groups include methyl, ethyl, n-propyl, isopropyl, n-butyl, iso-butyl, sec-butyl, t-butyl, n-pentyl, 4-methyl-butyl, n-hexyl, 2-ethyl-butyl groups. Among unbranched C₁-C₆alkyl groups, typical ones are methyl, ethyl, n-propyl, n-butyl, n-pentyl and n-hexyl groups. Among branched alkyl groups, there may be mentioned iso-propyl, iso-butyl, sec-butyl, t-butyl, 4-methyl-butyl and 2-ethyl-butyl groups.

As used herein, the term “C₁-C₃alkyl” means both linear and branched chain saturated hydrocarbon groups with 1 to 3 carbon atoms. Examples of C₁-C₃alkyl groups include methyl, ethyl, n-propyl and isopropyl groups.

As used herein, the term “C₁-C₆alkoxy” means the group O-alkyl, where “C₁-C₆alkyl” is used as described above. Examples of C₁-C₆alkoxy groups include, but are not limited to, methoxy, ethoxy, isopropoxy, n-propoxy, n-butoxy, n-hexoxy, 3-methyl-butoxy groups.

As used herein, the term “C₁-C₃alkoxy” means the group O-alkyl, where “C₁-C₃alkyl” is used as described above. Examples of C₁-C₃alkoxy groups include, but are not limited to, methoxy, ethoxy, isopropoxy and n-propoxy.

As used herein, the term “C₁-C₆haloalkyl” means both linear and branched chain saturated hydrocarbon groups, with 1 to 6 carbon atoms and with 1 to all hydrogens substituted by a halogen of different or same type. Examples of C₁-C₆haloalkyl groups include methyl substituted with 1 to 3 halogen atoms, ethyl substituted with 1 to 5 halogen atoms, n-propyl or iso-propyl substituted with 1 to 7 halogen atoms, n-butyl or iso-butyl substituted with 1 to 9 halogen atoms, and sec-butyl or t-butyl groups substituted with 1 to 9 halogen atoms.

As used herein, the term “C₁-C₃haloalkyl” means both linear and branched chain saturated hydrocarbon groups, with 1 to 3 carbon atoms and with 1 to all hydrogens substituted by a halogen of different or same type. Examples of C₁-C₃haloalkyl groups include methyl substituted with 1 to 3 halogen atoms, ethyl substituted with 1 to 5 halogen atoms, and n-propyl or iso-propyl substituted with 1 to 7 halogen atoms.

As used herein, the term “C₁-C₃haloalkoxy” means both linear and branched chain saturated alkoxy groups, with 1 to 3 carbon atoms and with 1 to all hydrogen atoms substituted by a halogen atom of different or same type. Examples of C₁-C₃haloalkoxy groups include methoxy substituted with 1 to 3 halogen atoms, ethoxy substituted with 1 to 5 halogen atoms, and n-propoxy or iso-propoxy substituted with 1 to 7 halogen atoms.

As used herein, the term “C₁-C₃fluorooalkyl” means both linear and branched chain saturated hydrocarbon groups, with 1 to 3 carbon atoms and with 1 to all hydrogen atoms substituted by a fluorine atom. Examples of C₁-C₃fluoroalkyl groups include methyl substituted with 1 to 3 fluorine atoms, ethyl substituted with 1 to 5 fluorine atoms, and n-propyl or iso-propyl substituted with 1 to 7 fluorine atoms.

As used herein, the term “C₁-C₃fluorooalkoxy” means both linear and branched chain saturated alkoxy groups, with 1 to 3 carbon atoms and with 1 to all hydrogen atoms substituted by a fluorine atom. Examples of C₁-C₃fluoroalkoxy groups include methoxy substituted with 1 to 3 fluorine atoms, ethoxy substituted with 1 to 5 fluorine atoms, and n-propoxy or iso-propoxy substituted with 1 to 7 fluorine atoms.

As used herein, the term “C₃-C₆cycloalkyl” means a cyclic saturated hydrocarbon group, with 3 to 6 carbon atoms. Examples of C₃-C₆cycloalkyl groups include cyclopropyl, cyclobutyl, cyclopentyl and cyclohexyl.

As used herein, the term “C₁-C₃alkoxyC₁-C₃alkyl” means both a both linear and branched chain saturated hydrocarbon group, with 1 to 3 carbon atoms, substituted with an alkoxy group with 1 to 3 carbon atoms. Examples of C₁-C₃alkoxyC₁-C₃alkyl groups are drawn below.

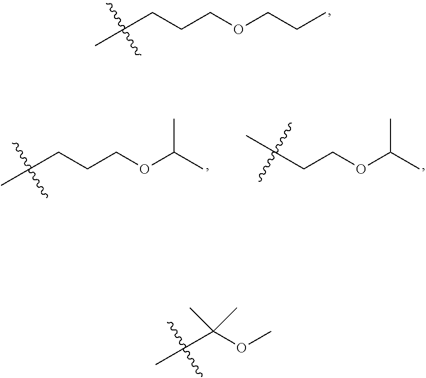

As used herein, the term “C₁-C₃cyanoalkyl” means both a linear and branched chain cyano (CN) derivative, with one to three carbon atoms including the carbon atom that is part of the cyano group. Examples of C₁-C₃cyanoalkyl groups are drawn below.

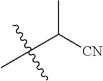 [SMILES: *C(C)C#N]

As used herein, the term “halogen” means fluorine, fluoro, chloro, bromine, bromo, iodine, or iodine.

As used herein, the term “aryl” means a monocyclic or bicyclic aromatic carbocyclic group. Examples of aryl groups include phenyl and naphthyl. A naphthyl group may be attached through the 1 or the 2 position. In a bicyclic aryl, one of the rings may be partially saturated. Examples of such groups include indanyl and tetrahydronaphthyl.

As used herein, the term “monocyclic aryl” means a monocyclic aromatic carbocyclic group. Examples of monocyclic aryl groups include phenyl.

As used herein, the term “heteroaryl” means a monocyclic or bicyclic aromatic group of carbon atoms wherein from one to three of the carbon atoms is/are replaced by one or more heteroatoms independently selected from nitrogen, oxygen or sulfur. In a bicyclic aryl, one of the rings may be partially saturated. Examples of such groups include indolinyl, dihydrobenzofuran and 1,3-benzodioxolyl.

As used herein, the term “monocyclic heteroaryl” means a monocyclic aromatic group of carbon atoms wherein from one to three of the carbon atoms is/are replaced by one or more heteroatoms independently selected from nitrogen, oxygen or sulfur.

Examples of monocyclic heteroaryl groups include, but are not limited to, furyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl.

Examples of bicyclic heteroaryl groups include, but are not limited to, quinoxalinyl, quinazolinyl, pyridopyrazinyl, benzoxazolyl, benzothiophenyl, benzimidazolyl, naphthyridinyl, quinolinyl, benzofuryl, indolyl, indazolyl, benzothiazolyl,

pyridopyrimidinyl, and isoquinolinyl.

As used herein, the term “heterocyclyl” means a cyclic group of carbon atoms wherein from one to three of the carbon atoms is/are replaced by one or more heteroatoms independently selected from nitrogen, oxygen and sulfur. Examples of heterocyclyl groups include, but are not limited to, tetrahydrofuryl, tetrahydropyranyl, pyrrolidinyl, piperidinyl, piperazinyl, morpholinyl and dioxanyl.

A “combination therapy” is a treatment that includes the administration of two or more therapeutic agents, e.g., a compound of Formula I and an antibiotic, a viral protease inhibitor, or an anti-viral nucleoside anti-metabolite, to a patient in need thereof.

“Disease,” “disorder,” and “condition” are used interchangeably herein.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The presently described compounds encompasses various stereoisomers of these compounds and mixtures thereof. Mixtures of enantiomers or diastereomers may be designated “(±)” in nomenclature, but the skilled artisan will recognize that a structure may denote a chiral center implicitly.

In the present specification, the term “therapeutically effective amount” means the amount of the subject compound that will elicit the biological or medical response of a tissue, system or animal, (e.g., mammal or human) that is being sought by the researcher, veterinarian, medical doctor or other clinician. The compounds described herein are administered in therapeutically effective amounts to treat a disorder.

“Treating” includes any effect, e.g., lessening, reducing, modulating, or eliminating, that results in the improvement of the condition, disease, disorder and the like.

The disclosure also embraces isotopically labeled compounds which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

### Compounds

In some embodiments, described herein is a compound of Formula I:

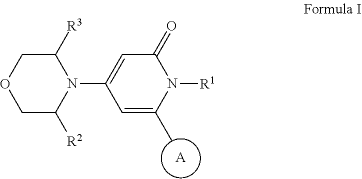

or a pharmaceutically acceptable salt, stereoisomer, or tautomer thereof, wherein: R¹, R², and R³ are independently selected from the group consisting of H, C₁-C₃haloalkyl, and C₁-C₃alkyl; A represents:

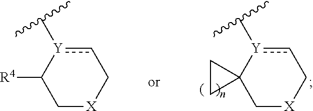

[symbol]

is a single bond or a double bond; X is selected from the group consisting of CH₂, S, SO, SO₂, NR⁵, NCOR⁵, NCOR⁹, NCOCH₂R⁹, O, and a bond; Y is selected from the group consisting of N, CH, and C, provided that, when Y is CH, [symbol] is a single bond; n is selected from 1, 2, 3 and 4; R⁴ is selected from the group consisting of H, halogen, COR⁶, C₁-C₆alkyl, C₁-C₃alkoxyC₁-C₃alkyl, C₁-C₆alkoxy, C₃-C₆cycloalkyl, C₃-C₆heterocyclyl, C₁-C₃cyanoalkyl, C₁-C₃haloalkyl, aryl, and heteroaryl, wherein said aryl and said heteroaryl are optionally substituted with one or more R⁷; R⁵ is selected from the group consisting of H, C₁-C₃fluoroalkyl, C₁-C₃alkyl, C₁-C₃alkoxyC₁-C₃alkyl, and C₃-C₆cycloalkyl; R⁶ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; each R⁷ is independently selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy and C₁-C₃alkoxy; R⁹ is selected from the group consisting of C₁-C₃alkyl, C₁-C₃alkoxy, C₃-C₆cycloalkyl, heterocyclyl, phenyl and a monocyclic heteroaryl, wherein said heterocyclyl, said phenyl and said monocyclic heteroaryl are optionally substituted with one or two R⁸; and each R⁸ is independently selected from the group consisting of halogen, C₁-C₃haloalkyl and C₁-C₃alkyl.

In some embodiments, R¹ is H. In some embodiments, R² is H. In some embodiments, R³ is C₁-C₃alkyl. In some embodiments, A is piperidinyl. In some embodiments, R⁴ is C₁-C₃haloalkyl.

In some embodiments, R⁴ is selected from the group consisting of hydrogen, halogen, COR⁶, C₁-C₆alkyl, C₁-C₃alkoxyC₁-C₃alkyl, C₁-C₆alkoxy, C₃-C₆cycloalkyl, C₁-C₃cyanoalkyl, C₁-C₃haloalkyl, aryl and heteroaryl, wherein said aryl and said heteroaryl are optionally substituted with one or more R⁷.

In some embodiments, Y is N. In some embodiments of this aspect, R¹ and re are independently selected from hydrogen and methyl. In some embodiments, R² is hydrogen. In some embodiments, R¹ is hydrogen. In some embodiments, R³ is methyl. In some embodiments, R³ is hydrogen. In some embodiments, R⁵ is C₁-C₃alkyl. In some embodiments, R⁶ is N—C₁-C₃alkylamino or N,N-diC₁-C₃alkylamino, such as N,N-diC₁-C₃alkylamino. In some embodiments, R⁶ is dimethylamino. In some embodiments, R⁷ is selected from the group consisting of halogen, C₁-C₃fluoroalkyl, C₁-C₃fluoroalkoxy, C₁-C₃alkoxy, C₁-C₃alkyl, C₃-C₆cycloalkyl and N,N-diC₁-C₃alkylamino. In some embodiments, R⁷ is selected from the group consisting of fluoro, chloro, trifluoromethyl, trifluoromethoxy, methoxy, methyl, ethyl, cyclopropyl and dimethylamino. In some embodiments, R⁹ is selected from the group consisting of C₁-C₃alkoxy, heterocyclyl, phenyl and a monocyclic heteroaryl, wherein said heterocyclyl, said phenyl and said nonocyclic heteroaryl are optionally substituted with one or two R⁸. In some embodiments, R⁹ is selected from the group consisting of heterocyclyl, phenyl and a monocyclic heteroaryl, wherein said heterocyclyl, said phenyl and said monocyclic heteroaryl are optionally substituted with one or two R⁸. In some embodiments, R⁹ is selected from the group consisting of tetrahydrofuryl, phenyl and pyridyl, each optionally substituted with one or two R⁸. In some embodiments, R⁸ is halogen. In some embodiments, said monocyclic heteroaryl in R⁴ is selected from the group consisting of pyridyl, furyl, isoxasolyl, pyrazolyl and thiazolyl, each optionally substituted with one or more R⁷.

In some embodiments R⁴ selected from the group consisting of:

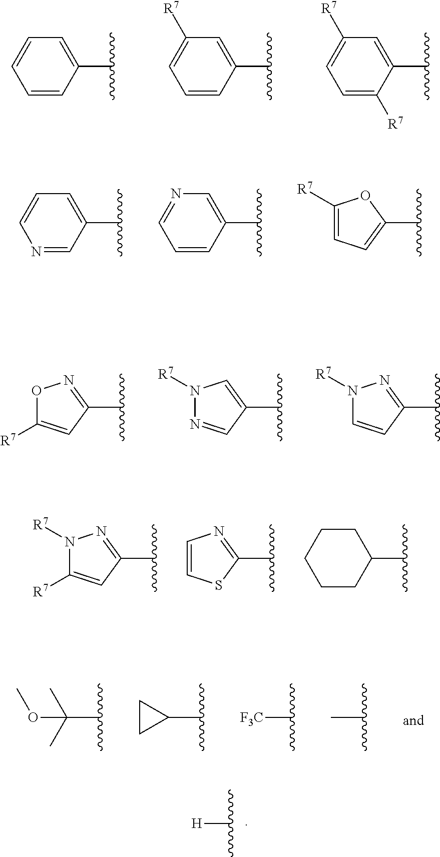

In some embodiments R⁷ is selected from the group consisting of fluoro, chloro, C₁-C₃alkoxy, C₁-C₃fluoroalkoxy, C₁-C₃fluoroalkyl, C₃-C₆cycloalkyl, N,N-diC₁-C₃alkylamino. In some embodiments, R⁷ is selected from the group consisting of fluoro, chloro, methyl, ethyl, methoxy, trifluoromethoxy, trifluoromethyl, cyclopropyl and N,N-dimethylamino. In some embodiments, X represents a bond. In some embodiments, R⁴ is selected from the group consisting of:

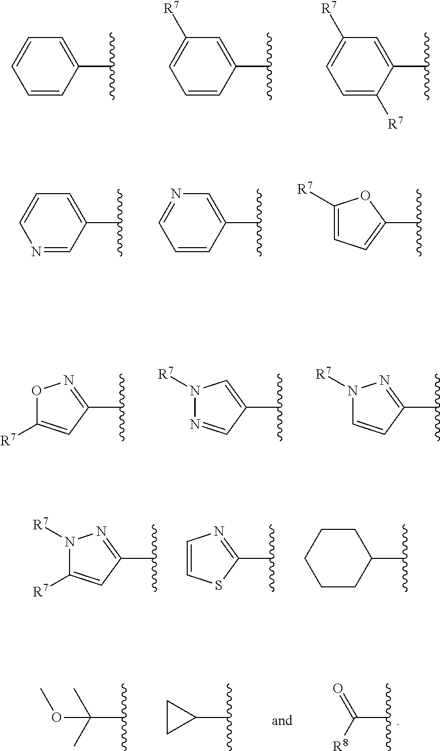

In some embodiments, A is selected from the group consisting of:

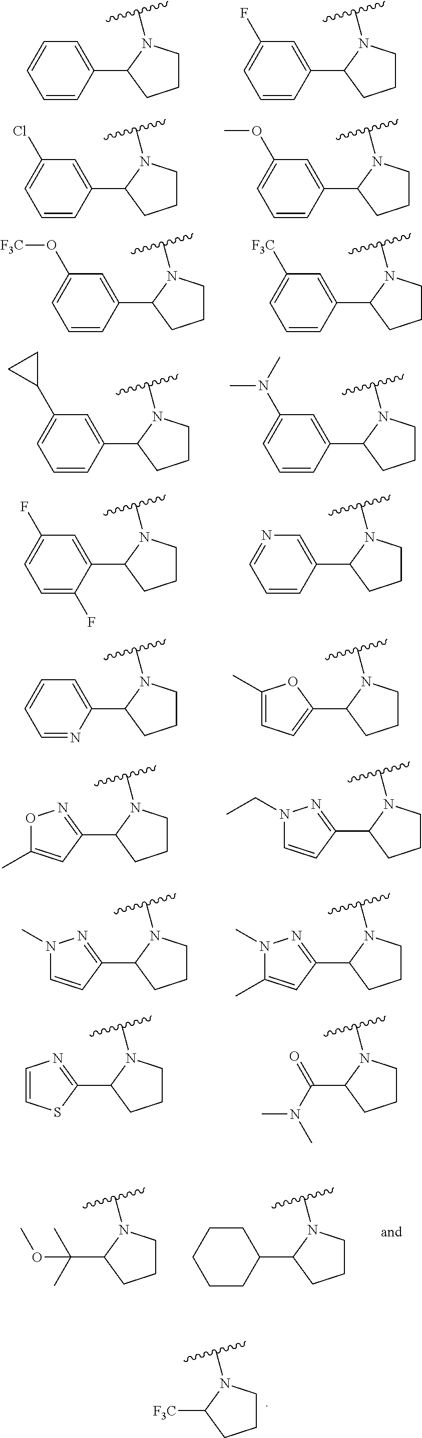

In some embodiments, X is selected from the group consisting of CH₂, SO, SO₂, NR⁵, NCOR⁵, NCOR⁹, NCOCH2R⁹ and O; and R⁵ is C₁-C₃alkyl.

In some embodiments, R⁴ is selected from the group consisting of hydrogen, C₁-C₆alkyl, C₃-C₆cycloalkyl, C₁-C₃haloalkyl and phenyl, wherein phenyl is optionally substituted with one, or more R⁷.

In some embodiments, A is selected from the group consisting of:

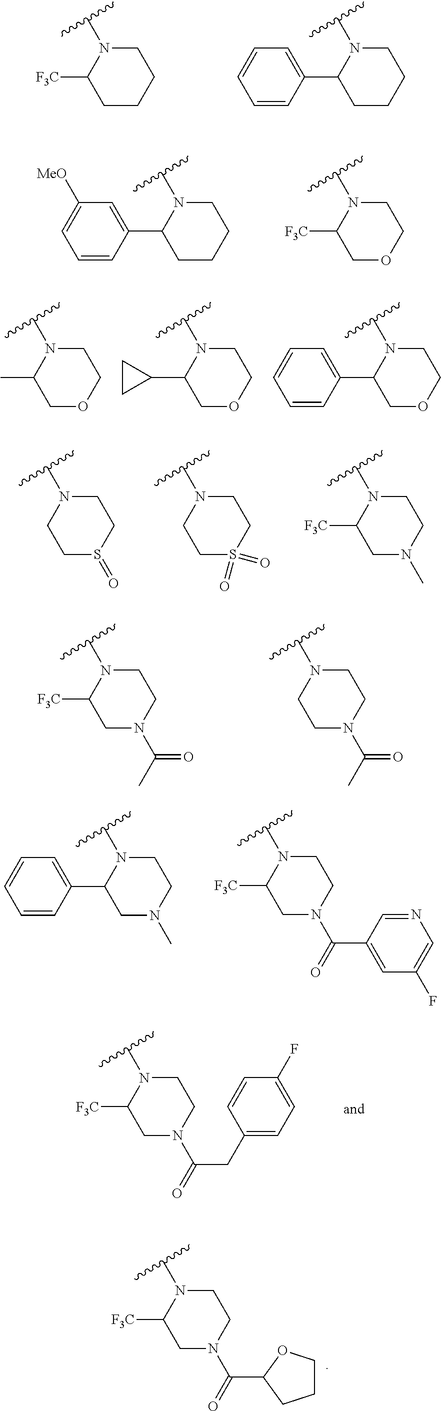

In some embodiments, X is selected from the group consisting of CH₂, SO, SO₂, NR⁵, NCOR⁵, NCOR⁹, NCOCH₂R⁹, O, and a bond; R⁴ is selected from the group consisting of hydrogen, COR⁶, C₁-C₃alkyl, methoxyC₁-C₃alkyl, C₃-C₆cycloalkyl, C₁-C₃fluoroalkyl, phenyl and a monocyclic heteroaryl, wherein said phenyl and said monocyclic heteroaryl are optionally substituted with one or two R⁷; R³ is C₁-C₃alkyl; R⁶ is N,N-diC₁-C₃alkylamino; and R⁷ is selected from fluoro, chloro, C₁-C₃alkyl, C₁-C₃alkoxy, C₁-C₃fluoroalkoxy, C₁-C₃fluoroalkyl, C₃-C₆cycloalkyl and N,N-diC₁-C₃alkylamino.

In some embodiments, Y is CH or C; X is O; and R⁴ is hydrogen. In some embodiments, R¹ and R² are hydrogen; R³ is methyl; X is selected from the group consisting of CH₂, O, NCOR⁵, NCOR⁹, NCOCH₂R⁹, and a bond; Y is N; R⁴ is selected from the group consisting of hydrogen, phenyl, and trifluoromethyl; R³ is methyl; R⁷ is methoxy; R⁹ is selected from the group consisting of pyridyl, phenyl; and R⁸ is fluoro. In some embodiments, R¹ and R² are hydrogen; R³ is methyl; X is selected from the group consisting of CH₂, O, NCOR⁵, NCOCH2R⁹, and a bond; Y is N; R⁴ is phenyl or trifluoromethyl, said phenyl being substituted with one or more R⁷; R⁵ is methyl; R⁷ is methoxy or halogen, such as methoxy or chloro; R⁹ is phenyl, said phenyl being optionally substituted by one or more R⁸; and R⁸ is halogen, such as fluoro. In some embodiments, R⁴ is selected from trifluoromethyl and phenyl, said phenyl being meta-substituted with methoxy or chloro. In some embodiments, R⁷ is methoxy or chloro; and R⁸ is fluoro.

In some embodiments, A represents

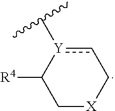

In some embodiments, A represents

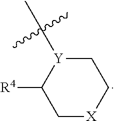

In some embodiments, R¹ and R² are hydrogen; R³ is methyl; X is selected from NCOR⁹ and NCOCH2R⁹; R⁴ is selected from trifluoromethyl and phenyl, said phenyl being optionally substituted with methoxy or chloro; R⁹ is selected from the group consisting of C₁-C₃alkyl, C₁-C₃alkoxy, C₃-C₆cycloalkyl, heterocyclyl, phenyl and a monocyclic heteroaryl, wherein said heterocyclyl, said phenyl and said monocyclic heteroaryl are optionally substituted with one or two R⁸; and R⁸ is selected from the group consisting of fluoro, chloro, C₁-C₃haloalkyl and C₁-C₃alkyl.

In some embodiments, R¹ and R² are hydrogen; R³ is methyl; X represents NCOR⁹ or NCOCH₂R⁹; R⁴ is trifluoromethyl; R⁹ is selected from the group consisting of C₁-C₃alkyl, C₁-C₃alkoxy, C₃-C₆cycloalkyl, oxazolyl, tetrahydrofuryl, morpholinyl, pyridyl and phenyl, wherein said oxazolyl, said tetrahydrofuryl, said morpholinyl, said pyridyl and said phenyl are optionally substituted with one or two R⁸; and R⁸ is selected from the group consisting of fluoro, chloro, C₁-C₃haloalkyl and C₁-C₃alkyl.

In some embodiments, X represents Cl₂, SO, SO₂, NR⁵, NCOR⁵, NCOR⁹, NCOCH2R⁹ or O; R¹ and R³ are independently selected from hydrogen and methyl; R² is hydrogen; R⁴ is selected from the group consisting of:

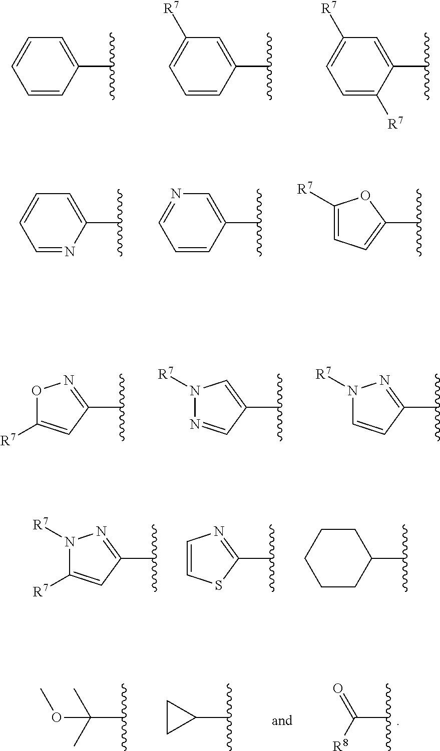

F₃C—R⁵ is C₁-C₃alkyl; R⁷ is selected from the group consisting of fluoro chloro, methyl, ethyl, methoxy, trifluoromethoxy, trifluoromethyl, cyclopropyl and N,N-dimethylamino; R⁹ is selected from the group consisting of tetrahydrofuryl, phenyl and pyridyl, each optionally substituted with one or two R⁸; and R⁸ is halogen.

In some embodiments, R¹, R² and R³ are independently selected from hydrogen and methyl; and A is selected from the group consisting of:

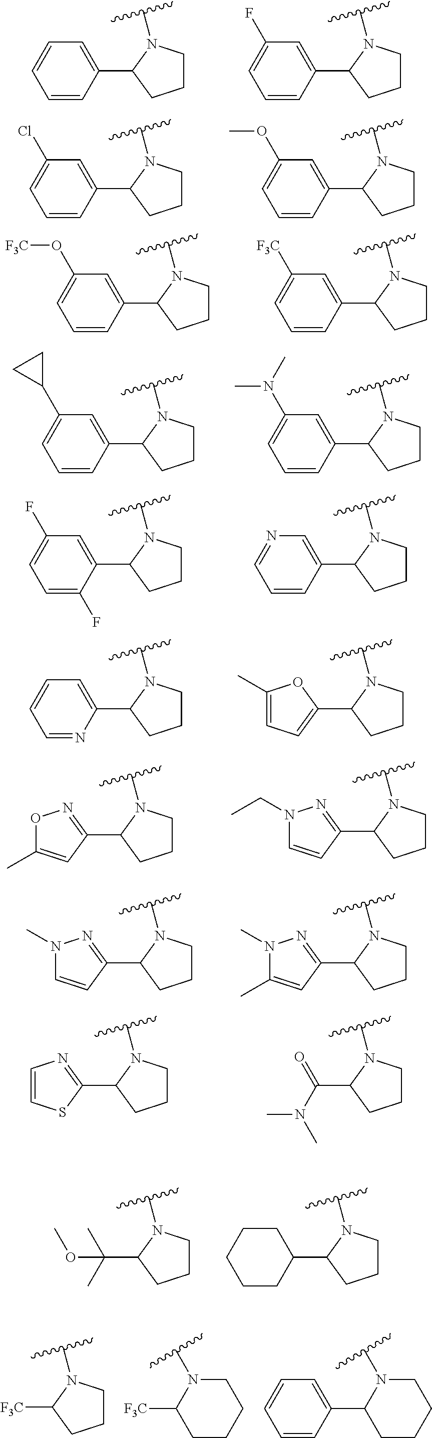

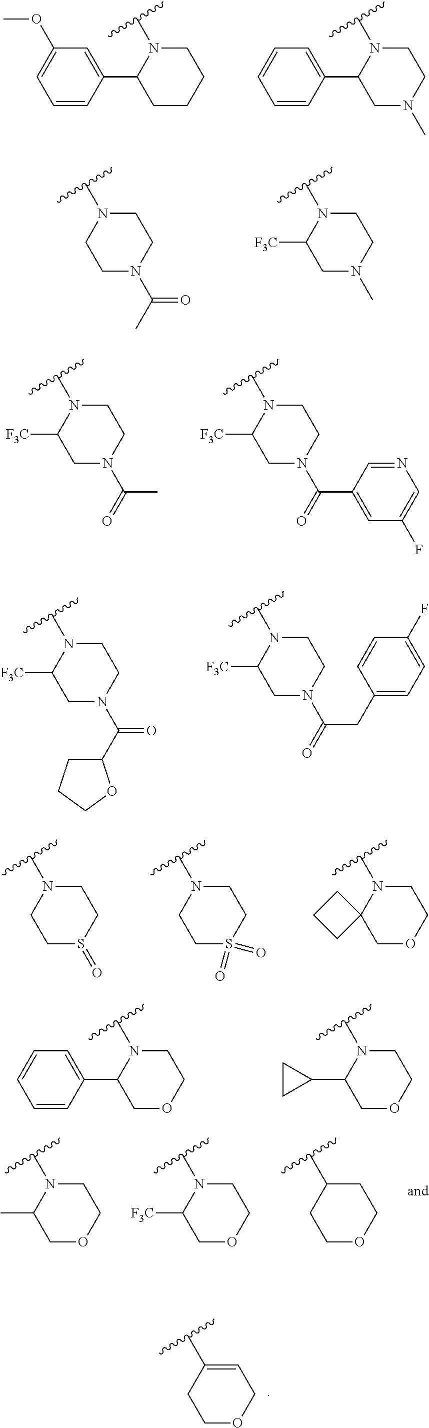

In some embodiments, the compound is selected from the group consisting of: 4-morpholino-6-(2-phenylpyrrolidin-1-yl)-1H-pyridin-2-one; 1-methyl-4-morpholino-6-(2-phenylpyrrolidin-1-yl)pyridin-2-one; 4-morpholino-6-[(2S)-2-phenylpyrrolidin-1-yl]-1H-pyridin-2-one; 4-morpholino-6-[(2R)-2-phenylpyrrolidin-1-yl]-1H-pyridin-2-one; 6-(3,6-dihydro-2H-pyran-4-yl)-4-(3-methylmorpholin-4-yl)-1H-pyridin-2-one; 4-(3-methylmorpholin-4-yl)-6-tetrahydropyran-4-yl-1H-pyridin-2-one; 6-[2-(3-methoxyphenyl)pyrrolidin-1-yl]-4-(3-methylmorpholin-4-yl)-1H-pyridin-2-one; 4-(3-methylmorpholin-4-yl)-6-[2-(3-pyridyl)pyrrolidin-1-yl]-1H-pyridin-2-one; 4-(3-methylmorpholin-4-yl)-6-(2-phenylpyrrolidin-1-yl)-1H-pyridin-2-one; N,N-dimethyl-1-[4-[(3R)-3-methylmorpholin-4-yl]-6-oxo-1H-pyridin-2-yl]pyrrolidine-2-carboxamide; 6-[2-(1-methoxy-1-methyl-ethyl)pyrrolidin-1-yl]-4-[(3R)-3-methylmorpholin-4-yl]-1H-pyridin-2-one; 6-(2-cyclohexylpyrrolidin-1-yl)-4-[(3R)-3-methylmorpholin-4-yl]-1H-pyridin-2-one; 6-[2-(3-fluorophenyl)pyrrolidin-1-yl]-4-[(3R)-3-methylmorpholin-4-yl]-1H-pyridin-2-one; 6-[2-(2,5-difluorophenyl)pyrrolidin-1-yl]-4-[(3R)-3-methylmorpholin-4-yl]-1H-pyridin-2-one; 4-[(3R)-3-methylmorpholin-4-yl]-6-[2-[3-(trifluoromethoxy)phenyl]pyrrolidin-1-yl]-1H-pyridin-2-one; 4-[(3R)-3-methylmorpholin-4-yl]-6-[2-[3-(trifluoromethyl)phenyl]pyrrolidin-1-yl]-1H-pyridin-2-one; 6-[2-(3-methoxyphenyl)pyrrolidin-1-yl]-4-[(3R)-3-methylmorpholin-4-yl]-1H-pyridin-2-one; 4-[(3R)-3-methylmorpholin-4-yl]-6-(2-phenylpyrrolidin-1-yl)-1H-pyridin-2-one; 4-[(3R)-3-methylmorpholin-4-yl]-6-[2-(1-methylpyrazol-4-yl)pyrrolidin-1-yl]-1H-pyridin-2-one; 6-[2-(1,5-dimethylpyrazol-3-yl)pyrrolidin-1-yl]-4-[(3R)-3-methylmorpholin-4-yl]-1H-pyridin-2-one; 6-[2-(1-ethylpyrazol-3-yl)pyrrolidin-1-yl]-4-[(3R)-3-methylmorpholin-4-yl]-1H-pyridin-2-one; 6-[2-(5-methyl-2-furyl)pyrrolidin-1-yl]-4-[(3R)-3-methylmorpholin-4-yl]-1H-pyridin-2-one; 6-[2-[3-(dimethylamino)phenyl]pyrrolidin-1-yl]-4-[(3R)-3-methylmorpholin-4-yl]-1H-pyridin-2-one; 4-[(3R)-3-methylmorpholin-4-yl]-6-(3-methylmorpholin-4-yl)-1H-pyridin-2-one; 4-[(3R)-3-methylmorpholin-4-yl]-6-[2-(trifluoromethyl)-1-piperidyl]-1H-pyridin-2-one; 4-[(3R)-3-methylmorpholin-4-yl]-6-(3-phenylmorpholin-4-yl)-1H-pyridin-2-one; 4-[(3R)-3-methylmorpholin-4-yl]-6-(1-oxo-1,4-thiazinan-4-yl)-1H-pyridin-2-one; 6-(1,1-dioxo-1,4-thiazinan-4-yl)-4-[(3R)-3-methylmorpholin-4-yl]-1H-pyridin-2-one; 6-(4-acetylpiperazin-1-yl)-4-[(3R)-3-methylmorpholin-4-yl]-1H-pyridin-2-one; 4-[(3R)-3-methylmorpholin-4-yl]-6-[(2R)-2-phenyl-1-piperidyl]-1H-pyridin-2-one; 4-[(3R)-3-methylmorpholin-4-yl]-6-(4-methyl-2-phenyl-piperazin-1-yl)-1H-pyridin-2-one; 4-[(3R)-3-methylmorpholin-4-yl]-6-[3-(trifluoromethyl)morpholin-4-yl]-1H-pyridin-2-one; 6-(3-cyclopropylmorpholin-4-yl)-4-[(3R)-3-methylmorpholin-4-yl]-1H-pyridin-2-one; 4-[(3R)-3-methylmorpholin-4-yl]-6-[(2S)-2-(trifluoromethyl)pyrrolidin-1-yl]-1H-pyridin-2-one; 4-[(3R)-3-methylmorpholin-4-yl]-6-[(2R)-2-(trifluoromethyl)pyrrolidin-1-yl]-1H-pyridin-2-one; 6-[2-(3-chlorophenyl)pyrrolidin-1-yl]-4-[(3R)-3-methylmorpholin-4-yl]-1H-pyridin-2-one; 6-[2-(3-cyclopropylphenyl)pyrrolidin-1-yl]-4-[(3R)-3-methylmorpholin-4-yl]-1H-pyridin-2-one; 4-[(3R)-3-methylmorpholin-4-yl]-6-[2-(2-pyridyl)pyrrolidin-1-yl]-1H-pyridin-2-one; 4-[(3R)-3-methylmorpholin-4-yl]-6-(2-thiazol-2-ylpyrrolidin-1-yl)-1H-pyridin-2-one; 6-[2-(5-methylisoxazol-3-yl)pyrrolidin-1-yl]-4-[(3R)-3-methylmorpholin-4-yl]-1H-pyridin-2-one; 1-methyl-4-[(3R)-3-methylmorpholin-4-yl]-6-[(2R)-2-(trifluoromethyl)-1-piperidyl]pyridin-2-one; 4-[(3R)-3-methylmorpholin-4-yl]-6-(8-oxa-5-azaspiro[3.5]nonan-5-yl)-1H-pyridin-2-one; 6-[2-(3-methoxyphenyl)-1-piperidyl]-4-[(3R)-3-methylmorpholin-4-yl]-1H-pyridin-2-one; 6-[4-acetyl-2-(trifluoromethyl)piperazin-1-yl]-4-[(3R)-3-methylmorpholin-4-yl]-1H pyridin-2-one; 6-[4-(5-fluoropyridine-3-carbonyl)-2-(trifluoromethyl)piperazin-1-yl]-4-[(3R)-3-methylmorpholin-4-yl]-1H-pyridin-2-one; 6-[4-[2-(4-fluorophenyl)acetyl]-2-(trifluoromethyl)piperazin-1-yl]-4-[(3R)-3-methylmorpholin-4-yl]-1H-pyridin-2-one; 4-[(3R)-3-methylmorpholin-4-yl]-6-[4-(tetrahydrofuran-2-carbonyl)-2-(trifluoromethyl)piperazin-1-yl]-1H-pyridin-2-one; 4-[(3R)-3-methylmorpholin-4-yl]-6-[4-methyl-2-(trifluoromethyl)piperazin-1-yl]-1H-pyridin-2-one; and pharmaceutically acceptable salts, tautomers, and stereoisomers thereof.

In some embodiments, the compound is selected from the group consisting of:

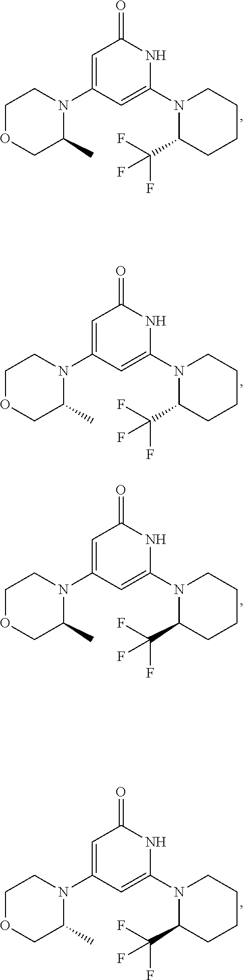

and pharmaceutically acceptable salts, stereoisomers, and tautomers thereof.

In some embodiments, the compound is selected from the group consisting of:

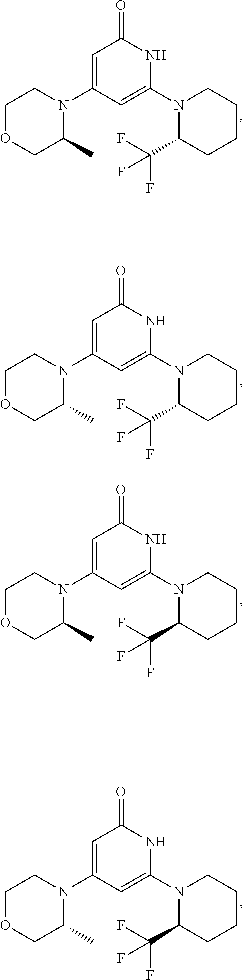

and pharmaceutically acceptable salts thereof.

In some embodiments, the compound is selected from the group consisting of:

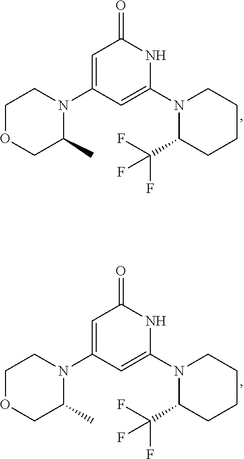 [SMILES: C[C@H]1COCCN1c1cc(N2CCCC[C@@H]2C(F)(F)F)[nH]c(=O)c1 ; C[C@@H]1COCCN1c1cc(N2CCCC[C@@H]2C(F)(F)F)[nH]c(=O)c1]

and pharmaceutically acceptable salts thereof.

In some embodiments, the compound is selected from the group consisting of:

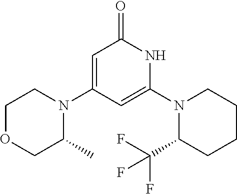 [SMILES: C[C@@H]1COCCN1c1cc(N2CCCC[C@@H]2C(F)(F)F)[nH]c(=O)c1]

and pharmaceutically acceptable salts thereof.

In another embodiment, described herein is a compound of Formula II:

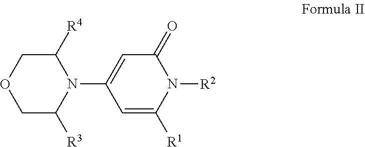

or a pharmaceutically acceptable salt, stereoisomer, or tautomer thereof, wherein: R¹ is selected from the group consisting of aryl and heteroaryl, wherein said aryl and said heteroaryl being mono- or bicyclic and each of aryl and heteroaryl is optionally substituted with one or more independent occurrences of a substituent selected from the group consisting of R⁵, R⁶, R⁷ and R⁸; each of R², R³, R⁴ is independently selected from the group consisting of H, C₁-C₃haloalkyl, and C₁-C₃alkyl; each of R⁵, R⁶, R⁷, and R⁸ is independently selected from the group consisting of halogen, C₁-C₆alkyl, C₁-C₆alkoxy, C₁-C₆haloalkyl, amino, —NHSO₂R⁹, hydroxy, phenyl, and a monocyclic heteroaryl; and R⁹ is selected from C₁-C₃haloalkyl and C₁-C₃alkyl.

In some embodiments, R¹ is aryl. In some embodiments, R¹ is phenyl. In some embodiments, R¹ is phenyl substituted with one occurrence of C₁-C₃haloalkyl. In some embodiments, R¹ is phenyl substituted with one occurrence of trifluoromethyl.

In some embodiments, R³ is H. In some embodiments, R⁴ is C₁-C₃alkyl. In some embodiments, R⁴ is —CH₃.

In some embodiments, R⁴ is C₁-C₃alkyl.

In some embodiments, R² is selected from hydrogen and methyl.

In some embodiments, R³ is hydrogen.

In some embodiments, R⁴ is methyl.

In some embodiments, R² is hydrogen.

In some embodiments, R¹ is selected from the group consisting of phenyl, furyl, thienyl, pyridyl, pyrimidinyl, naphtyl, quinolinyl, indazolyl, indolyl, 4-azaindolyl, benzoxazolyl, benzimidazolyl, benzothiophenyl, each optionally substituted with one or more of R⁵, R⁶, R⁷ and R⁸.

In some embodiments, R⁵, R⁶, R⁷ and R⁸ are independently selected from the group consisting of chloro, fluoro, C₁-C₃alkyl. C₁-C₃fluoroalkyl, phenyl, amino, —NHSO2CH₃, hydroxy, imidazolyl and pyrazolyl.

In some embodiments, R¹ is selected from the group consisting of phenyl, furyl, thienyl, pyridyl, pyrimidinyl, naphtyl, quinolinyl, indazolyl, indolyl, 4-azaindolyl, benzoxazolyl, benzimidazolyl, benzothiophenyl, each optionally substituted with one or more of R⁵, R⁶, R⁷ and R⁸; and R⁵, R⁶, R⁷ and R⁸ are independently selected from the group consisting of halogen, C₁-C₃alkyl, C₁-C₃haloalkyl, phenyl, amino, —NHSO₂CH₃, hydroxy, imidazolyl and pyrazolyl.

In some embodiments, R¹ is selected from the group consisting of phenyl, furyl, thienyl, pyridyl, pyrimidinyl and quinolinyl.

In some embodiments, R⁵ and R⁶ are independently selected from the group consisting of chloro, fluoro, trifluoromethyl, methyl, phenyl, —NHSO₂CH₃ and pyrazolyl.

In some embodiments, R¹ is selected from the group consisting of phenyl, furyl, thienyl, pyridyl, pyrimidinyl and quinolinyl.

In some embodiments, R¹ is selected from the group consisting of phenyl, furyl, thienyl, pyridyl, pyrimidinyl and quinolinyl, each optionally substituted with R⁵ and/or R⁶; and R⁵ and R⁶ are independently selected from the group consisting of chloro, fluoro, trifluoromethyl, methyl, phenyl, —NHSO₂CH₃ and pyrazolyl.

In some embodiments R¹ is selected from the group consisting of:

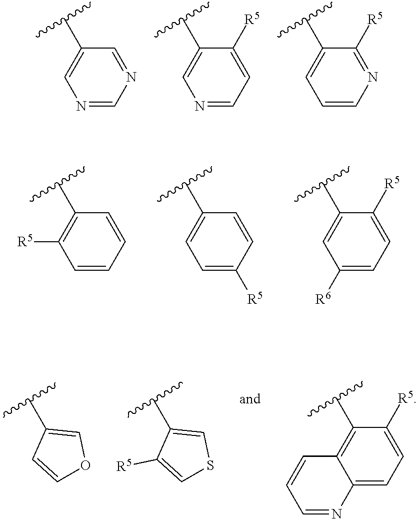

wherein R⁵ and R⁶ are independently selected from the group consisting of halogen, C₁-C₃alkyl, C₁-C₃haloalkyl, phenyl, pyrazolyl, and —NHSO₂CH₃.

In some embodiments, R¹ is a monocyclic aryl or heteroaryl.

In some embodiments, R¹ is selected from phenyl and pyridyl.

In some embodiments, R⁵ and R⁶ are independently selected from the group consisting of chloro, fluoro and trifluoromethyl, such as chloro and trifluoromethyl.

In some embodiments, R¹ is selected from phenyl and pyridyl, each optionally substituted with R⁵ and/or R⁶; and R⁵ and R⁶ are independently selected from the group consisting of chloro, fluoro and trifluoromethyl.

In some embodiments, R¹ is selected from the group consisting of:

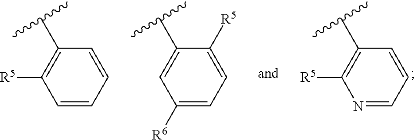

R⁴ is C₁-C₃alkyl; and R⁵ and R⁶ are independently selected from the group consisting of chloro, fluoro, and trifluoromethyl.

In some embodiments, R¹ is selected from the group consisting of:

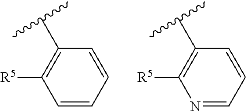

In some embodiments, R¹ is selected from:

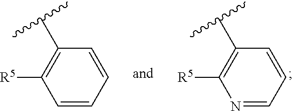

R² is hydrogen; and R⁵ is selected from chloro and trifluoromethyl.

In some embodiments, R¹ is selected from the group consisting of:

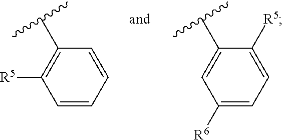

R² is hydrogen; R⁴ is C₁-C₃alkyl; and R⁵ and R⁰ are independently selected from chloro, fluoro and trifluoromethyl.

In some embodiments, R¹ is selected from the group consisting of:

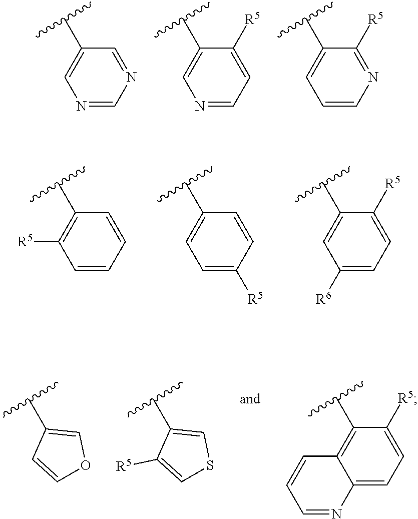

R² is hydrogen or methyl; R³ is hydrogen; R⁴ is methyl; R⁵ and R⁶ selected from the group consisting of chloro, fluoro, trifluoromethyl, methyl, phenyl pyrazolyl and —NHSO₂CH₃; and pharmaceutically acceptable salts and stereoisomers thereof.

In some embodiments, the compound is selected from the group consisting of:

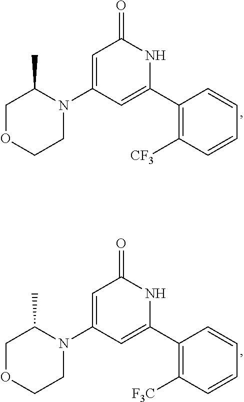 [SMILES: C[C@@H]1COCCN1c1cc(-c2ccccc2C(F)(F)F)[nH]c(=O)c1 ; C[C@H]1COCCN1c1cc(-c2ccccc2C(F)(F)F)[nH]c(=O)c1]

and pharmaceutically acceptable salts, stereoisomers, and tautomers thereof.

In some embodiments, the compound is selected from the group consisting of:

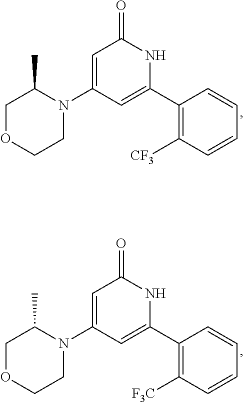 [SMILES: C[C@@H]1COCCN1c1cc(-c2ccccc2C(F)(F)F)[nH]c(=O)c1 ; C[C@H]1COCCN1c1cc(-c2ccccc2C(F)(F)F)[nH]c(=O)c1]

and pharmaceutically acceptable salts thereof.

In some embodiments, the compound is selected from the group consisting of: 6-(2-chlorophenyl)-4-morpholino-1H-pyridin-2-one; 6-(2-chlorophenyl)-1-methyl-4-morpholino-pyridin-2-one; 6-(2-chlorophenyl)-4-(3-methylmorpholin-4-yl)-1H-pyridin-2-one; 6-(2-chlorophenyl)-1-methyl-4-(3-methylmorpholin-4-yl)pyridin-2-one; 4-(3-methylmorpholin-4-yl)-6-(4-methyl-3-pyridyl)-1H-pyridin-2-one; 4-(3-methylmorpholin-4-yl)-6-pyrimidin-5-yl-1H-pyridin-2-one; 4-(3-methylmorpholin-4-yl)-6-(2-phenylphenyl)-1H-pyridin-2-one; 6-(2-chloro-5-fluoro-phenyl)-4-[(3R)-3-methylmorpholin-4-yl]-1H-pyridin-2-one; 4-[(3R)-3-methylmorpholin-4-yl]-6-(o-tolyl)-1H-pyridin-2-one; 4-[(3R)-3-methylmorpholin-4-yl]-6-[2-(trifluoromethyl)-3-pyridyl]-1H-pyridin-2-one; 6-(2-chlorophenyl)-4-[(3R)-3-methylmorpholin-4-yl]-1H-pyridin-2-one; 4-[(3R)-3-methylmorpholin-4-yl]-6-[2-(trifluoromethyl)phenyl]-1H-pyridin-2-one; 6-(3-furyl)-4-[(3R)-3-methylmorpholin-4-yl]-1H-pyridin-2-one; 4-[(3R)-3-methylmorpholin-4-yl]-6-(4-methyl-3-thienyl)-1H-pyridin-2-one; N-[2-[4-[(3R)-3-methylmorpholin-4-yl]-6-oxo-1H-pyridin-2-yl]phenyl]methanesulfonamide; 4-[(3R)-3-methylmorpholin-4-yl]-6-(4-(methylsulfonyl)-2-(trifluoromethyl)phenyl)-1H-pyridin-2-one; 4-[(3R)-3-methylmorpholin-4-yl]-6-(6-methyl-5-quinolyl)-1H-pyridin-2-one; 4-[(3R)-3-methylmorpholin-4-yl]-6-[4-(1H-pyrazol-5-yl)phenyl]-1H-pyridin-2-one; N,N-dimethyl-[4[4-[(3R)-3-methylmorpholin-4-yl]-6-oxo-1H-pyridin-2-yl]-3-(trifluoromethyl)]benzenesulfonamide; and pharmaceutically acceptable salts, tautomers, and stereoisomers thereof.

In some embodiments, described herein is a compound of Formula III:

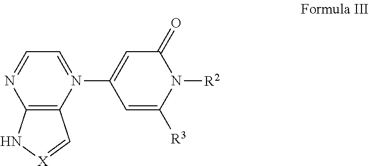

or a pharmaceutically acceptable salt, stereoisomer, or tautomer thereof, wherein: X is selected from N and CR¹; R¹ is selected from the group consisting of H, C₁-C₃alkyl, C₁-C₃haloalkyl, C₁-C₃alkoxyC₁-C₃alkyl, C₃-C₆cycloalkyl, cyano, phenyl, and monocyclic heteroaryl, wherein each of phenyl and monocyclic heteroaryl is optionally substituted with one or more substituents independently selected from the group consisting of halo, N—C₁-C₃alkylamino, N,N-diC₁-C₃alkylamino, C₃-C₆cycloalkyl, C₁-C₃alkoxyC₁-C₃alkyl, C₁-C₃haloalkyl, C₁-C₃haloalkoxy, C₁-C₃alkoxy, and C₁-C₃alkyl; R² is selected from the group consisting of H, C₁-C₃haloalkyl, and C₁-C₃alkyl; R³ is selected from the group consisting of A, phenyl, and monocyclic heteroaryl, wherein each of phenyl and monocyclic heteroaryl is optionally substituted with one or more occurrences of R⁴; each R⁴ is independently selected from the group consisting of COR⁵, halogen, C₁-C₆alkyl, C₁-C₆alkoxy, C₁-C₆haloalkoxy, amino N—C₁-C₃alkylamino, N,N-diC₁-C₃alkylamino, 1-pyrrolidinyl, 1-piperidinyl, 1-azetidinyl, NHSO₂R⁶, SO₂R⁷, hydroxy, C₃-C₆cycloalkyl, C₁-C₃alkoxyC₁-C₃alkyl, C₁-C₃cyanoalkyl and C₁-C₆haloalkyl; R⁵ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; R⁶ is selected from C₁-C₃haloalkyl and C₁-C₃alkyl; each R⁷ is independently selected from the group consisting of R⁸, C₁-C₆alkyl, N—C₁-C₃alkylamino, N,N-diC₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen; each R⁸ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁹; each R⁹ is independently selected from the group consisting of halo, N—C₁-C₃alkylamino, N,N-diC₁-C₃alkylamino, C₁-C₃alkoxyC₁-C₃alkyl, amino, C₁-C₃haloalkyl, C₁-C₃alkoxy, C₁-C₃haloalkoxy, C₃-C₆cycloalkyl and C₁-C₃alkyl; A is

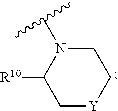

R¹⁰ is selected from the group consisting of H, halogen, COR¹¹, C₁-C₆alkyl, C₁-C₃alkoxyC₁-C₃alkyl, C₁-C₆alkoxy, C₃-C₆cycloalkyl, C₁-C₃cyanoalkyl, C₁-C₃haloalkyl, phenyl, and heteroaryl, wherein each of phenyl and heteroaryl is optionally substituted with one or more occurrences of R¹², and provided that when R¹⁰ is phenyl or heteroaryl, then X is N or CH; each R¹¹ is independently selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹³, NCOR⁷, NCOOR¹⁴, NSO₂R⁷, NCOCH₂R⁷, O, and a bond; R¹² is selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy, and C₁-C₃alkoxy; R¹³ is selected from H, C₁-C₃haloalkyl, C₁-C₃alkoxyC₁-C₃alkyl, C₁-C₃alkyl, C₃-C₆cycloalkyl; and R¹⁴ is selected from R⁸, C₁-C₆alkyl, C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen.

In some embodiments, R² is H. In some embodiments, R³ is A. In some embodiments, Y is CH₂. In some embodiments, X is N. In some embodiments, R¹⁰ is C₁-C₃haloalkyl.

In some embodiments, R² is hydrogen or C₁-C₃alkyl.

In some embodiments, R¹ is selected from the group consisting of H, C₁-C₃alkyl, C₁-C₃haloalkyl, C₃-C₆cycloalkyl, cyano, phenyl, heteroaryl, wherein said phenyl and said heteroaryl are optionally and independently substituted with one or more substituents selected from the group consisting of C₁-C₃haloalkyl, halo, C₃-C₆cycloalkyl and C₁-C₃alkyl.

In some embodiments, R² is hydrogen.

In some embodiments, said heteroaryl in R¹ is selected from the group consisting of pyridyl, oxazolyl, thienyl, and pyrimidinyl, each optionally and independently substituted with one or more substituents selected from halo, cyclopropyl, C₁-C₃fluoroalkyl and C₁-C₃alkyl.

In some embodiments, R¹ is selected from the group consisting of:

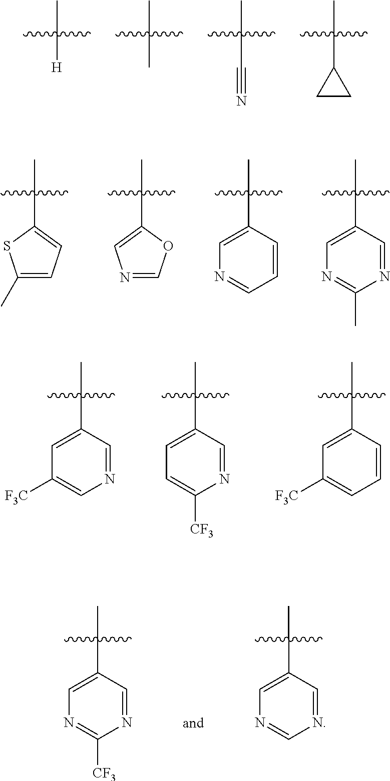

In some embodiments R¹ is selected from the group consisting of:

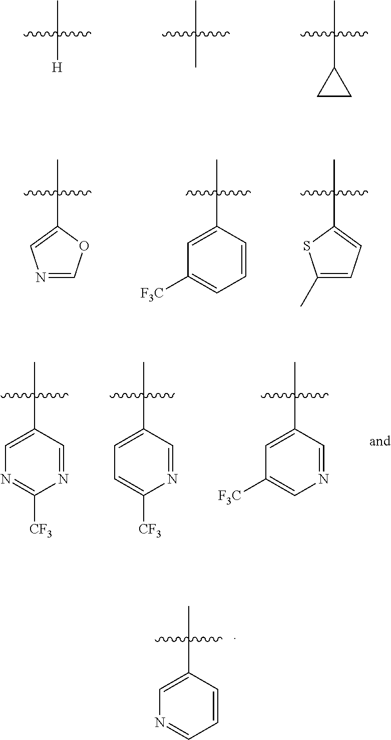

In some embodiments, R³ is selected from the group consisting of A, phenyl and monocyclic heteroaryl selected from pyridyl, thienyl, furyl, pyrimidinyl and pyrazolyl, wherein said phenyl and said heteroaryl are optionally and independently substituted with one or two R⁴.

In some embodiments, R³ is selected from the group consisting of A, phenyl and monocyclic heteroaryl selected from pyridyl, thienyl and pyrazolyl, wherein said phenyl and said heteroaryl are optionally and independently substituted with one or two R⁴.

In some embodiments, R³ is selected from the group consisting of A, phenyl and pyridyl, wherein said phenyl and said pyridyl are optionally substituted with one R⁴.

In some embodiments, R³ is selected from the group consisting of A, phenyl and pyridyl, wherein said phenyl and said pyridyl are optionally and independently substituted with one or two R⁴.

In some embodiments, R⁴ is selected from the group consisting of fluoro, chloro, C₁-C₃alkyl, C₃-C₆cycloalkyl, C₁-C₃fluoroalkyl and SO₂R⁷.

In some embodiments, R⁴ is selected from the group consisting of chloro, C₁-C₃alkyl, C₁-C₃fluoroalkyl and SO₂R⁷.

In some embodiments, Y is selected from the group consisting of CH2, NSO2R⁷, O and a bond.

In some embodiments, R¹ is selected from the group consisting of hydrogen, C₁-C₃alkyl, C₃-C₆cycloalkyl, phenyl, monocyclic heteroaryl and C₁-C₃haloalkyl, wherein said phenyl and said heteroaryl are optionally and independently substituted with one R¹²; and

R¹² is selected from the group consisting of C₁-C₃alkyl, cyclopropyl, CF₃, halogen, C₁-C₃haloalkoxy and C₁-C₃alkoxy.

In some embodiments, R¹⁰ is selected from the group consisting of hydrogen, C₁-C₃alkyl, phenyl, C₁-C₃haloalkyl.

In some embodiments, R⁷ is selected from the group consisting of R⁵, N,N-diC₁-C₃alkylamino, C₁-C₃alkyl and methoxyC₁-C₃alkyl, said C₁-C₃alkyl being optionally substituted with one R⁸.

In some embodiments, R⁷ is selected from C₁-C₃alkyl and fluorophenyl.

In some embodiments, R⁷ is selected from C₁-C₃alkyl and fluorobenzyl.

In some embodiments, R⁸ is selected from the group consisting of phenyl, pyridyl, imidazolyl, isoxazolyl, oxazolyl, cyclopropyl, cyclopentyl, pyrrolidinyl, tetrahydrofuryl, each optionally substituted with one or more substituents selected from cyclopropyl, methyl and fluoro.

In some embodiments, R³ is selected from the group consisting of:

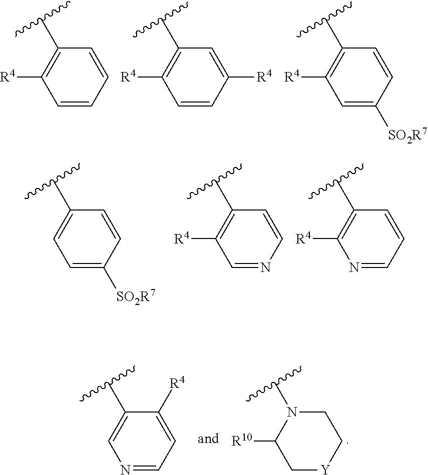

In some embodiments, R³ is selected from the group consisting of:

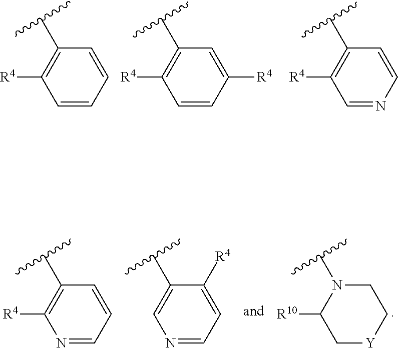

In some embodiments R³ is selected from the group consisting of:

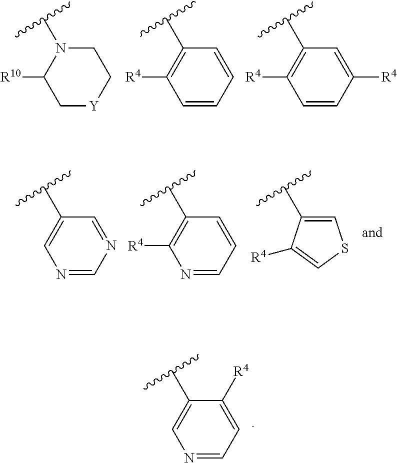

wherein Y is selected from the group consisting of CH₂, O and a bond; R⁴ is selected from CF₃, fluoro and chloro, cyclopropyl and methyl; and R¹⁰ is selected from the group consisting of cyclopropyl, methyl, fluorophenyl, chlorophenyl, methoxyphenyl and CF₃.

In some embodiments R³ is selected from the group consisting of:

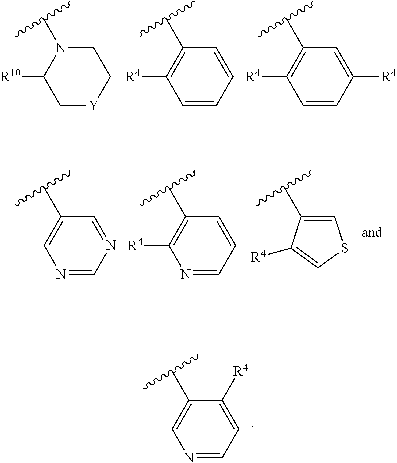

wherein Y is selected from the group) consisting of CH₂, O and a bond; R⁴ is selected from CF₃, chloro, and methyl; and R¹⁰ is selected from methyl, phenyl and CF₃.

In some embodiments, R³ is selected from the group consisting of:

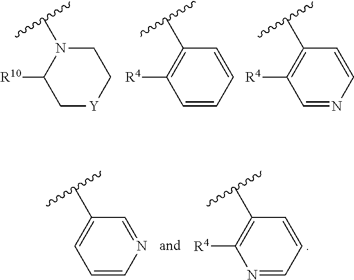

wherein Y is selected from CH₂, O, NSO₂R⁷ and a bond; R⁴ is selected from CF₃, fluoro, cyclopropyl and methyl; and R¹ is selected from hydrogen, phenyl, cyclopropyl, methyl, and CF₃.

In some embodiments, R³ is selected from the group consisting of:

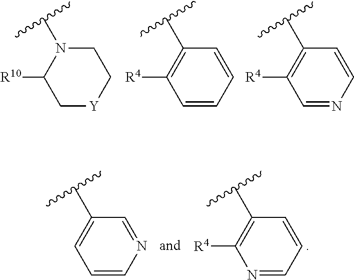

wherein Y is selected from the group consisting of CH₂, O, NSO₂R⁷ and a bond; R⁴ is selected from the group consisting of CF₃, chloro and methyl; and R¹⁰ is selected from hydrogen, phenyl, methyl, and CF₃.

In some embodiments, R³ is selected from the group consisting of:

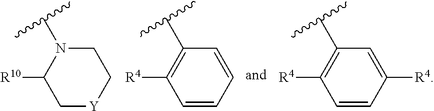

wherein Y is selected from the group consisting of CH₂, O and a bond; R⁴ is selected from the group consisting of CF₃, cyclopropyl, fluoro and chloro; and R¹⁰ is CF₃ or cyclopropyl.

In some embodiments, R³ is selected from

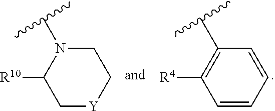

wherein Y is selected from the group consisting of CH, O and a bond; R⁴ is selected from the group consisting of CF₃ and chloro; and R¹⁰ is CF₃.

In some embodiments, X is N.

In some embodiments, X is CR¹.

In some embodiments, R¹ is selected from the group consisting of:

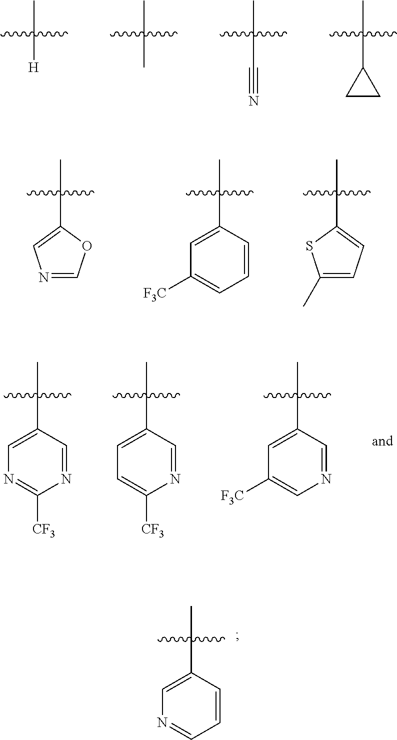

R² is hydrogen; and R³ is selected from the group consisting of:

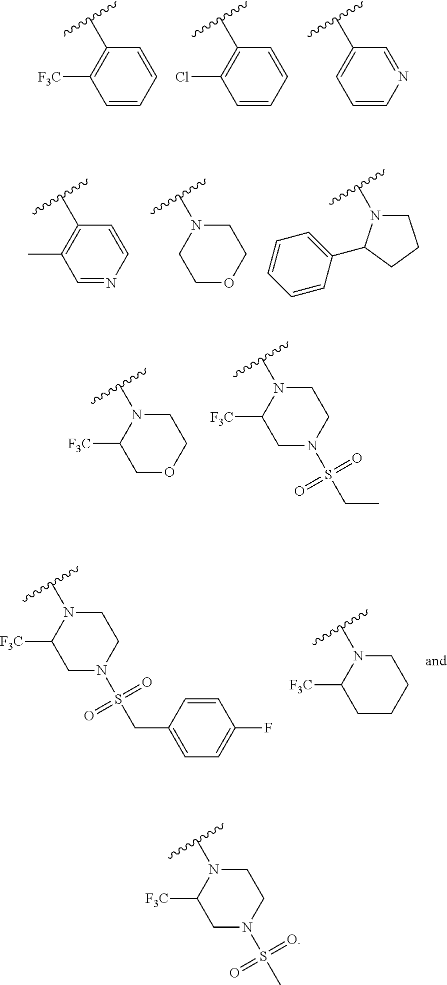

In some embodiments R¹ is selected from the group consisting of:

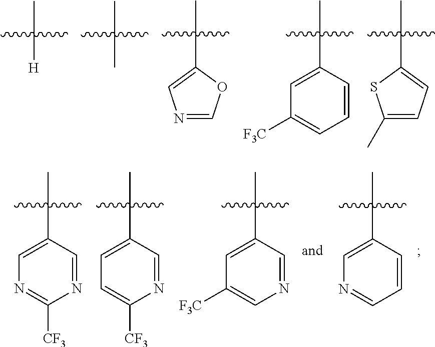

R² is hydrogen and R³ is selected from the group consisting of:

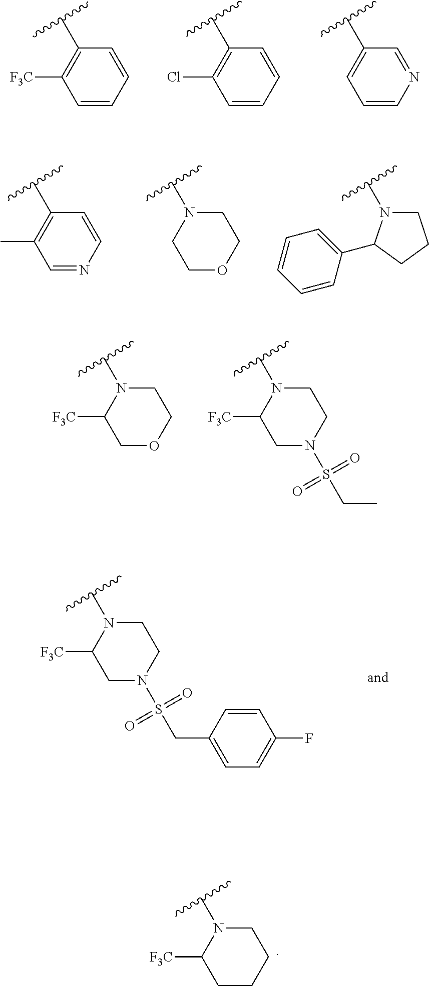

In some embodiments, R¹ is selected from the group consisting of:

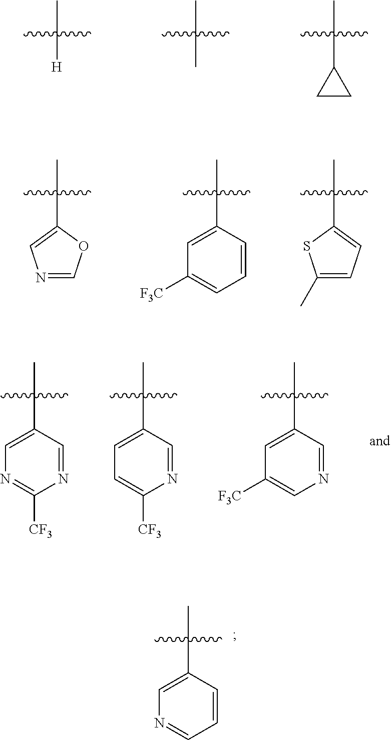

R² is hydrogen; and R³ is selected from the group consisting of:

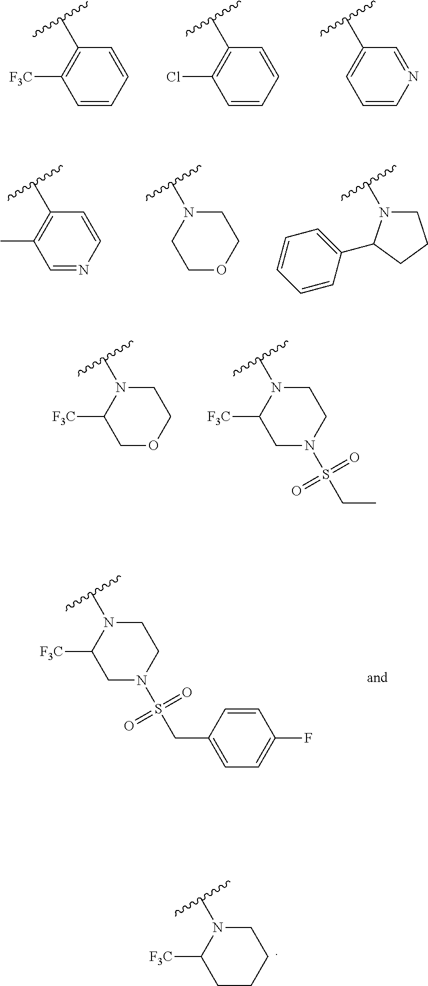

In some embodiments, X is N; R² is hydrogen; and R³ is selected from the group consisting of:

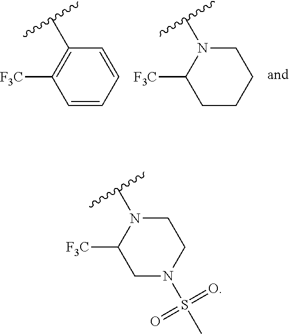

In some embodiments, the compound is selected from the group consisting of

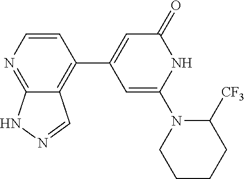 [SMILES: O=c1cc(-c2ccnc3[nH]ncc23)cc(N2CCCCC2C(F)(F)F)[nH]1]

and pharmaceutically acceptable salts, stereoisomers, and tautomers thereof.

In some embodiments, the compound is selected from the group consisting of:

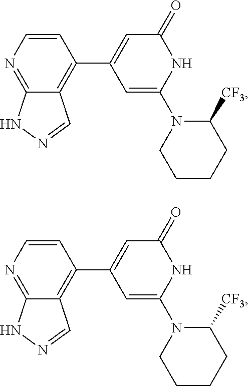

and pharmaceutically acceptable salts, stereoisomers, and tautomers thereof.

In some embodiments, the compound is selected from the group consisting of

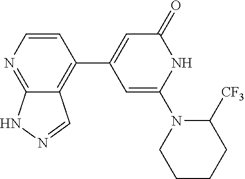 [SMILES: O=c1cc(-c2ccnc3[nH]ncc23)cc(N2CCCCC2C(F)(F)F)[nH]1]

and pharmaceutically acceptable salts thereof.

In some embodiments, the compound is selected from the group consisting of:

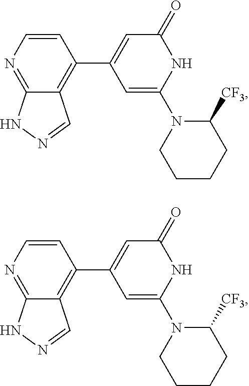

and pharmaceutically acceptable salts thereof.

In some embodiments, the compound is selected from the group consisting of: 4-(1H-pyrrolo[2,3-b]pyridin-4-yl)-6-[2-(trifluoromethyl)phenyl]-1H-pyridin-2-one; 6-(3-Methyl-4-pyridyl)-4-(1H-pyrrolo[2,3-b]pyridin-4-yl)-1H-pyridin-2-one; 6-(2-phenylpyrrolidin-1-yl)-4-(1H-pyrrolo[2,3-b]pyridin-4-yl)-1H-pyridin-2-one; 4-(2-Methyl-1H-pyrrolo[2,3-b]pyridin-4-yl)-6-(3-pyridyl)-1H-pyridin-2-one; 4-(2-Methyl-1H-pyrrolo[2,3-b]pyridin-4-yl)-6-morpholino-1H-pyridin-2-one; 6-(2-Chlorophenyl)-4-(2-oxazol-5-yl-1H-pyrrolo[2,3-b]pyridin-4-yl)-1H-pyridin-2-one; 6-(2-Chlorophenyl)-4-[2-(3-pyridyl)-1H-pyrrolo[2,3-b]pyridin-4-yl]-1H-pyridin-2-one; 6-(2-Chlorophenyl)-4-(2-methyl-1H-pyrrolo[2,3-b]pyridin-4-yl)-1H-pyridin-2-one; 4-(2-Methyl-1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[3-(trifluoromethyl)morpholin-4-yl]-1H-pyridin-2-one; 6-[3-(Trifluoromethyl)morpholin-4-yl]-4-[2-[3-(trifluoromethyl)phenyl]-1H-pyrrolo[2,3-b]pyridin-4-yl]-1H-pyridin-2-one; 4-[2-(5-Methyl-2-thienyl)-1H-pyrrolo[2,3-b]pyridin-4-yl]-6-[3-(trifluoromethyl)morpholin-4-yl]-1H-pyridin-2-one; 4-(1H-pyrazolo[3,4-b]pyridin-4-yl)-6-[2-(trifluoromethyl)-1-piperidyl]-1H-pyridin-2-one; 6-[2-(Trifluoromethyl)-1-piperidyl]-4-yl]-1H-pyrrolo[2,3-b]pyridin-4-yl]-1H-pyridin-2-one; 6-[2-(Trifluoromethyl)-1-piperidyl]-4-[2-[6-(trifluoromethyl)-3-pyridyl]-1H-pyrrolo[2,3-b]pyridin-4-yl]-1H-pyridin-2-one; 6-[2-(Trifluoromethyl)-1-piperidyl]-4-[2-[5-(trifluoromethyl)-3-pyridyl]-1H-pyrrolo[2,3-b]pyridin-4-yl]-1H-pyridin-2-one; 4-(2-cyclopropyl-1H-pyrrolo[2,3-b]pyridin-4-yl)-6-[4-ethylsulfonyl-2-(trifluoromethyl)piperazin-1-yl]-1H-pyridin-2-one; 6-[4-ethylsulfonyl-2-(trifluoromethyl)piperazin-1-yl]-4-(1H-pyrrolo[2,3-b]pyridin-4-yl)-1H-pyridin-2-one; 6-[4-[(4-fluorophenyl)methylsulfonyl]-2-(trifluoromethyl)piperazin-1-yl]-4-(1H-pyrrolo[2,3-b]pyridin-4-yl)-1H-pyridin-2-one; 6-[4-Ethylsulfonyl-2-(trifluoromethyl)piperazin-1-yl]-4-(2-methyl-1H-pyrrolo[2,3-b]pyridin-4-yl)-1H-pyridin-2-one; 4-[2-[4-Ethylsulfonyl-2-(trifluoromethyl)piperazin-1-yl]-6-oxo-1H-pyridin-4-yl]-1H-pyrrolo[2,3-b]pyridine-2-carbonitrile; 4-(2-cyclopropyl-1H-pyrrolo[2,3-b]pyridin-4-yl)-6-[2-(trifluoromethyl)phenyl]-1H-pyridin-2-one; 4-[2-Oxo-6-[2-(trifluoromethyl)phenyl]-1H-pyridin-4-yl]-1H-pyrrolo[2,3-b]pyridine-2-carbonitrile; 4-(1H-pyrazolo[3,4-b]pyridin-4-yl)-6-[2-(trifluoromethyl)phenyl]-1H-pyridin-2-one; 4-(6-(2-chlorophenyl)-2-oxo-1,2-dihydropyridin-4-yl)-N-ethyl-1H-pyrrolo[2,3-b]pyridine-2-carboxamide; 6-[4-Methylsulfonyl-2-(trifluoromethyl)piperazin-1-yl]-4-(1H-pyrazolo[3,4-b]pyridin-4-yl)-1H-pyridin-2-one; and pharmaceutically acceptable salts, stereoisomers, and tautomers thereof.

In some embodiments, the compound is selected from the group consisting of: 4-(1H-pyrrolo[2,3-b]pyridin-4-yl)-6-[2-(trifluoromethyl)phenyl]-1H-pyridin-2-one; 6-(3-Methyl-4-pyridyl)-4-(1H-pyrrolo[2,3-b]pyridin-4-yl)-1H-pyridin-2-one; 6-(2-phenylpyrrolidin-1-yl)-4-(1H-pyrrolo[2,3-b]pyridin-4-yl)-1H-pyridin-2-one; 4-(2-Methyl-1H-pyrrolo[2,3-b]pyridin-4-yl)-6-(3-pyridyl)-1H-pyridin-2-one; 4-(2-Methyl-1H-pyrrolo[2,3-b]pyridin-4-yl)-6-morpholino-1H-pyridin-2-one; 6-(2-Chlorophenyl)-4-(2-oxazol-5-yl-1H-pyrrolo[2,3-b]pyridin-4-yl)-1H-pyridin-2-one; 6-(2-Chlorophenyl)-4-[2-(3-pyridyl)-1H-pyrrolo[2,3-b]pyridin-4-yl]-1H-pyridin-2-one; 6-(2-Chlorophenyl)-4-(2-methyl-1H-pyrrolo[2,3-b]pyridin-4-yl)-1H-pyridin-2-one; 4-(2-Methyl-1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[3-(trifluoromethyl)morpholin-4-yl]-1H-pyridin-2-one; 6-[3-(Trifluoromethyl)morpholin-4-yl]-4-[2-[3-(trifluoromethyl)phenyl]-1H-pyrrolo[2,3-b]pyridin-4-yl]-1H-pyridin-2-one; 4-[2-(5-Methyl-2-thienyl)-1H-pyrrolo[2,3-b]pyridin-4-yl]-6-[3-(trifluoromethyl)morpholin-4-yl]-1H-pyridin-2-one; 4-(1H-pyrazolo[3,4-b]pyridin-4-yl)-6-[2-(trifluoromethyl)-1-pipendyl]-1H-pyridin-2-one; 6-[2-(Trifluoromethyl)-1-piperidyl]-4-yl]-1H-pyrrolo[2,3-b]pyridin-4-yl]-1H-pyridin-2-one; 6-[2-(Trifluoromethyl)-1-piperidyl]-4-[2-[6-(trifluoromethyl)-3-pyridyl]-1H-pyrrolo[2,3-b]pyridin-4-yl]-1H-pyridin-2-one; 6-[2-(Trifluoromethyl)-1-piperidyl]-4-[2-[5-(trifluoromethyl)-3-pyridyl]-1H-pyrrolo[2,3-b]pyridin-4-yl]-1H-pyridin-2-one; 4-(2-cyclopropyl-1H-pyrrolo[2,3-b]pyridin-4-yl)-6-[4-ethylsulfonyl-2-(trifluoromethyl)piperazin-1-yl]-1H-pyridin-2-one; 6-[4-ethylsulfonyl-2-(trifluoromethyl)piperazin-1-yl]-4-(1H-pyrrolo[2,3-b]pyridin-4-yl)-1H-pyridin-2-one; 6-[4-[(4-fluorophenyl)methylsulfonyl]-2-(trifluoromethyl)piperazin-1-yl]-4-(1H-pyrrolo[2,3-b]pyridin-4-yl)-1H-pyridin-2-one; and pharmaceutically acceptable salts, stereoisomers, and tautomers thereof.

In some embodiments, described herein is a compound of Formula IV:

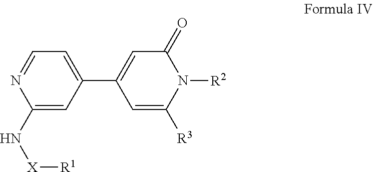

or a pharmaceutically acceptable salt, stereoisomer, or tautomer thereof, wherein: X is selected from —C(═O)— and a bond; R¹ is selected from the group consisting of H, C₁-C₃alkyl, C₁-C₃haloalkyl, C₁-C₃alkoxyC₁-C₃alkyl, C₃-C₆cycloalkyl, C₃-C₆cyclohaloalkyl, C₁-C₃alkoxy, C₁-C₃haloalkoxy, C₃-C₆cycloalkoxymethyl, N—C₁-C₃alkylamino, N,N-diC₁-C₃alkylamino, 1-pyrrolidinyl, 1-piperidinyl and 1-azetidinyl, provided that when R¹ is selected from the group consisting of C₁-C₃alkoxy, C₁-C₃haloalkoxy, N—C₁-C₃alkylamino, N,N-diC₁-C₃alkylamino, 1-pyrrolidinyl, 1-piperidinyl, and 1-azetidinyl, then X is C═O; R² is selected from the group consisting of H, C₁-C₃haloalkyl, and C₁-C₃alkyl; R³ is selected from the group consisting of A, phenyl and monocyclic heteroaryl, wherein each of phenyl and monocyclic heteroaryl is optionally substituted with one or more occurrences of a substituent independently selected from the group consisting of R⁴, R⁵, R⁶ and R⁷; each R⁴, R⁵, R⁶, and R⁷ is independently selected from the group consisting of halo, C₁-C₆alkyl, C₃-C₆cycloalkyl, C₁-C₆alkoxy, C₁-C₃haloalkoxy, N,N-diC₁-C₃alkylamino, N—C₁-C₃alkylamino, 1-azetidinyl, C₁-C₆haloalkyl, amino, NHSO₂R⁸, SO₂R⁹, and hydroxy; R⁸ is selected from C₁-C₃haloalkyl and C₁-C₃alkyl; each R⁹ is independently selected from the group consisting of R¹⁰, C₁-C₆alkyl, amino, N—C₁-C₃alkylamino, N,N-di-C₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; each R¹⁰ is independently selected from the group consisting of phenyl, monocyclic heteroaryl, C₃-C₆cycloalkyl, and heterocyclyl, wherein each phenyl, monocyclic heteroaryl, C₃-C₆cycloalkyl, and heterocyclyl is optionally substituted with one or more occurrences of R¹¹; each R¹¹ is independently selected from the group consisting of halo, C₁-C₃alkoxyC₁-C₃alkyl, amino, N—C₁-C₃alkylamino, N,N-diC₁-C₃alkylamino, C₁-C₃haloalkoxy, C₁-C₃alkoxy, C₃-C₆cycloalkyl, C₁-C₃haloalkyl, and C₁-C₃alkyl; A is:

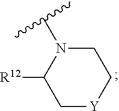

R¹² is selected from the group consisting of H, halo, COR¹³, C₁-C₆alkyl, C₁-C₃alkoxyC₁-C₃alkyl, C₁-C₆alkoxy, C₃-C₆cycloalkyl, C₁-C₃cyanoalkyl, and C₁-C₃haloalkyl; R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹⁵, NSO₂R⁹, NCOCH₂R⁹, O, and a bond; R¹⁴ is selected from the group consisting of H, C₁-C₃haloalkyl, C₁-C₃alkoxyC₁-C₃alkyl, C₁-C₃alkyl, and C₃-C₆cycloalkyl; and R¹⁵ is selected from the group consisting of R¹⁰, C₁-C₆alkyl and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen.

In some embodiments, X is —C(═O)—. In some embodiments, R¹ is C₁-C₃alkoxy. In some embodiments, R³ is aryl. In some embodiments, R³ is phenyl. In some embodiments, R³ is phenyl substituted with one occurrence of halo (e.g., chloro).

In some embodiments, R² is hydrogen or C₁-C₃alkyl, such as hydrogen or methyl, such as hydrogen.

In some embodiments, R¹ is selected from the group consisting of H, C₁-C₃alkyl, C₁-C₃alkoxy, C₁-C₃haloalkoxy, C₁-C₃alkoxyC₁-C₃alkyl, N,N-diC₁-C₃alkylamino, 1-pyrrolidinyl and C₃-C₆cycloalkyl.

In some embodiments, R¹ is selected from the group consisting of H, methyl, methoxy, methoxymethyl, N,N-dimethylamino, 1-pyrrolidinyl and cyclopropyl.

In some embodiments, R¹ is selected from the group consisting of H, methyl, methoxymethyl, N,N-dimethylamino, 1-pyrrolidinyl and cyclopropyl.

In some embodiments, R³ is selected from the group consisting of A, phenyl and monocyclic heteroaryl selected from pyridyl, thienyl, furyl, pyrimidinyl and pyrazolyl, wherein said phenyl and said heteroaryl are optionally substituted with R⁴ and/or R⁵.

In some embodiments, R³ is selected from the group consisting of A, phenyl and pyridyl, wherein said phenyl and said pyridyl are optionally and independently substituted with R⁴ and/or R⁵.

In some embodiments, R⁴, R⁵, R⁶ and R⁷ are independently selected from the group consisting of fluoro, chloro, C₁-C₃alkyl, C₃-C₆cycloalkyl, C₁-C₃fluoroalkyl and SO₂R⁹.

In some embodiments, Y is selected from the group consisting of CH₂, NSO₂R⁹, O and a bond.

In some embodiments, Y is selected from the group consisting of CH₂, O and a bond.

In some embodiments, R¹² is selected from the group consisting of hydrogen, C₁-C₃alkyl, C₁-C₃alkoxyC₁-C₃alkyl, C₁-C₃haloalkyl and C₃-C₆cycloalkyl.

In some embodiments, R¹² is selected from the group consisting of hydrogen, C₁-C₃alkyl, C₁-C₃haloalkyl and C₃-C₆cycloalkyl.

In some embodiments, R⁹ is selected from the group consisting of R¹⁰, N,N-diC₁-C₃alkylamino and methoxyC₁-C₃alkyl, said C₁-C₃alkyl being optionally substituted with one R¹⁰.

In some embodiments, R¹⁰ is selected from the group consisting of phenyl, pyridyl, imidazolyl, isoxazolyl, oxazolyl, cyclopropyl, cyclopentyl, pyrrolidinyl, tetrahydrofuryl, each optionally substituted with one or more methyl and/or fluoro.

In some embodiments, R³ is selected from the group consisting of:

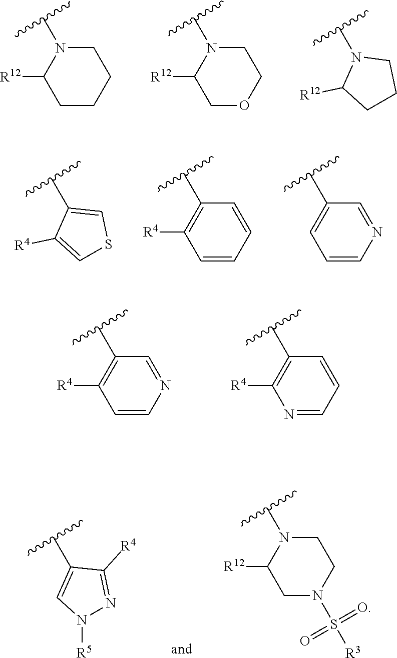

In some embodiments, R³ is selected from the group consisting of:

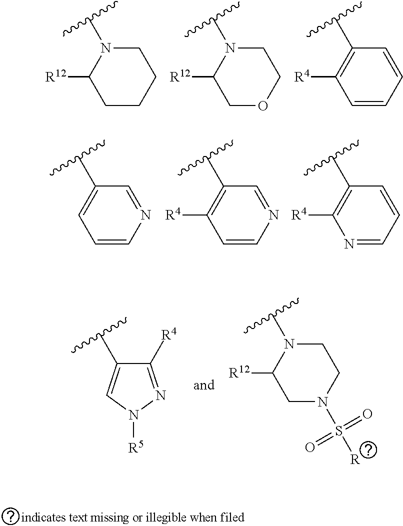

In some embodiments, R³ is selected from the group consisting of:

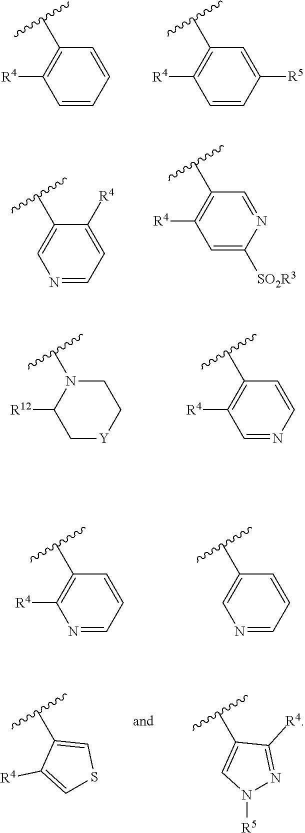

In some embodiments, R³ is selected from the group consisting of:

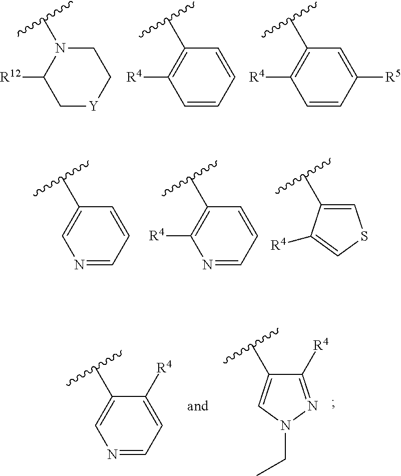

wherein Y is selected from the group consisting of CH₂, O and a bond; R⁴ is selected from CF₃, chloro, cyclopropyl and methyl; R⁵ is fluoro; and R¹² is selected from hydrogen, cyclopropyl, methyl, 1-methoxy-1-methyl-ethyl and CF₃.

In some embodiments, R³ is selected from the group consisting of:

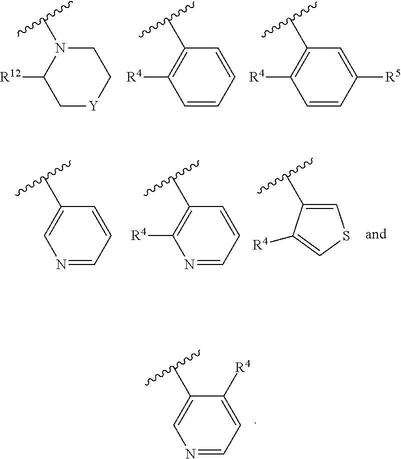

wherein Y is selected from the group consisting of CH₂, O and a bond; R⁴ is selected from the group consisting of CF₃, chloro, cyclopropyl and methyl; R⁵ is fluoro; and R¹² is selected from hydrogen, cyclopropyl, methyl and CF₃.

In some embodiments, R³ is selected from the group consisting of:

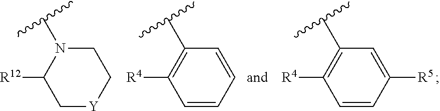

wherein Y is selected from CH₂ and O; R⁴ is selected from the group consisting of CF₃, chloro cyclopropyl and chloro; R⁵ is fluoro; and R¹² is CF₃ and cyclopropyl.

In some embodiments, R¹ is selected from the group consisting of H, methyl, methoxy, methoxymethyl, N,N-dimethylamino, 1-pyrrolidinyl and cyclopropyl; R² is hydrogen; and R³ is selected from the group consisting of:

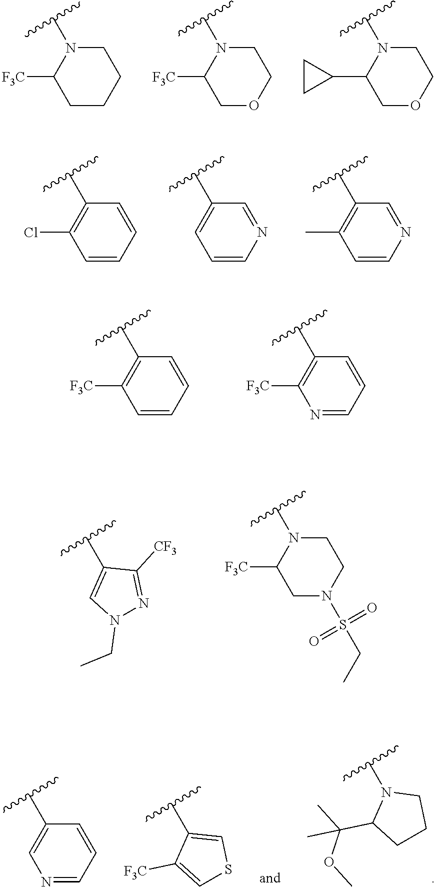

In some embodiments, R¹ is selected from the group consisting of H, methyl, methoxy, methoxymethyl, N,N-dimethylamino, 1-pyrrolidinyl and cyclopropyl; R² is hydrogen; and R³ is selected from the group consisting of:

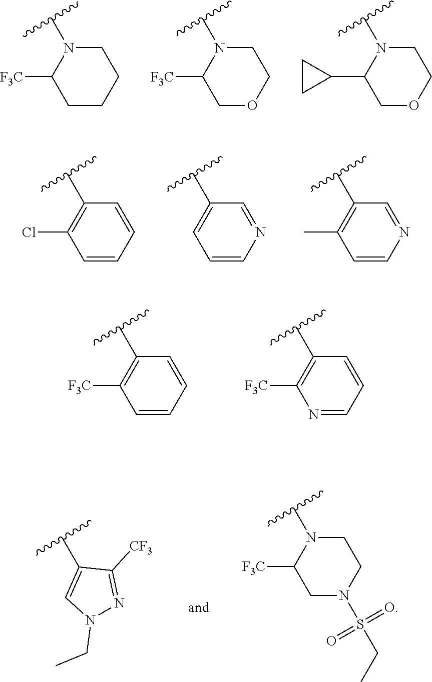

In some embodiments, R¹ is selected from the group consisting of H, methyl, methoxymethyl N,N-dimethylamino, 1-pyrrolidinyl and cyclopropyl; R² is hydrogen; and R³ is selected from the group consisting of:

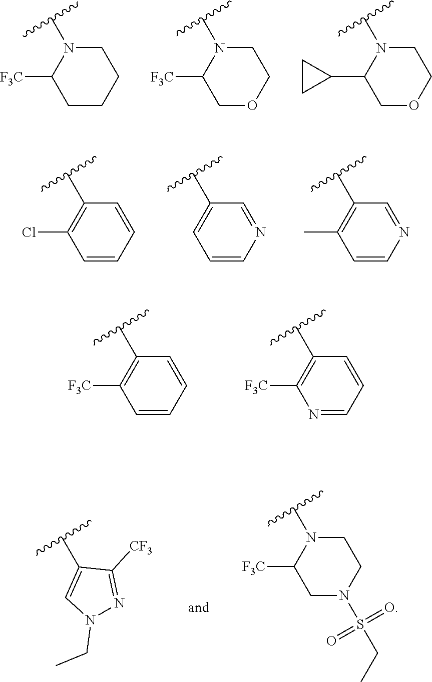

In some embodiments, the compound is selected from the group consisting of: N-[4-[2-(2-chlorophenyl)-6-oxo-1H-pyridin-4-yl]-2-pyridyl]acetamide; 4-(2-Amino-4-pyridyl)-6-(3-pyridyl)-1H-pyridin-2-one; 4-(2-Amino-4-pyridyl)-6-(2-chlorophenyl)-1H-pyridin-2-one; N-[4-[2-(2-chlorophenyl)-6-oxo-1H-pyridin-4-yl]-2-pyridyl]-2-methoxy-acetamide; N-[4-[2-oxo-6-[2-(trifluoromethyl)-1-piperidyl]-1H-pyridin-4-yl]-2-pyridyl]acetamide; N-[4-[2-oxo-6-[2-(trifluoromethyl)-1-piperidyl]-1H-pyridin-4-yl]-2-pyridyl]cyclopropanecarboxamide; N-[4-[2-oxo-6-[3-(trifluoromethyl)morpholin-4-yl]-1H-pyridin-4-yl]-2-pyridyl]acetamide; methyl N-[4-[2-(2-chlorophenyl)-6-oxo-1H-pyridin-4-yl]-2-pyridyl]carbamate; methyl N-[4-[2-[1-ethyl-3-(trifluoromethyl)pyrazol-4-yl]-6-oxo-1H-pyridin-4-yl]-2-pyridyl]carbamate; methyl N-[4-[2-oxo-6-[2-(trifluoromethyl)-3-pyridyl]-1H-pyridin-4-yl]-2-pyridyl]carbamate; methyl N-[4-[2-oxo-6-[2-(trifluoromethyl)phenyl]-1H-pyridin-4-yl]-2-pyridyl]carbamate; N-[4-[2-oxo-6-[2-(trifluoromethyl)phenyl]-1H-pyridin-4-yl]-2-pyridyl]acetamide; N-[4-[2-(4-methyl-3-pyridyl)-6-oxo-1H-pyridin-4-yl]-2-pyridyl]acetamide; N-[4-[2-oxo-6-[2-(trifluoromethyl)-3-pyridyl]-1H-pyridin-4-yl]-2-pyridyl]acetamide; N-[4-[2-[1-ethyl-3-(trifluoromethyl)pyrazol-4-yl]-6-oxo-1H-pyridin-4-yl]-2-pyridyl]acetamide; methyl N-[4-[2-oxo-6-[3-(trifluoromethyl)morpholin-4-yl]-1H-pyridin-4-yl]-2-pyridyl]carbamate; methyl N-[4-[2-oxo-6-[2-(trifluoromethyl)-1-piperidyl]-1H-pyridin-4-yl]-2-pyridyl]carbamate; N-[4-[2-(3-cyclopropylmorpholin-4-yl)-6-oxo-1H-pyridin-4-yl]-2-pyridyl]acetamide; N-[4-[2-[4-ethylsulfonyl-2-(trifluoromethyl)piperazin-1-yl]-6-oxo-1H-pyridin-4-yl]-2-pyridyl]acetamide; N-[4-[2-(2-methyl-3-pyridyl)-6-oxo-1H-pyridin-4-yl]-2-pyridyl]acetamide; N-[4-[2-oxo-6-[4-(trifluoromethyl)-3-thienyl]-1H-pyridin-4-yl]-2-pyridyl]acetamide; 1,1-Dimethyl-3-[4-[2-oxo-6-[2-(trifluoromethyl)phenyl]-1H-pyridin-4-yl]-2-pyridyl]urea; N-[4-[2-oxo-6-[2-(trifluoromethyl)phenyl]-1H-pyridin-4-yl]-2-pyridyl]pyrrolidine-1-carboxamide; N-[4-[2-[2-(1-methoxy-1-methyl-ethyl)pyrrolidin-1-yl]-6-oxo-1H-pyridin-4-yl]-2-pyridyl]acetamide; and pharmaceutically acceptable salts, tautomers, and stereoisomers thereof.

In some embodiments, the compound is selected from the group consisting of: 4-(2-Amino-4-pyridyl)-6-(3-pyridyl)-1H-pyridin-2-one; 4-(2-Amino-4-pyridyl)-6-(2-chlorophenyl)-1H-pyridin-2-one; N-[4-[2-(2-chlorophenyl)-6-oxo-1H-pyridin-4-yl]-2-pyridyl]-2-methoxy acetamide; N-[4-[2-oxo-6-[2-(trifluoromethyl)-1-piperidyl]-1H-pyridin-4-yl]-2-pyridyl]acetamide; N-[4-[2-oxo-6-[2-(trifluoromethyl)-1-piperidyl]-1H-pyridin-4-yl]-2-pyridyl]cyclopropanecarboxamide; N-[4-[2-oxo-6-[3-(trifluoromethyl)morpholin-4-yl]-1H-pyridin-4-yl]-2-pyridyl]acetamide; methyl N-[4-[2-(2-chlorophenyl)-6-oxo-1H-pyridin-4-yl]-2-pyridyl]carbamate; methyl N-[4-[2-[1-ethyl-3-(trifluoromethyl)pyrazol-4-yl]-6-oxo-1H-pyridin-4-yl]-2-pyridyl]carbamate; methyl N-[4-[2-oxo-6-[2-(trifluoromethyl)-3-pyridyl]-1H-pyridin-4-yl]-2 pyridyl]carbamate; methyl N-[4-[2-(4-methyl-3-pyridyl)-6-oxo-1H-pyridin-4-yl]-2-pyridyl]carbamate; methyl N-[4-[2-oxo-6-[2-(trifluoromethyl)phenyl]-1H-pyridin-4-yl]-2-pyridyl]carbamate; N-[4-[2-oxo-6-[2-(trifluoromethyl)phenyl]-1H-pyridin-4-yl]-2-pyridyl]acetamide; N-[4-[2-(4-methyl-3-pyridyl)-6-oxo-1H-pyridin-4-yl]-2-pyridyl]acetamide; N-[4-[2-oxo-6-[2-(trifluoromethyl)-3-pyridyl]-1H-pyridin-4-yl]-2-pyridyl]acetamide; N-[4-[2-[1-ethyl-3-(trifluoromethyl)pyrazol-4-yl]-6-oxo-1H-pyridin-4-yl]-2-pyridyl]acetamide; methyl N-[4-[2-oxo-6-[3-(trifluoromethyl)morpholin-4-yl]-1H-pyridin-4-yl]-2-pyridyl]carbamate; methyl N-[4-[2-oxo-6-[2-(trifluoromethyl)-1-piperidyl]-1H-pyridin-4-yl]-2-pyridyl]carbamate; methyl N-[4-[2-[4-ethylsulfonyl-2-(trifluoromethyl)piperazin-1-yl]-6-oxo-1H-pyridin-4-yl]-2-pyridyl]carbamate; methyl N-[4-[2-(3-cyclopropylmorpholin-4-yl)-6-oxo-1H-pyridin-4-yl]-2-pyridyl]carbamate; N-[4-[2-(3-cyclopropylmorpholin-4-yl)-6-oxo-1H-pyridin-4-yl]-2-pyridyl]acetamide; N-[4-[2-[4-ethylsulfonyl-2-(trifluoromethyl)piperazin-1-yl]-6-oxo-1H-pyridin-4-yl]-2-pyridyl]acetamide; 3-[4-[2-(2-chlorophenyl)-6-oxo-1H-pyridin-4-yl]-2-pyridyl]-1,1-dimethyl-urea; N-[4-[2-(2-chlorophenyl)-6-oxo-1H-pyridin-4-yl]-2-pyridyl]pyrrolidine-1-carboxamide; and pharmaceutically acceptable salts, tautomers, and stereoisomers thereof.

In some embodiments, the compound is selected from the group consisting of: N-[4-[2-(2-chlorophenyl)-6-oxo-1H-pyridin-4-yl]-2-pyridyl]acetamide; 4-(2-Amino-4-pyridyl)-6-(3-pyridyl)-1H-pyridin-2-one; 4-(2-Amino-4-pyridyl)-6-(2-chlorophenyl)-1H-pyridin-2-one; N-[4-[2-(2-chlorophenyl)-6-oxo-1H-pyridin-4-yl]-2-pyridyl]-2-methoxy-acetamide; N-[4-[2-oxo-6-[2-(trifluoromethyl)-1-piperidyl]-1H-pyridin-4-yl]-2-pyridyl]acetamide; N-[4-[2-oxo-6-[2-(trifluoromethyl)-1-piperidyl]-1H-pyridin-4-yl]-2-pyridyl]cyclopropanecarboxamide; N-[4-[2-oxo-6-[3-(trifluoromethyl)morpholin-4-yl]-1H-pyridin-4-yl]-2-pyridyl]acetamide; methyl N-[4-[2-(2-chlorophenyl)-6-oxo-1H-pyridin-4-yl]-2-pyridyl]carbamate; methyl N-[4-[2-[1-ethyl-3-(trifluoromethyl)pyrazol-4-yl]-6-oxo-1H-pyridin-4-yl]-2-pyridyl]carbamate; methyl N-[4-[2-oxo-6-[2-(trifluoromethyl)-3-pyridyl]-1H-pyridin-4-yl]-2-pyridyl]carbamate; methyl N-[4-[2-oxo-6-[2-(trifluoromethyl)phenyl]-1H-pyridin-4-yl]-2-pyridyl]carbamate; N-[4-[2-oxo-6-[2-(trifluoromethyl)phenyl]-1H-pyridin-4-yl]-2-pyridyl]acetamide; N-[4-[2-(4-methyl-3-pyridyl)-6-oxo-1H-pyridin-4-yl]-2-pyridyl]acetamide; N-[4-[2-oxo-6-[2-(trifluoromethyl)-3-pyridyl]-1H-pyridin-4-yl]-2-pyridyl]acetamide; N-[4-[2-[1-ethyl-3-(trifluoromethyl)pyrazol-4-yl]-6-oxo-1H-pyridin-4-yl]-2-pyridyl]acetamide; methyl N-[4-[2-oxo-6-[3-(trifluoromethyl)morpholin-4-yl]-1H-pyridin-4-yl]-2-pyridyl]carbamate; methyl N-[4-[2-oxo-6-[2-(trifluoromethyl)-1-piperidyl]-1H-pyridin-4-yl]-2-pyridyl]carbamate; N-[4-[2-(3-cyclopropylmorpholin-4-yl)-6-oxo-1H-pyridin-4-yl]-2-pyridyl]acetamide; N-[4-[2-[4-ethylsulfonyl-2-(trifluoromethyl)piperazin-1-yl]-6-oxo-1H-pyridin-4-yl]-2-pyridyl]acetamide; and pharmaceutically acceptable salts, tautomers, and stereoisomers thereof.

In some embodiments, the compound is selected from the group consisting of:

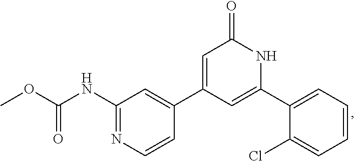 [SMILES: COC(=O)Nc1cc(-c2cc(-c3ccccc3Cl)[nH]c(=O)c2)ccn1]

and pharmaceutically acceptable salts and tautomers thereof.

In some embodiments, the compound is selected from the group consisting of:

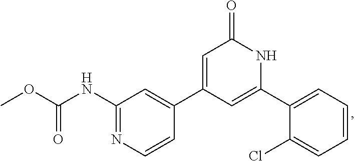 [SMILES: COC(=O)Nc1cc(-c2cc(-c3ccccc3Cl)[nH]c(=O)c2)ccn1]

and pharmaceutically acceptable salts thereof.

In some embodiments, described herein is a compound of Formula V:

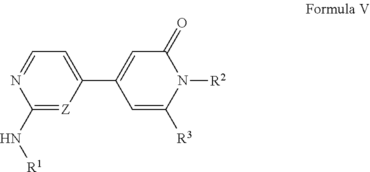

or a pharmaceutically acceptable salt, stereoisomer, or tautomer thereof, wherein: R¹ is selected from phenyl and monocyclic 5-6 membered heteroaryl, wherein each of phenyl and monocyclic 5-6 membered heteroaryl is optionally substituted with one or more substituents independently selected from the group consisting of halogen, C₁-C₆alkyl, C₃-C₄cycloalkyl, C₁-C₆alkoxy, C₁-C₆haloalkyl, C₁-C₆haloalkoxy, amino, N—C₁-C₃alkylamino and N,N-diC₁-C₃alkylamino; R² is selected from the group consisting of H, C₁-C₃haloalkyl, and C₁-C₃alkyl; R³ is selected from the group consisting of A, phenyl, and monocyclic heteroaryl, wherein each of phenyl and heteroaryl is optionally substituted with one or more occurrences of a substituent independently selected from the group consisting of R⁴, R⁵, R⁶, and R⁷; each of R⁴, R⁵, R⁶, and R⁷ is independently selected from the group consisting of halogen, C₁-C₆alkyl, C₃-C₄cycloalkyl, C₁-C₆alkoxy, C₁-C₆haloalkyl, C₁-C₆haloalkoxy, azetidine, amino, N—C₁-C₃alkylamino, N,N-diC₁-C₃alkylamino, NHSO₂R⁸, SO₂R⁹, and hydroxy; R⁸ is selected from C₁-C₃haloalkyl and C₁-C₃alkyl; each R⁹ is independently selected from the group consisting of R¹⁰, C₁-C₆alkyl, amino, N—C₁-C₃alkylamino, N,N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; each R¹⁰ is independently selected from the group consisting of phenyl, benzyl, monocyclic heteroaryl, C₃-C₆cycloalkyl, and heterocyclyl, wherein each of phenyl, benzyl, monocyclic heteroaryl, C₃-C₆cycloalkyl, and heterocyclyl is optionally substituted with one or more occurrences of R¹¹; each R¹¹ is independently selected from the group consisting of halogen, C₁-C₃haloalkyl, C₃-C₄cycloalkyl, C₁-C₃alkyl, amino, N—C₁-C₃alkylamino, N,N-diC₁-C₃alkylamino, and C₁-C₃alkoxyC₁-C₃alkyl; A is

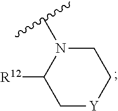

R¹² is selected from the group consisting of H, halogen, COR¹³, C₁-C₆alkyl, C₃-C₆cycloalkyl, C₁-C₃alkoxyC₁-C₃alkyl, C₁-C₆alkoxy, C₃-C₆cycloalkyl, C₁-C₃cyanoalkyl, and C₁-C₃haloalkyl; R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹⁵, NSO₂R⁹, NCOCH₂R⁹, O, and a bond; R¹⁴ is selected from H, C₁-C₃haloalkyl, C₁-C₃alkoxyC₁-C₃alkyl, C₁-C₃alkyl, and C₃-C₆cycloalkyl; and R¹⁵ is selected from R¹⁰, C₁-C₆alkyl, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and Z is selected from CH and N.

In some embodiments, R₂ is selected from hydrogen and C₁-C₃alkyl.

In some embodiments, R² is hydrogen.

In some embodiments, R¹ is selected from phenyl and a monocyclic 5-6 membered heteroaryl, each optionally substituted with one or more substituents selected from the group consisting of halogen, C₁-C₆alkyl, C₃-C₄cycloalkyl, C₁-C₆haloalkyl, C₁-C₆alkoxy, C₁-C₆haloalkoxy, amino, —N—C₁-C₃alkylamino, N,N-di-C₁-C₃alkylamino and halogen.

In some embodiments, R¹ is selected from phenyl and a monocyclic 5-6 membered heteroaryl, each optionally substituted with one or more substituents selected from the group consisting of C₁-C₆alkyl, C3-C₄cycloalkyl, and halogen.

In some embodiments, R³ is selected from the group consisting of:

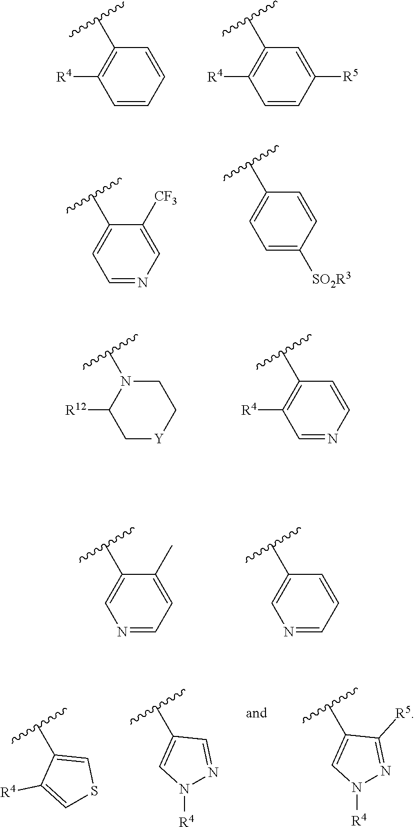

In some embodiments, R³ is selected from the group consisting of:

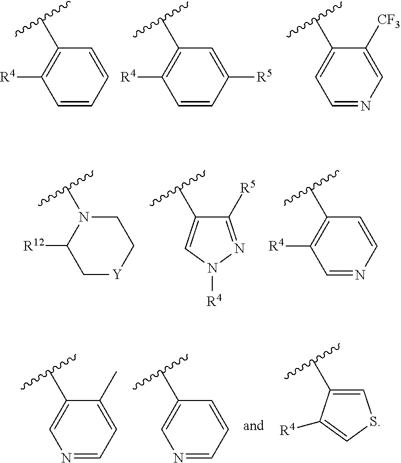

In some embodiments, R³ is selected from the group consisting of:

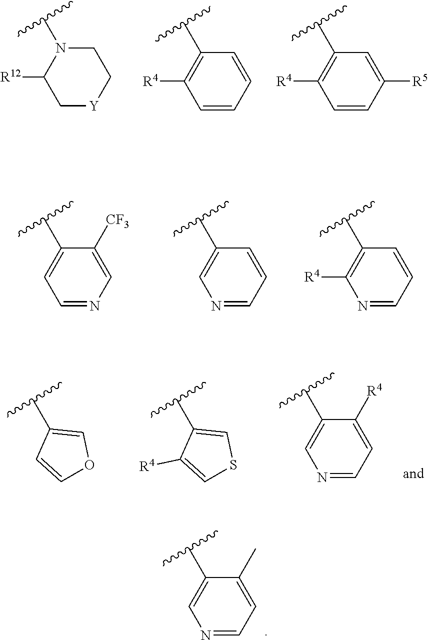

Y is selected from the group consisting of CH₂, NSO2R⁹, O and a bond; R⁴ is selected from the group consisting of CF₃, fluoro, cyclopropyl and methyl; R⁵ is fluoro; R⁹ is selected from the group consisting of C₁-C₆alkyl, phenyl, and benzyl, each optionally substituted with one or more halogen; and R¹² is selected from the group consisting of hydrogen, methyl, cyclopropyl and CF₃.

In some embodiments, R³ is selected from the group consisting of:

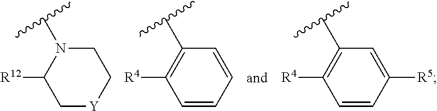

Y is selected from the group consisting of NSO2R⁹, CH₂ and O; R⁴ is selected from the group consisting of cyclopropyl, CF₃ and chloro; R⁵ is fluoro; R⁹ is selected from the group consisting of C₁-C₆alkyl, phenyl, and benzyl, each optionally substituted with one or more halogen; and R¹² is selected from cyclopropyl and CF₃.

In some embodiments, R³ is

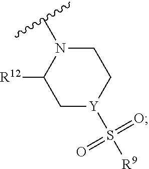

R⁹ is selected from the group consisting of C₁-C₆alkyl, phenyl, and benzyl, wherein said phenyl and benzyl group may optionally be substituted with one or more halogen, C₁-C₆alkyl, C₁-C₆haloalkyl and C₃-C₄cycloalkyl; and R¹² is selected from the group consisting of halogen, C₁-C₆alkyl, C₁-C₆haloalkyl and C3-C₄cycloalkyl.

In some embodiments, R¹ is selected from the group consisting of phenyl, pyrimidinyl, oxazolyl, imidazolyl, pyrazolyl and thiazolyl, each optionally substituted with one or more substituents selected from halogen, C₁-C₆alkyl, C3-C₄cycloalkyl, and C₁-C₆haloalkyl.

In some embodiments, R¹ is selected from the group consisting of phenyl, pyrimidinyl, oxazolyl, imidazolyl, and thiazolyl, each optionally substituted with one or more substituents selected from halogen, C₁-C₆alkyl, C3-C₄cycloalkyl, and C₁-C₆haloalkyl.

In some embodiments, R³ is selected from the group consisting of A, phenyl, pyridyl, thienyl, furyl, pyrimidinyl and pyrazolyl, each optionally and independently substituted with one or more R⁴ or R⁵.

In some embodiments, R³ is selected from the group consisting of A, phenyl and pyridyl, each optionally and independently substituted with one or more R⁴ or R⁵.

In some embodiments, R³ is selected from the group consisting of phenyl, pyridyl, morpholinyl, piperidyl, pyrrolidinyl, thienyl, and piperazinyl, each optionally substituted with one or more substituents selected from halogen, C₁-C₆alkyl, C₁-C₆haloalkyl and C3-C₄cycloalkyl.

In some embodiments, R⁴, R⁵, R⁶ and R⁷ are independently selected from the group consisting of fluoro, chloro, C₁-C₃alkyl. C₁-C₃fluoroalkyl, cyclopropyl and SO₂R⁹.

In some embodiments, Y is selected from the group consisting of CH2, O and a bond. In some embodiments, R¹² is selected from the group consisting of hydrogen, CON(CH₃)₂, C₁-C₃alkyl, CF₃ and cyclopropyl.

In some embodiments, R⁹ is selected from the group consisting of R¹⁰, N,N-diC₁-C₃alkylamino and methoxyC₁-C₃alkyl, said C₁-C₃alkyl being optionally substituted with one R¹⁰.

In some embodiments, R¹⁰ is selected from the group consisting of phenyl, benzyl, pyridyl, imidazolyl, isoxazolyl, oxazolyl, cyclopropyl, cyclopentyl, pyrrolidinyl, and tetrahydrofuryl, each optionally substituted with one or more methyl and/or fluoro.

In some embodiments, R³ is selected from the group consisting of phenyl, pyridyl, pyrrolidinyl and thienyl, each optionally substituted with one or more substituents selected from the group consisting of halogen, C₁-C₆alkyl, C₁-C₆haloalkyl and C₃-C₄cycloalkyl; or A; Y is CH2, O, NSO2-C₁-C₆alkyl or NSO2-benzyl, wherein said benzyl is optionally substituted by one or more halogen; and R¹² is selected from C₁-C₆alkyl and C₁-C₆haloalkyl.

In some embodiments, R¹ is selected from phenyl and a monocyclic 5-6 membered heteroaryl each optionally substituted with one or more substituents selected from the group consisting of halogen, C₁-C₆alkyl, C3-C₄cycloalkyl, C₁-C₆haloalkyl, C₁-C₆alkoxy, C₁-C₆haloalkoxy, amino, N—C₁-C₃alkylamino, N,N-di-C₁-C₃alkylamino and halogen; R² is hydrogen; R³ is selected from phenyl and a monocyclic 5-6 membered heteroaryl each optionally substituted with one or more substituents selected from the group consisting of halogen, C₁-C₆alkyl, C₁-C₆haloalkyl and C3-C₄cycloalkyl.

In some embodiments, R¹ is selected from phenyl and a monocyclic 5-6 membered heteroaryl each optionally substituted with one or more substituents selected from the group consisting of halogen, C₁-C₆alkyl, C₁-C₆haloalkyl, and C3-C₄cycloalkyl; R² is hydrogen; and R³ is selected from phenyl and monocyclic 5-6 membered heteroaryl each optionally substituted with one or more substituents selected from the group consisting of halogen, C₁-C₆alkyl, C₁-C₆haloalkyl and C3-C₄cycloalkyl.

In some embodiments, R¹ is selected from the group consisting of phenyl, pyrimidinyl, oxazolyl, imidazolyl, or thiazolyl, each optionally substituted with one or more substituents selected from the group consisting of halogen, C₁-C₆alkyl, C₁-C₆haloalkyl, and C₃-C₄cycloalkyl; R² is hydrogen; R³ is selected from the group consisting of the group consisting of phenyl, pyridyl, pyrazolyl pyrrolidinyl, and thienyl, each optionally substituted with one or more substituents selected from the group consisting of halogen, C₁-C₆alkyl, C₁-C₆haloalkyl, C3-C₄cycloalkyl; or A; Y is CH₂, O, NSO₂—C₁-C₆alkyl or NSO₂-benzyl, wherein said benzyl is optionally substituted by one or more halogen; and R¹² is C₁-C₆alkyl or C₁-C₆haloalkyl.

In some embodiments, R¹ is selected from the group consisting of phenyl, 4-pyrimidinyl, 2-methylpyrimidin-4-yl, 2-cyclopropyl-pyrimidin-4-yl, 2-oxazolyl, 1-methyl-imidazol-4-yl, 2-methyl-thiazol-4-yl, 3,5-difluorophenyl and 2-methylpyrazol-3-yl; R² is hydrogen; and R³ is selected from the group consisting of 2-chlorophenyl, 3-pyridyl, 4-pyridyl, 4-morpholinyl, 3-(trifluoromethyl)morpholin-4-yl, 2-(trifluoromethyl)-piperidin-1-yl, 2-(trifluoromethyl)phenyl, 4-methyl-pyridin-3-yl, 2-(trifluoromethyl)-pyridin-3-yl, 1-ethyl-3-(trifluoromethyl)pyrazol-4-yl, 3-cyclopropyl-morpholin-4-yl, 4-[(4-fluorophenyl)methylsulfonyl]-2-(trifluoromethyl)piperazin-1-yl, 4-ethylsulfonyl-2-(trifluoromethyl)piperazin-1-yl, 2-(trifluoromethyl)-pyrrolidin-1-yl, 2-chloro-5-fluorophenyl, 3-(trifluoromethyl)-pyrazolin-4-yl, 2-(trifluoromethyl)-pyridin-3-yl, 3-methyl-thien-4-yl, 2-methyl phenyl, 1-acetyl-3-trifluoromethyl-piperazin-4-yl, 2-methyl-piperidin-1-yl, 2-cyclopropyl-piperidin-1-yl, 2-methyl-morpholin-4-yl, 2-trifluoromethyl-morpholin-4-yl, and 2-cyclopropyl-morpholin-4-yl.

In some embodiments, R¹ is selected from the group consisting of phenyl, 4-pyrimidinyl, 2-methylpyrimidin-4-yl, 2-cyclopropyl-pyrimidin-4-yl, 2-oxazolyl, 1-methyl-imidazol-4-yl, 2-methyl-thiazol-4-yl, and 3,5-difluorophenyl; R² is hydrogen; and R³ is selected from the group consisting of 2-chlorophenyl, 3-pyridyl, 4-pyridyl, 4-morpholinyl, 3-(trifluoromethyl)morpholin-4-yl, 2-(trifluoromethyl)-piperidin-1-yl, 2-(trifluoromethyl)phenyl, 4-methyl-pyridin-3-yl, 2-(trifluoromethyl)-pyridin-3-yl, 1-ethyl-3-(trifluoromethyl)pyrazol-4-yl, 3-cyclopropyl-morpholin-4-yl, 4-[(4-fluorophenyl)methylsulfonyl-]2-(trifluoromethyl)piperazin-1-yl, 4-ethylsulfonyl-2-(trifluoromethyl)-piperazin-1-yl, 2-(trifluoromethyl)-pyrrolidin-1-yl, 2-chloro-5-fluorophenyl, 3-(trifluoromethyl)-pyrazolin-4-yl, 2-(trifluoromethyl)-pyridin-3-yl, 3-methyl-thien-4-yl, 2-methyl phenyl, 1-acetyl-3-trifluoromethyl-piperazin-4-yl, 2-methyl-piperidin-1-yl, 2-cyclopropyl-piperidin-1-yl, 2-methyl-morpholin-4-yl, 2-trifluoromethyl-morpholin-4-yl, and 2-cyclopropyl-morpholin-4-yl.

In some embodiments, R¹ is selected from phenyl and monocyclic 5-6 membered heteroaryl, each optionally substituted with one or more substituents selected from the group consisting of halogen, C₁-C₆alkyl, C₁-C₆haloalkyl, and C3-C₄cycloalkyl; R² is hydrogen; R³ is selected from phenyl and a monocyclic 5-6 membered heteroaryl, each optionally substituted with one or more substituents selected from the group consisting of halogen, C₁-C₆alkyl, C₁-C₆haloalkyl, and C₃-C₄cycloalkyl; and Z is CH or N; or a pharmaceutically acceptable salt thereof.

In some embodiments, R¹ is selected from phenyl and pyrimidinyl, each optionally substituted with one or more substituents selected from the group consisting of halogen, C₁-C₆alkyl, C₁-C₆haloalkyl, or C3-C₄cycloalkyl; R² is hydrogen; R³ is selected from the group consisting of phenyl, pyridyl and pyrazolyl, each optionally substituted with one or more substituents selected from halogen, C₁-C₆alkyl, C₁-C₆haloalkyl, and C₃-C₄cycloalkyl; or A; Y is selected from the group consisting of CH₂, O, NSO₂—C₁-C₆alkyl and NSO₇-benzyl, wherein said benzyl is optionally substituted by one or more halogen; R¹² is selected from C₁-C₆alkyl and C₁-C₆haloalkyl; and Z is selected from CH and N; or a pharmaceutically acceptable salt thereof.

In some embodiments, R¹ is selected from phenyl, 3,5-difluorophenyl and 2-methylpyrimidin-4-yl; R² is hydrogen; R³ is selected from the group consisting of 2-chlorophenyl, 3-pyridyl, 4-pyridyl, 1-morpholinyl, 2-(trifluoromethyl)-1-piperidyl, 3-(trifluoromethyl)morpholin-4-yl, 4-[(4-fluorophenyl)methylsulfonyl]-2-(trifluoromethyl)piperazin-1-yl, 4-ethylsulfonyl-2-(trifluoromethyl)piperazin-1-yl, 2-(trifluoromethyl)phenyl, 4-methyl-3-pyridyl, 2-(trifluoromethyl)-3-pyridyl, 1-ethyl-3-(trifluoromethyl)pyrazol-4-yl, and 3-cyclopropylmorpholin-4-yl; and Z is selected from CH and N; or a pharmaceutically acceptable salt thereof.

In some embodiments, R¹ is selected from phenyl and monocyclic 5-6 membered heteroaryl, each optionally substituted with one or more substituents selected from the group consisting of halogen, C₁-C₆alkyl, C₁-C₆haloalkyl, and C₃-C₄cycloalkyl; R² is hydrogen; R³ is selected from phenyl and a monocyclic 5-6 membered heteroaryl, each optionally substituted with one or more substituents selected from the group consisting of halogen, C₁-C₆alkyl, C₁-C₆haloalkyl, and C3-C₄cycloalkyl; and Z is CH or N; or a pharmaceutically acceptable salt thereof.

In some embodiments, R¹ is selected from phenyl and pyrimidinyl, each optionally substituted with one or more substituents selected from the group consisting of halogen, C₁-C₆alkyl, C₁-C₆haloalkyl, and C₃-C₄cycloalkyl; R² is hydrogen; R³ is selected from phenyl and pyridyl, each optionally substituted with one or more substituents selected from the group consisting of halogen, C₁-C₆alkyl, C₁-C₆haloalkyl, and C3-C₄cycloalkyl; or A; Y is selected from the group consisting of CH₂, O, NSO₂—C₁-C₆alkyl and NSO₂-benzyl, wherein said benzyl is optionally substituted by one or more halogen; R¹² is selected from C₁-C₆alkyl and C₁-C₆haloalkyl; and Z is selected from CH and N; or a pharmaceutically acceptable salt thereof.

In some embodiments, R¹ is selected from the group consisting of phenyl, 2-methylpyrimidin-4-yl, oxazol-2yl, 2-methylthiazol-4-yl, 2-methylpyrazol-3-yl, and 1-methyl imidazol-4-yl; R² is hydrogen; R³ is selected from the group consisting of 2-chlorophenyl, 3-pyridyl, 4-pyridyl, 1-morpholinyl, 2-(trifluoromethyl)-1-piperidyl, 3-(trifluoromethyl)morpholin-4-yl, 4-[(4-fluorophenyl)methylsulfonyl]-2-(trifluoromethyl)piperazin-1-yl, 4-ethylsulfonyl-2-(trifluoromethyl)piperazin-1-yl; 2-(trifluoromethyl)phenyl, 4-methyl-pyridin-3-yl, 2-(trifluoromethyl)-pyridin-3-yl, and 1-ethyl-3-(trifluoromethyl)pyrazol-4-yl; and Z is selected from CH and N; or a pharmaceutically acceptable salt thereof.

In some embodiments, R¹ is selected from phenyl and 2-methylpyrimidin-4-yl; R² is hydrogen; R³ is selected from the group consisting of 2-chlorophenyl, 3-pyridyl, 4-pyridyl, 1-morpholinyl, 2-(trifluoromethyl)-1-piperidyl, 3-(trifluoromethyl)morpholin-4-yl, 4-[(4-fluorophenyl)methylsulfonyl]-2-(trifluoromethyl)piperazin-1-yl, and 4-ethylsulfonyl-2-(trifluoromethyl)piperazin-1-yl; and Z is selected from CH and N; or a pharmaceutically acceptable salt thereof.

In some embodiments, the compound is selected from the group consisting of: 4-(2-anilinopyrimidin-4-yl)-6-(2-chlorophenyl)-1H-pyridin-2-one; 4-(2-anilinopyrimidin-4-yl)-6-(3-pyridyl)-1H-pyridin-2-one; 4-(2-anilinopyrimidin-4-yl)-6-(4-pyridyl)-1H-pyridin-2-one; 4-(2-anilinopyrimidin-4-yl)-6-morpholino-1H-pyridin-2-one; 4-[2-[(2-Methylpyrimidin-4-yl)amino]-4-pyridyl]-6-[2-(trifluoromethyl)-1-piperidyl]-1H-pyridin-2-one; 4-(2-anilinopyrimidin-4-yl)-6-[2-(trifluoromethyl)-1-piperidyl]-1H-pyridin-2-one; 4-[2-[(2-Methylpyrimidin-4-yl)amino]-4-pyridyl]-6-[3-(trifluoromethyl)morpholin-4-yl]-1H-pyridin-2-one; 4-(2-anilinopyrimidin-4-yl)-6-[3-(trifluoromethyl)morpholin-4-yl]-1H-pyridin-2-one; 6-[4-[(4-Fluorophenyl)methylsulfonyl]-2-(trifluoromethyl)piperazin-1-yl]-4-[2-[(2-methylpyrimidin-4-yl)amino]-4-pyridyl]-1H-pyridin-2-one; 6-[4-Ethylsulfonyl-2-(trifluoromethyl)piperazin-1-yl]-4-[2-[(2-methylpyrimidin-4-yl)amino]-4-pyridyl]-1H-pyridin-2-one; 4-[2-(Oxazol-2-ylamino)-4-pyridyl]-6-[3-(trifluoromethyl)morpholin-4-yl]-1H-pyridin-2-one; 4-[2-[(2-Methylthiazol-4-yl)amino]-4-pyridyl]-6-[3-(trifluoromethyl)morpholin-4-yl]-1H-pyridin-2-one; 4-[2-[(2-methylpyrimidin-4-yl)amino]-4-pyridyl]-6-[2-(trifluoromethyl)-phenyl]-1H-pyridin-2-one; 4-[2-[(2-Methylpyrazol-3-yl)amino]-4-pyridyl]-6-[2-(trifluoromethyl)phenyl]-1H-pyridin-2-one; 4-[2-[(2-Methylthiazol-4-yl)amino]-4-pyridyl]-6-[2-(trifluoromethyl)phenyl]-1H-pyridin-2-one; 6-(4-methyl-3-pyridyl)-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-3-pyridyl]-1H-pyridin-2-one; 6-[1-ethyl-3-(trifluoromethyl)pyrazol-4-yl]-4-[2-[(2-methylpyrimidin-4-yl)am 4-pyridyl]-1H-pyridin-2-one; 4-[2-[(1-Methylimidazol-4-yl)amino]-4-pyridyl]-6-[2-(trifluoromethyl)phenyl pyridin-2-one; 6-(2-chlorophenyl)-4-[2-[(2-methylpyrimidin-4-yl)amino]-4-pyridyl]-1H-pyridin-2-one, and pharmaceutically acceptable salts, stereoisomers, and tautomers thereof.

In some embodiments, the compound is selected from the group consisting of: 4-(2-anilinopyrimidin-4-yl)-6-(2-chlorophenyl)-1H-pyridin-2-one; 4-(2-anilinopyrimidin-4-yl)-6-(3-pyridyl)-1H-pyridin-2-one; 4-(2-anilinopyrimidin-4-yl)-6-(4-pyridyl)-1H-pyridin-2-one; 4-(2-anilinopyrimidin-4-yl)-6-morpholino-1H-pyridin-2-one; 4-[2-[(2-Methylpyrimidin-4-yl)amino]-4-pyridyl]-6-[2-(trifluoromethyl)-1-piperidyl]-1H-pyridin-2-one; 4-(2-anilinopyrimidin-4-yl)-6-[2-(trifluoromethyl)-1-piperidyl]-1H-pyridin-2-one; 4-[2-[(2-Methylpyrimidin-4-yl)amino]-4-pyridyl]-6-[3-(trifluoromethyl)morpholin-4-yl]-1H-pyridin-2-one; 4-(2-anilinopyrimidin-4-yl)-6-[3-(trifluoromethyl)morpholin-4-yl]-1H-pyridin-2-one; 6-[4-[(4-Fluorophenyl)methylsulfonyl]-2-(trifluoromethyl)piperazin-1-yl]-4-[2-[(2-methylpyrimidin-4-yl)amino]-4-pyridyl]-1H-pyridin-2-one; 6-[4-Ethylsulfonyl-2-(trifluoromethyl)piperazin-1-yl]-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-phenyl]-1H-pyridin-2-one; 6-(4-methyl-3-pyridyl)-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-3-pyridyl]-1H-pyridin-2-one; 6-[1-ethyl-3-(trifluoromethyl)pyrazol-4-yl]-4-[2-[(2-methylpyrimidin-4-yl)am 4-pyridyl]-1H-pyridin-2-one; 6-(2-chlorophenyl)-4-[2-[(2-methylpyrimidin-4-yl)amino]-4-pyridyl]-1H-pyridin-2-one; 6-(3-cyclopropylmorpholin-4-yl)-4-[2-[(2-methylpyrimidin-4-yl)amino]-4-pyridyl]-1H-pyridin-2-one, and pharmaceutically acceptable salts, stereoisomers, and tautomers thereof.

In some embodiments, the compound is selected from the group consisting of: 4-(2-anilinopyrimidin-4-yl)-6-(2-chlorophenyl)-1H-pyridin-2-one; 4-(2-anilinopyrimidin-4-yl)-6-(3-pyridyl)-1H-pyridin-2-one; 4-(2-anilinopyrimidin-4-yl)-6-(4-pyridyl)-1H-pyridin-2-one 4-(2-anilinopyrimidin-4-yl)-6-morpholino-1H-pyridin-2-one; 4-[2-[(2-Methylpyrimidin-4-yl)amino]-4-pyridyl]-6-[2-(trifluoromethyl)-1-piperidyl]-1H-pyridin-2-one; 4-(2-anilinopyrimidin-4-yl)-6-[2-(trifluoromethyl)-1-piperidyl]-1H-pyridin-2-one; 4-[2-[(2-Methylpyrimidin-4-yl)amino]-4-pyridyl]-6-[3-(trifluoromethyl)morpholin-4-yl]-1H-pyridin-2-one; 4-(2-anilinopyrimidin-4-yl)-6-[3-(trifluoromethyl)morpholin-4-yl]-1H-pyridin-2-one; 6-[4-[(4-Fluorophenyl)methylsulfonyl]-2-(trifluoromethyl)piperazin-1-yl]-4-[2-[(2-methylpyrimidin-4-yl)amino]-4-pyridyl]-1H-pyridin-2-one; 6-[4-Ethylsulfonyl-2-(trifluoromethyl)piperazin-1-yl]-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-phenyl]-1H-pyridin-2-one; 6-(4-methyl-3-pyridyl)-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-3-pyridyl]-1H-pyridin-2-one; 6-[1-ethyl-3-(trifluoromethyl)pyrazol-4-yl]-4-[2-[(2-methylpyrimidin-4-yl)amino]-4-pyridyl]-1H-pyridin-2-one; 6-(2-chlorophenyl)-4-[2-[(2-methylpyrimidin-4-yl)amino]-4-pyhdyl]-1H-pyridin-2-one, and pharmaceutically acceptable salts, stereoisomers, and tautomers thereof.

In some embodiments, described herein is a compound of Formula VI:

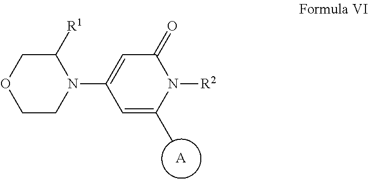

or a pharmaceutically acceptable salt, stereoisomer, or tautomer thereof, wherein: R¹ is selected from C₁-C₃alkyl and cyclopropyl; R² is selected from the group consisting of H, C₁-C₃haloalkyl, and C₁-C₃alkyl; A is selected from:

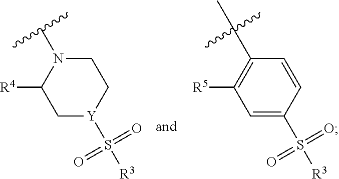

each R³ is independently selected from the group consisting of R⁶, C₁-C₆alkyl, amino N—C₁-C₃alkylamino, N, N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R⁶, and each of C₁-C₆alkyl and C₁-C₃alkoxyC₁-C₃alkyl is optionally substituted with or one or more independent occurrences of halogen; R⁴ is selected from the group consisting of C₁-C₆alkyl, C₁-C₆alkoxy, C₁-C₆haloalkyl, C₃-C₆cycloalkyl, and phenyl, wherein phenyl is optionally substituted with one or more occurrences of a substituent independently selected from the group consisting of fluoro, chloro, methyl, methoxy, dimethylamino, trifluoromethoxy, trifluoromethyl, and cyclopropyl; R⁵ is selected from the group consisting of halogen, C₁-C₆alkyl, C₁-C₆alkoxy, C₁-C₆haloalkyl, and C₃-C₆cycloalkyl; each R⁶ is independently selected from the group consisting of phenyl, monocyclic heteroaryl, C₃-C₆Cycloalkyl, and heterocyclyl, wherein each of phenyl, monocyclic heteroaryl, C₃-C₆cycloalkyl, and heterocyclyl is optionally substituted with one or more occurrences of R⁷; and each R⁷ is independently selected from the group consisting of halogen, amino, N—C₁-C₃alkylamino, N,N-diC₁-C₃alkylamino and C₁-C₃alkoxyC₁-C₃alkyl, C₁-C₃alkoxy, C₁-C₃haloalkoxy, C₃-C₆cycloalkyl, C₁-C₃haloalkyl, and C₁-C₃alkyl.

In some embodiments, R² is selected from hydrogen and C₁-C₃alkyl.

In some embodiments, R¹ is methyl.

In some embodiments, R⁷ is selected from the group consisting of fluoro, cyclopropyl and methyl.

In some embodiments, R⁷ is fluoro or methyl.

In some embodiments, R⁴ is selected from the group consisting of methyl, trifluoromethyl, cyclopropyl and phenyl, said phenyl being optionally meta-substituted with one of fluoro, chloro, methyl, methoxy, dimethylamino, trifluoromethoxy, trifluoromethyl and cyclopropyl; and

R⁵ is selected from the group consisting of chloro, cyclopropyl, methyl and trifluoromethyl.

In some embodiments, R⁴ and R⁵ are independently selected from the group consisting of C₁-C₃haloalkyl, such as C₁-C₃fluorooalkyl, such as monofluoromethyl, difluoromethyl, and trifluoromethyl.

In some embodiments, R⁴ is selected from the group consisting of methyl, trifluoromethyl and cyclopropyl; and R⁵ is selected from the group consisting of chloro, cyclopropyl, methyl and trifluoromethyl.

In some embodiments, R³ is selected from the group consisting of R⁶, C₁-C₃alkyl, N,N-diC₁-C₃alkylamino and methoxyC₁-C₃alkyl, said C₁-C₃alkyl being optionally substituted with one R⁶.

In some embodiments, R⁶ is selected from the group consisting of phenyl, pyridyl, morpholinyl, imidazolyl, isoxazolyl, pyrazolyl, oxazolyl, cyclopropyl, cyclopentyl, pyrrolidinyl and tetrahydrofuryl, each optionally substituted with one or more R⁷.

In some embodiments, R⁶ is selected from the group consisting of phenyl, pyridyl, morpholinyl, imidazolyl, pyrazolyl, cyclopropyl, pyrrolidinyl, piperidinyl, and tetrahydrofuryl, each optionally substituted with one or more R⁷

In some embodiments, R⁶ is selected from the group consisting of phenyl, pyridyl, pyrrolidinyl, pyrazolyl, tetrahydrofuryl, each optionally substituted with one or more R⁷.

In some embodiments, R⁶ is selected from the group consisting of:

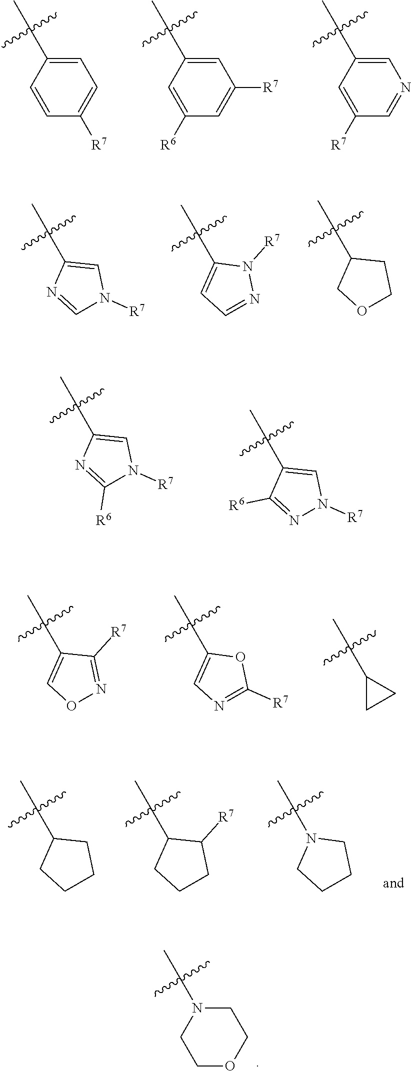

In some embodiments, R⁶ is selected from the group consisting of:

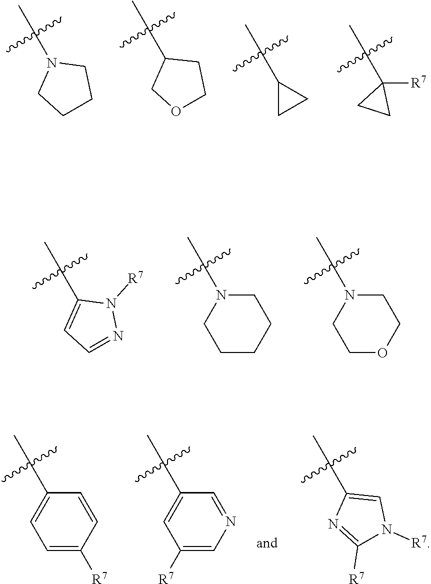

In some embodiments R⁶ is selected from the group consisting of:

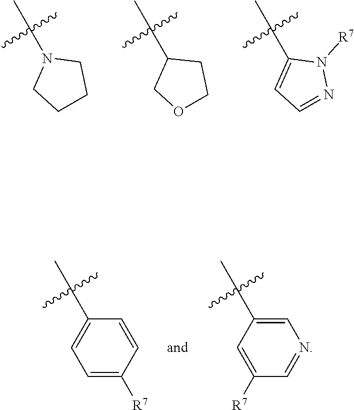

In some embodiments, R³ is selected from the group consisting of:

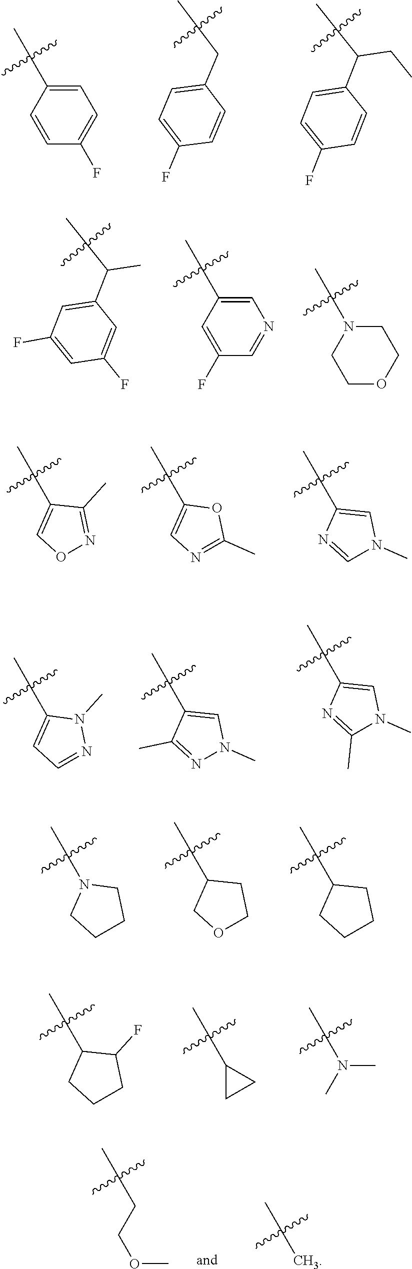

In some embodiments, R³ is selected from the group consisting of:

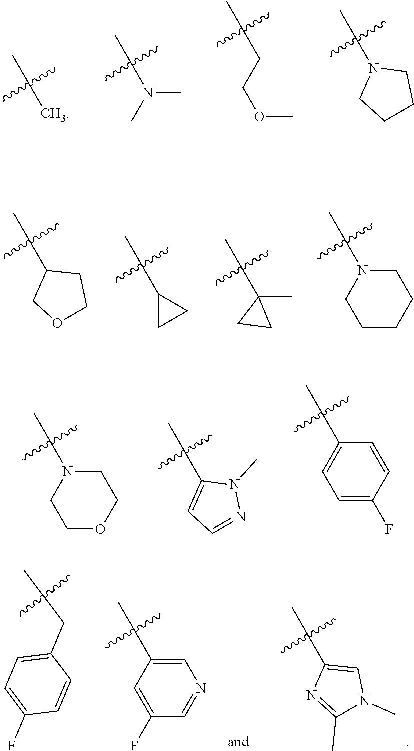

In some embodiments, R³ is selected from the group consisting of:

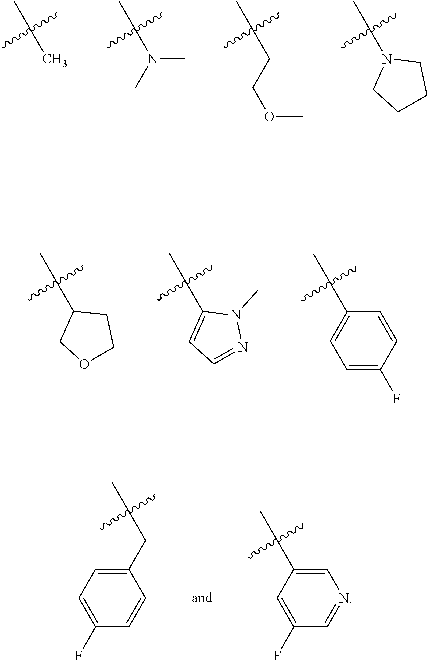

In some embodiments, A is

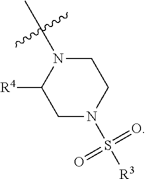

In some embodiments, A is

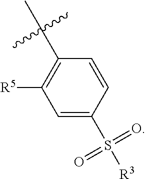

In some embodiments, R¹ is selected from the group consisting of methyl and cyclopropyl; R² is selected from the group consisting of hydrogen,

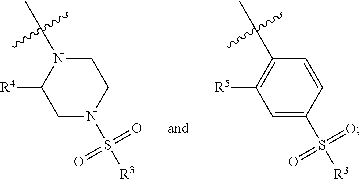

and R³ is selected from the group consisting of:

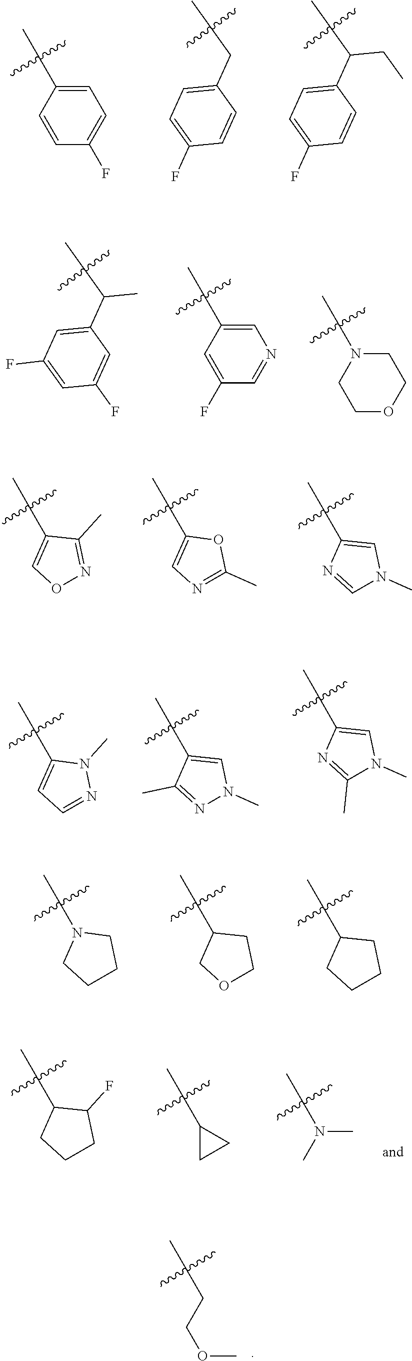

In some embodiments, R¹ is methyl; R² is hydrogen; R⁴ and R⁵ are CF₃; A is selected from:

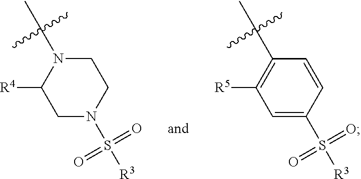

and R³ is selected from the group consisting of:

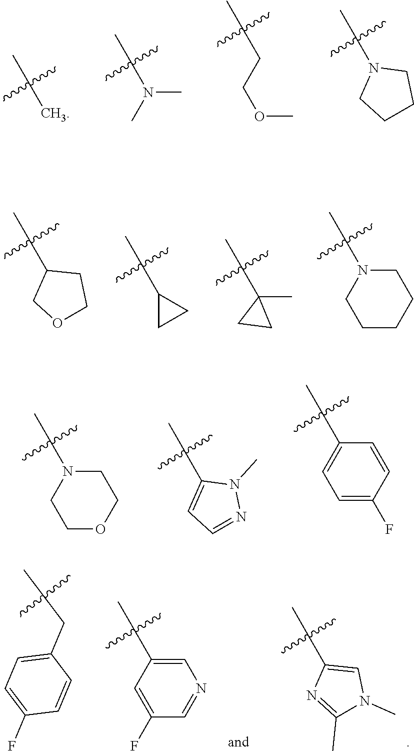

In some embodiments, R¹ is methyl; R² is hydrogen; R⁴ is CF₃; A is

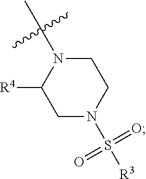

and
R³ is selected from the group consisting of:

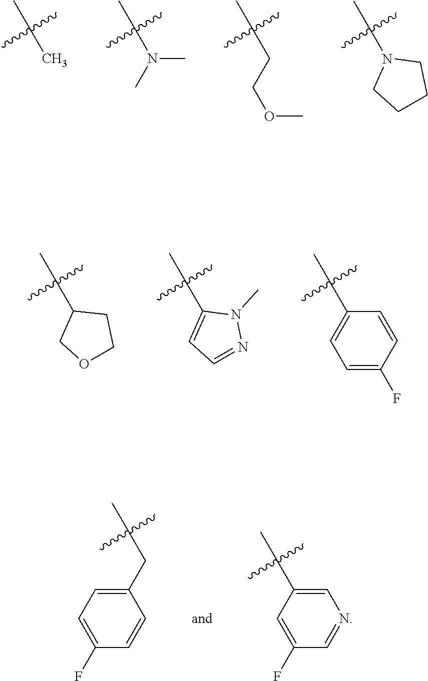

In some embodiments, R¹ is methyl or cyclopropyl; R² is hydrogen; R and R⁵ are CF₃; A is selected from:

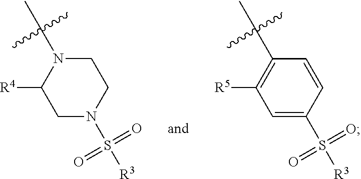

and R³ is selected from the group consisting of:

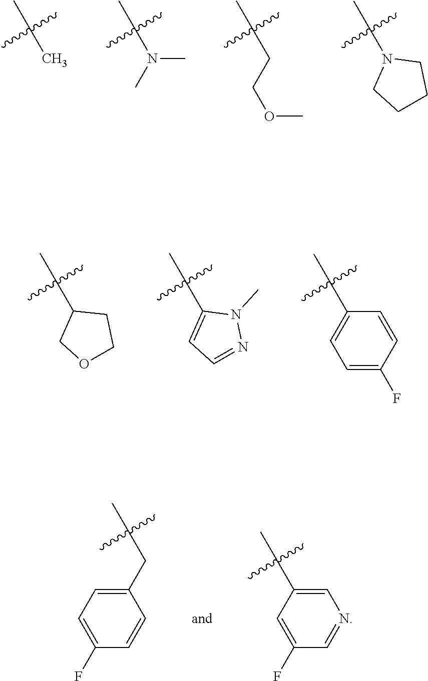

In some embodiments, the compound is selected from the group consisting of: 4-(3-methylmorpholin-4-yl)-6-[4-methylsulfonyl-2-(trifluoromethyl)piperazin-1-yl]-1H-pyridin-2-one; 6-[4-[(4-Fluorophenyl)methylsulfonyl]-2-(trifluoromethyl)piperazin-1-yl]-4-(3-methylmorpholin-4-yl)-1H-pyridin-2-one; 6-[4-[(5-Fluoro-3-pyridyl)sulfonyl]-2-(trifluoromethyl)piperazin-1-yl]-4-(3-methylmorpholin-4-yl)-1H-pyridin-2-one; 4-(3-methylmorpholin-4-yl)-6-[4-tetrahydrofuran-3-ylsulfonyl-2-(trifluoromethyl)piperazin-1-yl]-1H-pyridin-2-one; 4-(3-methylmorpholin-4-yl)-6-[4-pyrrolidin-1-ylsulfonyl-2-(trifluoromethyl)piperazin-1-yl]-1H-pyridin-2-one; N,N-dimethyl-4-[4-(3-methylmorpholin-4-yl)-6-oxo-1H-pyridin-2-yl]-3-(trifluoromethyl)piperazine-1-sulfonamide; 6-[4-(2-methoxyethylsulfonyl)-2-(trifluoromethyl)piperazin-1-yl]-4-(3-methylmorpholin-4-yl)-1H-pyridin-2-one; 6-[4-(4-fluorophenyl)sulfonyl-2-(trifluoromethyl)piperazin-1-yl]-4-(3-methylmorpholin-4-yl)-1H-pyridin-2-one; 4-(3-methylmorpholin-4-yl)-6-[4-(2-methylpyrazol-3-yl)sulfonyl-2-(trifluoromethyl)piperazin-1-yl]-1H-pyridin-2-one; 6-[4-Cyclopropylsulfonyl-2-(trifluoromethyl)piperazin-1-yl]-4-(3-methylmorpholin-4-yl)-1H-pyridin-2-one; 4-(3-methylmorpholin-4-yl)-6-[4-(1-piperidylsulfonyl)-2-(trifluoromethyl)piperazin-1-yl]-1H-pyridin-2-one; 4-(3-methylmorpholin-4-yl)-6-[4-morpholinosulfonyl-2-(trifluoromethyl)piperazin-1-yl]-1H-pyridin-2-one; 6-[4-(1,2-Dimethylimidazol-4-yl)sulfonyl-2-(trifluoromethyl)piperazin-1-yl]-4-(3-methylmorpholin-4-yl)-1H-pyridin-2-one; 6-[4-(1-methylcyclopropyl)sulfonyl-2-(trifluoromethyl)piperazin-1-yl]-4-(3-methylmorpholin-4-yl)-1H-pyridin-2-one; 4-(3-methylmorpholin-4-yl)-6-[4-methylsulfonyl-2-(trifluoromethyl)phenyl]-1H-pyridin-2-one; N,N-dimethyl-4-[4-(3-methylmorpholin-4-yl)-6-oxo-1H-pyridin-2-yl]-3-(trifluoromethyl)benzenesulfonamide; and pharmaceutically acceptable salts, stereoisomers, and tautomers thereof.

In some embodiments, the compound is selected from the group consisting of: 4-(3-methylmorpholin-4-yl)-6-[4-methylsulfonyl-2-(trifluoromethyl)piperazin-1-yl]-1H-pyridin-2-one; 6-[4-[(4-Fluorophenyl)methylsulfonyl]-2-(trifluoromethyl)piperazin-1-yl]-4-(3-methylmorpholin-4-yl)-1H-pyridin-2-one; 6-[4-[(5-Fluoro-3-pyridyl)sulfonyl]-2-(trifluoromethyl)piperazin-1-yl]-4-(3-methylmorpholin-4-yl)-1H-pyridin-2-one; 4-(3-methylmorpholin-4-yl)-6-[4-tetrahydrofuran-3-ylsulfonyl-2-(trifluoromethyl)piperazin-1-yl]-H-pyridin-2-one; 4-(3-methylmorpholin-4-yl)-6-[4-pyrrolidin-1-ylsulfonyl-2-(trifluoromethyl)piperazin-1-yl]-1H-pyridin-2-one; N,N-dimethyl-4-[4-(3-methylmorpholin-4-yl)-6-oxo-1H-pyridin-2-yl]-3-(trifluoromethyl)piperazine-1-sulfonamide; 6-[4-(2-methoxyethylsulfonyl)-2-(trifluoromethyl)piperazin-1-yl]-4-(3-methylmorpholin-4-yl)-1H-pyhdin-2-one; 6-[4-(4-fluorophenyl)sulfonyl-2-(trifluoromethyl)piperazin-1-yl]-4-(3-methylmorpholin-4-yl)-1H-pyridin-2-one; 4-(3-methylmorpholin-4-yl)-6-[4-(2-methylpyrazol-3-yl)sulfonyl-2-(trifluoromethyl)piperazin-1-yl]-1H-pyridin-2-one; and pharmaceutically acceptable salts, stereoisomers, and tautomers thereof.

### Methods of Treatment

Described herein, in another embodiment, is a method of treating a disorder in a patient in need thereof, comprising administering to the patient a therapeutically effective amount of a VPS34 inhibitor and one or more additional therapeutic agents. The disorder may be cancer.

Described herein, in another embodiment, is a method of treating a disorder in a patient in need thereof, comprising administering to the patient a therapeutically effective amount of a compound described herein and one or more additional therapeutic agents. The disorder may be cancer.

Non-limiting examples of cancers of the present disclosure include gastrointestinal stromal tumors, a esophageal cancer, a gastric cancer, a melanoma, a glioma, a glioblastoma, an ovarian cancer, a bladder cancer, a head cancer, a neck cancer, a urothelial cancer, a uterine cancer, a pancreatic cancer, a prostate cancer, a lung cancer, a breast cancer, a renal cancer, a hepatic cancer, an osteosarcoma, a sarcoma, a multiple myeloma, a cervical carcinoma, a cancer that is metastatic to bone, a papillary thyroid carcinoma, a non-small cell lung cancer, a lymphoma, a leukemia, and a colorectal cancer.

In some embodiments, the cancerous cell is of a cancer is selected from the group consisting of gastrointestinal stromal tumors, a esophageal cancer, a gastric cancer, a melanoma, a glioma, a glioblastoma, an ovarian cancer, a bladder cancer, a head cancer, a neck cancer, a urothelial cancer, a uterine cancer, a pancreatic cancer, a prostate cancer, a lung cancer, a breast cancer, a renal cancer, a hepatic cancer, an osteosarcoma, a sarcoma, a multiple myeloma, a cervical carcinoma, a cancer that is metastatic to bone, a papillary thyroid carcinoma, a non-small cell lung cancer, a lymphoma, a leukemia, and a colorectal cancer.

In some embodiments, the cancer is selected from the group consisting of a renal cancer and a melanoma. In some embodiments, the renal cancer is renal cell carcinoma such as clear-cell renal cell carcinoma.

In an aspect, provided herein is a method of treating cancer in a patient in need thereof, comprising: (i) administering to the patient a therapeutically effective amount of a compound represented by Formula I:

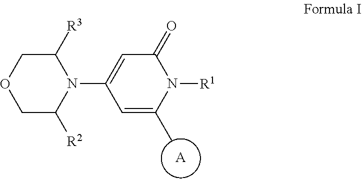

or a pharmaceutically acceptable salt, stereoisomer, or tautomer thereof, wherein: R¹, R², and R³ are independently selected from the group consisting of H, C₁-C₃haloalkyl, and C₁-C₃alkyl; A represents:

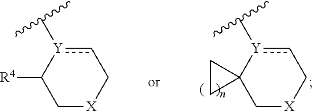

[symbol]

is a single bond or a double bond; X is selected from the group consisting of CH₂, S, SO, SO₂, NR⁵, NCOR⁵, NCOR⁹, NCOCH₂R⁹, O, and a bond; Y is selected from the group consisting of N, CH, and C, provided that, when Y is CH, [symbol] is a single bond; n is selected from 1, 2, 3 and 4; R⁴ is selected from the group consisting of H, halogen, COR⁶, C₁-C₆alkyl, C₁-C₃alkoxyC₁-C₃alkyl, C₁-C₆alkoxy, C₃-C₆cycloalkyl, C₃-C₆heterocyclyl, C₁-C₃cyanoalkyl, C₁-C₃haloalkyl, aryl, and heteroaryl, wherein said aryl and said heteroaryl are optionally substituted with one or more R⁷; R⁵ is selected from the group consisting of H, C₁-C₃fluoroalkyl, C₁-C₃alkyl, C₁-C₃alkoxyC₁-C₃alkyl, and C₃-C₆cycloalkyl; R⁶ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; each R⁷ is independently selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy and C₁-C₃alkoxy; R⁹ is selected from the group consisting of C₁-C₃alkyl, C₁-C₃alkoxy, C₃-C₆cycloalkyl, heterocyclyl, phenyl and a monocyclic heteroaryl, wherein said heterocyclyl, said phenyl and said monocyclic heteroaryl are optionally substituted with one or two R⁸; and each R⁸ is independently selected from the group consisting of halogen, C₁-C₃haloalkyl and C₁-C₃alkyl; and (ii) administering to the patient a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

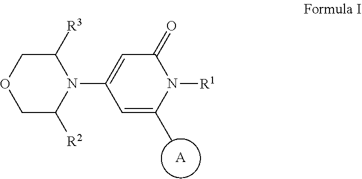

or a pharmaceutically acceptable salt, stereoisomer, or tautomer thereof, wherein: R¹, R², and R³ are independently selected from the group consisting of H, C₁-C₃haloalkyl, and C₁-C₃alkyl; A represents:

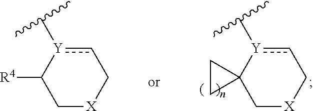

[symbol]

is a single bond or a double bond; X is selected from the group consisting of CH₂, S, SO, SO₂, NR⁵, NCOR⁵, NCOR⁹, NCOCH₂R⁹, O, and a bond; Y is selected from the group consisting of N, CH, and C, provided that, when Y is CH, [symbol] is a single bond; n is selected from 1, 2, 3 and 4; R⁴ is selected from the group consisting of H, halogen, COR⁶, C₁-C₆alkyl, C₁-C₃alkoxyC₁-C₃alkyl, C₁-C₆alkoxy, C₃-C₆cycloalkyl, C₃-C₆heterocyclyl, C₁-C₃cyanoalkyl, C₁-C₃haloalkyl, aryl, and heteroaryl, wherein said aryl and said heteroaryl are optionally substituted with one or more R⁷; R⁵ is selected from the group consisting of H, C₁-C₃fluoroalkyl, C₁-C₃alkyl, C₁-C₃alkoxyC₁-C₃alkyl, and C₃-C₆cycloalkyl; R⁶ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; each R⁷ is independently selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy and C₁-C₃alkoxy; R⁹ is selected from the group consisting of C₁-C₃alkyl, C₁-C₃alkoxy, C₃-C₆cycloalkyl, heterocyclyl, phenyl and a monocyclic heteroaryl, wherein said heterocyclyl, said phenyl and said monocyclic heteroaryl are optionally substituted with one or two R⁸; and each R⁸ is independently selected from the group consisting of halogen, C₁-C₃haloalkyl and C₁-C₃alkyl; in an amount sufficient to induce a Type I interferon response by the cell; and
(ii) a STING agonist in an amount sufficient to increase the expression level of the at least one chemokine in the cell.

In some embodiments, R¹ is H. In some embodiments, R² is H. In some embodiments, R³ is C₁-C₃alkyl. In some embodiments, A is piperidinyl. In some embodiments, R⁴ is C₁-C₃haloalkyl.

In some embodiments, the compound is selected from the group consisting of: 4-morpholino-6-(2-phenylpyrrolidin-1-yl)-1H-pyridin-2-one; 1-methyl-4-morpholino-6-(2-phenylpyrrolidin-1-yl)pyridin-2-one; 4-morpholino-6-[(2S)-2-phenylpyrrolidin-1-yl]-1H-pyridin-2-one; 4-morpholino-6-[(2R)-2-phenylpyrrolidin-1-yl]-1H-pyridin-2-one; 6-(3,6-dihydro-2H-pyran-4-yl)-4-(3-methylmorpholin-4-yl)-1H-pyridin-2-one; 4-(3-methylmorpholin-4-yl)-6-tetrahydropyran-4-yl-1H-pyridin-2-one; 6-[2-(3-methoxyphenyl)pyrrolidin-1-yl]-4-(3-methylmorpholin-4-yl)-1H-pyridin-2-one; 4-(3-methylmorpholin-4-yl)-6-[2-(3-pyridyl)pyrrolidin-1-yl]-1H-pyridin-2-one; 4-(3-methylmorpholin-4-yl)-6-(2-phenylpyrrolidin-1-yl)-1H-pyridin-2-one; N,N-dimethyl-1-[4-[(3R)-3-methylmorpholin-4-yl]-6-oxo-1H-pyridin-2-yl]pyrrolidine-2-carboxamide; 6-[2-(1-methoxy-1-methyl-ethyl)pyrrolidin-1-yl]-4-[(3R)-3-methylmorpholin-4-yl]-1H-pyridin-2-one; 6-(2-cyclohexylpyrrolidin-1-yl)-4-[(3R)-3-methylmorpholin-4-yl]-1H-pyridin-2-one; 6-[2-(3-fluorophenyl)pyrrolidin-1-yl]-4-[(3R)-3-methylmorpholin-4-yl]-1H-pyridin-2-one; 6-[2-(2,5-difluorophenyl)pyrrolidin-1-yl]-4-[(3R)-3-methylmorpholin-4-yl]-1H-pyridin-2-one; 4-[(3R)-3-methylmorpholin-4-yl]-6-[2-[3-(trifluoromethoxy)phenyl]pyrrolidin-1-yl]-1H-pyridin-2-one; 4-[(3R)-3-methylmorpholin-4-yl]-6-[2-[3-(trifluoromethyl)phenyl]pyrrolidin-1-yl]-1H-pyridin-2-one; 6-[2-(3-methoxyphenyl)pyrrolidin-1-yl]-4-[(3R)-3-methylmorpholin-4-yl]-1H-pyridin-2-one; 4-[(3R)-3-methylmorpholin-4-yl]-6-(2-phenylpyrrolidin-1-yl)-1H-pyridin-2-one; 4-[(3R)-3-methylmorpholin-4-yl]-6-[2-(1-methylpyrazol-4-yl)pyrrolidin-1-yl]-1H-pyridin-2-one; 6-[2-(1,5-dimethylpyrazol-3-yl)pyrrolidin-1-yl]-4-[(3R)-3-methylmorpholin-4-yl]-1H-pyridin-2-one; 6-[2-(1-ethylpyrazol-3-yl)pyrrolidin-1-yl]-4-[(3R)-3-methylmorpholin-4-yl]-1H-pyridin-2-one; 6-[2-(5-methyl-2-furyl)pyrrolidin-1-yl]-4-[(3R)-3-methylmorpholin-4-yl]-1H-pyridin-2-one; 6-[2-[3-(dimethylamino)phenyl]pyrrolidin-1-yl]-4-[(3R)-3-methylmorpholin-4-yl]-1H-pyridin-2-one; 4-[(3R)-3-methylmorpholin-4-yl]-6-(3-methylmorpholin-4-yl)-1H-pyridin-2-one; 4-[(3R)-3-methylmorpholin-4-yl]-6-[2-(trifluoromethyl)-1-piperidyl]-1H-pyridin-2-one; 4-[(3R)-3-methylmorpholin-4-yl]-6-(3-phenylmorpholin-4-yl)-1H-pyridin-2-one; 4-[(3R)-3-methylmorpholin-4-yl]-6-(1-oxo-1,4-thiazinan-4-yl)-1H-pyridin-2-one; 6-(1,1-dioxo-1,4-thiazinan-4-yl)-4-[(3R)-3-methylmorpholin-4-yl]-1H-pyridin-2-one; 6-(4-acetylpiperazin-1-yl)-4-[(3R)-3-methylmorpholin-4-yl]-1H-pyridin-2-one; 4-[(3R)-3-methylmorpholin-4-yl]-6-[(2R)-2-phenyl-1-piperidyl]-1H-pyridin-2-one; 4-[(3R)-3-methylmorpholin-4-yl]-6-(4-methyl-2-phenyl-piperazin-1-yl)-1H-pyridin-2-one; 4-[(3R)-3-methylmorpholin-4-yl]-6-[3-(trifluoromethyl)morpholin-4-yl]-1H-pyridin-2-one; 6-(3-cyclopropylmorpholin-4-yl)-4-[(3R)-3-methylmorpholin-4-yl]-1H-pyridin-2-one; 4-[(3R)-3-methylmorpholin-4-yl]-6-[(2S)-2-(trifluoromethyl)pyrrolidin-1-yl]-1H-pyridin-2-one; 4-[(3R)-3-methylmorpholin-4-yl]-6-[(2R)-2-(trifluoromethyl)pyrrolidin-1-yl]-1H-pyridin-2-one; 6-[2-(3-chlorophenyl)pyrrolidin-1-yl]-4-[(3R)-3-methylmorpholin-4-yl]-1H-pyridin-2-one; 6-[2-(3-cyclopropylphenyl)pyrrolidin-1-yl]-4-[(3R)-3-methylmorpholin-4-yl]-1H-pyridin-2-one; 4-[(3R)-3-methylmorpholin-4-yl]-6-[2-(2-pyridyl)pyrrolidin-1-yl]-1H-pyridin-2-one; 4-[(3R)-3-methylmorpholin-4-yl]-6-(2-thiazol-2-ylpyrrolidin-1-yl)-1H-pyridin-2-one; 6-[2-(5-methylisoxazol-3-yl)pyrrolidin-1-yl]-4-[(3R)-3-methylmorpholin-4-yl]-1H-pyridin-2-one; 1-methyl-4-[(3R)-3-methylmorpholin-4-yl]-6-[(2R)-2-(trifluoromethyl)-1-piperidyl]pyridin-2-one; 4-[(3R)-3-methylmorpholin-4-yl]-6-(8-oxa-5-azaspiro[3.5]nonan-5-yl)-1H-pyridin-2-one; 6-[2-(3-methoxyphenyl)-1-piperidyl]-4-[(3R)-3-methylmorpholin-4-yl]-1H-pyridin-2-one; 6-[4-acetyl-2-(trifluoromethyl)piperazin-1-yl]-4-[(3R)-3-methylmorpholin-4-yl]-1H pyridin-2-one; 6-[4-(5-fluoropyridine-3-carbonyl)-2-(trifluoromethyl)piperazin-1-yl]-4-[(3R)-3-methylmorpholin-4-yl]-1H-pyridin-2-one; 6-[4-[2-(4-fluorophenyl)acetyl]-2-(trifluoromethyl)piperazin-1-yl]-4-[(3R)-3-methylmorpholin-4-yl]-1H-pyridin-2-one; 4-[(3R)-3-methylmorpholin-4-yl]-6-[4-(tetrahydrofuran-2-carbonyl)-2-(trifluoromethyl)piperazin-1-yl]-1H-pyridin-2-one; 4-[(3R)-3-methylmorpholin-4-yl]-6-[4-methyl-2-(trifluoromethyl)piperazin-1-yl]-1H-pyridin-2-one; and pharmaceutically acceptable salts, tautomers, and stereoisomers thereof.

In some embodiments, the compound is selected from the group consisting of:

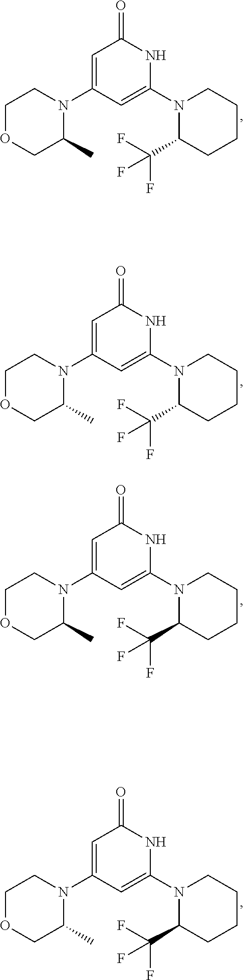

and pharmaceutically acceptable salts thereof.

In some embodiments, the compound is selected from the group consisting of:

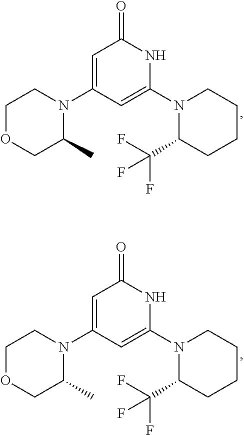 [SMILES: C[C@H]1COCCN1c1cc(N2CCCC[C@@H]2C(F)(F)F)[nH]c(=O)c1 ; C[C@@H]1COCCN1c1cc(N2CCCC[C@@H]2C(F)(F)F)[nH]c(=O)c1]

and pharmaceutically acceptable salts thereof.

In some embodiments, the compound is selected from the group consisting of:

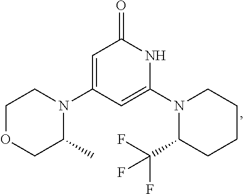 [SMILES: C[C@@H]1COCCN1c1cc(N2CCCC[C@@H]2C(F)(F)F)[nH]c(=O)c1]

and pharmaceutically acceptable salts thereof.

In some embodiments, the at least one chemokine is selected from the group consisting of CCL5 and CXCL10.

In an aspect, provided herein is a method of treating cancer in a patient in need thereof, comprising administering to the patient: (i) a therapeutically effective amount of a compound represented by Formula II:

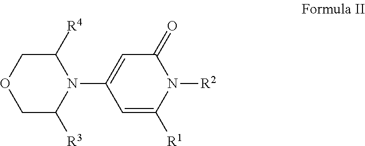

or a pharmaceutically acceptable salt, stereoisomer, or tautomer thereof, wherein: R¹ is selected from the group consisting of aryl and heteroaryl, wherein said aryl and said heteroaryl being mono- or bicyclic and each of aryl and heteroaryl is optionally substituted with one or more independent occurrences of a substituent selected from the group consisting of R⁵, R⁶, R⁷ and R⁸; each of R², R³, R⁴ is independently selected from the group consisting of H, C₁-C₃haloalkyl, and C₁-C₃alkyl; each of R⁵, R⁶, R⁷, and R⁸ is independently selected from the group consisting of halogen, C₁-C₆alkyl, C₁-C₆alkoxy, C₁-C₆haloalkyl, amino, —NHSO₂R⁹, hydroxy, phenyl, and a monocyclic heteroaryl; and R⁹ is selected from C₁-C₃haloalkyl and C₁-C₃alkyl; and (ii) a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

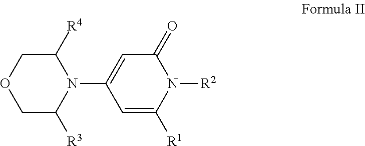

or a pharmaceutically acceptable salt, stereoisomer, or tautomer thereof, wherein: R¹ is selected from the group consisting of aryl and heteroaryl, wherein said aryl and said heteroaryl being mono- or bicyclic and each of aryl and heteroaryl is optionally substituted with one or more independent occurrences of a substituent selected from the group consisting of R⁵, R⁶, R⁷ and R⁸; each of R², R³, R⁴ is independently selected from the group consisting of H, C₁-C₃haloalkyl, and C₁-C₃alkyl; each of R⁵, R⁶, R⁷, and R⁸ is independently selected from the group consisting of halogen, C₁-C₆alkyl, C₁-C₆alkoxy, C₁-C₆haloalkyl, amino, —NHSO₂R⁹, hydroxy, phenyl, and a monocyclic heteroaryl; and R⁹ is selected from C₁-C₃haloalkyl and C₁-C₃alkyl; in an amount sufficient to induce a Type I interferon response by the cell; and (ii) a STING agonist in an amount sufficient to increase the expression level of the at least one chemokine in the cell.

In some embodiments, the compound is selected from the group consisting of:

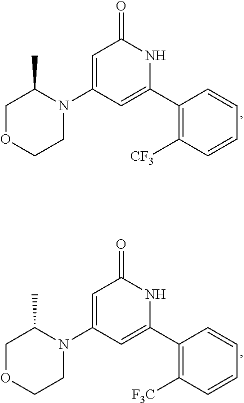 [SMILES: C[C@@H]1COCCN1c1cc(-c2ccccc2C(F)(F)F)[nH]c(=O)c1 ; C[C@H]1COCCN1c1cc(-c2ccccc2C(F)(F)F)[nH]c(=O)c1]

and pharmaceutically acceptable salts thereof.

In some embodiments, the compound is selected from the group consisting of: 6-(2-chlorophenyl)-4-morpholino-1H-pyridin-2-one; 6-(2-chlorophenyl)-1-methyl-4-morpholino-pyridin-2-one; 6-(2-chlorophenyl)-4-(3-methylmorpholin-4-yl)-1H-pyridin-2-one; 6-(2-chlorophenyl)-1-methyl-4-(3-methylmorpholin-4-yl)pyridin-2-one; 4-(3-methylmorpholin-4-yl)-6-(4-methyl-3-pyridyl)-1H-pyridin-2-one; 4-(3-methylmorpholin-4-yl)-6-pyrimidin-5-yl-1H-pyridin-2-one; 4-(3-methylmorpholin-4-yl)-6-(2-phenylphenyl)-1H-pyridin-2-one; 6-(2-chloro-5-fluoro-phenyl)-4-[(3R)-3-methylmorpholin-4-yl]-1H-pyridin-2-one; 4-[(3R)-3-methylmorpholin-4-yl]-6-(o-tolyl)-1H-pyridin-2-one; 4-[(3R)-3-methylmorpholin-4-yl]-6-[2-(trifluoromethyl)-3-pyridyl]-1H-pyridin-2-one; 6-(2-chlorophenyl)-4-[(3R)-3-methylmorpholin-4-yl]-1H-pyridin-2-one; 4-[(3R)-3-methylmorpholin-4-yl]-6-[2-(trifluoromethyl)phenyl]-1H-pyridin-2-one; 6-(3-furyl)-4-[(3R)-3-methylmorpholin-4-yl]-1H-pyridin-2-one; 4-[(3R)-3-methylmorpholin-4-yl]-6-(4-methyl-3-thienyl)-1H-pyridin-2-one; N-[2-[4-[(3R)-3-methylmorpholin-4-yl]-6-oxo-1H-pyridin-2-yl]phenyl]methanesulfonamide; 4-[(3R)-3-methylmorpholin-4-yl]-6-(4-(methylsulfonyl)-2-(trifluoromethyl)phenyl)-1H-pyridin-2-one; 4-[(3R)-3-methylmorpholin-4-yl]-6-(6-methyl-5-quinolyl)-1H-pyridin-2-one; 4-[(3R)-3-methylmorpholin-4-yl]-6-[4-(1H-pyrazol-5-yl)phenyl]-1H-pyridin-2-one; N,N-dimethyl-[4[4-[(3R)-3-methylmorpholin-4-yl]-6-oxo-1H-pyridin-2-yl]-3-(trifluoromethyl)]benzenesulfonamide; and pharmaceutically acceptable salts, tautomers, and stereoisomers thereof.

In some embodiments, the at least one chemokine is selected from the group consisting of CCL5 and CXCL10.

In an aspect, provided herein is a method of treating cancer in a patient in need thereof, comprising administering to the patient: (i) a therapeutically effective amount of a compound represented by Formula III:

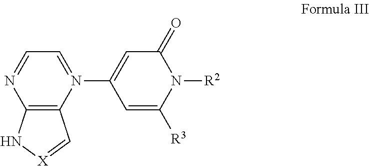

or a pharmaceutically acceptable salt, stereoisomer, or tautomer thereof, wherein: X is selected from N and CR¹; R¹ is selected from the group consisting of H, C₁-C₃alkyl, C₁-C₃haloalkyl, C₁-C₃alkoxyC₁-C₃alkyl, C₃-C₆cycloalkyl, cyano, phenyl, and monocyclic heteroaryl, wherein each of phenyl and monocyclic heteroaryl is optionally substituted with one or more substituents independently selected from the group consisting of halo, N—C₁-C₃alkylamino, N,N-diC₁-C₃alkylamino, C₃-C₆cycloalkyl, C₁-C₃alkoxyC₁-C₃alkyl, C₁-C₃haloalkyl, C₁-C₃haloalkoxy, C₁-C₃alkoxy, and C₁-C₃alkyl; R² is selected from the group consisting of H, C₁-C₃haloalkyl, and C₁-C₃alkyl; R³ is selected from the group consisting of A, phenyl, and monocyclic heteroaryl, wherein each of phenyl and monocyclic heteroaryl is optionally substituted with one or more occurrences of R⁴; each R⁴ is independently selected from the group consisting of COR⁵, halogen, C₁-C₆alkyl, C₁-C₆alkoxy, C₁-C₆haloalkoxy, amino N—C₁-C₃alkylamino, N,N-diC₁-C₃alkylamino, 1-pyrrolidinyl, 1-piperidinyl, 1-azetidinyl, NHSO₂R⁶, SO₂R⁷, hydroxy, C₃-C₆cycloalkyl, C₁-C₃alkoxyC₁-C₃alkyl, C₁-C₃cyanoalkyl and C₁-C₆haloalkyl; R⁵ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; R⁶ is selected from C₁-C₃haloalkyl and C₁-C₃alkyl; each R⁷ is independently selected from the group consisting of R⁸, C₁-C₆alkyl, N—C₁-C₃alkylamino, N,N-diC₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen; each R⁸ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁹; each R⁹ is independently selected from the group consisting of halo, N—C₁-C₃alkylamino, N,N-diC₁-C₃alkylamino, C₁-C₃alkoxyC₁-C₃alkyl, amino, C₁-C₃haloalkyl, C₁-C₃alkoxy, C₁-C₃haloalkoxy, C₃-C₆cycloalkyl and C₁-C₃alkyl; A is

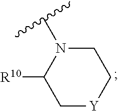

R¹⁰ is selected from the group consisting of H, halogen, COR¹¹, C₁-C₆alkyl, C₁-C₃alkoxyC₁-C₃alkyl, C₁-C₆alkoxy, C₃-C₆cycloalkyl, C₁-C₃cyanoalkyl, C₁-C₃haloalkyl, phenyl, and heteroaryl, wherein each of phenyl and heteroaryl is optionally substituted with one or more occurrences of R¹², and provided that when R¹⁰ is phenyl or heteroaryl, then X is N or CH; each R¹¹ is independently selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹³, NCOR⁷, NCOOR¹⁴, NSO₂R⁷, NCOCH₂R⁷, O, and a bond; R¹² is selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy, and C₁-C₃alkoxy; R¹³ is selected from H, C₁-C₃haloalkyl, C₁-C₃alkoxyC₁-C₃alkyl, C₁-C₃alkyl, C₃-C₆cycloalkyl; and R¹⁴ is selected from R⁸, C₁-C₆alkyl, C₁-C₃alkoxyC₁-C₃alkyl, wherein each of C₁-C₃alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen; and (ii) a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

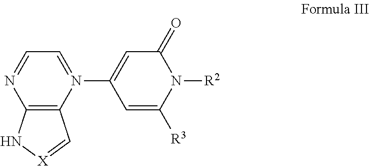

or a pharmaceutically acceptable salt, stereoisomer, or tautomer thereof, wherein: X is selected from N and CR¹; R¹ is selected from the group consisting of H, C₁-C₃alkyl, C₁-C₃haloalkyl, C₁-C₃alkoxyC₁-C₃alkyl, C₃-C₆cycloalkyl, cyano, phenyl, and monocyclic heteroaryl, wherein each of phenyl and monocyclic heteroaryl is optionally substituted with one or more substituents independently selected from the group consisting of halo, N—C₁-C₃alkylamino, N,N-diC₁-C₃alkylamino, C₃-C₆cycloalkyl, C₁-C₃alkoxyC₁-C₃alkyl, C₁-C₃haloalkyl, C₁-C₃haloalkoxy, C₁-C₃alkoxy, and C₁-C₃alkyl; R² is selected from the group consisting of H, C₁-C₃haloalkyl, and C₁-C₃alkyl; R³ is selected from the group consisting of A, phenyl, and monocyclic heteroaryl, wherein each of phenyl and monocyclic heteroaryl is optionally substituted with one or more occurrences of R⁴; each R⁴ is independently selected from the group consisting of COR⁵, halogen, C₁-C₆alkyl, C₁-C₆alkoxy, C₁-C₆haloalkoxy, amino N—C₁-C₃alkylamino, N,N-diC₁-C₃alkylamino, 1-pyrrolidinyl, 1-piperidinyl, 1-azetidinyl, NHSO₂R⁶, SO₂R⁷, hydroxy, C₃-C₆cycloalkyl, C₁-C₃alkoxyC₁-C₃alkyl, C₁-C₃cyanoalkyl and C₁-C₆haloalkyl; R⁵ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; R⁶ is selected from C₁-C₃haloalkyl and C₁-C₃alkyl; each R⁷ is independently selected from the group consisting of R⁸, C₁-C₆alkyl, N—C₁-C₃alkylamino, N,N-diC₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen; each R⁸ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁹; each R⁹ is independently selected from the group consisting of halo, N—C₁-C₃alkylamino, N,N-diC₁-C₃alkylamino, C₁-C₃alkoxyC₁-C₃alkyl, amino, C₁-C₃haloalkyl, C₁-C₃alkoxy, C₁-C₃haloalkoxy, C₃-C₆cycloalkyl and C₁-C₃alkyl; A is

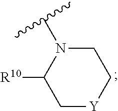

R¹⁰ is selected from the group consisting of H, halogen, COR¹¹, C₁-C₆alkyl, C₁-C₃alkoxyC₁-C₃alkyl, C₁-C₆alkoxy, C₃-C₆cycloalkyl, C₁-C₃cyanoalkyl, C₁-C₃haloalkyl, phenyl, and heteroaryl, wherein each of phenyl and heteroaryl is optionally substituted with one or more occurrences of R¹², and provided that when R¹⁰ is phenyl or heteroaryl, then X is N or CH; each R¹¹ is independently selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹³, NCOR⁷, NCOOR¹⁴, NSO₂R⁷, NCOCH₂R⁷, O, and a bond; R¹² is selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy, and C₁-C₃alkoxy; R¹³ is selected from H, C₁-C₃haloalkyl, C₁-C₃alkoxyC₁-C₃alkyl, C₁-C₃alkyl, C₃-C₆cycloalkyl; and R¹⁴ is selected from R⁸, C₁-C₆alkyl, C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen; and in an amount sufficient to induce a Type I interferon response by the cell; and (ii) a STING agonist in an amount sufficient to increase the expression level of the at least one chemokine in the cell.

In some embodiments, the compound is selected from the group consisting of:

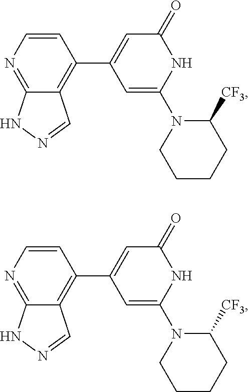

and pharmaceutically acceptable salts thereof.

In some embodiments, the compound is selected from the group consisting of: 4-(1H-pyrrolo[2,3-b]pyridin-4-yl)-6-[2-(trifluoromethyl)phenyl]-1H-pyridin-2-one; 6-(3-Methyl-4-pyridyl)-4-(1H-pyrrolo[2,3-b]pyridin-4-yl)-1H-pyridin-2-one; 6-(2-phenylpyrrolidin-1-yl)-4-(1H-pyrrolo[2,3-b]pyridin-4-yl)-1H-pyridin-2-one; 4-(2-Methyl-1H-pyrrolo[2,3-b]pyridin-4-yl)-6-(3-pyridyl)-1H-pyridin-2-one; 4-(2-Methyl-1H-pyrrolo[2,3-b]pyridin-4-yl)-6-morpholino-1H-pyridin-2-one; 6-(2-Chlorophenyl)-4-(2-oxazol-5-yl-1H-pyrrolo[2,3-b]pyridin-4-yl)-1H-pyridin-2-one; 6-(2-Chlorophenyl)-4-[2-(3-pyridyl)-1H-pyrrolo[2,3-b]pyridin-4-yl]-1H-pyridin-2-one; 6-(2-Chlorophenyl)-4-(2-methyl-1H-pyrrolo[2,3-b]pyridin-4-yl)-1H-pyridin-2-one; 4-(2-Methyl-1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[3-(trifluoromethyl)morpholin-4-yl]-1H-pyridin-2-one; 6-[3-(Trifluoromethyl)morpholin-4-yl]-4-[2-[3-(trifluoromethyl)phenyl]-1H-pyrrolo[2,3-b]pyridin-4-yl]-1H-pyridin-2-one; 4-[2-(5-Methyl-2-thienyl)-1H-pyrrolo[2,3-b]pyridin-4-yl]-6-[3-(trifluoromethyl)morpholin-4-yl]-1H-pyridin-2-one; 4-(1H-pyrazolo[3,4-b]pyridin-4-yl)-6-[2-(trifluoromethyl)-1-piperidyl]-1H-pyridin-2-one; 6-[2-(Trifluoromethyl)-1-piperidyl]-4-yl]-1H-pyrrolo[2,3-b]pyridin-4-yl]-1H-pyridin-2-one; 6-[2-(Trifluoromethyl)-1-piperidyl]-4-[2-[6-(trifluoromethyl)-3-pyridyl]-1H-pyrrolo[2,3-b]pyridin-4-yl]-1H-pyridin-2-one; 6-[2-(Trifluoromethyl)-1-piperidyl]-4-[2-[5-(trifluoromethyl)-3-pyridyl]-1H-pyrrolo[2,3-b]pyridin-4-yl]-1H-pyridin-2-one; 4-(2-cyclopropyl-1H-pyrrolo[2,3-b]pyridin-4-yl)-6-[4-ethylsulfonyl-2-(trifluoromethyl)piperazin-1-yl]-1H-pyridin-2-one; 6-[4-ethylsulfonyl-2-(trifluoromethyl)piperazin-1-yl]-4-(1H-pyrrolo[2,3-b]pyridin-4-yl)-1H-pyridin-2-one; 6-[4-[(4-fluorophenyl)methylsulfonyl]-2-(trifluoromethyl)piperazin-1-yl]-4-(1H-pyrrolo[2,3-b]pyridin-4-yl)-1H-pyridin-2-one; 6-[4-Ethylsulfonyl-2-(trifluoromethyl)piperazin-1-yl]-4-(2-methyl-1H-pyrrolo[2,3-b]pyridin-4-yl)-1H-pyridin-2-one; 4-[2-[4-Ethylsulfonyl-2-(trifluoromethyl)piperazin-1-yl]-6-oxo-1H-pyridin-4-yl]-1H-pyrrolo[2,3-b]pyridine-2-carbonitrile; 4-(2-cyclopropyl-1H-pyrrolo[2,3-b]pyridin-4-yl)-6-[2-(trifluoromethyl)phenyl]-1H-pyridin-2-one; 4-[2-Oxo-6-[2-(trifluoromethyl)phenyl]-1H-pyridin-4-yl]-1H-pyrrolo[2,3-b]pyridine-2-carbonitrile; 4-(1H-pyrazolo[3,4-b]pyridin-4-yl)-6-[2-(trifluoromethyl)phenyl]-1H-pyridin-2-one; 4-(6-(2-chlorophenyl)-2-oxo-1,2-dihydropyridin-4-yl)-N-ethyl-1H-pyrrolo[2,3-b]pyridine-2-carboxamide; 6-[4-Methylsulfonyl-2-(trifluoromethyl)piperazin-1-yl]-4-(1H-pyrazolo[3,4-b]pyridin-4-yl)-1H-pyridin-2-one; and pharmaceutically acceptable salts, stereoisomers, and tautomers thereof.

In some embodiments, the compound is selected from the group consisting of: 4-(1H-pyrrolo[2,3-b]pyridin-4-yl)-6-[2-(trifluoromethyl)phenyl]-1H-pyridin-2-one; 6-(3-Methyl-4-pyridyl)-4-(1H-pyrrolo[2,3-b]pyridin-4-yl)-1H-pyridin-2-one; 6-(2-phenylpyrrolidin-1-yl)-4-(1H-pyrrolo[2,3-b]pyridin-4-yl)-1H-pyridin-2-one; 4-(2-Methyl-1H-pyrrolo[2,3-b]pyridin-4-yl)-6-(3-pyridyl)-1H-pyridin-2-one; 4-(2-Methyl-1H-pyrrolo[2,3-b]pyridin-4-yl)-6-morpholino-1H-pyridin-2-one; 6-(2-Chlorophenyl)-4-(2-oxazol-5-yl-1H-pyrrolo[2,3-b]pyridin-4-yl)-1H-pyridin-2-one; 6-(2-Chlorophenyl)-4-[2-(3-pyridyl)-1H-pyrrolo[2,3-b]pyridin-4-yl]-1H-pyridin-2-one; 6-(2-Chlorophenyl)-4-(2-methyl-1H-pyrrolo[2,3-b]pyridin-4-yl)-1H-pyridin-2-one; 4-(2-Methyl-1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[3-(trifluoromethyl)morpholin-4-yl]-1H-pyridin-2-one; 6-[3-(Trifluoromethyl)morpholin-4-yl]-4-[2-[3-(trifluoromethyl)phenyl]-1H-pyrrolo[2,3-b]pyridin-4-yl]-1H-pyridin-2-one; 4-[2-(5-Methyl-2-thienyl)-1H-pyrrolo[2,3-b]pyridin-4-yl]-6-[3-(trifluoromethyl)morpholin-4-yl]-1H-pyridin-2-one; 4-(1H-pyrazolo[3,4-b]pyridin-4-yl)-6-[2-(trifluoromethyl)-1-pipendyl]-1H-pyridin-2-one; 6-[2-(Trifluoromethyl)-1-piperidyl]-4-yl]-1H-pyrrolo[2,3-b]pyridin-4-yl]-1H-pyridin-2-one; 6-[2-(Trifluoromethyl)-1-piperidyl]-4-[2-[6-(trifluoromethyl)-3-pyridyl]-1H-pyrrolo[2,3-b]pyridin-4-yl]-1H-pyridin-2-one; 6-[2-(Trifluoromethyl)-1-piperidyl]-4-[2-[5-(trifluoromethyl)-3-pyridyl]-1H-pyrrolo[2,3-b]pyridin-4-yl]-1H-pyridin-2-one; 4-(2-cyclopropyl-1H-pyrrolo[2,3-b]pyridin-4-yl)-6-[4-ethylsulfonyl-2-(trifluoromethyl)piperazin-1-yl]-1H-pyridin-2-one; 6-[4-ethylsulfonyl-2-(trifluoromethyl)piperazin-1-yl]-4-(1H-pyrrolo[2,3-b]pyridin-4-yl)-1H-pyridin-2-one; 6-[4-[(4-fluorophenyl)methylsulfonyl]-2-(trifluoromethyl)piperazin-1-yl]-4-(1H-pyrrolo[2,3-b]pyridin-4-yl)-1H-pyridin-2-one; and pharmaceutically acceptable salts, stereoisomers, and tautomers thereof.

In some embodiments, the at least one chemokine is selected from the group consisting of CCL5 and CXCL10.

In an aspect, provided herein is a method of treating cancer in a patient in need thereof, comprising: (i) administering to the patient a therapeutically effective amount of a compound represented by Formula IV:

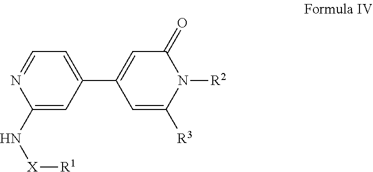

or a pharmaceutically acceptable salt, stereoisomer, or tautomer thereof, wherein: X is selected from —C(═O)— and a bond; R¹ is selected from the group consisting of H, C₁-C₃alkyl, C₁-C₃haloalkyl, C₁-C₃alkoxyC₁-C₃alkyl, C₃-C₆cycloalkyl, C₃-C₆cyclohaloalkyl, C₁-C₃alkoxy, C₁-C₃haloalkoxy, C₃-C₆cycloalkoxymethyl, N—C₁-C₃alkylamino, N,N-diC₁-C₃alkylamino, 1-pyrrolidinyl, 1-piperidinyl and 1-azetidinyl, provided that when R¹ is selected from the group consisting of C₁-C₃alkoxy, C₁-C₃haloalkoxy, N—C₁-C₃alkylamino, N,N-diC₁-C₃alkylamino, 1-pyrrolidinyl, 1-piperidinyl, and 1-azetidinyl, then X is C═O; R² is selected from the group consisting of H, C₁-C₃haloalkyl, and C₁-C₃alkyl; R³ is selected from the group consisting of A, phenyl and monocyclic heteroaryl, wherein each of phenyl and monocyclic heteroaryl is optionally substituted with one or more occurrences of a substituent independently selected from the group consisting of R⁴, R⁵, R⁶ and R⁷; each R⁴, R⁵, R⁶, and R⁷ is independently selected from the group consisting of halo, C₁-C₆alkyl, C₃-C₆cycloalkyl, C₁-C₆alkoxy, C₁-C₃haloalkoxy, N,N-diC₁-C₃alkylamino, N—C₁-C₃alkylamino, 1-azetidinyl, C₁-C₆haloalkyl, amino, NHSO₂R⁸, SO₂R⁹, and hydroxy; R⁸ is selected from C₁-C₃haloalkyl and C₁-C₃alkyl; each R⁹ is independently selected from the group consisting of R¹⁰, C₁-C₆alkyl, amino, N—C₁-C₃alkylamino, N,N-di-C₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; each R¹⁰ is independently selected from the group consisting of phenyl, monocyclic heteroaryl, C₃-C₆cycloalkyl, and heterocyclyl, wherein each phenyl, monocyclic heteroaryl, C₃-C₆cycloalkyl, and heterocyclyl is optionally substituted with one or more occurrences of R¹¹; each R¹¹ is independently selected from the group consisting of halo, C₁-C₃alkoxyC₁-C₃alkyl, amino, N—C₁-C₃alkylamino, N,N-diC₁-C₃alkylamino, C₁-C₃haloalkoxy, C₁-C₃alkoxy, C₃-C₆cycloalkyl, C₁-C₃haloalkyl, and C₁-C₃alkyl; A is:

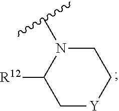

R¹² is selected from the group consisting of H, halo, COR¹³, C₁-C₆alkyl, C₁-C₃alkoxyC₁-C₃alkyl, C₁-C₆alkoxy, C₃-C₆cycloalkyl, C₁-C₃cyanoalkyl, and C₁-C₃haloalkyl; R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹⁵, NSO₂R⁹, NCOCH₂R⁹, O, and a bond; R¹⁴ is selected from the group consisting of H, C₁-C₃haloalkyl, C₁-C₃alkoxyC₁-C₃alkyl, C₁-C₃alkyl, and C₃-C₆cycloalkyl; and R¹⁵ is selected from the group consisting of R¹⁰, C₁-C₆alkyl and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and (ii) administering to the patient a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

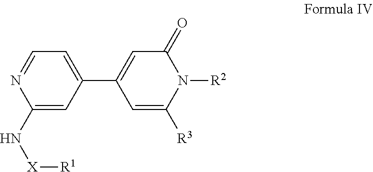

or a pharmaceutically acceptable salt, stereoisomer, or tautomer thereof, wherein: X is selected from —C(═O)— and a bond; R¹ is selected from the group consisting of H, C₁-C₃alkyl, C₁-C₃haloalkyl, C₁-C₃alkoxyC₁-C₃alkyl, C₃-C₆cycloalkyl, C₃-C₆cyclohaloalkyl, C₁-C₃alkoxy, C₁-C₃haloalkoxy, C₃-C₆cycloalkoxymethyl, N—C₁-C₃alkylamino, N,N-diC₁-C₃alkylamino, 1-pyrrolidinyl, 1-piperidinyl and 1-azetidinyl, provided that when R¹ is selected from the group consisting of C₁-C₃alkoxy, C₁-C₃haloalkoxy, N—C₁-C₃alkylamino, N,N-diC₁-C₃alkylamino, 1-pyrrolidinyl, 1-piperidinyl, and 1-azetidinyl, then X is C═O; R² is selected from the group consisting of H, C₁-C₃haloalkyl, and C₁-C₃alkyl; R³ is selected from the group consisting of A, phenyl and monocyclic heteroaryl, wherein each of phenyl and monocyclic heteroaryl is optionally substituted with one or more occurrences of a substituent independently selected from the group consisting of R⁴, R⁵, R⁶ and R⁷; each R⁴, R⁵, R⁶, and R⁷ is independently selected from the group consisting of halo, C₁-C₆alkyl, C₃-C₆cycloalkyl, C₁-C₆alkoxy, C₁-C₃haloalkoxy, N,N-diC₁-C₃alkylamino, N—C₁-C₃alkylamino, 1-azetidinyl, C₁-C₆haloalkyl, amino, NHSO₂R⁸, SO₂R⁹, and hydroxy; R⁸ is selected from C₁-C₃haloalkyl and C₁-C₃alkyl; each R⁹ is independently selected from the group consisting of R¹⁰, C₁-C₆alkyl, amino, N—C₁-C₃alkylamino, N,N-di-C₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; each R¹⁰ is independently selected from the group consisting of phenyl, monocyclic heteroaryl, C₃-C₆cycloalkyl, and heterocyclyl, wherein each phenyl, monocyclic heteroaryl, C₃-C₆cycloalkyl, and heterocyclyl is optionally substituted with one or more occurrences of R¹¹; each R¹¹ is independently selected from the group consisting of halo, C₁-C₃alkoxyC₁-C₃alkyl, amino, N—C₁-C₃alkylamino, N,N-diC₁-C₃alkylamino, C₁-C₃haloalkoxy, C₁-C₃alkoxy, C₃-C₆cycloalkyl, C₁-C₃haloalkyl, and C₁-C₃alkyl; A is:

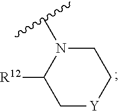

R¹² is selected from the group consisting of H, halo, COR¹³, C₁-C₆alkyl, C₁-C₃alkoxyC₁-C₃alkyl, C₁-C₆alkoxy, C₃-C₀cycloalkyl, C₁-C₃cyanoalkyl, and C₁-C₃haloalkyl; R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹⁵, NSO₂R⁹, NCOCH₂R⁹, O, and a bond; R¹⁴ is selected from the group consisting of H, C₁-C₃haloalkyl, C₁-C₃alkoxyC₁-C₃alkyl, C₁-C₃alkyl, and C₃-C₆cycloalkyl; and R¹⁵ is selected from the group consisting of R¹⁰, C₁-C₆alkyl and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and in an amount sufficient to induce a Type I interferon response by the cell; and (ii) a STING agonist in an amount sufficient to increase the expression level of the at least one chemokine in the cell.

In some embodiments, the compound is selected from the group consisting of:

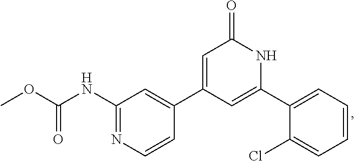 [SMILES: COC(=O)Nc1cc(-c2cc(-c3ccccc3Cl)[nH]c(=O)c2)ccn1]

and pharmaceutically acceptable salts thereof.

In some embodiments, the compound is selected from the group consisting of: N-[4-[2-(2-chlorophenyl)-6-oxo-1H-pyridin-4-yl]-2-pyridyl]acetamide; 4-(2-Amino-4-pyridyl)-6-(3-pyridyl)-1H-pyridin-2-one; 4-(2-Amino-4-pyridyl)-6-(2-chlorophenyl)-1H-pyridin-2-one; N-[4-[2-(2-chlorophenyl)-6-oxo-1H-pyridin-4-yl]-2-pyridyl]-2-methoxy-acetamide; N-[4-[2-oxo-6-[2-(trifluoromethyl)-1-piperidyl]-1H-pyridin-4-yl]-2-pyridyl]acetamide; N-[4-[2-oxo-6-[2-(trifluoromethyl)-1-piperidyl]-1H-pyridin-4-yl]-2-pyridyl]cyclopropanecarboxamide; N-[4-[2-oxo-6-[3-(trifluoromethyl)morpholin-4-yl]-1H-pyridin-4-yl]-2-pyridyl]acetamide; methyl N-[4-[2-(2-chlorophenyl)-6-oxo-1H-pyridin-4-yl]-2-pyridyl]carbamate; methyl N-[4-[2-[1-ethyl-3-(trifluoromethyl)pyrazol-4-yl]-6-oxo-1H-pyridin-4-yl]-2-pyridyl]carbamate; methyl N-[4-[2-oxo-6-[2-(trifluoromethyl)-3-pyridyl]-1H-pyridin-4-yl]-2-pyridyl]carbamate; methyl N-[4-[2-oxo-6-[2-(trifluoromethyl)phenyl]-1H-pyridin-4-yl]-2-pyridyl]carbamate; N-[4-[2-oxo-6-[2-(trifluoromethyl)phenyl]-1H-pyridin-4-yl]-2-pyridyl]acetamide; N-[4-[2-(4-methyl-3-pyridyl)-6-oxo-1H-pyridin-4-yl]-2-pyridyl]acetamide; N-[4-[2-oxo-6-[2-(trifluoromethyl)-3-pyridyl]-1H-pyridin-4-yl]-2-pyridyl]acetamide; N-[4-[2-[1-ethyl-3-(trifluoromethyl)pyrazol-4-yl]-6-oxo-1H-pyridin-4-yl]-2-pyridyl]acetamide; methyl N-[4-[2-oxo-6-[3-(trifluoromethyl)morpholin-4-yl]-1H-pyridin-4-yl]-2-pyridyl]carbamate; methyl N-[4-[2-oxo-6-[2-(trifluoromethyl)-1-piperidyl]-1H-pyridin-4-yl]-2-pyridyl]carbamate; N-[4-[2-(3-cyclopropylmorpholin-4-yl)-6-oxo-1H-pyridin-4-yl]-2-pyridyl]acetamide; N-[4-[2-[4-ethylsulfonyl-2-(trifluoromethyl)piperazin-1-yl]-6-oxo-1H-pyridin-4-yl]-2-pyridyl]acetamide; N-[4-[2-(2-methyl-3-pyridyl)-6-oxo-1H-pyridin-4-yl]-2-pyridyl]acetamide; N-[4-[2-oxo-6-[4-(trifluoromethyl)-3-thienyl]-1H-pyridin-4-yl]-2-pyridyl]acetamide; 1,1-Dimethyl-3-[4-[2-oxo-6-[2-(trifluoromethyl)phenyl]-1H-pyridin-4-yl]-2-pyridyl]urea; N-[4-[2-oxo-6-[2-(trifluoromethyl)phenyl]-1H-pyridin-4-yl]-2-pyridyl]pyrrolidine-1-carboxamide; N-[4-[2-[2-(1-methoxy-1-methyl-ethyl)pyrrolidin-1-yl]-6-oxo-1H-pyridin-4-yl]-2-pyridyl]acetamide; and pharmaceutically acceptable salts, tautomers, and stereoisomers thereof.

In some embodiments, the compound is selected from the group consisting of: 4-(2-Amino-4-pyridyl)-6-(3-pyridyl)-1H-pyridin-2-one; 4-(2-Amino-4-pyridyl)-6-(2-chlorophenyl)-1H-pyridin-2-one; N-[4-[2-(2-chlorophenyl)-6-oxo-1H-pyridin-4-yl]-2-pyridyl]-2-methoxy acetamide; N-[4-[2-oxo-6-[2-(trifluoromethyl)-1-piperidyl]-1H-pyridin-4-yl]-2-pyridyl]acetamide; N-[4-[2-oxo-6-[2-(trifluoromethyl)-1-piperidyl]-1H-pyridin-4-yl]-2-pyridyl]cyclopropanecarboxamide; N-[4-[2-oxo-6-[3-(trifluoromethyl)morpholin-4-yl]-1H-pyridin-4-yl]-2-pyridyl]acetamide; methyl N-[4-[2-(2-chlorophenyl)-6-oxo-1H-pyridin-4-yl]-2-pyridyl]carbamate; methyl N-[4-[2-[1-ethyl-3-(trifluoromethyl)pyrazol-4-yl]-6-oxo-1H-pyridin-4-yl]-2-pyridyl]carbamate; methyl N-[4-[2-oxo-6-[2-(trifluoromethyl)-3-pyridyl]-1H-pyridin-4-yl]-2 pyridyl]carbamate; methyl N-[4-[2-(4-methyl-3-pyridyl)-6-oxo-1H-pyridin-4-yl]-2-pyridyl]carbamate; methyl N-[4-[2-oxo-6-[2-(trifluoromethyl)phenyl]-1H-pyridin-4-yl]-2-pyridyl]carbamate; N-[4-[2-oxo-6-[2-(trifluoromethyl)phenyl]-1H-pyridin-4-yl]-2-pyridyl]acetamide; N-[4-[2-(4-methyl-3-pyridyl)-6-oxo-1H-pyridin-4-yl]-2-pyridyl]acetamide; N-[4-[2-oxo-6-[2-(trifluoromethyl)-3-pyridyl]-1H-pyridin-4-yl]-2-pyridyl]acetamide; N-[4-[2-[1-ethyl-3-(trifluoromethyl)pyrazol-4-yl]-6-oxo-1H-pyridin-4-yl]-2-pyridyl]acetamide; methyl N-[4-[2-oxo-6-[3-(trifluoromethyl)morpholin-4-yl]-1H-pyridin-4-yl]-2-pyridyl]carbamate; methyl N-[4-[2-oxo-6-[2-(trifluoromethyl)-1-piperidyl]-1H-pyridin-4-yl]-2-pyridyl]carbamate; methyl N-[4-[2-[4-ethylsulfonyl-2-(trifluoromethyl)piperazin-1-yl]-6-oxo-1H-pyridin-4-yl]-2-pyridyl]carbamate; methyl N-[4-[2-(3-cyclopropylmorpholin-4-yl)-6-oxo-1H-pyridin-4-yl]-2-pyridyl]carbamate; N-[4-[2-(3-cyclopropylmorpholin-4-yl)-6-oxo-1H-pyridin-4-yl]-2-pyridyl]acetamide; N-[4-[2-[4-ethylsulfonyl-2-(trifluoromethyl)piperazin-1-yl]-6-oxo-1H-pyridin-4-yl]-2-pyridyl]acetamide; 3-[4-[2-(2-chlorophenyl)-6-oxo-1H-pyridin-4-yl]-2-pyridyl]-1,1-dimethyl-urea; N-[4-[2-(2-chlorophenyl)-6-oxo-1H-pyridin-4-yl]-2-pyridyl]pyrrolidine-1-carboxamide; and pharmaceutically acceptable salts, tautomers, and stereoisomers thereof.

In some embodiments, the compound is selected from the group consisting of: N-[4-[2-(2-chlorophenyl)-6-oxo-1H-pyridin-4-yl]-2-pyridyl]acetamide; 4-(2-Amino-4-pyridyl)-6-(3-pyridyl)-1H-pyridin-2-one; 4-(2-Amino-4-pyridyl)-6-(2-chlorophenyl)-1H-pyridin-2-one; N-[4-[2-(2-chlorophenyl)-6-oxo-1H-pyridin-4-yl]-2-pyridyl]-2-methoxy-acetamide; N-[4-[2-oxo-6-[2-(trifluoromethyl)-1-piperidyl]-1H-pyridin-4-yl]-2-pyridyl]acetamide; N-[4-[2-oxo-6-[2-(trifluoromethyl)-1-piperidyl]-1H-pyridin-4-yl]-2-pyridyl]cyclopropanecarboxamide; N-[4-[2-oxo-6-[3-(trifluoromethyl)morpholin-4-yl]-1H-pyridin-4-yl]-2-pyridyl]acetamide; methyl N-[4-[2-(2-chlorophenyl)-6-oxo-1H-pyridin-4-yl]-2-pyridyl]carbamate; methyl N-[4-[2-[1-ethyl-3-(trifluoromethyl)pyrazol-4-yl]-6-oxo-1H-pyridin-4-yl]-2-pyridyl]carbamate; methyl N-[4-[2-oxo-6-[2-(trifluoromethyl)-3-pyridyl]-1H-pyridin-4-yl]-2-pyridyl]carbamate; methyl N-[4-[2-oxo-6-[2-(trifluoromethyl)phenyl]-1H-pyridin-4-yl]-2-pyridyl]carbamate; N-[4-[2-oxo-6-[2-(trifluoromethyl)phenyl]-1H-pyridin-4-yl]-2-pyridyl]acetamide; N-[4-[2-(4-methyl-3-pyridyl)-6-oxo-1H-pyridin-4-yl]-2-pyridyl]acetamide; N-[4-[2-oxo-6-[2-(trifluoromethyl)-3-pyridyl]-1H-pyridin-4-yl]-2-pyridyl]acetamide; N-[4-[2-[1-ethyl-3-(trifluoromethyl)pyrazol-4-yl]-6-oxo-1H-pyridin-4-yl]-2-pyridyl]acetamide; methyl N-[4-[2-oxo-6-[3-(trifluoromethyl)morpholin-4-yl]-1H-pyridin-4-yl]-2-pyridyl]carbamate; methyl N-[4-[2-oxo-6-[2-(trifluoromethyl)-1-piperidyl]-1H-pyridin-4-yl]-2-pyridyl]carbamate; N-[4-[2-(3-cyclopropylmorpholin-4-yl)-6-oxo-1H-pyridin-4-yl]-2-pyridyl]acetamide; N-[4-[2-[4-ethylsulfonyl-2-(trifluoromethyl)piperazin-1-yl]-6-oxo-1H-pyridin-4-yl]-2-pyridyl]acetamide; and pharmaceutically acceptable salts, tautomers, and stereoisomers thereof.

In some embodiments, the at least one chemokine is selected from the group consisting of CCL5 and CXCL10.

In an aspect, provided herein is a method of treating cancer in a patient in need thereof, comprising: (i) administering to the patient a therapeutically effective amount of a compound represented by Formula V:

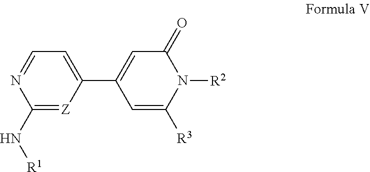

or a pharmaceutically acceptable salt, stereoisomer, or tautomer thereof, wherein: R¹ is selected from phenyl and monocyclic 5-6 membered heteroaryl, wherein each of phenyl and monocyclic 5-6 membered heteroaryl is optionally substituted with one or more substituents independently selected from the group consisting of halogen, C₁-C₆alkyl, C₃-C₄cycloalkyl, C₁-C₆alkoxy, C₁-C₆haloalkyl, C₁-C₆haloalkoxy, amino, N—C₁-C₃alkylamino and N,N-diC₁-C₃alkylamino; R² is selected from the group consisting of H, C₁-C₃haloalkyl, and C₁-C₃alkyl; R³ is selected from the group consisting of A, phenyl, and monocyclic heteroaryl, wherein each of phenyl and heteroaryl is optionally substituted with one or more occurrences of a substituent independently selected from the group consisting of R⁴, R⁵, R⁶, and R⁷; each of R⁴, R⁵, R⁶, and R⁷ is independently selected from the group consisting of halogen, C₁-C₆alkyl, C₃-C₄cycloalkyl, C₁-C₆alkoxy, C₁-C₆haloalkyl, C₁-C₆haloalkoxy, azetidine, amino, N—C₁-C₃alkylamino, N,N-diC₁-C₃alkylamino, NHSO₂R⁸, SO₂R⁹, and hydroxy; R⁸ is selected from C₁-C₃haloalkyl and C₁-C₃alkyl; each R⁹ is independently selected from the group consisting of R¹⁰, C₁-C₆alkyl, amino, N—C₁-C₃alkylamino, N,N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; each R¹⁰ is independently selected from the group consisting of phenyl, benzyl, monocyclic heteroaryl, C₃-C₆cycloalkyl, and heterocyclyl, wherein each of phenyl, benzyl, monocyclic heteroaryl, C₃-C₆cycloalkyl, and heterocyclyl is optionally substituted with one or more occurrences of R¹¹; each R¹¹ is independently selected from the group consisting of halogen, C₁-C₃haloalkyl, C₃-C₄cycloalkyl, C₁-C₃alkyl, amino, N—C₁-C₃alkylamino, N,N-diC₁-C₃alkylamino, and C₁-C₃alkoxyC₁-C₃alkyl; A is

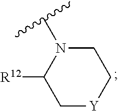

R¹² is selected from the group consisting of H, halogen, COR¹³, C₁-C₆alkyl, C₃-C₆cycloalkyl, C₁-C₃alkoxyC₁-C₃alkyl, C₁-C₆alkoxy, C₃-C₆cycloalkyl, C₁-C₃cyanoalkyl, and C₁-C₃haloalkyl; R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹⁵, NSO₂R⁹, NCOCH₂R⁹, O, and a bond; R¹⁴ is selected from H, C₁-C₃haloalkyl, C₁-C₃alkoxyC₁-C₃alkyl, C₁-C₃alkyl, and C₃-C₆cycloalkyl; and R¹⁵ is selected from R¹⁰, C₁-C₆alkyl, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and Z is selected from CH and N; and (ii) administering to the patient a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

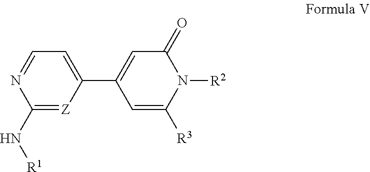

or a pharmaceutically acceptable salt, stereoisomer, or tautomer thereof, wherein: R¹ is selected from phenyl and monocyclic 5-6 membered heteroaryl, wherein each of phenyl and monocyclic 5-6 membered heteroaryl is optionally substituted with one or more substituents independently selected from the group consisting of halogen, C₁-C₆alkyl, C₃-C₄cycloalkyl, C₁-C₆alkoxy, C₁-C₆haloalkyl, C₁-C₆haloalkoxy, amino, N—C₁-C₃alkylamino and N,N-diC₁-C₃alkylamino; R² is selected from the group consisting of H, C₁-C₃haloalkyl, and C₁-C₃alkyl; R³ is selected from the group consisting of A, phenyl, and monocyclic heteroaryl, wherein each of phenyl and heteroaryl is optionally substituted with one or more occurrences of a substituent independently selected from the group consisting of R⁴, R⁵, R⁶, and R⁷; each of R⁴, R⁵, R⁶, and R⁷ is independently selected from the group consisting of halogen, C₁-C₆alkyl, C₃-C₄cycloalkyl, C₁-C₆alkoxy, C₁-C₆haloalkyl, C₁-C₆haloalkoxy, azetidine, amino, N—C₁-C₃alkylamino, N,N-diC₁-C₃alkylamino, NHSO₂R⁸, SO₂R⁹, and hydroxy; R⁸ is selected from C₁-C₃haloalkyl and C₁-C₃alkyl; each R⁹ is independently selected from the group consisting of R¹⁰, C₁-C₆alkyl, amino, N—C₁-C₃alkylamino, N,N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; each R¹⁰ is independently selected from the group consisting of phenyl, benzyl, monocyclic heteroaryl, C₃-C₆cycloalkyl, and heterocyclyl, wherein each of phenyl, benzyl, monocyclic heteroaryl, C₃-C₆cycloalkyl, and heterocyclyl is optionally substituted with one or more occurrences of R¹¹; each R¹¹ is independently selected from the group consisting of halogen, C₁-C₃haloalkyl, C₃-C₄cycloalkyl, C₁-C₃alkyl, amino, N—C₁-C₃alkylamino, N,N-diC₁-C₃alkylamino, and C₁-C₃alkoxyC₁-C₃alkyl; A is

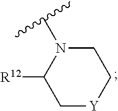

R¹² is selected from the group consisting of H, halogen, COR¹³, C₁-C₆alkyl, C₃-C₆cycloalkyl, C₁-C₃alkoxyC₁-C₃alkyl, C₁-C₆alkoxy, C₃-C₆cycloalkyl, C₁-C₃cyanoalkyl, and C₁-C₃haloalkyl; R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹⁵, NSO₂R⁹, NCOCH₂R⁹, O, and a bond; R¹⁴ is selected from H, C₁-C₃haloalkyl, C₁-C₃alkoxyC₁-C₃alkyl, C₁-C₃alkyl, and C₃-C₆cycloalkyl; and R¹⁵ is selected from R¹⁰, C₁-C₆alkyl, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and Z is selected from CH and N; and in an amount sufficient to induce a Type I interferon response by the cell; and (ii) a STING agonist in an amount sufficient to increase the expression level of the at least one chemokine in the cell.

In some embodiments, the compound is selected from the group consisting of: 4-(2-anilinopyrimidin-4-yl)-6-(2-chlorophenyl)-1H-pyridin-2-one; 4-(2-anilinopyrimidin-4-yl)-6-(3-pyridyl)-1H-pyridin-2-one; 4-(2-anilinopyrimidin-4-yl)-6-(4-pyridyl)-1H-pyridin-2-one; 4-(2-anilinopyrimidin-4-yl)-6-morpholino-1H-pyridin-2-one; 4-[2-[(2-Methylpyrimidin-4-yl)amino]-4-pyridyl]-6-[2-(trifluoromethyl)-1-piperidyl]-1H-pyridin-2-one; 4-(2-anilinopyrimidin-4-yl)-6-[2-(trifluoromethyl)-1-piperidyl]-1H-pyridin-2-one; 4-[2-[(2-Methylpyrimidin-4-yl)amino]-4-pyridyl]-6-[3-(trifluoromethyl)morpholin-4-yl]-1H-pyridin-2-one; 4-(2-anilinopyrimidin-4-yl)-6-[3-(trifluoromethyl)morpholin-4-yl]-1H-pyridin-2-one; 6-[4-[(4-Fluorophenyl)methylsulfonyl]-2-(trifluoromethyl)piperazin-1-yl]-4-[2-[(2-methylpyrimidin-4-yl)amino]-4-pyridyl]-1H-pyridin-2-one; 6-[4-Ethylsulfonyl-2-(trifluoromethyl)piperazin-1-yl]-4-[2-[(2-methylpyrimidin-4-yl)amino]-4-pyridyl]-1H-pyridin-2-one; 4-[2-(Oxazol-2-ylamino)-4-pyridyl]-6-[3-(trifluoromethyl)morpholin-4-yl]-1H-pyridin-2-one; 4-[2-[(2-Methylthiazol-4-yl)amino]-4-pyridyl]-6-[3-(trifluoromethyl)morpholin-4-yl]-1H-pyridin-2-one; 4-[2-[(2-methylpyrimidin-4-yl)amino]-4-pyridyl]-6-[2-(trifluoromethyl)-phenyl]-1H-pyridin-2-one; 4-[2-[(2-Methylpyrazol-3-yl)amino]-4-pyridyl]-6-[2-(trifluoromethyl)phenyl]-1H-pyridin-2-one; 4-[2-[(2-Methylthiazol-4-yl)amino]-4-pyridyl]-6-[2-(trifluoromethyl)phenyl]-1H-pyridin-2-one; 6-(4-methyl-3-pyridyl)-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-3-pyridyl]-1H-pyridin-2-one; 6-[1-ethyl-3-(trifluoromethyl)pyrazol-4-yl]-4-[2-[(2-methylpyrimidin-4-yl)am 4-pyridyl]-1H-pyridin-2-one; 4-[2-[(1-Methylimidazol-4-yl)amino]-4-pyridyl]-6-[2-(trifluoromethyl)phenyl pyridin-2-one; 6-(2-chlorophenyl)-4-[2-[(2-methylpyrimidin-4-yl)amino]-4-pyridyl]-1H-pyridin-2-one, and pharmaceutically acceptable salts, stereoisomers, and tautomers thereof.

In some embodiments, the compound is selected from the group consisting of: 4-(2-anilinopyrimidin-4-yl)-6-(2-chlorophenyl)-1H-pyridin-2-one; 4-(2-anilinopyrimidin-4-yl)-6-(3-pyridyl)-1H-pyridin-2-one; 4-(2-anilinopyrimidin-4-yl)-6-(4-pyridyl)-1H-pyridin-2-one; 4-(2-anilinopyrimidin-4-yl)-6-morpholino-1H-pyridin-2-one; 4-[2-[(2-Methylpyrimidin-4-yl)amino]-4-pyridyl]-6-[2-(trifluoromethyl)-1-piperidyl]-1H-pyridin-2-one; 4-(2-anilinopyrimidin-4-yl)-6-[2-(trifluoromethyl)-1-piperidyl]-1H-pyridin-2-one; 4-[2-[(2-Methylpyrimidin-4-yl)amino]-4-pyridyl]-6-[3-(trifluoromethyl)morpholin-4-yl]-1H-pyridin-2-one; 4-(2-anilinopyrimidin-4-yl)-6-[3-(trifluoromethyl)morpholin-4-yl]-1H-pyridin-2-one; 6-[4-[(4-Fluorophenyl)methylsulfonyl]-2-(trifluoromethyl)piperazin-1-yl]-4-[2-[(2-methylpyrimidin-4-yl)amino]-4-pyridyl]-1H-pyridin-2-one; 6-[4-Ethylsulfonyl-2-(trifluoromethyl)piperazin-1-yl]-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-phenyl]-1H-pyridin-2-one; 6-(4-methyl-3-pyridyl)-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-3-pyridyl]-1H-pyridin-2-one; 6-[1-ethyl-3-(trifluoromethyl)pyrazol-4-yl]-4-[2-[(2-methylpyrimidin-4-yl)am 4-pyridyl]-1H-pyridin-2-one; 6-(2-chlorophenyl)-4-[2-[(2-methylpyrimidin-4-yl)amino]-4-pyridyl]-1H-pyridin-2-one; 6-(3-cyclopropylmorpholin-4-yl)-4-[2-[(2-methylpyrimidin-4-yl)amino]-4-pyridyl]-1H-pyridin-2-one, and pharmaceutically acceptable salts, stereoisomers, and tautomers thereof.

In some embodiments, the compound is selected from the group consisting of: 4-(2-anilinopyrimidin-4-yl)-6-(2-chlorophenyl)-1H-pyridin-2-one; 4-(2-anilinopyrimidin-4-yl)-6-(3-pyridyl)-1H-pyridin-2-one; 4-(2-anilinopyrimidin-4-yl)-6-(4-pyridyl)-1H-pyridin-2-one 4-(2-anilinopyrimidin-4-yl)-6-morpholino-1H-pyridin-2-one; 4-[2-[(2-Methylpyrimidin-4-yl)amino]-4-pyridyl]-6-[2-(trifluoromethyl)-1-piperidyl]-1H-pyridin-2-one; 4-(2-anilinopyrimidin-4-yl)-6-[2-(trifluoromethyl)-1-piperidyl]-1H-pyridin-2-one; 4-[2-[(2-Methylpyrimidin-4-yl)amino]-4-pyridyl]-6-[3-(trifluoromethyl)morpholin-4-yl]-1H-pyridin-2-one; 4-(2-anilinopyrimidin-4-yl)-6-[3-(trifluoromethyl)morpholin-4-yl]-1H-pyridin-2-one; 6-[4-[(4-Fluorophenyl)methylsulfonyl]-2-(trifluoromethyl)piperazin-1-yl]-4-[2-[(2-methylpyrimidin-4-yl)amino]-4-pyridyl]-1H-pyridin-2-one; 6-[4-Ethylsulfonyl-2-(trifluoromethyl)piperazin-1-yl]-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-phenyl]-1H-pyridin-2-one; 6-(4-methyl-3-pyridyl)-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-3-pyridyl]-1H-pyridin-2-one; 6-[1-ethyl-3-(trifluoromethyl)pyrazol-4-yl]-4-[2-[(2-methylpyrimidin-4-yl)amino]-4-pyridyl]-1H-pyridin-2-one; 6-(2-chlorophenyl)-4-[2-[(2-methylpyrimidin-4-yl)amino]-4-pyhdyl]-1H-pyridin-2-one, and pharmaceutically acceptable salts, stereoisomers, and tautomers thereof.

In some embodiments, the at least one chemokine is selected from the group consisting of CCL5 and CXCL10.

In an aspect, provided herein is a method of treating cancer in a patient in need thereof, comprising: (i) administering to the patient a therapeutically effective amount of a compound represented by Formula VI:

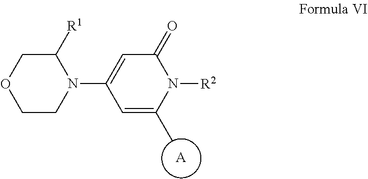

or a pharmaceutically acceptable salt, stereoisomer, or tautomer thereof, wherein: R¹ is selected from C₁-C₃alkyl and cyclopropyl; R² is selected from the group consisting of H, C₁-C₃haloalkyl, and C₁-C₃alkyl; A is selected from:

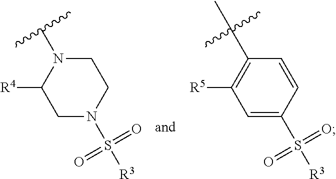

each R³ is independently selected from the group consisting of R⁶, C₁-C₆alkyl, amino N—C₁-C₃alkylamino, N, N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R⁶, and each of C₁-C₆alkyl and C₁-C₃alkoxyC₁-C₃alkyl is optionally substituted with or one or more independent occurrences of halogen; R⁴ is selected from the group consisting of C₁-C₆alkyl, C₁-C₆alkoxy, C₁-C₆haloalkyl, C₃-C₆cycloalkyl, and phenyl, wherein phenyl is optionally substituted with one or more occurrences of a substituent independently selected from the group consisting of fluoro, chloro, methyl, methoxy, dimethylamino, trifluoromethoxy, trifluoromethyl, and cyclopropyl; R⁵ is selected from the group consisting of halogen, C₁-C₆alkyl, C₁-C₆alkoxy, C₁-C₆haloalkyl, and C₃-C₆cycloalkyl; each R⁶ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁷; and each R⁷ is independently selected from the group consisting of halogen, amino, N—C₁-C₃alkylamino, N,N-diC₁-C₃alkylamino and C₁-C₃alkoxyC₁-C₃alkyl, C₁-C₃alkoxy, C₁-C₃haloalkoxy, C₃-C₆cycloalkyl, C₁-C₃haloalkyl, and C₁-C₃alkyl; and (ii) administering to the patient a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In an aspect, provided herein is a method of upregulating at least one chemokine in a cell comprising: contacting the cell sample with: (i) a compound represented by:

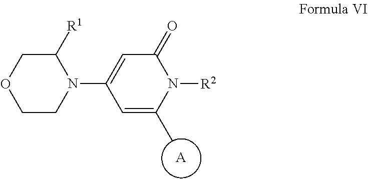

or a pharmaceutically acceptable salt, stereoisomer, or tautomer thereof, wherein: R¹ is selected from C₁-C₃alkyl and cyclopropyl; R² is selected from the group consisting of H, C₁-C₃haloalkyl, and C₁-C₃alkyl; A is selected from:

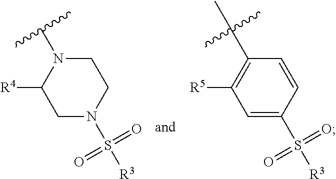

each R³ is independently selected from the group consisting of R⁶, C₁-C₆alkyl, amino N—C₁-C₃alkylamino, N, N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R⁶, and each of C₁-C₆alkyl and C₁-C₃alkoxyC₁-C₃alkyl is optionally substituted with or one or more independent occurrences of halogen; R⁴ is selected from the group consisting of C₁-C₆alkyl, C₁-C₆alkoxy, C₁-C₆haloalkyl, C₃-C₆cycloalkyl, and phenyl, wherein phenyl is optionally substituted with one or more occurrences of a substituent independently selected from the group consisting of fluoro, chloro, methyl, methoxy, dimethylamino, trifluoromethoxy, trifluoromethyl, and cyclopropyl; R⁵ is selected from the group consisting of halogen, C₁-C₆alkyl, C₁-C₆alkoxy, C₁-C₆haloalkyl, and C₃-C₆cycloalkyl; each R⁶ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁷; and each R⁷ is independently selected from the group consisting of halogen, amino, N—C₁-C₃alkylamino, N,N-diC₁-C₃alkylamino and C₁-C₃alkoxyC₁-C₃alkyl, C₁-C₃alkoxy, C₁-C₃haloalkoxy, C₃-C₆cycloalkyl, C₁-C₃haloalkyl, and C₁-C₃alkyl; in an amount sufficient to induce a Type I interferon response by the cell; and (ii) a STING agonist in an amount sufficient to increase the expression level of the at least one chemokine in the cell.

In some embodiments, the compound is selected from the group consisting of: 4-(3-methylmorpholin-4-yl)-6-[4-methylsulfonyl-2-(trifluoromethyl)piperazin-1-yl]-1H-pyridin-2-one; 6-[4-[(4-Fluorophenyl)methylsulfonyl]-2-(trifluoromethyl)piperazin-1-yl]-4-(3-methylmorpholin-4-yl)-1H-pyridin-2-one; 6-[4-[(5-Fluoro-3-pyridyl)sulfonyl]-2-(trifluoromethyl)piperazin-1-yl]-4-(3-methylmorpholin-4-yl)-1H-pyridin-2-one; 4-(3-methylmorpholin-4-yl)-6-[4-tetrahydrofuran-3-ylsulfonyl-2-(trifluoromethyl)piperazin-1-yl]-1H-pyridin-2-one; 4-(3-methylmorpholin-4-yl)-6-[4-pyrrolidin-1-ylsulfonyl-2-(trifluoromethyl)piperazin-1-yl]-1H-pyridin-2-one; N,N-dimethyl-4-[4-(3-methylmorpholin-4-yl)-6-oxo-1H-pyridin-2-yl]-3-(trifluoromethyl)piperazine-1-sulfonamide; 6-[4-(2-methoxyethylsulfonyl)-2-(trifluoromethyl)piperazin-1-yl]-4-(3-methylmorpholin-4-yl)-1H-pyridin-2-one; 6-[4-(4-fluorophenyl)sulfonyl-2-(trifluoromethyl)piperazin-1-yl]-4-(3-methylmorpholin-4-yl)-1H-pyridin-2-one; 4-(3-methylmorpholin-4-yl)-6-[4-(2-methylpyrazol-3-yl)sulfonyl-2-(trifluoromethyl)piperazin-1-yl]-1H-pyridin-2-one; 6-[4-Cyclopropylsulfonyl-2-(trifluoromethyl)piperazin-1-yl]-4-(3-methylmorpholin-4-yl)-1H-pyridin-2-one; 4-(3-methylmorpholin-4-yl)-6-[4-(1-piperidylsulfonyl)-2-(trifluoromethyl)piperazin-1-yl]-1H-pyridin-2-one; 4-(3-methylmorpholin-4-yl)-6-[4-morpholinosulfonyl-2-(trifluoromethyl)piperazin-1-yl]-1H-pyridin-2-one; 6-[4-(1,2-Dimethylimidazol-4-yl)sulfonyl-2-(trifluoromethyl)piperazin-1-yl]-4-(3-methylmorpholin-4-yl)-1H-pyridin-2-one; 6-[4-(1-methylcyclopropyl)sulfonyl-2-(trifluoromethyl)piperazin-1-yl]-4-(3-methylmorpholin-4-yl)-1H-pyridin-2-one; 4-(3-methylmorpholin-4-yl)-6-[4-methylsulfonyl-2-(trifluoromethyl)phenyl]-1H-pyridin-2-one; N,N-dimethyl-4-[4-(3-methylmorpholin-4-yl)-6-oxo-1H-pyridin-2-yl]-3-(trifluoromethyl)benzenesulfonamide; and pharmaceutically acceptable salts, stereoisomers, and tautomers thereof.

In some embodiments, the compound is selected from the group consisting of: 4-(3-methylmorpholin-4-yl)-6-[4-methylsulfonyl-2-(trifluoromethyl)piperazin-1-yl]-1H-pyridin-2-one; 6-[4-[(4-Fluorophenyl)methylsulfonyl]-2-(trifluoromethyl)piperazin-1-yl]-4-(3-methylmorpholin-4-yl)-1H-pyridin-2-one; 6-[4-[(5-Fluoro-3-pyridyl)sulfonyl]-2-(trifluoromethyl)piperazin-1-yl]-4-(3-methylmorpholin-4-yl)-1H-pyridin-2-one; 4-(3-methylmorpholin-4-yl)-6-[4-tetrahydrofuran-3-ylsulfonyl-2-(trifluoromethyl)piperazin-1-yl]-H-pyridin-2-one; 4-(3-methylmorpholin-4-yl)-6-[4-pyrrolidin-1-ylsulfonyl-2-(trifluoromethyl)piperazin-1-yl]-1H-pyridin-2-one; N,N-dimethyl-4-[4-(3-methylmorpholin-4-yl)-6-oxo-1H-pyridin-2-yl]-3-(trifluoromethyl)piperazine-1-sulfonamide; 6-[4-(2-methoxyethylsulfonyl)-2-(trifluoromethyl)piperazin-1-yl]-4-(3-methylmorpholin-4-yl)-1H-pyhdin-2-one; 6-[4-(4-fluorophenyl)sulfonyl-2-(trifluoromethyl)piperazin-1-yl]-4-(3-methylmorpholin-4-yl)-1H-pyridin-2-one; 4-(3-methylmorpholin-4-yl)-6-[4-(2-methylpyrazol-3-yl)sulfonyl-2-(trifluoromethyl)piperazin-1-yl]-1H-pyridin-2-one; and pharmaceutically acceptable salts, stereoisomers, and tautomers thereof.

In some embodiments, the at least one chemokine is selected from the group consisting of CCL5 and CXCL10.

In an aspect, provided herein is a method of treating cancer in a patient in need thereof, comprising: (i) means for inducing a Type I interferon response in a cancerous cell in the patient; and (ii) administering a therapeutically effective amount of a STING agonist to the patient;
wherein the therapeutically effective amount of the STING agonist results in an increased expression level of at least one chemokine in the patient as compared to any increase in the expression level of the at least one chemokine resulting from administering the compound to the patient.

In some embodiments, the methods described herein may further comprise administering an additional therapeutic agent to the patient. In some embodiments, the additional therapeutic agent is selected from the group consisting of a PD-1 pathway antagonist, a TIM-3 pathway antagonist, a Vista pathway antagonist, a BTLA pathway antagonist, a LAG-3 pathway antagonist, a TIGIT pathway antagonist, and a CTLA4 pathway antagonist.

In some embodiments, the STING agonist is selected from the group consisting of 5,6-dimethylxanthenone-4-acetic acid (DMXAA), ADU-S100, MK-1454, MK-2118, BMS-986301, GSK3745417, SB-11285, BI1387446 (BI-STING), E7766, TAK-676, SNX281, SYNB1891, JNJ-67544412, JNJ-′6196, GSK532, TTI-10001, ALG-031048, MSA-1, MSA-2, CRD-5500, MV-626, SR-8314, SR-8291, SR8541A, SR-717, STING antibody-drug conjugates (ADC), and IMSA-101, and pharmaceutically acceptable salts thereof. In some embodiments, the STING agonist is selected from the group consisting of ADU-S100, MK-1454, MK-2118, BMS-986301, GSK3745417, SB-11285, B11387446 (BI-STING), E7766, TAK-676, SNX281, SYNB1891, and IMSA-101, and pharmaceutically acceptable salts thereof. In some embodiments, the STING agonist is selected from ADU-S100 and pharmaceutically acceptable salts thereof.

### Combination Therapy

Compounds described herein, e.g., a compound of Formula I, Formula II, Formula III, Formula IV, Formula V, or Formula VI as defined herein, can be administered in combination with one or more additional therapeutic agents (e.g., one or more other additional agents described herein) to treat a disorder described herein, such as a cancer described herein. For example, provided in the present disclosure is a pharmaceutical composition comprising a compound described herein, e.g., a compound of Formula I, Formula II, Formula III, Formula IV, Formula V, or Formula VI as defined herein, one or more additional therapeutic agents, and a pharmaceutically acceptable excipient. In some embodiments, a compound of Formula I, Formula II, Formula III, Formula IV, Formula V, or Formula VI as defined herein and one additional therapeutic agent is administered. In some embodiments, a compound of Formula I, Formula II, Formula III, Formula IV, Formula V, or Formula VI as defined herein and two additional therapeutic agents are administered. In some embodiments, a compound of Formula I, Formula II, Formula III, Formula IV, Formula V, or Formula VI as defined herein and three additional therapeutic agents are administered. Combination therapy can be achieved by administering two or more therapeutic agents, each of which is formulated and administered separately. For example, a compound of Formula I, Formula II, Formula III, Formula IV, Formula V, or Formula VI as defined herein and an additional therapeutic agent can be formulated and administered separately. Combination therapy can also be achieved by administering two or more therapeutic agents in a single formulation, for example a pharmaceutical composition comprising a compound of Formula I, Formula II, Formula III, Formula IV, Formula V, or Formula VI as one therapeutic agent and one or more additional therapeutic agents such as an antibiotic, a viral protease inhibitor, or an anti-viral nucleoside anti-metabolite. For example, a compound of Formula I, Formula II, Formula III, Formula IV, Formula V, or Formula VI as defined herein and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y-Y-X, X-X-Y-Y, etc.

Examples of additional therapeutics agents that can be used in combination with a VPS34 inhibitor such as a compound of Formula I, Formula II, Formula III, Formula IV, Formula V, or Formula VI as defined herein, includes, but not limited to, a STING agonist (e.g., DMXAA, ADU-S100, or pharmaceutically acceptable salt thereof), an anti-PD-1 therapeutic, an anti PD-L1 therapeutic, or a CTLA4 inhibitor.

### Pharmaceutical Compositions and Kits

Another aspect of this disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions may be used in the form of a pharmaceutical preparation, for example, in solid, semisolid or liquid form, which contains one or more of the compounds described herein, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound provided herein, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of the present disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another aspect, provided are enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5.

Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives described herein.

Advantageously, provided herein are kits for use by a e.g. a consumer in need of treatment of a disease or disorder described herein, such as an infection caused by a pathogen described herein, e.g., a virus, fungus, or protozoan. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### EXAMPLES

The compounds described herein can be prepared in a number of ways based on the teachings contained herein and disclosures of synthetic procedures in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

### Example 1. Exemplary Synthesis of Compound 1

Compound 1 was prepared according to synthetic procedures described in WO 2017/140843.

### Example 2. Compound 1 or Compound 2 Upregulate Type I Interferon Response Gene Expression in A498 Renal Cancer Cells

### Quantitative Reverse-Transcriptase Polymerase Chain Reaction (RT-PCR) Assay

A498 cells were grown in Minimal Essential Medium containing 10% fetal bovine serum. Cells were then incubated for 2 days at 37 degrees Celsius, 5% CO₂, and 95% humidity. A498 cells were incubated with 2 μM of compound 1 for 24 hr. Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Human primers | Forward primer sequence | Reverse primer sequence |
|---|---|---|
| IFNB1 | GCTTGGATTCCTACAAA | ATAGATGGTCAATGCGGCGT |
| | GAAGCA | C |
| IRF1 | CTGTGCGAGTGTACCGG | ATCCCCACATGACTTCCTCT |
| | ATG | T |
| IRF7 | GCTGGACGTGACCATCA | GGGCCGTATAGGAACGTGC |
| | TGTA | |
| IRF9 | GCCCTACAAGGTGTATC | TGCTGTCGCTTTGATGGTAC |
| | AGTTG | T |
| Tubulin (housekeeping gene) | GAAGCAGCAACCATGC | GGCATTGCCAATCTGGACAC |
| | GTGA | |

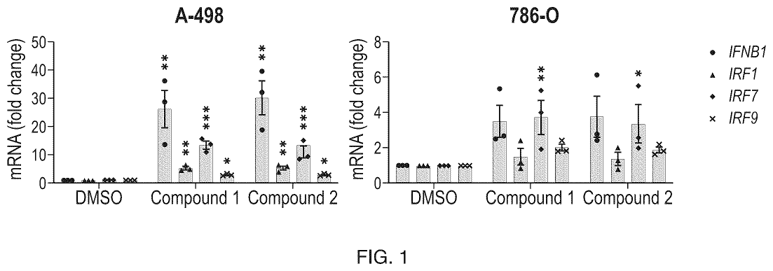

A study was performed to test whether Compound 1 or Compound 2 activated the STING pathway through increased Type 1 Interferon pathway genes in A-498 renal cell carcinoma cells. FIG. 1, left panel, is a graphical representation of the increase in the mRNA expression of IFNB1 (filled circle), IRF1 (filled triangle), IRF7 (filled diamond) and IRF9 (cross) in response to Compound 1 (middle set of bars) or Compound 2 (rightmost set of bars). Compounds 1 and 2 increased mRNA expression of IFNB1 and IRF7 by more than 10-fold, and IRF1 and IRF9 approximately 2 to 5-fold.

### Example 3. Compound 1 or Compound 2 Upregulates Type 1 Interferon Response Gene Expression in 786-O Renal Cancer Cells

### Quantitative Reverse-Transcriptase Polymerase Chain Reaction (RT-PCR) Assay

786-O cells were grown in RPMI-1640 medium containing 10% fetal bovine serum. Cells were then incubated for 2 days at 37 degrees Celsius, 5% CO₂, and 95% humidity. 786-O cells were incubated with 2 μM of compound 1 for 24 hr. Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Human primers | Forward primer sequence | Reverse primer sequence |
|---|---|---|
| IFNB1 | GCTTGGATTCCTACAAA | ATAGATGGTCAATGCGGCGT |
| | GAAGCA | C |
| IRF1 | CTGTGCGAGTGTACCGG | ATCCCCACATGACTTCCTCT |
| | ATG | T |
| IRF7 | GCTGGACGTGACCATCA | GGGCCGTATAGGAACGTGC |
| | TGTA | |
| IRF9 | GCCCTACAAGGTGTATC | TGCTGTCGCTTTGATGGTAC |
| | AGTTG | T |
| Tubulin (housekeeping gene) | GAAGCAGCAACCATGC | GGCATTGCCAATCTGGACAC |
| | GTGA | |

A study was performed to test whether Compound 1 or Compound 2 activated the STING pathway through increased Type 1 Interferon pathway genes in 786-O renal cell carcinoma cells. FIG. 1, right panel, is a graphical representation of the increase in the mRNA expression of IFNB1 (filled circle), IRF1 (filled triangle), IRF7 (filled diamond) and IRF9 (cross) in response to Compound 1 (middle set of bars) or Compound 2 (rightmost set of bars). Compounds 1 and 2 increased mRNA expression of IFNB1 and IRF7 by more than 3-fold, and IRF9 approximately 2-fold.

### Example 4. Compound 1 Activates the STAT1 Signaling Pathway

### Western Blot Assay

A-498 cells were seeded at 1.5×10⁵ cells/well in 6-well plates and cultured overnight. Cells were treated with 10 μg/mL mouse monoclonal antibody to human interferon α/β receptor chain 2 (α-IFNAR2) (clone: MMHAR-2, #21385-1, PBL Assay Science) or 10 μg/mL mouse IgG2a isotype control antibody (clone: eBM2a, #16-4724-81, Thermo Fisher Scientific). After 1 h, DMSO or 2 μM Compound 1 was added and cells were incubated for another 24 h. Treatment with 5 ng/mL recombinant IFNβ (#300-02BC, PeproTech) was used as a positive control. A-498 cells were washed in PBS and lysed in RIPA buffer (50 mM Tris HCl pH=7.4, 150 mM NaCl, 1% Triton X-100, 0.5% Na-deoxycholate, 0.1% SDS) containing protease inhibitor cocktail (#11836170001, Roche), and phosphatase inhibitor (#11836170001, Roche) for 10 min at 4° C. After centrifugation at 15000 rpm for 10 min at 4° C., the supernatant was obtained, and protein concentration was quantified using BCA assay (#23225, Thermo Fisher). Equal amounts were mixed with 0.1 M DTT and LDS Sample Buffer (#84788, Thermo Fisher), heated for 5 min at 90° C. before loading onto 4-12% Bis-Tris gels. Proteins were transferred to nitrocellulose membranes. Membranes were blocked in 5% non-fat dry milk in TBS with 0.05% Tween (TBS-T) for 1 h at RT, washed in TBS-T, and probed with primary antibodies shaking overnight at 4° C. After washing in TBS-T, membranes were incubated with IRDye 800CW goat anti-rabbit or IRDye 680RD donkey anti-mouse IgG (H+L) (#926-32211 and #926-68072, respectively, LI-COR, 1:10,000 dilution) secondary antibody in Intercept blocking buffer (#927-60001, LI-COR) for 1 h at RT. After washing in TBS-T, membranes were imaged using the Odyssey CLx (LI-COR). Band intensities were quantified using Image Studio Lite software version 5.2.5 (LI-COR).

| | | Diluent (in | Cat. | |
|---|---|---|---|---|
| Antibody | Dilution | TBS-Tween) | number | Supplier |
| STAT1 | 1:1000 | 5% milk | 9172 | Cell Signaling Technology |
| phospho-STAT1 | 1:1000 | 5% BSA | 7649 | Cell Signaling Technology |
| Tyr701 | | | | |
| Actin | 1:10,000 | 5% milk | A5441 | Sigma-Aldrich |

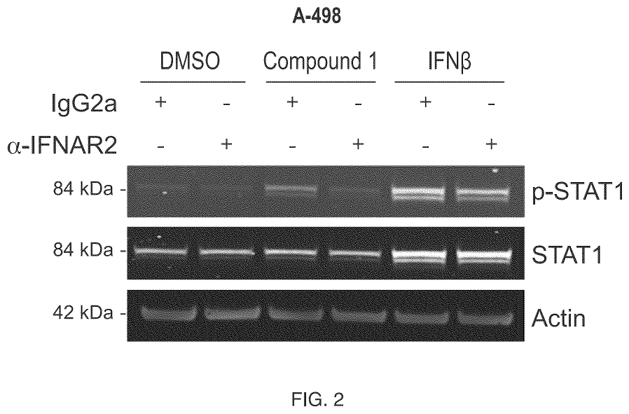

A study was performed to test whether Compound 1 activated the STING pathway through increased STAT1 phosphorylation. FIG. 2 is an image of a western blot that shows Compound 1 increased phosphorylation of STAT1 (top image, Lane 3), which is inhibited by the addition of α-IFNAR2 (top image, Lane 4), demonstrating specificity to the STING pathway. Compound 1 did not alter total levels of STAT1 (middle image, Lane 3). IFNβ was used as a positive control, with lane 5 showing increased STAT1 phosphorylation. Actin was used as a loading control (bottom image).

### Example 5. VPS34 Inhibitors Compound 1 or Compound 2 Activate the STING Pathway Through p-TKB1, p-IRF3 and p-STAT1 in A-498 Renal Cancer Cells. siSTING or siCGAS Reverses the Effects of VPS34 Inhibitors

### Western Blot Assay

A-498 cells were transfected with scrambled siRNA control (siSCR) or siRNA targeting TBK1 (siTBK1), STING (siSTING) or CGAS (siCGAS). After 48 h cells were treated with DMSO or 2 μM of VPS34 inhibitor (Compound 1 or Compound 2) for 24 h. A-498 cells were washed in PBS and lysed in RIPA buffer (50 mM Tris HCl pH=7.4, 150 mM NaCl, 1% Triton X-100, 0.5% Na-deoxycholate, 0.1% SDS) containing protease inhibitor cocktail (#11836170001, Roche), and phosphatase inhibitor (#11836170001, Roche) for 10 min at 4° C. After centrifugation at 15000 rpm for 10 min at 4° C., the supernatant was obtained, and protein concentration was quantified using BCA assay (#23225, Thermo Fisher). Equal amounts were mixed with 0.1 M DTT and LDS Sample Buffer (#84788, Thermo Fisher), heated for 5 min at 90° C. before loading onto 4-12% Bis-Tris gels. Proteins were transferred to nitrocellulose membranes. Membranes were blocked in 5% non-fat dry milk in TBS with 0.05% Tween (TBS-T) for 1 h at RT, washed in TBS-T, and probed with primary antibodies shaking overnight at 4° C. After washing in TBS-T, membranes were incubated with IRDye 800CW goat anti-rabbit or IRDye 680RD donkey anti-mouse IgG (H+L) (#926-32211 and #926-68072, respectively, LI-COR, 1:10,000 dilution) secondary antibody in Intercept blocking buffer (#927-60001, LI-COR) for 1 h at RT. After washing in TBS-T, membranes were imaged using the Odyssey CLx (LI-COR). Band intensities were quantified using Image Studio Lite software version 5.2.5 (LI-COR).

| | | Diluent (in | Cat. | |
|---|---|---|---|---|
| Antibody | Dilution | TBS-Tween) | number | Supplier |
| STING | 1:1000 | 5% milk | 13647 | Cell Signaling Technology |
| cGAS | 1:1000 | 5% milk | 15102 | Cell Signaling Technology |
| TBK1 | 1:1000 | 5% milk | 3504 | Cell Signaling Technology |
| phospho-TBK1 Ser172 | 1:1000 | 5% BSA | 5483 | Cell Signaling Technology |
| phospho-IRF3 Ser386 | 1:500 | 5% BSA | 37829 | Cell Signaling Technology |
| STAT1 | 1:1000 | 5% milk | 9172 | Cell Signaling Technology |
| phospho-STAT1 | 1:1000 | 5% BSA | 7649 | Cell Signaling Technology |
| Tyr701 | | | | |
| Actin | 1:10,000 | 5% milk | A5441 | Sigma-Aldrich |

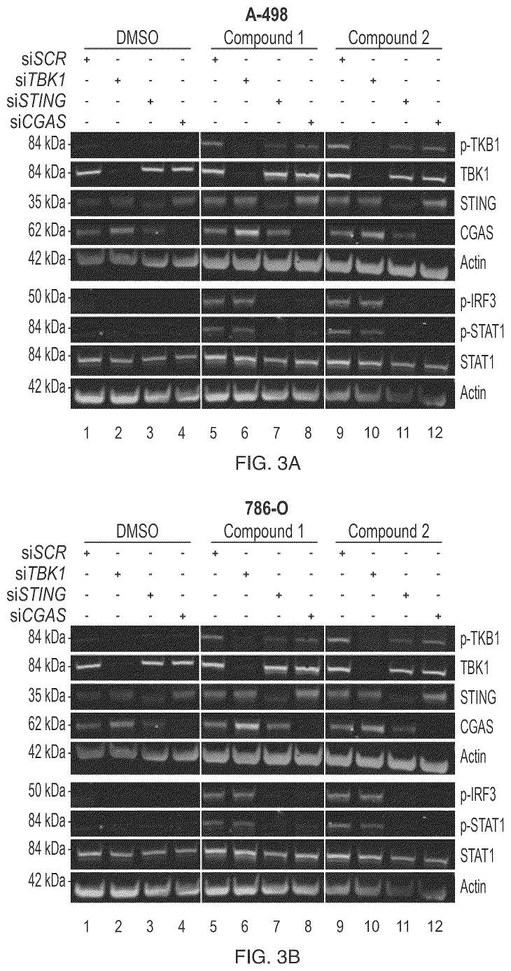

A study was performed to test whether Compound 1 or Compound 2 increased activation of the STING pathway in A-498 renal cell carcinoma cells. Levels of phosphorylated TBK1, IRF3, and STAT1, downstream of STING and CGAS, were measured by Western blot. FIG. 3A is an image of a Western blot in which lane 1 shows baseline control (DMSO) levels of p-TBK1, TBK1, STING, CGAS, pIRF3, p-STAT1 and STAT1 along with the loading control, Actin. Lane 2 shows the effect of siRNA knockdown of TBK1, which results in complete loss of TBK1 and a slight increase in CGAS. Lane 3 shows the effect of siRNA knockdown of STING, which results in nearly complete loss of STING and a decrease in CGAS. Lane 4 shows the effect of siRNA knockdown of CGAS, which results in complete loss of CGAS. Thus, Lanes 2-4 demonstrate the effectiveness of the siRNA in knockdown of the respective RNA, and hence depletion of the targeted protein.

Lanes 5-8 show the effects of Compound 1 either alone or in conjunction with siRNA knockdown of STING pathway genes. Lane 5 shows the effect of Compound 1 which results in an increase in p-TKB1, increase in STING in comparison to DMSO alone (lane 1), a mild increase in CGAS and strong increases in p-IRF3 and p-STAT1 in comparison to DMSO alone, demonstrating an effect of Compound 1 on stimulation of the STING pathway. Lane 6 shows the effect of Compound 1 in conjunction with siRNA knockdown of TBK1 which results in complete loss of p-TBK1 and total TBK1, and an increase in STING, similar to that of Compound 1 alone (lane 5), an increase in CGAS compared to DMSO Lanes 1 and 2 and to Compound 1 alone (lane 5). Lane 6 also shows similar levels of p-IRF3 and p-STAT1 as Compound 1 alone (lane 5), indicating that Compound 1 can still activate downstream STING pathway signaling through p-IRF3 and p-STAT1. Lane 7 shows the effect of Compound 1 in conjunction with siRNA knockdown of STING which results in nearly complete loss of STING in addition to a decrease in p-TBK1 in comparison to Compound 1 alone (lane 5). Lane 7 also shows complete loss of p-IRF3 and p-STAT1 in comparison to Compound 1 alone (lane 5), demonstrating that the effects of Compound 1 to increase p-IRF3 and p-STAT1 levels are mediated through STING. Lane 8 shows the effect of Compound 1 in conjunction with siRNA knockdown of CGAS which results in a complete loss of CGAS, a decrease in p-TKB1, and complete loss of p-IRF3 and p-STAT1 in comparison to Compound 1 alone (lane 5), again confirming that the effects of Compound 1 to stimulate p-IRF3 and p-STAT1 are mediated through the CGAS/STING pathway.

Lanes 9-12 show the effects of Compound 2 either alone or in conjunction with siRNA knockdown of STING pathway genes. Lane 9 shows the effect of Compound 2 which results in an increase in p-TKB1, increase in STING, a mild increase in CGAS and strong increases in p-IRF3 and p-STAT1 in comparison to DMSO alone (lane 1), demonstrating an effect of Compound 2 on stimulation of the STING pathway. Lane 10 shows the effect of Compound 2 in conjunction with siRNA knockdown of TBK1 which results in complete loss of p-TBK1 and total TBK1 and an increase in STING in comparison to DMSO alone (lane 1), similar to that of Compound 2 alone (lane 9), an increase in CGAS compared to DMSO Lane 1 and similar to Compound 2 alone (lane 9). Lane 10 also shows similar levels of p-IRF3 and p-STAT1 as Compound 2 alone (lane 9), indicating that Compound 2 can still activate downstream STING pathway signaling through p-IRF3 and p-STAT1. Lane 11 shows the effect of Compound 2 in conjunction with siRNA knockdown of STING which results in complete loss of STING in addition to a decrease in p-TBK1 in comparison to Compound 2 alone (lane 9). Lane 11 also shows complete loss of p-IRF3 and p-STAT1 in comparison to Compound 2 alone (lane 9), demonstrating that the effects of Compound 2 to increase p-IRF3 and p-STAT1 levels are mediated through STING. Lane 12 shows the effect of Compound 2 in conjunction with siRNA knockdown of CGAS which results in a complete loss of CGAS, a decrease in p-TKB1, and complete loss of p-IRF3 and p-STAT1 in comparison to Compound 2 alone (lane 9), again confirming that the effects of Compound 2 to stimulate p-IRF3 and p-STAT1 are mediated through the CGAS/STING pathway.

### Example 6. VPS34 Inhibitors Compound 1 or Compound 2 Activate the STING Pathway Through p-TKB1, p-IRF3 and p-STAT1 in 786-O Renal Cancer Cells. siSTING or siCGAS Reverses the Effects of VPS34 Inhibitors

### Western Blot Assay

786-O cells were transfected with scrambled siRNA control (siSCR) or siRNA targeting TBK1 (siTBK1), STING (siSTING) or CGAS (siCGAS). After 48 h cells were treated with DMSO or 2 μM of VPS34 inhibitor (Compound 1 or Compound 2) for 24 h. 786-O cells were washed in PBS and lysed in RIPA buffer (50 mM Tris HCl pH=7.4, 150 mM NaCl, 1% Triton X-100, 0.5% Na-deoxycholate, 0.1% SDS) containing protease inhibitor cocktail (#11836170001, Roche), and phosphatase inhibitor (#11836170001, Roche) for 10 min at 4° C. After centrifugation at 15000 rpm for 10 min at 4° C., the supernatant was obtained, and protein concentration was quantified using BCA assay (#23225, Thermo Fisher). Equal amounts were mixed with 0.1 M DTT and LDS Sample Buffer (#84788, Thermo Fisher), heated for 5 min at 90° C. before loading onto 4-12% Bis-Tris gels. Proteins were transferred to nitrocellulose membranes. Membranes were blocked in 5% non-fat dry milk in TBS with 0.05% Tween (TBS-T) for 1 h at RT, washed in TBS-T, and probed with primary antibodies shaking overnight at 4° C. After washing in TBS-T, membranes were incubated with IRDye 800CW goat anti-rabbit or IRDye 680RD donkey anti-mouse IgG (H+L) (#926-32211 and #926-68072, respectively, LI-COR, 1:10,000 dilution) secondary antibody in Intercept blocking buffer (#927-60001, LI-COR) for 1 h at RT. After washing in TBS-T, membranes were imaged using the Odyssey CLx (LI-COR). Band intensities were quantified using Image Studio Lite software version 5.2.5 (LI-COR).

| | | Diluent (in | Cat. | |
|---|---|---|---|---|
| Antibody | Dilution | TBS-Tween) | number | Supplier |
| STING | 1:1000 | 5% milk | 13647 | Cell Signaling Technology |
| cGAS | 1:1000 | 5% milk | 15102 | Cell Signaling Technology |
| TBK1 | 1:1000 | 5% milk | 3504 | Cell Signaling Technology |
| phospho-TBK1 Ser172 | 1:1000 | 5% BSA | 5483 | Cell Signaling Technology |
| phospho-IRF3 Ser386 | 1:500 | 5% BSA | 37829 | Cell Signaling Technology |
| STAT1 | 1:1000 | 5% milk | 9172 | Cell Signaling Technology |
| phospho-STAT1 | 1:1000 | 5% BSA | 7649 | Cell Signaling Technology |
| Tyr701 | | | | |
| Actin | 1:10,000 | 5% milk | A5441 | Sigma-Aldrich |

A study was performed to test whether Compound 1 or Compound 2 increased activation of the STING pathway in 786-O renal cell carcinoma cells. Levels of phosphorylated TBK1, IRF3, and STAT1, downstream of STING and CGAS were measured by Western blot. FIG. 3A is an image of a Western blot in which lane 1 shows baseline control (DMSO) levels of p-TBK1, TBK1, STING, CGAS, pIRF3, p-STAT1 and STAT1 along with the loading control, Actin. Lane 2 shows the effect of siRNA knockdown of TBK1, which results in complete loss of TBK1 and a slight increase in CGAS. Lane 3 shows the effect of siRNA knockdown of STING, which results in nearly complete loss of STING and a decrease in CGAS. Lane 4 shows the effect of siRNA knockdown of CGAS, which results in complete loss of CGAS. Thus, Lanes 2-4 demonstrate the effectiveness of the siRNA in knockdown of the respective RNA, and hence depletion of the targeted protein.

Lanes 5-8 show the effects of Compound 1 either alone or in conjunction with siRNA knockdown of STING pathway genes. Lane 5 shows the effect of Compound 1 which results in an increase in p-TKB1, increase in STING in comparison to DMSO alone (lane 1), a mild increase in CGAS and strong increases in p-IRF3 and p-STAT1 in comparison to DMSO alone, demonstrating an effect of Compound 1 on stimulation of the STING pathway. Lane 6 shows the effect of Compound 1 in conjunction with siRNA knockdown of TBK1 which results in complete loss of p-TBK1 and total TBK1 and an increase in STING, similar to that of Compound 1 alone (lane 5), an increase in CGAS compared to DMSO Lane 1 and 2 and to Compound 1 alone (lane 5). Lane 6 also shows similar levels of p-IRF3 and p-STAT1 as Compound 1 alone (lane 5), indicating that Compound 1 can still activate downstream STING pathway signaling through p-IRF3 and p-STAT1. Lane 7 shows the effect of Compound 1 in conjunction with siRNA knockdown of STING which results in nearly complete loss of STING in addition to a decrease in p-TBK1 in comparison to Compound 1 alone (lane 5). Lane 7 also shows complete loss of p-IRF3 and p-STAT1 in comparison to Compound 1 alone (lane 5), demonstrating that the effects of Compound 1 to increase p-IRF3 and p-STAT1 levels are mediated through STING. Lane 8 shows the effect of Compound 1 in conjunction with siRNA knockdown of CGAS which results in a complete loss of CGAS, a decrease in p-TKB1, and complete loss of p-IRF3 and p-STAT1 in comparison to Compound 1 alone (lane 5), again confirming that the effects of Compound 1 to stimulate p-IRF3 and p-STAT1 are mediated through the CGAS/STING pathway.

Lanes 9-12 show the effects of Compound 2 either alone or in conjunction with siRNA knockdown of STING pathway genes. Lane 9 shows the effect of Compound 2 which results in an increase in p-TKB1, increase in STING, a mild increase in CGAS and strong increases in p-IRF3 and p-STAT1 in comparison to DMSO alone, demonstrating an effect of Compound 2 on stimulation of the STING pathway. Lane 10 shows the effect of Compound 2 in conjunction with siRNA knockdown of TBK1 which results in complete loss of p-TBK1 and total TBK1 and an increase in STING in comparison to DMSO alone (lane 1), similar to that of Compound 2 alone (lane 9), an increase in CGAS compared to DMSO Lane 1 and similar to Compound 2 alone (lane 9). Lane 10 also shows similar levels of p-IRF3 and p-STAT1 as Compound 2 alone (lane 9), indicating that Compound 2 can still activate downstream STING pathway signaling through p-IRF3 and p-STAT1. Lane 11 shows the effect of Compound 2 in conjunction with siRNA knockdown of STING which results in complete loss of STING in addition to a decrease in p-TBK1 in comparison to Compound 2 alone (lane 9). Lane 11 also shows complete loss of p-IRF3 and p-STAT1 in comparison to Compound 2 alone (lane 9), demonstrating that the effects of Compound 2 to increase p-IRF3 and p-STAT1 levels are mediated through STING. Lane 12 shows the effect of Compound 2 in conjunction with siRNA knockdown of CGAS which results in a complete loss of CGAS, a decrease in p-TKB1, and complete loss of p-IRF3 and p-STAT1 in comparison to Compound 2 alone (lane 9), again confirming that the effects of Compound 1 to stimulate p-IRF3 and p-STAT1 are mediated through the CGAS/STING pathway.

### Example 7. Compound 1 or Compound 2 Upregulates Type I Interferon Response Gene Expression in A498 Renal Cancer Cells which is Reversed by siSTING or siCGAS

### Quantitative Reverse-Transcriptase Polymerase Chain Reaction (RT-PCR) Assay

A-498 cells were grown in Minimal Essential Medium containing 10% fetal bovine serum. Cells were then incubated for 2 days at 37 degrees Celsius, 5% CO₂, and 95% humidity. A498 cells were transfected with scrambled siRNA control (siSCR) or siRNA targeting TBK1 (siTBK1), STING (siSTING) or CGAS (siCGAS). After 48 h cells were treated with DMSO or 2 μM of VPS34 inhibitor (Compound 1 or Compound 2) for 24 h. Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Human primers | Forward primer sequence | Reverse primer sequence |
|---|---|---|
| IFNB1 | GCTTGGATTCCTACAAAGAAGCA | ATAGATGGTCAATGCGGCGTC |
| IRF7 | GCTGGACGTGACCATCATGTA | GGGCCGTATAGGAACGTGC |
| CCL5 | CTCCCCATATTCCTCGGACA | ACTCCTTGATGTGGGCACG |
| CXCL10 | TGGCATTCAAGGAGTACCTCTC | ATGCTGATGCAGGTACAGCG |
| Tubulin (house- | GAAGCAGCAACCATGCGTGA | GGCATTGCCAATCTGGACAC |
| keeping gene) | | |

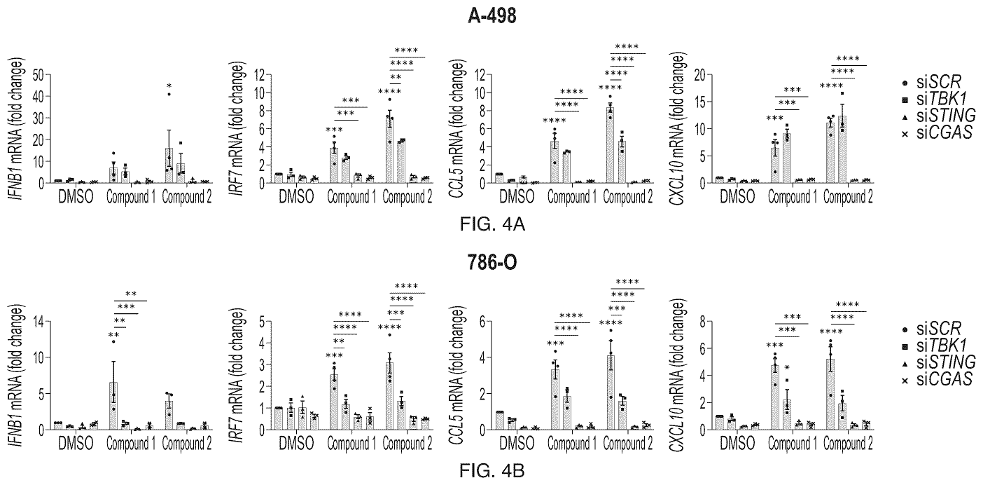

A study was performed to test whether Compound 1 or Compound 2 activated the STING pathway in A-498 renal cell carcinoma cells through increased Type 1 Interferon pathway genes and whether this is ablated through the siRNA knockdown of STING pathway genes. FIG. 4A is a graphical representation of the increase in mRNA expression of IFNB1 (left graph), IRF7 (2nd graph from the left), CCL5 (3rd graph from the left) and CXCL10 (right graph) in response to Compound 1 (middle set of bars on each graph) and Compound 2 (right most set of bars on each graph). The symbols represent the effects of siRNA knockdown of scrambled control (filled circles), siTBK1 (filled square), siSTING (filled triangle) and siCGAS (cross).

Compound 1 increases mRNA expression of IFNB1 (>5-fold), IRF7 (˜4-fold), CCL5 (>4-fold) and CXCL10 (>5-fold). Compound 2 increases mRNA expression of IFNB1 (>10-fold), IRF7 (˜7-fold), CCL5 (˜8-fold) and CXCL10 (˜10-fold). siRNA knockdown of TBK1 (filled squares) in combination with Compound 1 or Compound 2 results in moderate or negligible changes in IFNB1, IRF7, CCL5 or CXCL10 in comparison to Compound 1 or Compound 2 alone. siRNA knockdown of STING (filled triangles) in combination with Compound 1 or Compound 2 results in complete ablation of IFNB1, IRF7, CCL5 and CXCL10 expression in comparison to Compound 1 or Compound 2 alone. siRNA knockdown of CGAS (cross) in combination with Compound 1 or Compound 2 results in complete ablation of IFNB1, IRF7, CCL5 and CXCL10 expression in comparison to Compound 1 or Compound 2 alone. In composite, these data demonstrate that Compound 1 and Compound 2 mediate the increase in type 1 interferon response genes through activation of the CGAS/STING pathway.

### Example 8. Compound 1 or Compound 2 Upregulates Type I Interferon Response Gene Expression in 786-O Renal Cancer Cells which is Reversed by siSTING or siCGAS

Quantitative reverse-transcriptase polymerase chain reaction (RT-PCR) assay 786-O cells were grown in RPMI-1640 medium containing 10% fetal bovine serum. Cells were then incubated for 2 days at 37 degrees Celsius, 5% CO₂, and 95% humidity. A498 cells were transfected with scrambled siRNA control (siSCR) or siRNA targeting TBK1 (siTBK1), STING (siSTING) or CGAS (siCGAS). After 48 h cells were treated with DMSO or 2 μM of VPS34 inhibitor (Compound 1 or Compound 2) for 24 h. Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Human primers | Forward primer sequence | Reverse primer sequence |
|---|---|---|
| IFNB1 | GCTTGGATTCCTACAAAGAAGCA | ATAGATGGTCAATGCGGCGTC |
| IRF7 | GCTGGACGTGACCATCATGTA | GGGCCGTATAGGAACGTGC |
| CCL5 | CTCCCCATATTCCTCGGACA | ACTCCTTGATGTGGGCACG |
| CXCL10 | TGGCATTCAAGGAGTACCTCTC | ATGCTGATGCAGGTACAGCG |
| Tubulin (house- | GAAGCAGCAACCATGCGTGA | GGCATTGCCAATCTGGACAC |
| keeping gene) | | |

A study was performed to test whether Compound 1 or Compound 2 activated the STING pathway in 786-O renal cell carcinoma cells through increased Type 1 Interferon pathway genes and whether this is ablated through the siRNA knockdown of STING pathway genes. FIG. 4B is a graphical representation of the increase in mRNA expression of IFNB1 (left graph), IRF7 (2nd graph from the left), CCL5 (3rd graph from the left) and CXCL10 (right graph) in response to Compound 1 (middle set of bars on each graph) and Compound 2 (right most set of bars on each graph). The symbols represent the effects of siRNA knockdown of scrambled control (filled circles), siTBK1 (filled square), siSTING (filled triangle) and siCGAS (cross).

Compound 1 increases mRNA expression of IFNB1 (>5-fold), IRF7 (>2-fold), CCL5 (>3-fold) and CXCL10 (˜5-fold). Compound 2 increases mRNA expression of IFNB1 (˜4-fold), IRF7 (˜3-fold), CCL5 (˜4-fold) and CXCL10 (˜5-fold). siRNA knockdown of TBK1 (filled squares) in combination with Compound 1 or Compound 2 results in near complete ablation of IFNB1, and modest decreases in IRF7, CCL5 and CXCL10 in comparison to Compound 1 or Compound 2 alone. siRNA knockdown of STING (filled triangles) in combination with Compound 1 or Compound 2 results in complete ablation of IFNB1, CCL5 and CXCL10 expression and significant decreases in IRF7 (similar to DMSO controls) expression in comparison to Compound 1 or Compound 2 alone. siRNA knockdown of CGAS (cross) in combination with Compound 1 or Compound 2 results in complete ablation of IFNB1, CCL5 and CXCL10 expression and significant decreases in IRF7 (similar to DMSO controls) expression in comparison to Compound 1 or Compound 2 alone. In composite, these data demonstrate that Compound 1 and Compound 2 mediate the increase in type 1 interferon response genes through activation of the CGAS/STING pathway.

### Example 9. Compound 1 or Compound 2 Increases CCL5 and CXCL10 Secreted Proteins which is Reversed by siSTING or siCGAS in A-498 Renal Cancer Cells

### Cytokine Secretion Assay

A-498 cells were transfected with scrambled siRNA control (siSCR) or siRNA targeting TBK1 (siTBK1), STING (siSTING) or CGAS (siCGAS). After 48 h cells were treated with DMSO or 2 μM of VPS34 inhibitor (Compound 1 or Compound 2) for 24 h. Cell culture medium was centrifuged at 1600 rpm for 10 min at 4° C. and obtained supernatant stored at −80° C. Cytokine levels were quantified using following Meso Scale Discovery (MSD) assays: human R-PLEX RANTES/CCL5 (#F21ZN), U-PLEX CXCL10/IP-10 (#K151UFK). Assays were run on the SECTOR Quickplex Imager and analyzed using Discovery Workbench version 4.0 (MSD).

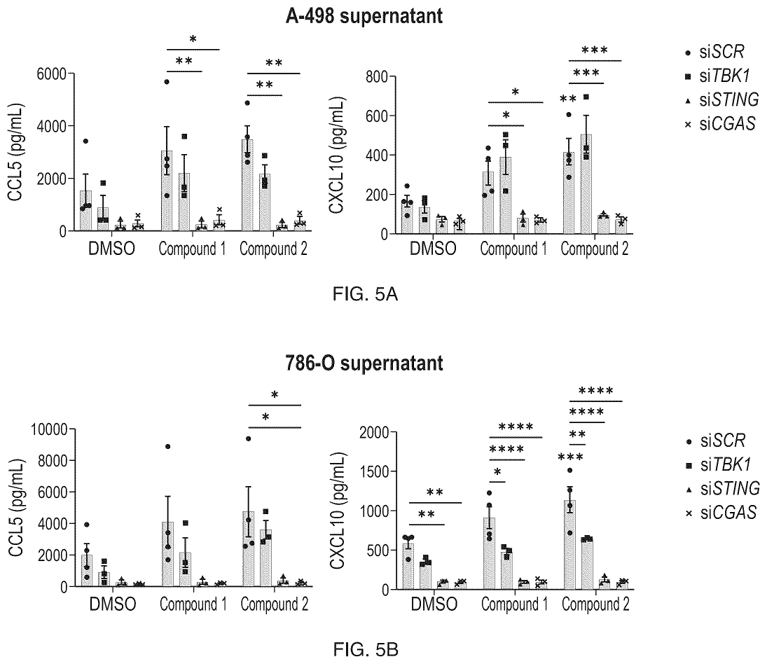

A study was performed to test whether Compound 1 or Compound 2 increased the secretion of CCL5 and CXCL10 in A-498 renal cell carcinoma cells and whether this is ablated through siRNA knockdown of STING pathway proteins. FIG. 5A is a graphical representation of the increased CCL5 protein secretion (left graph), and CXCL10 protein secretion (right graph) in response to Compound 1 (middle set of bars on each graph) and Compound 2 (right most set of bars on each graph). The symbols represent the effects of siRNA knockdown of scrambled control (siSCR; filled circles), siTBK1 (filled square), siSTING (filled triangle) and siCGAS (cross).

Compound 1 and Compound 2 increased CCL5 and CXCL10 protein secretion ˜2-fold in combination with siRNA against non-specific controls (filled circles). siRNA knockdown of TBK1 (filled squares) in combination with Compound 1 or Compound 2 resulted in non-significant changes CCL5 or CXCL10 protein secretion in comparison to Compound 1 or Compound 2 alone. siRNA knockdown of STING (filled triangles) in combination with Compound 1 or Compound 2 resulted in decreases in CCL5 and CXCL10 protein secretion down to the levels of DMSO controls in comparison to Compound 1 or Compound 2 alone. siRNA knockdown of CGAS (cross) in combination with Compound 1 or Compound 2 resulted in decreased CCL5 and CXCL10 secretion down to the levels of DMSO controls in comparison to Compound 1 or Compound 2 alone. In composite, these data demonstrate that Compound 1 and Compound 2 mediate the increase in CCL5 and CXCL10 cytokines through activation of the CGAS/STING pathway.

### Example 10. Compound 1 or Compound 2 Increases CCL5 and CXCL10 Secreted Proteins which is Reversed by siSTING or siCGAS in 786-O Renal Cancer Cells

### Cytokine Secretion Assay

786-O cells were transfected with scrambled siRNA control (siSCR) or siRNA targeting TBK1 (siTBK1), STING (siSTING) or CGAS (siCGAS). After 48 h cells were treated with DMSO or 2 μM of VPS34 inhibitor (Compound 1 or Compound 2) for 24 h. Cell culture medium was centrifuged at 1600 rpm for 10 min at 4° C. and obtained supernatant stored at −80° C. Cytokine levels were quantified using following Meso Scale Discovery (MSD) assays: human R-PLEX RANTES/CCL5 (#F21ZN), U-PLEX CXCL10/IP-10 (#K151UFK). Assays were run on the SECTOR Quickplex Imager and analyzed using Discovery Workbench version 4.0 (MSD).

A study was performed to test whether Compound 1 or Compound 2 increased the secretion of CCL5 and CXCL10 in 786-O renal cell carcinoma cells and whether this is ablated through the siRNA knockdown of STING pathway proteins. FIG. 5B is a graphical representation of the increased CCL5 protein secretion (left graph), and CXCL10 protein secretion (right graph) in response to Compound 1 (middle set of bars on each graph) and Compound 2 (right most set of bars on each graph). The symbols represent the effects of siRNA knockdown of scrambled control (filled circles), siTBK1 (filled square), siSTING (filled triangle) and siCGAS (cross).

Compound 1 and Compound 2 increased CCL5 and CXCL10 protein secretion ˜2-fold in combination with siRNA against non-specific controls (filled circles). siRNA knockdown of TBK1 (filled squares) in combination with Compound 1 or Compound 2 results in decreased secretion of CCL5 or CXCL10 in comparison to Compound 1 or Compound 2 alone. siRNA knockdown of STING (filled triangles) in combination with Compound 1 or Compound 2 resulted in near complete ablation of CCL5 and CXCL10 protein secretion in comparison to Compound 1 or Compound 2 alone. siRNA knockdown of CGAS (cross) in combination with Compound 1 or Compound 2 resulted in near complete ablation of CCL5 and CXCL10 secretion in comparison to Compound 1 or Compound 2 alone. In composite, these data demonstrate that Compound 1 and Compound 2 mediate the increase in CCL5 and CXCL10 cytokines through activation of the CGAS/STING pathway.

### Example 11. siRNA Mediated Knockdown of VPS34 Increases STING Pathway Activation of p-IRF3 and p-STAT1 Signaling in A-498 Renal Cancer Cells, which is Reversed by siRNA Knockdown with siCGAS or siSTING

### Western Blot Assay

A-498 cells were reversely transfected with scrambled siRNA control (siSCR) or siRNA targeting STING (siSTING) or cGAS (siCGAS). After 24 h, cells were forwardly transfected with siSCR control or siRNA targeting VPS34 (siVPS34) for 48 h. A-498 cells were washed in PBS and lysed in RIPA buffer (50 mM Tris HCl pH=7.4, 150 mM NaCl, 1% Triton X-100, 0.5% Na-deoxycholate, 0.1% SDS) containing protease inhibitor cocktail (#11836170001, Roche), and phosphatase inhibitor (#11836170001, Roche) for 10 min at 4° C. After centrifugation at 15000 rpm for 10 min at 4° C., the supernatant was obtained, and protein concentration was quantified using BCA assay (#23225, Thermo Fisher). Equal amounts were mixed with 0.1 M DTT and LDS Sample Buffer (#84788, Thermo Fisher), heated for 5 min at 90° C. before loading onto 4-12% Bis-Tris gels. Proteins were transferred to nitrocellulose membranes. Membranes were blocked in 5% non-fat dry milk in TBS with 0.05% Tween (TBS-T) for 1 h at RT, washed in TBS-T, and probed with primary antibodies shaking overnight at 4° C. After washing in TBS-T, membranes were incubated with IRDye 800CW goat anti-rabbit or IRDye 680RD donkey anti-mouse IgG (H+L) (#926-32211 and #926-68072, respectively, LI-COR, 1:10,000 dilution) secondary antibody in Intercept blocking buffer (#927-60001, LI-COR) for 1 h at RT. After washing in TBS-T, membranes were imaged using the Odyssey CLx (LI-COR). Band intensities were quantified using Image Studio Lite software version 5.2.5 (LI-COR).

| | | Diluent (in | Cat. | |
|---|---|---|---|---|
| Antibody | Dilution | TBS-Tween) | number | Supplier |
| STING | 1:1000 | 5% milk | 13647 | Cell Signaling Technology |
| cGAS | 1:1000 | 5% milk | 15102 | Cell Signaling Technology |
| VPS34 | 1:1000 | 5% BSA in | GTX129528 | GeneTex |
| | | TBS-T | | |
| phospho-IRF3 Ser386 | 1:500 | 5% BSA | 37829 | Cell Signaling Technology |
| STAT1 | 1:1000 | 5% milk | 9172 | Cell Signaling Technology |
| phospho-STAT1 | 1:1000 | 5% BSA | 7649 | Cell Signaling Technology |
| Tyr701 | | | | |
| Actin | 1:10,000 | 5% milk | A5441 | Sigma-Aldrich |

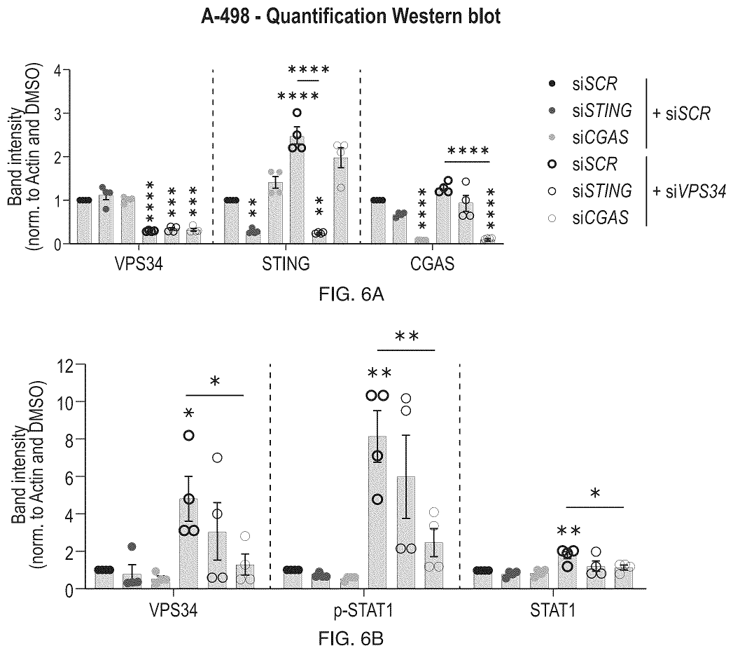

A study was performed to test whether siRNA knockdown of VPS34 mimicked the effects of Compound 1 or Compound 2 in activating the STING pathway in A-498 renal cell carcinoma cells. FIG. 6A/B is a graphical representation of quantified Western blot data measuring protein levels of VPS34 (FIG. 6A, first set of bars), STING (FIG. 6A, second set of bars), CGAS (FIG. 6A, third set of bars), p-IRF3 (FIG. 6B, first set of bars), p-STAT1 (FIG. 6B, second set of bars) and STAT1 (FIG. 6B, third set of bars). The symbols represent the effects of siRNA knockdown of scrambled control (siSCR)+scrambled control (siSCR; black filled circles), siSTING+scrambled control (grey filled circles) and siCGAS+scrambled controls (light grey filled circles), scrambled control+siVPS34 (black open circles), siSTING+siVPS34 (grey open circles) and siCGAS+siVPS34 (light grey open circles). All data has been normalized to actin and DMSO controls.

In FIG. 6A, siRNA knockdown of VPS34 alone (black open circles), resulted in decreased VPS34 protein expression (bar 4) and increased STING protein expression (bar 10). In FIG. 6B, siRNA knockdown of VPS34 alone resulted in increased p-IRF3 (bar 4) and p-STAT1 (bar 10), demonstrating that VPS34 knockdown leads to an increase in STING pathway signaling. siRNA knockdown of VPS34 in combination with siSTING (grey open circles) resulted in decreased VPS34 (FIG. 6A, bar 5) and STING (FIG. 6A, bar 11) protein expression, demonstrating the efficiency of the respective siRNAs. siSTING partially reversed the effects of siVPS34, resulting in decreased levels of p-IRF3 and p-STAT1 levels (FIG. 6B, compare bar 5 with bar 4 and bar 11 with bar 10). siRNA knockdown of VPS34 in combination with siCGAS resulted in decreased VPS34 (FIG. 6A, bar 6) and CGAS (FIG. 6A, bar 18) protein expression, demonstrating the efficiency of the respective siRNAs. siCGAS dramatically reversed the effects of siVPS34, decreasing p-IRF3 and p-STAT1 levels (FIG. 6B, compare bar 6 with bar 4 and bar 12 with bar 10). In composite, these results demonstrate that knockdown of VPS34 mimics pharmacologic inhibition of VPS34 by Compound 1 or Compound 2, and that the effects of VPS34 knockdown to increase signaling through the STING signaling pathway are blocked by knockdown of STING or CGAS.

### Example 12. siRNA Mediated Knockdown of VPS34 Upregulates Type 1 Interferon Response, CCL5 and CXCL10 Gene Expression in A-498 Renal Cancer Cells, which is Reversed by siCGAS

### Quantitative Reverse-Transcriptase Polymerase Chain Reaction (RT-PCR) Assay

A-498 cells were grown in Minimal Essential Medium containing 10% fetal bovine serum. Cells were then incubated for 2 days at 37 degrees Celsius, 5% CO₂, and 95% humidity. A-498 cells were reversely transfected with scrambled siRNA control (siSCR) or siRNA targeting STING (siSTING) or cGAS (siCGAS). After 24 h, cells were forwardly transfected with siSCR control or siRNA targeting VPS34 (siVPS34) for 48 h. Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Human primers | Forward primer sequence | Reverse primer sequence |
|---|---|---|
| IFNB1 | GCTTGGATTCCTACAAAGAAGCA | ATAGATGGTCAATGCGGCGTC |
| IRF7 | GCTGGACGTGACCATCATGTA | GGGCCGTATAGGAACGTGC |
| CCL5 | CTCCCCATATTCCTCGGACA | ACTCCTTGATGTGGGCACG |
| CXCL10 | TGGCATTCAAGGAGTACCTCTC | ATGCTGATGCAGGTACAGCG |
| Tubulin (house- | GAAGCAGCAACCATGCGTGA | GGCATTGCCAATCTGGACAC |
| keeping gene) | | |

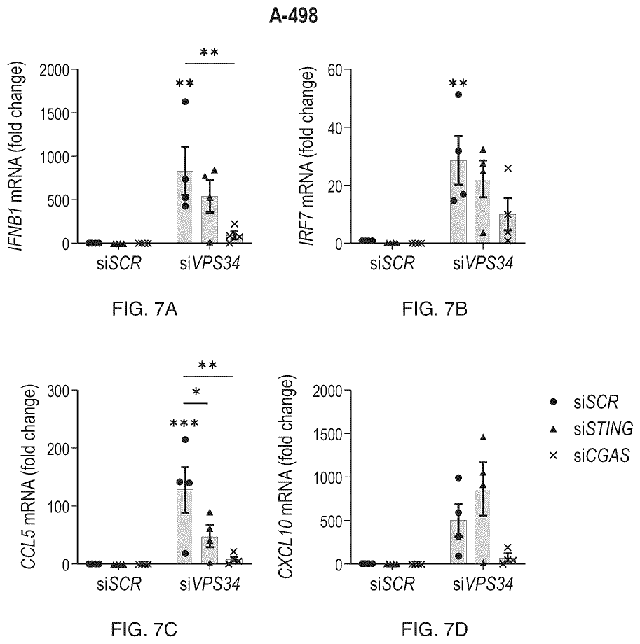

A study was performed to test whether siRNA knockdown of VPS34 increased the mRNA expression of Type 1 interferon genes IFNB1 and IRF7, as well as CCL5 and CXCL10 and whether knockdown of STING pathway genes modulated that increase in A-498 renal cell carcinoma cells. FIG. 7 is a graphical representation of mRNA expression of IFNB1 (FIG. 7A), IRF7 (FIG. 7B), CCL5 (FIG. 7C) and CXCL10 (FIG. 7D) in response to siRNA knockdown of scrambled control (siSCR; circles), siSTING (triangles) and siCGAS (cross). All data is normalized to the house-keeping gene control, Tubulin.

In FIG. 7A, siRNA knockdown of VPS34 resulted in increased expression of IFNB1 (>500-fold), which was mildly reduced in combination with siSTING but nearly completely abolished in combination with siCGAS. In FIG. 7B, siRNA knockdown of VPS34 resulted in increased expression of IRF7 (˜30-fold), which was not significantly decreased in combination with siSTING and decreased by ˜70% in combination with siCGAS. In FIG. 7C, siRNA knockdown of VPS34 resulted in increased expression of CCL5 (>100-fold), which was significantly decreased in combination with siSTING, and nearly completely abolished in combination with siCGAS. In FIG. 7D, siRNA knockdown of VPS34 results in increased CXCL10 expression (˜500-fold), which was not significantly altered in combination with siSTING but nearly abolished in combination with siCGAS. In composite, these data demonstrate that knockdown of VPS34 phenocopies the effect of pharmacological inhibition of VPS34 by Compound 1 or Compound 2 to stimulate type 1 interferon response genes and cytokines. These effects of VPS34 knockdown are reversed by knockdown of STING or CGAS, again phenocopying the effects of STING or CGAS knockdown to reverse the effects of Compound 1 or Compound 2.

### Example 13. siRNA Mediated Knockdown of VPS34 Upregulates IFNβ1, CCL5 and CXCL10 Secreted Proteins in A-498 Renal Cancer Cells, which is Reversed by siCGAS

### Cytokine Secretion Assay

A-498 cells were reversely transfected with scrambled siRNA control (siSCR) or siRNA targeting STING (siSTING) or cGAS (siCGAS). After 24 h, cells were forwardly transfected with siSCR control or siRNA targeting VPS34 (siVPS34) for 48 h. Cell culture medium was centrifuged at 1600 rpm for 10 min at 4° C. and obtained supernatant stored at −80° C. Cytokine levels were quantified using following Meso Scale Discovery (MSD) assays: human R-PLEX RANTES/CCL5 (#F21ZN), U-PLEX CXCL10/IP-10 (#K151UFK), U-PLEX IFNβ (#K151VIK). Assays were run on the SECTOR Quickplex Imager and analyzed using Discovery Workbench version 4.0 (MSD).

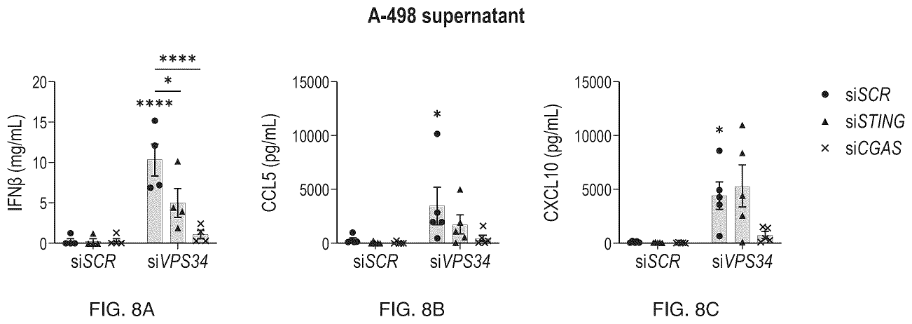

A study was performed to test whether siRNA knockdown of VPS34 increased the secretion of IFNβ, CCL5, and CXCL10 and whether knockdown of STING pathway genes abolished that increase in A-498 renal cell carcinoma cells. FIG. 8 is a graphical representation of protein secretion of IFNβ (FIG. 8A), CCL5 (FIG. 8B) and CXCL10 (FIG. 8C) in response to siRNA knockdown of scrambled control siSCR (circles), siSTING (triangles) and siCGAS (cross).

In FIG. 8A, siRNA knockdown of VPS34 resulted in increased secretion of IFNβ (˜10-fold), which was mildly reduced in combination with siSTING but nearly completely abolished in combination with siCGAS. In FIG. 8B, siRNA knockdown of VPS34 resulted in increased expression of CCL5 (˜4000-fold), which was not significantly decreased in combination with siSTING but abolished with siCGAS. In FIG. 8C, siRNA knockdown of VPS34 resulted in increased expression of CXCL10 (˜5000-fold), which was not altered in combination with siSTING, but nearly completely abolished in combination with siCGAS.

### Example 14. Compound 1 or Compound 2 Exhibits Additivity or Synergy in Combination with a STING Agonist to Upregulate IFNβ, CCL5 and CXCL10 Gene Expression in A-498 Renal Cancer Cells

### Quantitative Reverse-Transcriptase Polymerase Chain Reaction (RT-PCR) Assay

A-498 cells were grown in Minimal Essential Medium containing 10% fetal bovine serum. Cells were then incubated for 2 days at 37 degrees Celsius, 5% CO₂, and 95% humidity. A-498 cells were treated with DMSO control, 2 μM Compound 1 or 2 μM Compound 2 in combination with 50 μM STING agonist ADU-S100 for 24 h. Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Human primers | Forward primer sequence | Reverse primer sequence |
|---|---|---|
| IFNB1 | GCTTGGATTCCTACAAAGAAGCA | ATAGATGGTCAATGCGGCGTC |
| CCL5 | CTCCCCATATTCCTCGGACA | ACTCCTTGATGTGGGCACG |
| CXCL10 | TGGCATTCAAGGAGTACCTCTC | ATGCTGATGCAGGTACAGCG |
| Tubulin (house- | GAAGCAGCAACCATGCGTGA | GGCATTGCCAATCTGGACAC |
| keeping gene) | | |

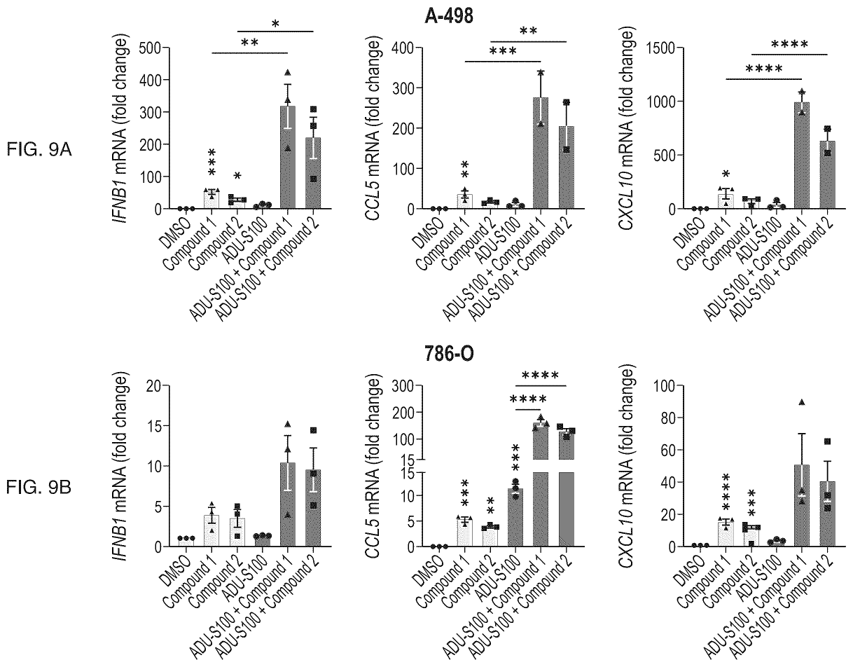

A study was performed to test whether Compound 1 or Compound 2 would augment the mRNA expression of STING pathway gene IFNB1 and CCL5 and CXCL10 expression in combination with the STING agonist, ADU-S100, in A-498 renal cell carcinoma cells. FIG. 9A is a graphical representation of the mRNA expression of IFNB1 (1st graph), CCL5 (2nd graph) and CXCL10 (3rd graph) with Compound 1 (2nd bar), Compound 2 (3rd bar), ADU-S100 (4th bar), Compound 1+ADU-S100 (5th bar) or Compound 2+ADU-S100 (6th bar).

Compound 1 mildly increased IFNB1, CCL5 and CXCL10 expression. Compound 2 mildly increased IFNB1, CCL5 and CXCL10 expression. ADU-S100 has no effect on IFNB1 expression, and increased CCL5 and CXCL10 expression. The combination of Compound 1 and ADU-S100 synergistically increased the expression of IFNB1, CCL5 and CXCL10 in comparison to single agents alone (compare bar 5 to bars 2 and 4). The combination of Compound 2 and ADU-S100 synergistically increased the expression of IFNB1, CCL5 and CXCL10 in comparison to single agents alone (compare bar 6 to bars 3 and 4).

### Example 15. Compound 1 or Compound 2 Exhibits Additivity or Synergy in Combination with a STING Agonist to Upregulate IFNβ, CCL5 and CXCL10 Gene Expression in 786-O Renal Cancer Cells

### Quantitative Reverse-Transcriptase Polymerase Chain Reaction (RT-PCR) Assay

786-O cells were grown in RPMI-1640 medium containing 10% fetal bovine serum. Cells were then incubated for 2 days at 37 degrees Celsius, 5% CO₂, and 95% humidity. A-498 cells were treated with DMSO control, 2 μM Compound 1 or 2 μM Compound 2 in combination with 50 μM STING agonist ADU-S100 for 24 h. Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Human primers | Forward primer sequence | Reverse primer sequence |
|---|---|---|
| IFNB1 | GCTTGGATTCCTACAAAGAAGCA | ATAGATGGTCAATGCGGCGTC |
| CCL5 | CTCCCCATATTCCTCGGACA | ACTCCTTGATGTGGGCACG |
| CXCL10 | TGGCATTCAAGGAGTACCTCTC | ATGCTGATGCAGGTACAGCG |
| Tubulin (house- | GAAGCAGCAACCATGCGTGA | GGCATTGCCAATCTGGACAC |
| keeping gene) | | |

A study was performed to test whether Compound 1 or Compound 2 would augment the mRNA expression of STING pathway gene IFNB1 and CCL5 and CXCL10 expression in combination with the STING agonist, ADU-S100 in 786-O renal cell carcinoma cells. FIG. 9B is a graphical representation of the mRNA expression of IFNB1 (1st graph), CCL5 (2nd graph) and CXCL10 (3rd graph) with Compound 1 (2nd bar), Compound 2 (3rd bar), ADU-S100 (4th bar), Compound 1+ADU-S100 (5th bar) or Compound 2+ADU-S100 (6th bar).

Compound 1 as a single agent increased IFNB1, CCL5 and CXCL10 expression. Compound 2 as a single agent increased IFNB1, CCL5 and CXCL10 expression. ADU-S100 had no effect on IFNB1 and CXCL10 expression, and increased CCL5 expression. The combination of Compound 1 and ADU-S100 synergistically increased the expression of IFNB1, CCL5 and CXCL10 in comparison to single agents alone (compare bar 5 to bars 2 and 4). The combination of Compound 2 and ADU-S100 synergistically increased the expression of IFNB1, CCL5 and CXCL10 in comparison to single agents alone (compare bar 6 to bars 3 and 4).

### Example 16. Compound 1 or Compound 2 Improves Proinflammatory Cytokine Response in Combination with STING Agonist in A-498 Renal Cancer Cells in Comparison to Either Single Agent Alone

### Cytokine Secretion Assay

A-498 cells were treated with DMSO control, 2 μM Compound 1 or 2 μM Compound 2 in combination with 50 μM STING agonist ADU-S100 for 24 h. Cell culture medium was centrifuged at 1600 rpm for 10 min at 4° C. and obtained supernatant stored at −80° C. Cytokine levels were quantified using following Meso Scale Discovery (MSD) assays: human R-PLEX RANTES/CCL5 (#F21ZN), U-PLEX CXCL10/IP-10 (#K151UFK). Assays were run on the SECTOR Quickplex Imager and analyzed using Discovery Workbench version 4.0 (MSD). Low concentrations of human IFNβ were determined using High Sensitivity IFN Beta ELISA Kit, (PBL assay science, #41415-1).

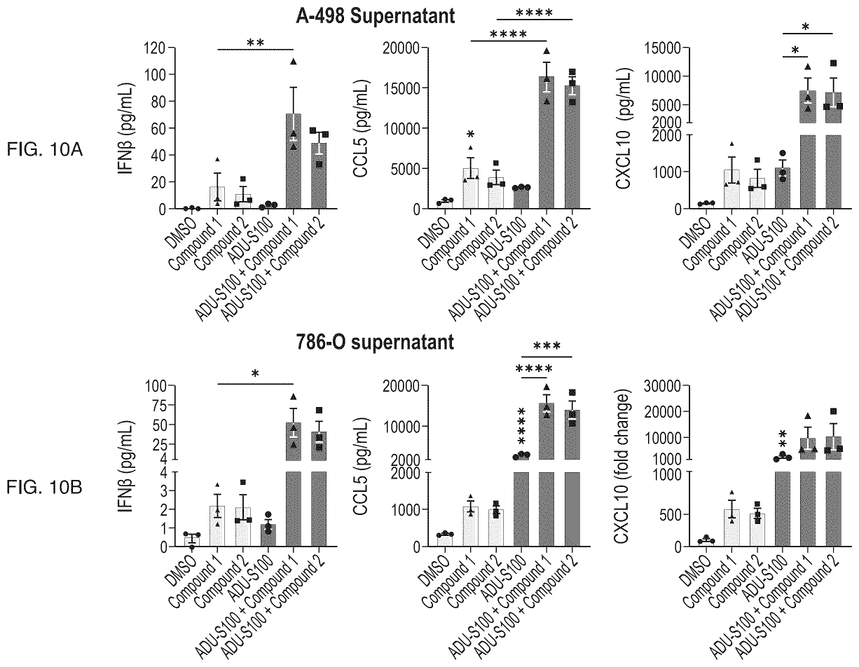

A study was performed to test whether Compound 1 or Compound 2 would augment the STING pathway and CCL5 and CXCL10 secretion in combination with the STING agonist, ADU-S100 in A-498 renal cell carcinoma cells. FIG. 10A is a graphical representation of IFNβ (1st graph), CCL5 (2nd graph) and CXCL10 (3rd graph) protein secretion with Compound 1 (2nd bar), Compound 2 (3rd bar), ADU-S100 (4th bar), Compound 1+ADU-S100 (5th bar) or Compound 2+ADU-S100 (6th bar).

Compound 1 as a single agent increased IFNβ, CCL5 and CXCL10 secretion. Compound 2 as a single agent increased IFNβ, CCL5 and CXCL10 secretion. ADU-S100 had no effect on IFNβ secretion, and mildly increased CCL5 and CXCL10 secretion. The combination of Compound 1 and ADU-S100 synergistically increased the secretion of IFNβ, CCL5 and CXCL10 in comparison to single agents alone (compare bar 5 to bars 2 and 4). The combination of Compound 2 and ADU-S100 synergistically increased the secretion of IFNβ, CCL5 and CXCL10 in comparison to single agents alone (compare bar 6 to bars 3 and 4).

### Example 17. Compound 1 or Compound 2 Improves Proinflammatory Cytokine Response in Combination with STING Agonist in 786-O Renal Cancer Cells in Comparison to Either Single Agent Alone

### Cytokine Secretion Assay

786-O cells were treated with DMSO control, 2 μM Compound 1 or 2 μM Compound 2 in combination with 50 μM STING agonist ADU-S100 for 24 h. Cell culture medium was centrifuged at 1600 rpm for 10 min at 4° C. and obtained supernatant stored at −80° C. Cytokine levels were quantified using following Meso Scale Discovery (MSD) assays: human R-PLEX RANTES/CCL5 (#F21ZN), U-PLEX CXCL10/IP-10 (#K151UFK). Assays were run on the SECTOR Quickplex Imager and analyzed using Discovery Workbench version 4.0 (MSD). Low concentrations of human IFNβ were determined using High Sensitivity IFN Beta ELISA Kit, (PBL assay science, #41415-1).

A study was performed to test whether Compound 1 or Compound 2 would augment the STING pathway and CCL5 and CXCL10 secretion in combination with the STING agonist, ADU-S100 in 786-O renal cell carcinoma cells. FIG. 10B is a graphical representation of IFNβ (1st graph), CCL5 (2nd graph) and CXCL10 (3rd graph) protein secretion with Compound 1 (2nd bar), Compound 2 (3rd bar), ADU-S100 (4th bar), Compound 1+ADU-S100 (5th bar) or Compound 2+ADU-S100 (6th bar).

Compound 1 as a single agent increased IFNβ, CCL5 and CXCL10 secretion. Compound 2 as a single agent increased IFNβ, CCL5 and CXCL10 secretion. ADU-S100 had a mild effect on IFNβ secretion, and significantly increased CCL5 and CXCL10 secretion. The combination of Compound 1 and ADU-S100 further increased the secretion of IFNβ, CCL5, and CXCL10 in comparison to single agents alone (compare bar 5 to bars 2 and 4). The combination of Compound 2 and ADU-S100 increased the secretion of IFNβ, CCL5 and CXCL10 in comparison to single agents alone (compare bar 6 to bars 3 and 4).

### Example 18. Compound 1 Upregulates Ifnβ, Ccl5 and Cxcl10 Gene Expression in Combination with STING Agonist in Renca Renal Cancer Cells

### Quantitative Reverse-Transcriptase Polymerase Chain Reaction (RT-PCR) Assay

Renca cells were grown in RPMI-1640 medium containing 10% fetal bovine serum. Cells were then incubated for 2 days at 37 degrees Celsius, 5% CO₂, and 95% humidity. Renca cells were reversely transfected with scrambled siRNA control (siSCR) or siRNA targeting STING (siSTING). After transfection for 48 h, cells were treated with DMSO control, 2 μM Compound 1 or 2 μM Compound 2 in combination with 10 μM ADU-S100 for 4 h (for Ifnb1) or 24 h (Ccl5 and Cxcl10). Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Mouse primers | Forward primer sequence | Reverse primer sequence |
|---|---|---|
| Ccl5 | GCTGCTTTGCCTACCTCTCC | TCGAGTGACAAACACGACTGC |
| Cxcl10 | CCAAGTGCTGCCGTCATTTTC | GGCTCGCAGGGATGATTTCAA |
| Ifnb1 | CAGCTCCAAGAAAGGACGAAC | GGCAGTGTAACTCTTCTGCAT |
| Rps18 (house | AGTTCCAGCACATTTTGCGAG | TCATCCTCCGTGAGTTCTCCA |
| keeping gene) | | |

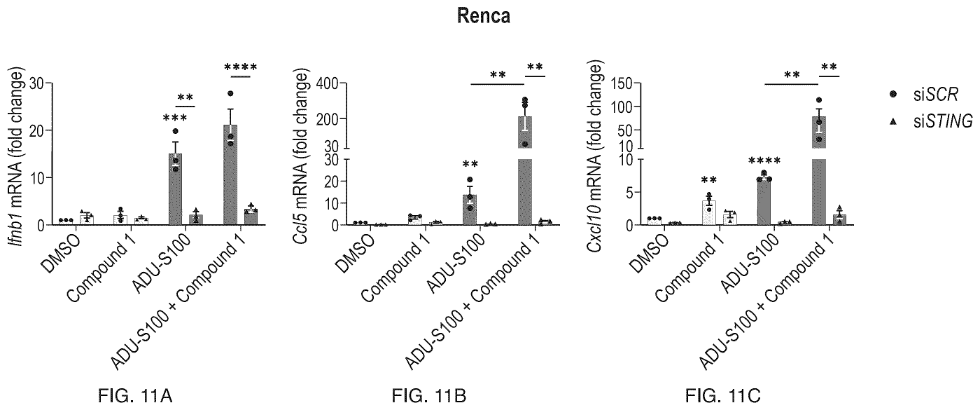

A study was performed to test whether combining Compound 1 with a STING agonist, ADU-S100 would increase the mRNA expression of IFNB1, CCL5 and CXCL10 and if siRNA knockdown of STING would abolish that increase in Renca renal cell carcinoma cells. FIGS. 11A-11C are a graphical representation of the mRNA expression of IFNB1 (FIG. 11A), CCL5 (FIG. 11B) and CXCL10 (FIG. 11C) and the effect of Compound 1 (2nd set of bars), ADU-S100 (3rd set of bars) or the combination of Compound 1+ADU-S100 (4th set of bars).

Compound 1 as a single agent did not significantly increase IFNB1 expression but did increase CCL5 and CXCL10 expression. ADU-S100 as a single agent increased the expression of IFNB1, CCL5 and CXCL10. The combination of Compound 1 and ADU-S100 further increased IFNB1, and synergistically increased CCL5 and CXCL10 expression.

SiRNA knockdown of STING blocked the increase in IFNB1, CCL5 and CXCL10 by Compound 1, ADU-S100, and the combination in all cases.

### Example 19. Compound 1 or Compound 2 Upregulates Ifnβ, Ccl5 and Cxcl10 Gene Expression in Combination with STING Agonist in B16-F10 Murine Melanoma Cells in Comparison to Either Single Agent Alone

### Quantitative Reverse-Transcriptase Polymerase Chain Reaction (RT-PCR) Assay

B16-F10 cells were grown in DMEM/F12 medium containing 10% fetal bovine serum. Cells were then incubated for 2 days at 37 degrees Celsius, 5% CO₂, and 95% humidity. Renca cells were reversely transfected with scrambled siRNA control (siSCR) or siRNA targeting STING (siSTING). After transfection for 48 h, cells were treated with DMSO control, 2 μM Compound 1 or 2 μM Compound 2 in combination with 10 μM ADU-S100 for 4 h (for Ifnb1) or 24 h (Ccl5 and Cxcl10). Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Mouse primers | Forward primer sequence | Reverse primer sequence |
|---|---|---|
| Ccl5 | GCTGCTTTGCCTACCTCTCC | TCGAGTGACAAACACGACTGC |
| Cxcl10 | CCAAGTGCTGCCGTCATTTTC | GGCTCGCAGGGATGATTTCAA |
| Ifnb1 | CAGCTCCAAGAAAGGACGAAC | GGCAGTGTAACTCTTCTGCAT |
| Rps18 (house | AGTTCCAGCACATTTTGCGAG | TCATCCTCCGTGAGTTCTCCA |
| keeping gene) | | |

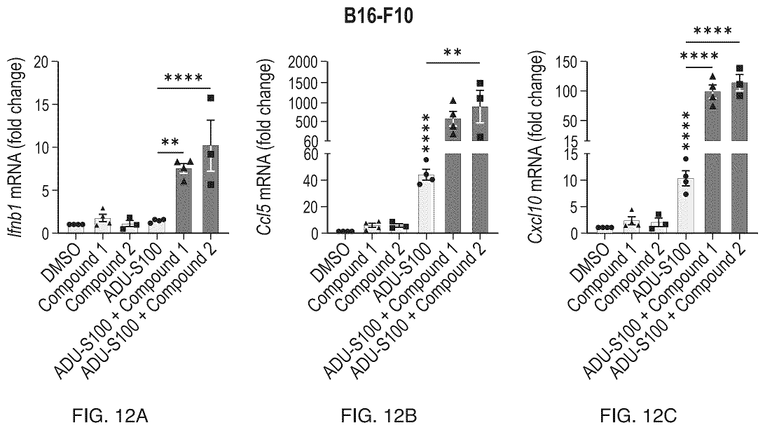

A study was performed to test whether Compound 1 or Compound 2 upregulated IFNB1, CCL5 and CXCL10 in combination with a STING agonist, ADU-S100, in B16-F10 melanoma cells. FIGS. 12A-12C is a graphical representation of mRNA expression of IFNB1 (FIG. 12A), CCL5 (FIG. 12B) and CXCL10 (FIG. 12C) and the effect of Compound 1 (2nd set of bars), Compound 2 (3^{rd} set of bars), ADU-S100 (4th set of bars), the combination of Compound 1+ADU-S100 (5th set of bars), or the combination of Compound 2+ADU-S100 (6th set of bars).

Compound 1 as a single agent did not increase IFNB1 expression but did mildly increase CCL5 and CXCL10 expression. Compound 2 as a single agent did not increase IFNB1 expression but did mildly increase CCL5 and CXCL10 expression. ADU-S100 did not increase the expression of IFNB1 but did increase the expression of CCL5 and CXCL10. The combination of Compound 1 and ADU-S100 synergistically increased IFNB1, CCL5 and CXCL10 expression. The combination of Compound 2 and ADU-S100 synergistically increased IFNB1, CCL5 and CXCL10 expression.

### Example 20. Combination of Compound 1 and STING Agonist Inhibits Cell Growth and Induces Apoptosis in Renca Renal Cancer Cells

### IncuCyte Live-Cell Imaging

Renca cells were treated with a combination of DMSO or 2 μM Compound 1 alone or in combination with STING agonist ADU-S100 for 24 hours. Treatment with 1 μM staurosporine was used as a positive control for apoptosis induction. To assess cell proliferation, 1000 cells/well were plated in 96-well plates. After 24 h, cells were treated with compounds in 100 μL complete medium. Phase-contrast images (10×) were acquired every 3 h for 24-72 h and cell confluence (%) was calculated using the IncuCyte S3 live-cell analysis system (Sartorius). For monitoring apoptosis, 5 μM of IncuCyte Caspase-3/7 Green Dye (#4440, Sartorius) was added to the medium at the time of treatment. Both phase-contrast and green fluorescent images were captured. Caspase 3/7 induction was calculated by counting green fluorescent objects per mm² and dividing it by the percentage of cell confluency.

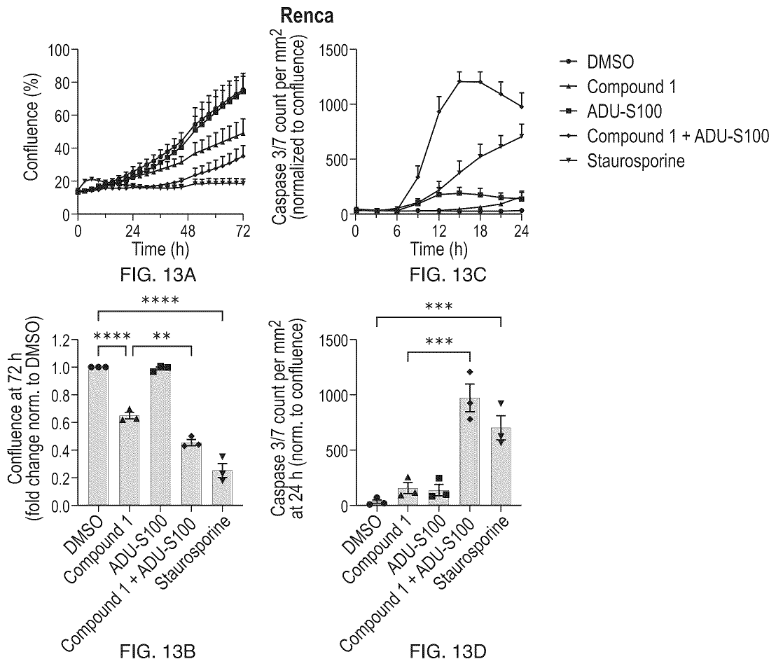

A study was performed to test whether Compound 1, in combination with a STING agonist, ADU-S100, would inhibit cell growth and induce apoptosis in Renca renal cell carcinoma cells. FIGS. 13A-13D are graphical representations of cell confluence (FIGS. 13A and 13B) or Caspase 3/7 activity (FIGS. 13C and 13D). In FIG. 13A, cell confluence is depicted after cells were treated with DMSO (circles), Compound 1 (triangles), ADU-S100 (squares), the combination of Compound 1 and ADU-S100 (diamond) or staurosporine as a control (upside-down triangle). Treatment with Compound 1 decreased the confluence of Renca cells in comparison to cells treated with DMSO alone. Treatment with ADU-S100 had no effect on the confluence of Renca cells. The combination of Compound 1 and ADU-S100 further decreased the confluence of Renca cells when compared to single agent alone (either Compound 1 or ADU-S100). These data at 72 hours are depicted as a bar graph in FIG. 13B.

In FIG. 13C, Caspase 3/7 activity is depicted after cells were treated with DMSO (circles), Compound 1 (triangles), ADU-S100 (squares), the combination of Compound 1 and ADU-S100 (diamond) or staurosporine (upside-down triangle). Treatment of Renca cells with Compound 1 increased Caspase 3/7 activity in comparison to cells treated with DMSO control. Treatment with ADU-S100 had mild effects on Caspase 3/7 activity. The combination of Compound 1 and ADU-S100 synergistically increased Caspase 3/7 activity in comparison to single agent treatments. These data at 24 hours are depicted as a bar graph in FIG. 13D.

### Example 21. Compound 1 or Compound 2 Increases IFNB1, CCL5 and CXCL10 Expression which is Reversed by siSTING in Me30966 Melanoma Cells

### Quantitative Reverse-Transcriptase Polymerase Chain Reaction (RT-PCR) Assay

Me30966 cells were transfected with siSCR or siSTING. After 48 h, cells were treated with DMSO or 2 μM of VPS34 inhibitors (Compound 1 or Compound 2) for 24 h. Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Human primers | Forward primer sequence | Reverse primer sequence |
|---|---|---|
| IFNB1 | GCTTGGATTCCTACAAAGAAGCA | ATAGATGGTCAATGCGGCGTC |
| CCL5 | CTCCCCATATTCCTCGGACA | ACTCCTTGATGTGGGCACG |
| CXCL10 | TGGCATTCAAGGAGTACCTCTC | ATGCTGATGCAGGTACAGCG |
| Tubulin (house- | GAAGCAGCAACCATGCGTGA | GGCATTGCCAATCTGGACAC |
| keeping gene) | | |

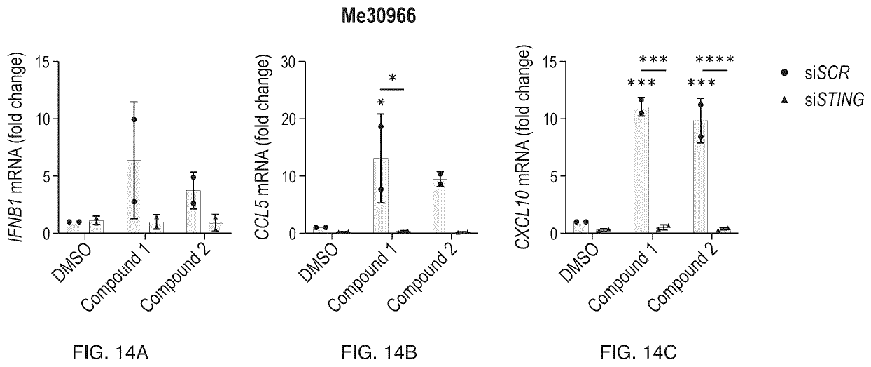

A study was performed to test whether Compound 1 (Compound 1) or Compound 2 (Compound 2) increased IFNB1, CCL5 and CXCL10 mRNA expression and if siRNA knockdown of STING would reverse that effect in Me30966 melanoma cells. FIG. 14A-14C is a graphical representation of the mRNA expression of IFNB1 (FIG. 14A), CCL5 (FIG. 14B) and CXCL10 (FIG. 14C) and the effect of Compound 1 (2nd set of bars) or Compound 2 (3rd set of bars) on mRNA expression of IFNB1, CCL5 and CXCL10. Each set of experiments were modulated by siRNA knockdown of scrambled control (siSCR, circles) or siSTING (triangle) in Me30966 melanoma cells.

In FIG. 14A, Compound 1 and Compound 2 increased IFNB1 mRNA expression (˜5-fold) which was abolished by siSTING. In FIG. 14B, Compound 1 and Compound 2 increased CCL5 mRNA expression (˜10-fold) which was abolished by siSTING. In FIG. 14C, Compound 1 and Compound 2 increased CXCL10 mRNA expression (˜10-fold) which was abolished by siSTING.

### Example 22. Compound 1 or Compound 2 Increases CCL5 and CXCL10 Gene Expression and Protein Secretion and Enhances Activity of Endogenous STING Agonist cGAMP in CT26 Colorectal Cancer Cells

### Quantitative Reverse-Transcriptase Polymerase Chain Reaction (RT-PCR) Assay and Cytokine Secretion Assay

CT26 cells were treated for 24 hours with DMSO or VPS34 inhibitors (5 μM Compound 1 or 10 μM Compound 2) in combination with 10 mg/mL STING agonist cGAMP (green bars). Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Mouse primers | Forward primer sequence | Reverse primer sequence |
|---|---|---|
| Ccl5 | GCTGCTTTGCCTACCTCTCC | TCGAGTGACAAACACGACTGC |
| Cxcl10 | CCAAGTGCTGCCGTCATTTTC | GGCTCGCAGGGATGATTTCAA |
| Rps18 (house- | AGTTCCAGCACATTTTGCGAG | TCATCCTCCGTGAGTTCTCCA |
| keeping gene) | | |

Cell culture medium was centrifuged at 1600 rpm for 10 min at 4° C. and obtained supernatant stored at −80° C. Cytokine levels were quantified using following Meso Scale Discovery (MSD) assays: mouse U-PLEX RANTES/CCL5 (#K152A2K) and U-PLEX IP-10/CXCL10 (#K152UFK). Assays were run on the SECTOR Quickplex Imager and analyzed using Discovery Workbench version 4.0 (MSD).

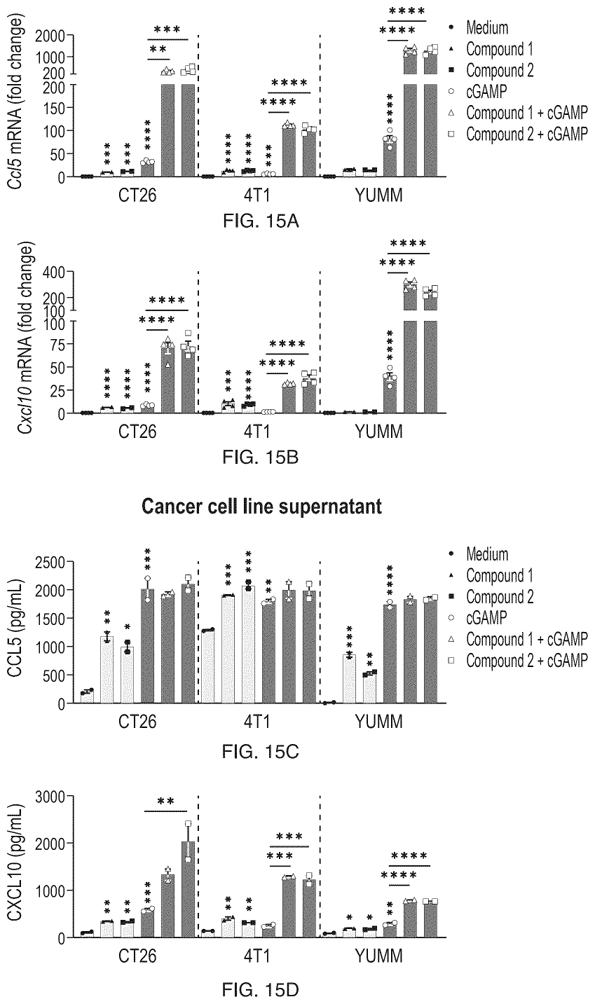

A study was performed to test whether Compound 1 or Compound 2 increase CCL5 and CXCL10 gene expression alone and in combination with an endogenous STING agonist, cGAMP. FIGS. 15A-15D is a graphical representation of the mRNA expression of CCL5 (FIG. 15A) and CXCL10 (FIG. 15B) and protein secretion of CCL5 (FIG. 15C) and CXCL10 (FIG. 15D) treated with medium control (first bar, circle), Compound 1 (2nd bar, triangle), Compound 2 (3rd bar, square), cGAMP (4th bar, circle), the combination of Compound 1+cGAMP (5th bar, triangle) and the combination of Compound 2+cGAMP (6th bar, square) in CT26 colorectal cancer cells (first set of bars)

In FIG. 15A, Compound 1, Compound 2 and cGAMP increased CCL5 mRNA expression in CT26 which was further synergistically increased by the combination treatment of Compound 1+cGAMP or Compound 2+cGAMP. In FIG. 15B, Compound 1, Compound 2 and cGAMP and increased CXCL10 mRNA expression in CT26 cells which was synergistically increased by the combination treatment of Compound 1+cGAMP or Compound 2+cGAMP. In FIG. 15C, Compound 1, Compound 2 and cGMP increased CCL5 protein secretion in CT26 cells which was not significantly further augmented by the combination treatment of Compound 1+cGAMP or Compound 2+cGAMP. In FIG. 15D, Compound 1, Compound 2 and cGAMP increased CXCL10 protein secretion in CT26 cells which was further synergistically increased by the combination treatment of Compound 1+cGAMP or Compound 2+cGAMP.

### Example 23. Compound 1 or Compound 2 Increases CCL5 and CXCL10 Gene Expression and Protein Secretion and Enhances Activity of Endogenous STING Agonist cGAMP in 4T1 Breast Cancer Cells

### Quantitative Reverse-Transcriptase Polymerase Chain Reaction (RT-PCR) Assay and Cytokine Secretion Assay

4T1 cells were treated for 24 hours with DMSO or VPS34 inhibitors (5 μM Compound 1 or 10 μM Compound 2) in combination with 10 mg/ml STING agonist cGAMP (green bars). Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Mouse primers | Forward primer sequence | Reverse primer sequence |
|---|---|---|
| Ccl5 | GCTGCTTTGCCTACCTCTCC | TCGAGTGACAAACACGACTGC |
| Cxcl10 | CCAAGTGCTGCCGTCATTTTC | GGCTCGCAGGGATGATTTCAA |
| Rps18 (house- | AGTTCCAGCACATTTTGCGAG | TCATCCTCCGTGAGTTCTCCA |
| keeping gene) | | |

Cell culture medium was centrifuged at 1600 rpm for 10 min at 4° C. and obtained supernatant stored at −80° C. Cytokine levels were quantified using following Meso Scale Discovery (MSD) assays: mouse U-PLEX RANTES/CCL5 (#K152A2K) and U-PLEX IP-10/CXCL10 (#K152UFK). Assays were run on the SECTOR Quickplex Imager and analyzed using Discovery Workbench version 4.0 (MSD).

A study was performed to test whether Compound 1 or Compound 2 increase CCL5 and CXCL10 gene expression alone and in combination with a STING agonist, ADU-S100 in addition to enhancing the effects of an endogenous STING agonist, cGAMP. FIGS. 15A-15D is a graphical representation of the mRNA expression of CCL5 (FIG. 15A) and CXCL10 (FIG. 15B) and protein secretion of CCL5 (FIG. 15C) and CXCL10 (FIG. 15D) in 4T1 cells (second set of bars) treated with medium control (first bar, circle), Compound 1 (2^{nd} bar, triangle), Compound 2 (3^{rd} bar, square), cGAMP (4^{th} bar, circle), the combination of Compound 1+cGAMP (5^{th} bar, triangle) and the combination of Compound 2+cGAMP (6^{th} bar, square).

In FIG. 15A, Compound 1, Compound 2 and cGAMP increased CCL5 mRNA expression in 4T1 cells which was further synergistically increased by the combination treatment of Compound 1+cGAMP or Compound 2+cGAMP. In FIG. 15B, Compound 1, Compound 2 and cGAMP and increased CXCL10 mRNA expression in 4T1 cells which was further synergistically increased by the combination treatment of Compound 1+cGAMP or Compound 2+cGAMP. In FIG. 15C, Compound 1, Compound 2 and cGMP increased CCL5 protein secretion in 4T1 cells which was not further augmented by the combination treatment of Compound 1+cGAMP or Compound 2+cGAMP. In FIG. 15D, Compound 1, Compound 2 and cGAMP increased CXCL10 protein secretion in 4T1 cells which was further synergistically increased by the combination treatment of Compound 1+cGAMP or Compound 2+cGAMP.

### Example 24. Compound 1 Increases CCL5 and CXCL10 Gene Expression and Protein Secretion and Enhances Activity of Endogenous STING Agonist cGAMP in YUMM 1.7 Melanoma Cells

### Quantitative Reverse-Transcriptase Polymerase Chain Reaction (RT-PCR) Assay and Cytokine Secretion Assay

YUMM1.7 cells were treated for 24 hours with DMSO or VPS34 inhibitors (5 μM Compound 1 or 10 μM Compound 2) in combination with 10 mg/ml STING agonist cGAMP (green bars). Cells were washed with PBS and RNA was isolated using the PureLink RNA kit (#12183018A, Thermo Fisher Scientific) according to the manufacturer's protocol. Genomic DNA was removed using DNA-binding columns (RNeasy plus kit, #74134, Qiagen) and/or DNase treatment (#12185010, Thermo Fisher Scientific). RNA was quantified using NanoDrop (Thermo Fisher Scientific) and 50 ng/μL RNA was used for cDNA synthesis with SuperScript IV VILO Master Mix (#11756050, Thermo Fisher Scientific). Quantitative RT-PCR (qRT-PCR) was run with diluted cDNA (1:25 or 1:12.5), PowerUp SYBR Green Master Mix (#A25741, Thermo Fisher Scientific), and 200 nM primers (see below) on a CFX Connect RT-PCR system (BioRad). Data was analyzed using the ΔΔCT method. Results are mean±SEM of three independent experiments.

| Mouse primers | Forward primer sequence | Reverse primer sequence |
|---|---|---|
| Ccl5 | GCTGCTTTGCCTACCTCTCC | TCGAGTGACAAACACGACTGC |
| Cxcl10 | CCAAGTGCTGCCGTCATTTTC | GGCTCGCAGGGATGATTTCAA |
| Rps18 (house- | AGTTCCAGCACATTTTGCGAG | TCATCCTCCGTGAGTTCTCCA |
| keeping gene) | | |

Cell culture medium was centrifuged at 1600 rpm for 10 min at 4° C. and obtained supernatant stored at −80° C. Cytokine levels were quantified using following Meso Scale Discovery (MSD) assays: mouse U-PLEX RANTES/CCL5 (#K152A2K) and U-PLEX IP-10/CXCL10 (#K152UFK). Assays were run on the SECTOR Quickplex Imager and analyzed using Discovery Workbench version 4.0 (MSD).

A study was performed to test whether Compound 1 or Compound 2 increase CCL5 and CXCL10 gene expression alone and in combination with an endogenous STING agonist, cGAMP. FIGS. 15A-15D is a graphical representation of the mRNA expression of CCL5 (FIG. 15A) and CXCL10 (FIG. 15B) and protein secretion of CCL5 (FIG. 15C) and CXCL10 (FIG. 15D) in YUMM cells (third set of bars) treated with medium control (first bar, circle), Compound 1 (2nd bar, triangle), Compound 2 (3rd bar, square), cGAMP (4th bar, circle), the combination of Compound 1+cGAMP (5th bar, triangle) or the combination of Compound 2+cGAMP (6th bar, square).

In FIG. 15A, Compound 1, Compound 2 and cGAMP increased CCL5 mRNA expression in YUMM cells which was further synergistically increased by the combination treatment of Compound 1+cGAMP or Compound 2+cGAMP. In FIG. 15B, Compound 1 and Compound 2 did not increase CXCL10 mRNA expression. cGAMP increased CXCL10 mRNA expression in YUMM cells. Combination treatment of Compound 1+cGAMP or Compound 2+cGAMP resulted in synergistic increases in CXCL10 mRNA expression. In FIG. 15C, Compound 1, Compound 2 and cGAMP increased CCL5 protein secretion in YUMM cells which was not further augmented by the combination treatment of Compound 1+cGAMP or Compound 2+cGAMP. In FIG. 15D, Compound 1, Compound 2 and cGAMP increased CXCL10 protein secretion in YUMM cells which was further synergistically increased by the combination treatment of Compound 1+cGAMP or Compound 2+cGAMP.

### Example 25. Compound 1 Slows Degradation of Activated STING

### Western Blot Assay

B16-F10, CT26, DC2.4 or Renca cells were seeded at 1×10⁶ cells/well in 6-well plates and cultured overnight. Cells were treated with DMSO, 10 μg/mL ADU-S100, 2 μM Compound 1 or the combination for the times indicated in the figure. Cells were washed in PBS and lysed in RIPA buffer (#89901, Pierce) containing protease inhibitor cocktail (#78401, Pierce), phosphatase inhibitor (#78420, Pierce), and 0.5M EDTA solution (#1861276, Thermo Fisher) for 10 min at 4° C. After centrifugation at 21000 rpm for 10 min at 4° C., the supernatant was obtained, and protein concentration was quantified using BCA assay (#23225, Thermo Fisher). Equal amounts were mixed with Sample Reducing agent (#NP0009, Thermo Fisher) and LDS Sample Buffer (#NP0007, Thermo Fisher), heated for 15 min at 90° C. before loading onto 10% Bis-Tris Midi gels. Proteins were transferred to polyvinylidene fluoride (PVDF) membranes. Membranes were blocked with 5% BSA in 1×TBS solution for 1 h at RT, washed in TBS-T, and probed with primary antibodies shaking overnight at 4° C. After washing in TBS-T, membranes were incubated with IRDye 800CW Goat anti-Rabbit (#926-32211, LI-COR, 1:10,000 dilution) secondary antibody in Intercept Blocking Buffer (#927-70001, LI-COR) for 1 h at RT. After washing in TBS-T, membranes were imaged using the Odyssey CLx (LI-COR). Band intensities were quantified using Image Studio software version 5.2.5 (LI-COR).

| | | Diluent (in | Cat. | |
|---|---|---|---|---|
| Antibody | Dilution | TBS-Tween) | number | Supplier |
| STING | 1:1000 | 5% BSA in | 13647S | Cell Signaling |
| | | 1xTBS | | Technology |
| | | blocking | | |
| | | buffer | | |
| Vinculin | 1:3,000 | 5% BSA in | 219649 | Abcam |
| | | 1xTBS | | |
| | | blocking | | |
| | | buffer | | |

### THP-1 Dual Reporter Assay

THP1-Dual™ Cells lines were purchased from InvivoGen (WT (thpd-nfis) and STING-KO (thpd-kostg)) and cultured as recommended by manufacturer: in RPMI media (Gibco 22400-071) supplemented with 10% heat-inactivated Fetal Bovine Serum (FBS) (Gibco A38400-02) and 1× Pen-Strep-Glutamine (Gibco 10378-016). Cells were treated with selective antibiotic every other passage using 100 μg/ml Normocin™ (InvivoGen ant-nr-1), 10 μg/ml of blasticidin (InvivoGen ant-bl-05) and 100 μg/ml of Zeocin™ (InvivoGen ant-zn-05). THP1-Dual™ Cells (wild-type or STING-KO) were seeded at 1.5×105 cells/well in 96-well plates. Cells were treated with DMSO, 5 μg/mL ADU-S100, 2 μM Compound 1 or 2 or the combinations overnight. The next day, 10 μL of the media from the treated cells were mixed with 50 μL of QUANTI-Luc™ Gold (Invivogen rep-qlcg2) in a white 96-well plate (Thermo Scientific 9502887) to measure the interferon-sensitive response element (ISRE) promoter activity through Luciferase signal using a plate reader. For measuring NF-κB promoter activity, 10 μL of the media from the treated cells were mixed with 90 μL of QUANTI-Blue™ (Invivogen rep-qbs3) in white-96-well plates (Corning-Costar 3610), then incubated at 37° C. for one hour. The signal was then measured using a plate reader with detection at 640 nm wavelength.

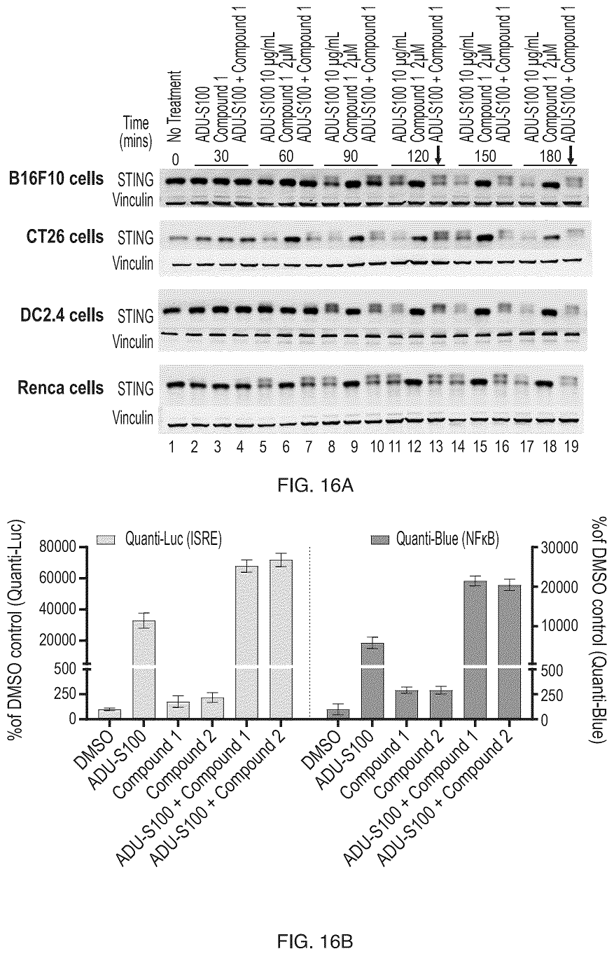

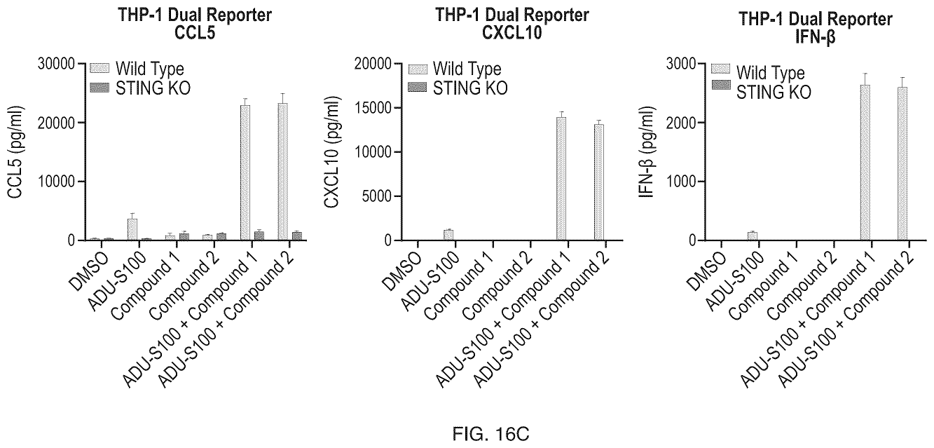

This study was performed to identify whether the combination of a STING agonist and a VPS34 inhibitor resulted in enhanced activation of STING due to the delayed degradation of activated STING in multiple cancer cell lines. FIG. 16A is an image of a Western blot in which lane 1 shows baseline levels (0 mins) of STING protein. Lane 2, 3 and 4 is at 30 mins of treatment with ADU-S100, Compound 1 or the combination, respectively. Lane 5, 6 and 7 is at 60 mins of treatment with ADU-S100, Compound 1 or the combination, respectively. Lane 8, 9 and 10 is at 90 mins of treatment with ADU-S100, Compound 1 or the combination, respectively. Lane 11, 12 and 13 is at 120 mins of treatment with ADU-S100, Compound 1 or the combination, respectively. Lane 14, 15 and 16 is at 150 mins of treatment with ADU-S100, Compound 1 or the combination, respectively. Lane 17, 18 and 19 is at 180 mins of treatment with ADU-S100, Compound 1 or the combination, respectively. The upper band of STING (phosphorylated, activated STING) does not degrade as quickly in cells treated with the combination when compared to cells treated with ADU-S100 alone (compare lanes 13, 16 and 19 (identified with arrows) vs. lanes 11, 14 or 17). FIG. 16B is a graphical representation of the effect of Compound 1, Compound 2, ADU-S100 or the combinations of Compound 1+ADU-S100 or Compound 2+ADU-S100 on activating the reporter in THP-1 dual reporter cells. The THP-1 dual reporter cells were derived from human THP-1 cells by stable integration of two inducible reporter constructs. These reporters read out interferon activity through the interferon sensitive response element (ISRE)(QUANTI-Luc) or NFκB activity (QUANTI-Blue). ADU-S100 stimulates ISRE activity (left bars, light grey) and NFκB activity (right bars, dark grey) and the addition of Compound 1 or Compound 2 increases both signals demonstrating an additive effect with VPS34 inhibition. FIG. 16C is a graphical representation of the effect of Compound 1, Compound 2, ADU-S100 or the combinations of Compound 1+ADU-S100 or Compound 2+ADU-S100 on stimulating the secretion of CCL5 (left graph), CXCL10 (middle graph) or IFNβ (right graph) in wild-type THP-1 dual reporter cells or in THP-1 dual reporter cells in which the STING gene has been deleted, resulting in no STING protein generation. ADU-S100 stimulates CCL5, CXCL10 and IFNβ in wild-type THP-1 dual reporter cells and the combination with Compound 1 or Compound 2 enhances that response (light grey bars). However, there is little to no secretion of CCL5, CXCL10 or IFNβ in the THP-1 dual reporter STING knockout cells (dark grey bars) demonstrating STING dependency for compound-mediated cytokine secretion.

### Example 26. In Vivo Studies of B16-F10 Tumors

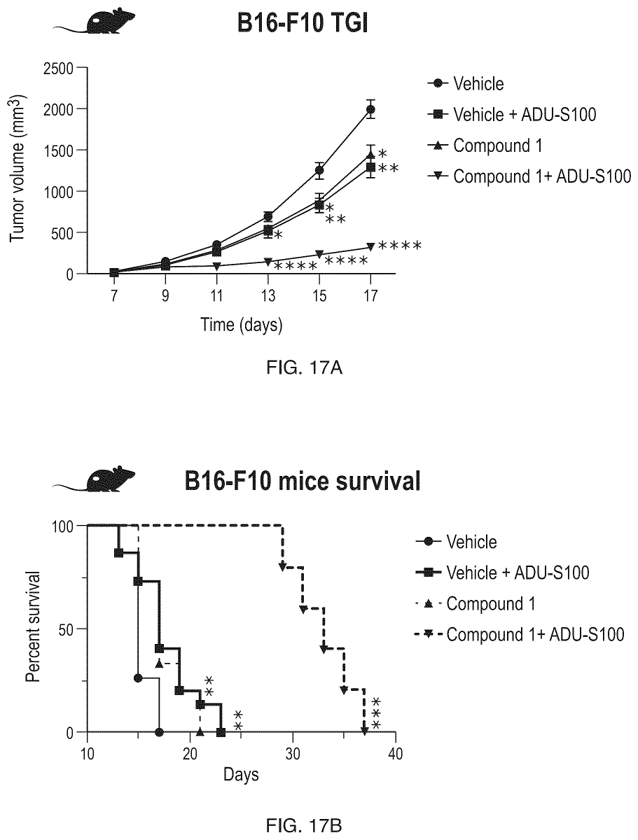

B16-F10 tumor-bearing C57BI/6 mice were treated with vehicle or 20 mg/kg Compound 1 p.o. (daily from day 7-16) in combination with PBS or 10 μg ADU-S100 i.t. (on days 9, 11, 13, and 15). FIG. 17A shows tumor growth curves of B16-F10 tumors in the mice. FIG. 17B shows Kaplan-Meier mice survival curves of the B16-F10 tumor bearing mice for various treatments studied. The data show that Compound 1 sensitized B16-F10 tumors to STING agonist treatment in vivo.

### EQUIVALENTS

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

What is claimed is:

1. A method of treating cancer in a patient in need thereof, comprising:
(i) administering to the patient a therapeutically effective amount of a compound represented by Formula I: 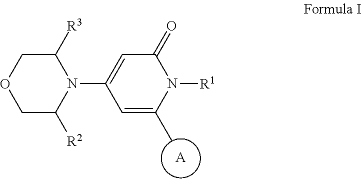
or a pharmaceutically acceptable salt, stereoisomer, or tautomer thereof, wherein:
R¹, R², and R³ are independently selected from the group consisting of H, C₁-C₃haloalkyl, and C₁-C₃alkyl;
A represents: 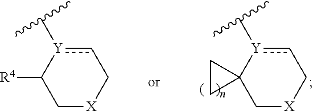 [symbol]
is a single bond or a double bond;
X is selected from the group consisting of CH₂, S, SO, SO₂, NR⁵, NCOR⁵, NCOR⁹, NCOCH₂R⁹, O, and a bond;
Y is selected from the group consisting of N, CH, and C, provided that, when Y is CH, [symbol] is a single bond;
n is selected from 1, 2, 3 and 4;
R⁴ is selected from the group consisting of H, halogen, COR⁶, C₁-C₆alkyl, C₁-C₃alkoxyC₁-C₃alkyl, C₁-C₆alkoxy, C₃-C₆cycloalkyl, C₃-C₆heterocyclyl, C₁-C₃cyanoalkyl, C₁-C₃haloalkyl, aryl, and heteroaryl, wherein said aryl and said heteroaryl are optionally substituted with one or more R⁷;
R⁵ is selected from the group consisting of H, C₁-C₃fluoroalkyl, C₁-C₃alkyl, C₁-C₃alkoxyC₁-C₃alkyl, and C₃-C₆cycloalkyl;
R⁶ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl;
each R⁷ is independently selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy and C₁-C₃alkoxy;
R⁹ is selected from the group consisting of C₁-C₃alkyl, C₁-C₃alkoxy, C₃-C₆cycloalkyl, heterocyclyl, phenyl and a monocyclic heteroaryl, wherein said heterocyclyl, said phenyl and said monocyclic heteroaryl are optionally substituted with one or two R⁸; and
each R⁸ is independently selected from the group consisting of halogen, C₁-C₃haloalkyl and C₁-C₃alkyl; and
(ii) administering to the patient a therapeutically effective amount of a STING agonist;
wherein administering the therapeutically effective amount of the STING agonist and the compound results in an increased expression level of at least one chemokine in the patient as compared to any increase in the expression level of the at least one chemokine resulting from administering the compound alone to the patient.

2. A method of upregulating at least one chemokine in a cell comprising: contacting the cell sample with:
(i) a compound represented by: 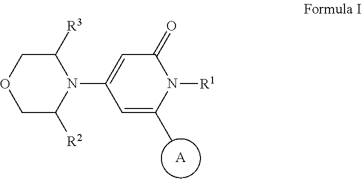
or a pharmaceutically acceptable salt, stereoisomer, or tautomer thereof, wherein:
R¹, R², and R³ are independently selected from the group consisting of H, C₁-C₃haloalkyl, and C₁-C₃alkyl;
A represents: 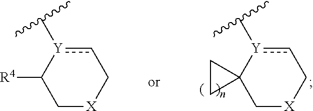 [symbol]
is a single bond or a double bond;
X is selected from the group consisting of CH₂, S, SO, SO₂, NR⁵, NCOR⁵, NCOR⁹, NCOCH₂R⁹, O, and a bond;
Y is selected from the group consisting of N, CH, and C, provided that, when Y is CH, [symbol] is a single bond;
n is selected from 1, 2, 3 and 4;
R⁴ is selected from the group consisting of H, halogen, COR⁶, C₁-C₆alkyl, C₁-C₃alkoxyC₁-C₃alkyl, C₁-C₆alkoxy, C₃-C₆cycloalkyl, C₃-C₆heterocyclyl, C₁-C₃cyanoalkyl, C₁-C₃haloalkyl, aryl, and heteroaryl, wherein said aryl and said heteroaryl are optionally substituted with one or more R⁷;
R⁵ is selected from the group consisting of H, C₁-C₃fluoroalkyl, C₁-C₃alkyl, C₁-C₃alkoxyC₁-C₃alkyl, and C₃-C₆cycloalkyl;
R⁶ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl;
each R⁷ is independently selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy and C₁-C₃alkoxy;
R⁹ is selected from the group consisting of C₁-C₃alkyl, C₁-C₃alkoxy, C₃-C₆cycloalkyl, heterocyclyl, phenyl and a monocyclic heteroaryl, wherein said heterocyclyl, said phenyl and said monocyclic heteroaryl are optionally substituted with one or two R⁸; and
each R⁸ is independently selected from the group consisting of halogen, C₁-C₃haloalkyl and C₁-C₃alkyl;
in an amount sufficient to induce a Type I interferon response by the cell; and
(ii) a STING agonist in an amount sufficient to increase the expression level of the at least one chemokine in the cell.

3. The method of claim 1 or 2, wherein R¹ is H.

4. The method of any one of claims 1-3, wherein R² is H.

5. The method of any one of claims 1-4, wherein R³ is C₁-C₃alkyl.

6. The method of any one of claims 1-5, wherein A is piperidinyl.

7. The method of any one of claims 1-6, wherein R⁴ is C₁-C₃haloalkyl.

8. The method of claim 1 or 2, wherein the compound is selected from the group consisting of: 4-morpholino-6-(2-phenylpyrrolidin-1-yl)-1H-pyridin-2-one; 1-methyl-4-morpholino-6-(2-phenylpyrrolidin-1-yl)pyridin-2-one; 4-morpholino-6-[(2S)-2-phenylpyrrolidin-1-yl]-1H-pyridin-2-one; 4-morpholino-6-[(2R)-2-phenylpyrrolidin-1-yl]-1H-pyridin-2-one; 6-(3,6-dihydro-2H-pyran-4-yl)-4-(3-methylmorpholin-4-yl)-1H-pyridin-2-one; 4-(3-methylmorpholin-4-yl)-6-tetrahydropyran-4-yl-1H-pyridin-2-one; 6-[2-(3-methoxyphenyl)pyrrolidin-1-yl]-4-(3-methylmorpholin-4-yl)-1H-pyridin-2-one; 4-(3-methylmorpholin-4-yl)-6-[2-(3-pyridyl)pyrrolidin-1-yl]-1H-pyridin-2-one; 4-(3-methylmorpholin-4-yl)-6-(2-phenylpyrrolidin-1-yl)-1H-pyridin-2-one; N,N-dimethyl-1-[4-[(3R)-3-methylmorpholin-4-yl]-6-oxo-1H-pyridin-2-yl]pyrrolidine-2-carboxamide; 6-[2-(1-methoxy-1-methyl-ethyl)pyrrolidin-1-yl]-4-[(3R)-3-methylmorpholin-4-yl]-1H-pyridin-2-one; 6-(2-cyclohexylpyrrolidin-1-yl)-4-[(3R)-3-methylmorpholin-4-yl]-1H-pyridin-2-one; 6-[2-(3-fluorophenyl)pyrrolidin-1-yl]-4-[(3R)-3-methylmorpholin-4-yl]-1H-pyridin-2-one; 6-[2-(2,5-difluorophenyl)pyrrolidin-1-yl]-4-[(3R)-3-methylmorpholin-4-yl]-1H-pyridin-2-one; 4-[(3R)-3-methylmorpholin-4-yl]-6-[2-[3-(trifluoromethoxy)phenyl]pyrrolidin-1-yl]-1H-pyridin-2-one; 4-[(3R)-3-methylmorpholin-4-yl]-6-[2-[3-(trifluoromethyl)phenyl]pyrrolidin-1-yl]-1H-pyridin-2-one; 6-[2-(3-methoxyphenyl)pyrrolidin-1-yl]-4-[(3R)-3-methylmorpholin-4-yl]-1H-pyridin-2-one; 4-[(3R)-3-methylmorpholin-4-yl]-6-(2-phenylpyrrolidin-1-yl)-1H-pyridin-2-one; 4-[(3R)-3-methylmorpholin-4-yl]-6-[2-(1-methylpyrazol-4-yl)pyrrolidin-1-yl]-1H-pyridin-2-one; 6-[2-(1,5-dimethylpyrazol-3-yl)pyrrolidin-1-yl]-4-[(3R)-3-methylmorpholin-4-yl]-1H-pyridin-2-one; 6-[2-(1-ethylpyrazol-3-yl)pyrrolidin-1-yl]-4-[(3R)-3-methylmorpholin-4-yl]-1H-pyridin-2-one; 6-[2-(5-methyl-2-furyl)pyrrolidin-1-yl]-4-[(3R)-3-methylmorpholin-4-yl]-1H-pyridin-2-one; 6-[2-[3-(dimethylamino)phenyl]pyrrolidin-1-yl]-4-[(3R)-3-methylmorpholin-4-yl]-1H-pyridin-2-one; 4-[(3R)-3-methylmorpholin-4-yl]-6-(3-methylmorpholin-4-yl)-1H-pyridin-2-one; 4-[(3R)-3-methylmorpholin-4-yl]-6-[2-(trifluoromethyl)-1-piperidyl]-1H-pyridin-2-one; 4-[(3R)-3-methylmorpholin-4-yl]-6-(3-phenylmorpholin-4-yl)-1H-pyridin-2-one; 4-[(3R)-3-methylmorpholin-4-yl]-6-(1-oxo-1,4-thiazinan-4-yl)-1H-pyridin-2-one; 6-(1,1-dioxo-1,4-thiazinan-4-yl)-4-[(3R)-3-methylmorpholin-4-yl]-1H-pyridin-2-one; 6-(4-acetylpiperazin-1-yl)-4-[(3R)-3-methylmorpholin-4-yl]-1H-pyridin-2-one; 4-[(3R)-3-methylmorpholin-4-yl]-6-[(2R)-2-phenyl-1-piperidyl]-1H-pyridin-2-one; 4-[(3R)-3-methylmorpholin-4-yl]-6-(4-methyl-2-phenyl-piperazin-1-yl)-1H-pyridin-2-one; 4-[(3R)-3-methylmorpholin-4-yl]-6-[3-(trifluoromethyl)morpholin-4-yl]-1H-pyridin-2-one; 6-(3-cyclopropylmorpholin-4-yl)-4-[(3R)-3-methylmorpholin-4-yl]-1H-pyridin-2-one; 4-[(3R)-3-methylmorpholin-4-yl]-6-[(2S)-2-(trifluoromethyl)pyrrolidin-1-yl]-1H-pyridin-2-one; 4-[(3R)-3-methylmorpholin-4-yl]-6-[(2R)-2-(trifluoromethyl)pyrrolidin-1-yl]-1H-pyridin-2-one; 6-[2-(3-chlorophenyl)pyrrolidin-1-yl]-4-[(3R)-3-methylmorpholin-4-yl]-1H-pyridin-2-one; 6-[2-(3-cyclopropylphenyl)pyrrolidin-1-yl]-4-[(3R)-3-methylmorpholin-4-yl]-1H-pyridin-2-one; 4-[(3R)-3-methylmorpholin-4-yl]-6-[2-(2-pyridyl)pyrrolidin-1-yl]-1H-pyridin-2-one; 4-[(3R)-3-methylmorpholin-4-yl]-6-(2-thiazol-2-ylpyrrolidin-1-yl)-1H-pyridin-2-one; 6-[2-(5-methylisoxazol-3-yl)pyrrolidin-1-yl]-4-[(3R)-3-methylmorpholin-4-yl]-1H-pyridin-2-one; 1-methyl-4-[(3R)-3-methylmorpholin-4-yl]-6-[(2R)-2-(trifluoromethyl)-1-piperidyl]pyridin-2-one; 4-[(3R)-3-methylmorpholin-4-yl]-6-(8-oxa-5-azaspiro[3.5]nonan-5-yl)-1H-pyridin-2-one; 6-[2-(3-methoxyphenyl)-1-piperidyl]-4-[(3R)-3-methylmorpholin-4-yl]-1H-pyridin-2-one; 6-[4-acetyl-2-(trifluoromethyl)piperazin-1-yl]-4-[(3R)-3-methylmorpholin-4-yl]-1H pyridin-2-one; 6-[4-(5-fluoropyridine-3-carbonyl)-2-(trifluoromethyl)piperazin-1-yl]-4-[(3R)-3-methylmorpholin-4-yl]-1H-pyridin-2-one; 6-[4-[2-(4-fluorophenyl)acetyl]-2-(trifluoromethyl)piperazin-1-yl]-4-[(3R)-3-methylmorpholin-4-yl]-1H-pyridin-2-one; 4-[(3R)-3-methylmorpholin-4-yl]-6-[4-(tetrahydrofuran-2-carbonyl)-2-(trifluoromethyl)piperazin-1-yl]-1H-pyridin-2-one; 4-[(3R)-3-methylmorpholin-4-yl]-6-[4-methyl-2-(trifluoromethyl)piperazin-1-yl]-1H-pyridin-2-one; and
pharmaceutically acceptable salts, tautomers, and stereoisomers thereof.

9. A method of treating cancer in a patient in need thereof, comprising administering to the patient:
(i) a therapeutically effective amount of a compound represented by Formula II: 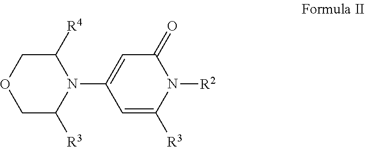
or a pharmaceutically acceptable salt, stereoisomer, or tautomer thereof, wherein:
R¹ is selected from the group consisting of aryl and heteroaryl, wherein said aryl and said heteroaryl being mono- or bicyclic and each of aryl and heteroaryl is optionally substituted with one or more independent occurrences of a substituent selected from the group consisting of R⁵, R⁶, R⁷ and R⁸;
each of R², R³, R⁴ is independently selected from the group consisting of H, C₁-C₃haloalkyl, and C₁-C₃alkyl;
each of R⁵, R⁶, R⁷, and R⁸ is independently selected from the group consisting of halogen, C₁-C₆alkyl, C₁-C₆alkoxy, C₁-C₆haloalkyl, amino, —NHSO₂R⁹, hydroxy, phenyl, and a monocyclic heteroaryl; and
R⁹ is selected from C₁-C₃haloalkyl and C₁-C₃alkyl; and
(ii) a therapeutically effective amount of a STING agonist;
wherein administering the therapeutically effective amount of the STING agonist and the compound results in an increased expression level of at least one chemokine in the patient as compared to any increase in the expression level of the at least one chemokine resulting from administering the compound alone to the patient.

10. A method of upregulating at least one chemokine in a cell comprising: contacting the cell sample with:
(i) a compound represented by: 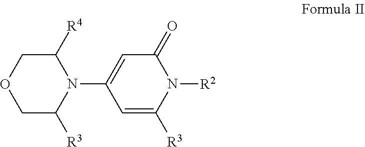
or a pharmaceutically acceptable salt, stereoisomer, or tautomer thereof, wherein:
R¹ is selected from the group consisting of aryl and heteroaryl, wherein said aryl and said heteroaryl being mono- or bicyclic and each of aryl and heteroaryl is optionally substituted with one or more independent occurrences of a substituent selected from the group consisting of R⁵, R⁶, R⁷ and R⁸;
each of R², R³, R⁴ is independently selected from the group consisting of H, C₁-C₃haloalkyl, and C₁-C₃alkyl;
each of R⁵, R⁶, R⁷, and R⁸ is independently selected from the group consisting of halogen, C₁-C₆alkyl, C₁-C₆alkoxy, C₁-C₆haloalkyl, amino, —NHSO₂R⁹, hydroxy, phenyl, and a monocyclic heteroaryl; and
R⁹ is selected from C₁-C₃haloalkyl and C₁-C₃alkyl;
in an amount sufficient to induce a Type I interferon response by the cell; and
(ii) a STING agonist in an amount sufficient to increase the expression level of the at least one chemokine in the cell.

11. The method of claim 9 or 10, wherein the compound is selected from the group consisting of: 6-(2-chlorophenyl)-4-morpholino-1H-pyridin-2-one; 6-(2-chlorophenyl)-1-methyl-4-morpholino-pyridin-2-one; 6-(2-chlorophenyl)-4-(3-methylmorpholin-4-yl)-1H-pyridin-2-one; 6-(2-chlorophenyl)-1-methyl-4-(3-methylmorpholin-4-yl)pyridin-2-one; 4-(3-methylmorpholin-4-yl)-6-(4-methyl-3-pyridyl)-1H-pyridin-2-one; 4-(3-methylmorpholin-4-yl)-6-pyrimidin-5-yl-1H-pyridin-2-one; 4-(3-methylmorpholin-4-yl)-6-(2-phenylphenyl)-1H-pyridin-2-one; 6-(2-chloro-5-fluoro-phenyl)-4-[(3R)-3-methylmorpholin-4-yl]-1H-pyridin-2-one; 4-[(3R)-3-methylmorpholin-4-yl]-6-(o-tolyl)-1H-pyridin-2-one; 4-[(3R)-3-methylmorpholin-4-yl]-6-[2-(trifluoromethyl)-3-pyridyl]-1H-pyridin-2-one; 6-(2-chlorophenyl)-4-[(3R)-3-methylmorpholin-4-yl]-1H-pyridin-2-one; 4-[(3R)-3-methylmorpholin-4-yl]-6-[2-(trifluoromethyl)phenyl]-1H-pyridin-2-one; 6-(3-furyl)-4-[(3R)-3-methylmorpholin-4-yl]-1H-pyridin-2-one; 4-[(3R)-3-methylmorpholin-4-yl]-6-(4-methyl-3-thienyl)-1H-pyridin-2-one; N-[2-[4-[(3R)-3-methylmorpholin-4-yl]-6-oxo-1H-pyridin-2-yl]phenyl]methanesulfonamide; 4-[(3R)-3-methylmorpholin-4-yl]-6-(4-(methylsulfonyl)-2-(trifluoromethyl)phenyl)-1H-pyridin-2-one; 4-[(3R)-3-methylmorpholin-4-yl]-6-(6-methyl-5-quinolyl)-1H-pyridin-2-one; 4-[(3R)-3-methylmorpholin-4-yl]-6-[4-(1H-pyrazol-5-yl)phenyl]-1H-pyridin-2-one; N,N-dimethyl-[4[4-[(3R)-3-methylmorpholin-4-yl]-6-oxo-1H-pyridin-2-yl]-3-(trifluoromethyl)]benzenesulfonamide; and pharmaceutically acceptable salts, tautomers, and stereoisomers thereof.

12. A method of treating cancer in a patient in need thereof, comprising administering to the patient:
(i) a therapeutically effective amount of a compound represented by Formula III: 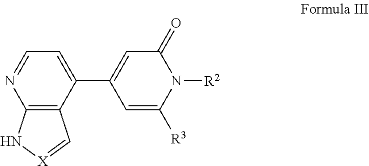
or a pharmaceutically acceptable salt, stereoisomer, or tautomer thereof, wherein:
X is selected from N and CR¹;
R¹ is selected from the group consisting of H, C₁-C₃alkyl, C₁-C₃haloalkyl, C₁-C₃alkoxyC₁-C₃alkyl, C₃-C₆cycloalkyl, cyano, phenyl, and monocyclic heteroaryl, wherein each of phenyl and monocyclic heteroaryl is optionally substituted with one or more substituents independently selected from the group consisting of halo, N—C₁-C₃alkylamino, N,N-diC₁-C₃alkylamino, C₃-C₆cycloalkyl, C₁-C₃alkoxyC₁-C₃alkyl, C₁-C₃haloalkyl, C₁-C₃haloalkoxy, C₁-C₃alkoxy, and C₁-C₃alkyl;
R² is selected from the group consisting of H, C₁-C₃haloalkyl, and C₁-C₃alkyl;
R³ is selected from the group consisting of A, phenyl, and monocyclic heteroaryl, wherein each of phenyl and monocyclic heteroaryl is optionally substituted with one or more occurrences of R⁴;
each R⁴ is independently selected from the group consisting of COR⁵, halogen, C₁-C₆alkyl, C₁-C₆alkoxy, C₁-C₆haloalkoxy, amino N—C₁-C₃alkylamino, N,N-diC₁-C₃alkylamino, 1-pyrrolidinyl, 1-piperidinyl, 1-azetidinyl, NHSO₂R⁶, SO₂R⁷, hydroxy, C₃-C₆cycloalkyl, C₁-C₃alkoxyC₁-C₃alkyl, C₁-C₃cyanoalkyl and C₁-C₆haloalkyl;
R⁵ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl;
R⁶ is selected from C₁-C₃haloalkyl and C₁-C₃alkyl;
each R⁷ is independently selected from the group consisting of R⁸, C₁-C₆alkyl, N—C₁-C₃alkylamino, N,N-diC₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen;
each R⁸ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁹;
each R⁹ is independently selected from the group consisting of halo, N—C₁-C₃alkylamino, N,N-diC₁-C₃alkylamino, C₁-C₃alkoxyC₁-C₃alkyl, amino, C₁-C₃haloalkyl, C₁-C₃alkoxy, C₁-C₃haloalkoxy, C₃-C₆cycloalkyl and C₁-C₃alkyl;
A is 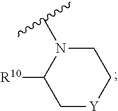
R¹⁰ is selected from the group consisting of H, halogen, COR¹¹, C₁-C₆alkyl, C₁-C₃alkoxyC₁-C₃alkyl, C₁-C₆alkoxy, C₃-C₆cycloalkyl, C₁-C₃cyanoalkyl, C₁-C₃haloalkyl, phenyl, and heteroaryl, wherein each of phenyl and heteroaryl is optionally substituted with one or more occurrences of R¹², and provided that when R¹⁰ is phenyl or heteroaryl, then X is N or CH;
each R¹¹ is independently selected from the group consisting of C₁-C₃alkoxy, N—C₁-C₃alkylamino, N,N-diC₁-C₃alkylamino, 1-pyrrolidinyl, 1-piperidinyl, and 1-azetidinyl;
Y is selected from the group consisting of CH₂, S, SO, SO₂, NR¹³, NCOR⁷, NCOOR¹⁴, NSO₂R⁷, NCOCH₂R⁷, O, and a bond;
R¹² is selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy, and C₁-C₃alkoxy;
R¹³ is selected from H, C₁-C₃haloalkyl, C₁-C₃alkoxyC₁-C₃alkyl, C₁-C₃alkyl, C₃-C₆cycloalkyl; and
R¹⁴ is selected from R⁸, C₁-C₆alkyl, C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen; and
(ii) a therapeutically effective amount of a STING agonist;
wherein administering the therapeutically effective amount of the STING agonist and the compound results in an increased expression level of at least one chemokine in the patient as compared to any increase in the expression level of the at least one chemokine resulting from administering the compound alone to the patient.

13. A method of upregulating at least one chemokine in a cell comprising: contacting the cell sample with:
(i) a compound represented by: 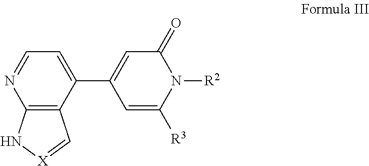
or a pharmaceutically acceptable salt, stereoisomer, or tautomer thereof, wherein:
X is selected from N and CR¹;
R¹ is selected from the group consisting of H, C₁-C₃alkyl, C₁-C₃haloalkyl, C₁-C₃alkoxyC₁-C₃alkyl, C₃-C₆cycloalkyl, cyano, phenyl, and monocyclic heteroaryl, wherein each of phenyl and monocyclic heteroaryl is optionally substituted with one or more substituents independently selected from the group consisting of halo, N—C₁-C₃alkylamino, N,N-diC₁-C₃alkylamino, C₃-C₆cycloalkyl, C₁-C₃alkoxyC₁-C₃alkyl, C₁-C₃haloalkyl, C₁-C₃haloalkoxy, C₁-C₃alkoxy, and C₁-C₃alkyl;
R² is selected from the group consisting of H, C₁-C₃haloalkyl, and C₁-C₃alkyl;
R³ is selected from the group consisting of A, phenyl, and monocyclic heteroaryl, wherein each of phenyl and monocyclic heteroaryl is optionally substituted with one or more occurrences of R⁴;
each R⁴ is independently selected from the group consisting of COR⁵, halogen, C₁-C₆alkyl, C₁-C₆alkoxy, C₁-C₆haloalkoxy, amino N—C₁-C₃alkylamino, N,N-diC₁-C₃alkylamino, 1-pyrrolidinyl, 1-piperidinyl, 1-azetidinyl, NHSO₂R⁶, SO₂R⁷, hydroxy, C₃-C₆cycloalkyl, C₁-C₃alkoxyC₁-C₃alkyl, C₁-C₃cyanoalkyl and C₁-C₆haloalkyl;
R⁵ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl;
R⁶ is selected from C₁-C₃haloalkyl and C₁-C₃alkyl;
each R⁷ is independently selected from the group consisting of R⁸, C₁-C₆alkyl, N—C₁-C₃alkylamino, N,N-diC₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen;
each R⁸ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁹;
each R⁹ is independently selected from the group consisting of halo, N—C₁-C₃alkylamino, N,N-diC₁-C₃alkylamino, C₁-C₃alkoxyC₁-C₃alkyl, amino, C₁-C₃haloalkyl, C₁-C₃alkoxy, C₁-C₃haloalkoxy, C₃-C₆cycloalkyl and C₁-C₃alkyl;
A is 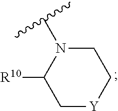
R¹⁰ is selected from the group consisting of H, halogen, COR¹¹, C₁-C₆alkyl, C₁-C₃alkoxyC₁-C₃alkyl, C₁-C₆alkoxy, C₃-C₆cycloalkyl, C₁-C₃cyanoalkyl, C₁-C₃haloalkyl, phenyl, and heteroaryl, wherein each of phenyl and heteroaryl is optionally substituted with one or more occurrences of R¹², and provided that when R¹⁰ is phenyl or heteroaryl, then X is N or CH;
each R¹¹ is independently selected from the group consisting of C₁-C₃alkoxy, N—C₁-C₃alkylamino, N,N-diC₁-C₃alkylamino, 1-pyrrolidinyl, 1-piperidinyl, and 1-azetidinyl;
Y is selected from the group consisting of CH₂, S, SO, SO₂, NR¹³, NCOR⁷, NCOOR¹⁴, NSO₂R⁷, NCOCH₂R⁷, O, and a bond;
R¹² is selected from the group consisting of C₁-C₆alkyl, C₃-C₆cycloalkyl, C₁-C₃alkoxyC₁-C₃alkyl, C₁-C₃haloalkyl, halogen, N—C₁-C₃alkylamino, N,N-diC₁-C₃alkylamino, C₁-C₃haloalkoxy, and C₁-C₃alkoxy;
R¹³ is selected from H, C₁-C₃haloalkyl, C₁-C₃alkoxyC₁-C₃alkyl, C₁-C₃alkyl, C₃-C₆cycloalkyl; and
R¹⁴ is selected from R⁸, C₁-C₆alkyl, C₁-C₃alkoxyC₁-C₃alkyl, wherein each of C₁-C₆alkyl and C₁-C₃alkoxyC₁-C₃alkyl is optionally substituted with one occurrence of R⁸, and each of C₁-C₆alkyl and C₁-C₃alkoxyC₁-C₃alkyl is optionally substituted with or one or more independent occurrences of halogen; and
in an amount sufficient to induce a Type I interferon response by the cell; and
(ii) a STING agonist in an amount sufficient to increase the expression level of the at least one chemokine in the cell.

14. The method of claim 12 or 13, wherein the compound is selected from the group consisting of: 4-(1H-pyrrolo[2,3-b]pyridin-4-yl)-6-[2-(trifluoromethyl)phenyl]-1H-pyridin-2-one; 6-(3-Methyl-4-pyridyl)-4-(1H-pyrrolo[2,3-b]pyridin-4-yl)-1H-pyridin-2-one; 6-(2-phenylpyrrolidin-1-yl)-4-(1H-pyrrolo[2,3-b]pyridin-4-yl)-1H-pyridin-2-one; 4-(2-Methyl-1H-pyrrolo[2,3-b]pyridin-4-yl)-6-(3-pyridyl)-1H-pyridin-2-one; 4-(2-Methyl-1H-pyrrolo[2,3-b]pyridin-4-yl)-6-morpholino-1H-pyridin-2-one; 6-(2-Chlorophenyl)-4-(2-oxazol-5-yl-1H-pyrrolo[2,3-b]pyridin-4-yl)-1H-pyridin-2-one; 6-(2-Chlorophenyl)-4-[2-(3-pyridyl)-1H-pyrrolo[2,3-b]pyridin-4-yl]-1H-pyridin-2-one; 6-(2-Chlorophenyl)-4-(2-methyl-1H-pyrrolo[2,3-b]pyridin-4-yl)-1H-pyridin-2-one; 4-(2-Methyl-1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[3-(trifluoromethyl)morpholin-4-yl]-1H-pyridin-2-one; 6-[3-(Trifluoromethyl)morpholin-4-yl]-4-[2-[3-(trifluoromethyl)phenyl]-1H-pyrrolo[2,3-b]pyridin-4-yl]-1H-pyridin-2-one; 4-[2-(5-Methyl-2-thienyl)-1H-pyrrolo[2,3-b]pyridin-4-yl]-6-[3-(trifluoromethyl)morpholin-4-yl]-1H-pyridin-2-one; 4-(1H-pyrazolo[3,4-b]pyridin-4-yl)-6-[2-(trifluoromethyl)-1-piperidyl]-1H-pyridin-2-one; 6-[2-(Trifluoromethyl)-1-piperidyl]-4-yl]-1H-pyrrolo[2,3-b]pyridin-4-yl]-1H-pyridin-2-one; 6-[2-(Trifluoromethyl)-1-piperidyl]-4-[2-[6-(trifluoromethyl)-3-pyridyl]-1H-pyrrolo[2,3-b]pyridin-4-yl]-1H-pyridin-2-one; 6-[2-(Trifluoromethyl)-1-piperidyl]-4-[2-[5-(trifluoromethyl)-3-pyridyl]-1H-pyrrolo[2,3-b]pyridin-4-yl]-1H-pyridin-2-one; 4-(2-cyclopropyl-1H-pyrrolo[2,3-b]pyridin-4-yl)-6-[4-ethylsulfonyl-2-(trifluoromethyl)piperazin-1-yl]-1H-pyridin-2-one; 6-[4-ethylsulfonyl-2-(trifluoromethyl)piperazin-1-yl]-4-(1H-pyrrolo[2,3-b]pyridin-4-yl)-1H-pyridin-2-one; 6-[4-[(4-fluorophenyl)methylsulfonyl]-2-(trifluoromethyl)piperazin-1-yl]-4-(1H-pyrrolo[2,3-b]pyridin-4-yl)-1H-pyridin-2-one; 6-[4-Ethylsulfonyl-2-(trifluoromethyl)piperazin-1-yl]-4-(2-methyl-1H-pyrrolo[2,3-b]pyridin-4-yl)-1H-pyridin-2-one; 4-[2-[4-Ethylsulfonyl-2-(trifluoromethyl)piperazin-1-yl]-6-oxo-1H-pyridin-4-yl]-1H-pyrrolo[2,3-b]pyridine-2-carbonitrile; 4-(2-cyclopropyl-1H-pyrrolo[2,3-b]pyridin-4-yl)-6-[2-(trifluoromethyl)phenyl]-1H-pyridin-2-one; 4-[2-Oxo-6-[2-(trifluoromethyl)phenyl]-1H-pyridin-4-yl]-1H-pyrrolo[2,3-b]pyridine-2-carbonitrile; 4-(1H-pyrazolo[3,4-b]pyridin-4-yl)-6-[2-(trifluoromethyl)phenyl]-1H-pyridin-2-one; 4-(6-(2-chlorophenyl)-2-oxo-1,2-dihydropyridin-4-yl)-N-ethyl-1H-pyrrolo[2,3-b]pyridine-2-carboxamide; 6-[4-Methylsulfonyl-2-(trifluoromethyl)piperazin-1-yl]-4-(1H-pyrazolo[3,4-b]pyridin-4-yl)-1H-pyridin-2-one; and pharmaceutically acceptable salts, stereoisomers, and tautomers thereof.

15. The method of claim 12 or 13, wherein the compound is selected from the group consisting of: 4-(1H-pyrrolo[2,3-b]pyridin-4-yl)-6-[2-(trifluoromethyl)phenyl]-1H-pyridin-2-one; 6-(3-Methyl-4-pyridyl)-4-(1H-pyrrolo[2,3-b]pyridin-4-yl)-1H-pyridin-2-one; 6-(2-phenylpyrrolidin-1-yl)-4-(1H-pyrrolo[2,3-b]pyridin-4-yl)-1H-pyridin-2-one; 4-(2-Methyl-1H-pyrrolo[2,3-b]pyridin-4-yl)-6-(3-pyridyl)-1H-pyridin-2-one; 4-(2-Methyl-1H-pyrrolo[2,3-b]pyridin-4-yl)-6-morpholino-1H-pyridin-2-one; 6-(2-Chlorophenyl)-4-(2-oxazol-5-yl-1H-pyrrolo[2,3-b]pyridin-4-yl)-1H-pyridin-2-one; 6-(2-Chlorophenyl)-4-[2-(3-pyridyl)-1H-pyrrolo[2,3-b]pyridin-4-yl]-1H-pyridin-2-one; 6-(2-Chlorophenyl)-4-(2-methyl-1H-pyrrolo[2,3-b]pyridin-4-yl)-1H-pyridin-2-one; 4-(2-Methyl-1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[2-(trifluoromethyl)-1-piperidyl]-1H-pyridin-2-one; 4-(1H-pyrrolo[2,3-b]pyridin-4-yl)-6-[3-(trifluoromethyl)morpholin-4-yl]-1H-pyridin-2-one; 6-[3-(Trifluoromethyl)morpholin-4-yl]-4-[2-[3-(trifluoromethyl)phenyl]-1H-pyrrolo[2,3-b]pyridin-4-yl]-1H-pyridin-2-one; 4-[2-(5-Methyl-2-thienyl)-1H-pyrrolo[2,3-b]pyridin-4-yl]-6-[3-(trifluoromethyl)morpholin-4-yl]-1H-pyridin-2-one; 4-(1H-pyrazolo[3,4-b]pyridin-4-yl)-6-[2-(trifluoromethyl)-1-pipendyl]-1H-pyridin-2-one; 6-[2-(Trifluoromethyl)-1-piperidyl]-4-yl]-1H-pyrrolo[2,3-b]pyridin-4-yl]-1H-pyridin-2-one; 6-[2-(Trifluoromethyl)-1-piperidyl]-4-[2-[6-(trifluoromethyl)-3-pyridyl]-1H-pyrrolo[2,3-b]pyridin-4-yl]-1H-pyridin-2-one; 6-[2-(Trifluoromethyl)-1-piperidyl]-4-[2-[5-(trifluoromethyl)-3-pyridyl]-1H-pyrrolo[2,3-b]pyridin-4-yl]-1H-pyridin-2-one; 4-(2-cyclopropyl-1H-pyrrolo[2,3-b]pyridin-4-yl)-6-[4-ethylsulfonyl-2-(trifluoromethyl)piperazin-1-yl]-1H-pyridin-2-one; 6-[4-ethylsulfonyl-2-(trifluoromethyl)piperazin-1-yl]-4-(1H-pyrrolo[2,3-b]pyridin-4-yl)-1H-pyridin-2-one; 6-[4-[(4-fluorophenyl)methylsulfonyl]-2-(trifluoromethyl)piperazin-1-yl]-4-(1H-pyrrolo[2,3-b]pyridin-4-yl)-1H-pyridin-2-one; and pharmaceutically acceptable salts, stereoisomers, and tautomers thereof.

16. A method of treating cancer in a patient in need thereof, comprising:
(i) administering to the patient a therapeutically effective amount of a compound represented by Formula IV: 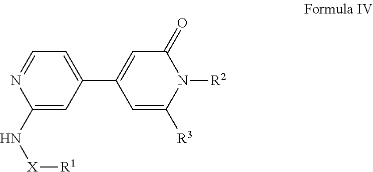
or a pharmaceutically acceptable salt, stereoisomer, or tautomer thereof, wherein:
X is selected from —C(═O)— and a bond;
R¹ is selected from the group consisting of H, C₁-C₃alkyl, C₁-C₃haloalkyl, C₁-C₃alkoxyC₁-C₃alkyl, C₃-C₆cycloalkyl, C₃-C₆cyclohaloalkyl, C₁-C₃alkoxy, C₁-C₃haloalkoxy, C₃-C₆cycloalkoxymethyl, N—C₁-C₃alkylamino, N,N-diC₁-C₃alkylamino, 1-pyrrolidinyl, 1-piperidinyl and 1-azetidinyl, provided that when R¹ is selected from the group consisting of C₁-C₃alkoxy, C₁-C₃haloalkoxy, N—C₁-C₃alkylamino, N,N-diC₁-C₃alkylamino, 1-pyrrolidinyl, 1-piperidinyl, and 1-azetidinyl, then X is C═O;
R² is selected from the group consisting of H, C₁-C₃haloalkyl, and C₁-C₃alkyl;
R³ is selected from the group consisting of A, phenyl and monocyclic heteroaryl, wherein each of phenyl and monocyclic heteroaryl is optionally substituted with one or more occurrences of a substituent independently selected from the group consisting of R⁴, R⁵, R⁶ and R⁷;
each R⁴, R⁵, R⁶, and R⁷ is independently selected from the group consisting of halo, C₁-C₆alkyl, C₃-C₆cycloalkyl, C₁-C₆alkoxy, C₁-C₃haloalkoxy, N,N-diC₁-C₃alkylamino, N—C₁-C₃alkylamino, 1-azetidinyl, C₁-C₆haloalkyl, amino, NHSO₂R⁸, SO₂R⁹, and hydroxy;
R⁸ is selected from C₁-C₃haloalkyl and C₁-C₃alkyl;
each R⁹ is independently selected from the group consisting of R¹⁰, C₁-C₆alkyl, amino, N—C₁-C₃alkylamino, N,N-di-C₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen;
each R¹⁰ is independently selected from the group consisting of phenyl, monocyclic heteroaryl, C₃-C₆cycloalkyl, and heterocyclyl, wherein each phenyl, monocyclic heteroaryl, C₃-C₆cycloalkyl, and heterocyclyl is optionally substituted with one or more occurrences of R¹¹;
each R¹¹ is independently selected from the group consisting of halo, C₁-C₃alkoxyC₁-C₃alkyl, amino, N—C₁-C₃alkylamino, N,N-diC₁-C₃alkylamino, C₁-C₃haloalkoxy, C₁-C₃alkoxy, C₃-C₆cycloalkyl, C₁-C₃haloalkyl, and C₁-C₃alkyl;
A is: 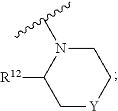
R¹² is selected from the group consisting of H, halo, COR¹³, C₁-C₆alkyl, C₁-C₃alkoxyC₁-C₃alkyl, C₁-C₆alkoxy, C₃-C₆cycloalkyl, C₁-C₃cyanoalkyl, and C₁-C₃haloalkyl;
R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl;
Y is selected from the group consisting of CH₂, S, SO, SO₂, NR¹⁴, NCOR⁹, NCOOR¹⁵, NSO₂R⁹, NCOCH₂R⁹, O, and a bond;
R¹⁴ is selected from the group consisting of H, C₁-C₃haloalkyl, C₁-C₃alkoxyC₁-C₃alkyl, C₁-C₃alkyl, and C₃-C₆cycloalkyl; and
R¹⁵ is selected from the group consisting of R¹⁰, C₁-C₆alkyl and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and
(ii) administering to the patient a therapeutically effective amount of a STING agonist;
wherein administering the therapeutically effective amount of the STING agonist and the compound results in an increased expression level of at least one chemokine in the patient as compared to any increase in the expression level of the at least one chemokine resulting from administering the compound alone to the patient.

17. A method of upregulating at least one chemokine in a cell comprising: contacting the cell sample with:
(i) a compound represented by: 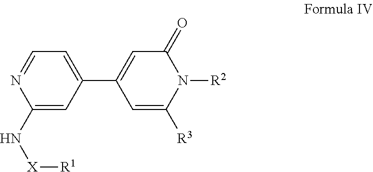
or a pharmaceutically acceptable salt, stereoisomer, or tautomer thereof, wherein:
X is selected from —C(═O)— and a bond;
R¹ is selected from the group consisting of H, C₁-C₃alkyl, C₁-C₃haloalkyl, C₁-C₃alkoxyC₁-C₃alkyl, C₃-C₆cycloalkyl, C₃-C₆cyclohaloalkyl, C₁-C₃alkoxy, C₁-C₃haloalkoxy, C₃-C₆cycloalkoxymethyl, N—C₁-C₃alkylamino, N,N-diC₁-C₃alkylamino, 1-pyrrolidinyl, 1-piperidinyl and 1-azetidinyl, provided that when R¹ is selected from the group consisting of C₁-C₃alkoxy, C₁-C₃haloalkoxy, N—C₁-C₃alkylamino, N,N-diC₁-C₃alkylamino, 1-pyrrolidinyl, 1-piperidinyl, and 1-azetidinyl, then X is C═O;
R² is selected from the group consisting of H, C₁-C₃haloalkyl, and C₁-C₃alkyl;
R³ is selected from the group consisting of A, phenyl and monocyclic heteroaryl, wherein each of phenyl and monocyclic heteroaryl is optionally substituted with one or more occurrences of a substituent independently selected from the group consisting of R⁴, R⁵, R⁶ and R⁷;
each R⁴, R⁵, R⁶, and R⁷ is independently selected from the group consisting of halo, C₁-C₆alkyl, C₃-C₆cycloalkyl, C₁-C₆alkoxy, C₁-C₃haloalkoxy, N,N-diC₁-C₃alkylamino, N—C₁-C₃alkylamino, 1-azetidinyl, C₁-C₆haloalkyl, amino, NHSO₂R⁸, SO₂R⁹, and hydroxy;
R⁸ is selected from C₁-C₃haloalkyl and C₁-C₃alkyl;
each R⁹ is independently selected from the group consisting of R¹⁰, C₁-C₆alkyl, amino, N—C₁-C₃alkylamino, N,N-di-C₁-C₃alkylamino and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen;
each R¹⁰ is independently selected from the group consisting of phenyl, monocyclic heteroaryl, C₃-C₆cycloalkyl, and heterocyclyl, wherein each phenyl, monocyclic heteroaryl, C₃-C₆cycloalkyl, and heterocyclyl is optionally substituted with one or more occurrences of R¹¹;
each R¹¹ is independently selected from the group consisting of halo, C₁-C₃alkoxyC₁-C₃alkyl, amino, N—C₁-C₃alkylamino, N,N-diC₁-C₃alkylamino, C₁-C₃haloalkoxy, C₁-C₃alkoxy, C₃-C₆cycloalkyl, C₁-C₃haloalkyl, and C₁-C₃alkyl;
A is: 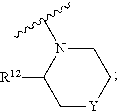
R¹² is selected from the group consisting of H, halo, COR¹³, C₁-C₆alkyl, C₁-C₃alkoxyC₁-C₃alkyl, C₁-C₆alkoxy, C₃-C₆cycloalkyl, C₁-C₃cyanoalkyl, and C₁-C₃haloalkyl;
R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl;
Y is selected from the group consisting of CH₂, S, SO, SO₂, NR¹⁴, NCOR⁹, NCOOR¹⁵, NSO₂R⁹, NCOCH₂R⁹, O, and a bond;
R¹⁴ is selected from the group consisting of H, C₁-C₃haloalkyl, C₁-C₃alkoxyC₁-C₃alkyl, C₁-C₃alkyl, and C₃-C₆cycloalkyl; and
R¹⁵ is selected from the group consisting of R¹⁰, C₁-C₆alkyl and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₆alkoxyC₁-C₃alkyl is optionally substituted with or one or more independent occurrences of halogen; and
in an amount sufficient to induce a Type I interferon response by the cell; and
(ii) a STING agonist in an amount sufficient to increase the expression level of the at least one chemokine in the cell.

18. The method of claim 16 or 17, wherein the compound is selected from the group consisting of: N-[4-[2-(2-chlorophenyl)-6-oxo-1H-pyridin-4-yl]-2-pyridyl]acetamide; 4-(2-Amino-4-pyridyl)-6-(3-pyridyl)-1H-pyridin-2-one; 4-(2-Amino-4-pyridyl)-6-(2-chlorophenyl)-1H-pyridin-2-one; N-[4-[2-(2-chlorophenyl)-6-oxo-1H-pyridin-4-yl]-2-pyridyl]-2-methoxy-acetamide; N-[4-[2-oxo-6-[2-(trifluoromethyl)-1-piperidyl]-1H-pyridin-4-yl]-2-pyridyl]acetamide; N-[4-[2-oxo-6-[2-(trifluoromethyl)-1-piperidyl]-1H-pyridin-4-yl]-2-pyridyl]cyclopropanecarboxamide; N-[4-[2-oxo-6-[3-(trifluoromethyl)morpholin-4-yl]-1H-pyridin-4-yl]-2-pyridyl]acetamide; methyl N-[4-[2-(2-chlorophenyl)-6-oxo-1H-pyridin-4-yl]-2-pyridyl]carbamate; methyl N-[4-[2-[1-ethyl-3-(trifluoromethyl)pyrazol-4-yl]-6-oxo-1H-pyridin-4-yl]-2-pyridyl]carbamate; methyl N-[4-[2-oxo-6-[2-(trifluoromethyl)-3-pyridyl]-1H-pyridin-4-yl]-2-pyridyl]carbamate; methyl N-[4-[2-oxo-6-[2-(trifluoromethyl)phenyl]-1H-pyridin-4-yl]-2-pyridyl]carbamate; N-[4-[2-oxo-6-[2-(trifluoromethyl)phenyl]-1H-pyridin-4-yl]-2-pyridyl]acetamide; N-[4-[2-(4-methyl-3-pyridyl)-6-oxo-1H-pyridin-4-yl]-2-pyridyl]acetamide; N-[4-[2-oxo-6-[2-(trifluoromethyl)-3-pyridyl]-1H-pyridin-4-yl]-2-pyridyl]acetamide; N-[4-[2-[1-ethyl-3-(trifluoromethyl)pyrazol-4-yl]-6-oxo-1H-pyridin-4-yl]-2-pyridyl]acetamide; methyl N-[4-[2-oxo-6-[3-(trifluoromethyl)morpholin-4-yl]-1H-pyridin-4-yl]-2-pyridyl]carbamate; methyl N-[4-[2-oxo-6-[2-(trifluoromethyl)-1-piperidyl]-1H-pyridin-4-yl]-2-pyridyl]carbamate; N-[4-[2-(3-cyclopropylmorpholin-4-yl)-6-oxo-1H-pyridin-4-yl]-2-pyridyl]acetamide; N-[4-[2-[4-ethylsulfonyl-2-(trifluoromethyl)piperazin-1-yl]-6-oxo-1H-pyridin-4-yl]-2-pyridyl]acetamide; N-[4-[2-(2-methyl-3-pyridyl)-6-oxo-1H-pyridin-4-yl]-2-pyridyl]acetamide; N-[4-[2-oxo-6-[4-(trifluoromethyl)-3-thienyl]-1H-pyridin-4-yl]-2-pyridyl]acetamide; 1,1-Dimethyl-3-[4-[2-oxo-6-[2-(trifluoromethyl)phenyl]-1H-pyridin-4-yl]-2-pyridyl]urea; N-[4-[2-oxo-6-[2-(trifluoromethyl)phenyl]-1H-pyridin-4-yl]-2-pyridyl]pyrrolidine-1-carboxamide; N-[4-[2-[2-(1-methoxy-1-methyl-ethyl)pyrrolidin-1-yl]-6-oxo-1H-pyridin-4-yl]-2-pyridyl]acetamide; and pharmaceutically acceptable salts, tautomers, and stereoisomers thereof.

19. The method of claim 16 or 17, wherein the compound is selected from the group consisting of: 4-(2-Amino-4-pyridyl)-6-(3-pyridyl)-1H-pyridin-2-one; 4-(2-Amino-4-pyridyl)-6-(2-chlorophenyl)-1H-pyridin-2-one; N-[4-[2-(2-chlorophenyl)-6-oxo-1H-pyridin-4-yl]-2-pyridyl]-2-methoxy acetamide; N-[4-[2-oxo-6-[2-(trifluoromethyl)-1-piperidyl]-1H-pyridin-4-yl]-2-pyridyl]acetamide; N-[4-[2-oxo-6-[2-(trifluoromethyl)-1-piperidyl]-1H-pyridin-4-yl]-2-pyridyl]cyclopropanecarboxamide; N-[4-[2-oxo-6-[3-(trifluoromethyl)morpholin-4-yl]-1H-pyridin-4-yl]-2-pyridyl]acetamide; methyl N-[4-[2-(2-chlorophenyl)-6-oxo-1H-pyridin-4-yl]-2-pyridyl]carbamate; methyl N-[4-[2-[1-ethyl-3-(trifluoromethyl)pyrazol-4-yl]-6-oxo-1H-pyridin-4-yl]-2-pyridyl]carbamate; methyl N-[4-[2-oxo-6-[2-(trifluoromethyl)-3-pyridyl]-1H-pyridin-4-yl]-2 pyridyl]carbamate; methyl N-[4-[2-(4-methyl-3-pyridyl)-6-oxo-1H-pyridin-4-yl]-2-pyridyl]carbamate; methyl N-[4-[2-oxo-6-[2-(trifluoromethyl)phenyl]-1H-pyridin-4-yl]-2-pyridyl]carbamate; N-[4-[2-oxo-6-[2-(trifluoromethyl)phenyl]-1H-pyridin-4-yl]-2-pyridyl]acetamide; N-[4-[2-(4-methyl-3-pyridyl)-6-oxo-1H-pyridin-4-yl]-2-pyridyl]acetamide; N-[4-[2-oxo-6-[2-(trifluoromethyl)-3-pyridyl]-1H-pyridin-4-yl]-2-pyridyl]acetamide; N-[4-[2-[1-ethyl-3-(trifluoromethyl)pyrazol-4-yl]-6-oxo-1H-pyridin-4-yl]-2-pyridyl]acetamide; methyl N-[4-[2-oxo-6-[3-(trifluoromethyl)morpholin-4-yl]-1H-pyridin-4-yl]-2-pyridyl]carbamate; methyl N-[4-[2-oxo-6-[2-(trifluoromethyl)-1-piperidyl]-1H-pyridin-4-yl]-2-pyridyl]carbamate; methyl N-[4-[2-[4-ethylsulfonyl-2-(trifluoromethyl)piperazin-1-yl]-6-oxo-1H-pyridin-4-yl]-2-pyridyl]carbamate; methyl N-[4-[2-(3-cyclopropylmorpholin-4-yl)-6-oxo-1H-pyridin-4-yl]-2-pyridyl]carbamate; N-[4-[2-(3-cyclopropylmorpholin-4-yl)-6-oxo-1H-pyridin-4-yl]-2-pyridyl]acetamide; N-[4-[2-[4-ethylsulfonyl-2-(trifluoromethyl)piperazin-1-yl]-6-oxo-1H-pyridin-4-yl]-2-pyridyl]acetamide; 3-[4-[2-(2-chlorophenyl)-6-oxo-1H-pyridin-4-yl]-2-pyridyl]-1,1-dimethyl-urea; N-[4-[2-(2-chlorophenyl)-6-oxo-1H-pyridin-4-yl]-2-pyridyl]pyrrolidine-1-carboxamide; and pharmaceutically acceptable salts, tautomers, and stereoisomers thereof.

20. The method of claim 16 or 17, wherein the compound is selected from the group consisting of: N-[4-[2-(2-chlorophenyl)-6-oxo-1H-pyridin-4-yl]-2-pyridyl]acetamide; 4-(2-Amino-4-pyridyl)-6-(3-pyridyl)-1H-pyridin-2-one; 4-(2-Amino-4-pyridyl)-6-(2-chlorophenyl)-1H-pyridin-2-one; N-[4-[2-(2-chlorophenyl)-6-oxo-1H-pyridin-4-yl]-2-pyridyl]-2-methoxy-acetamide; N-[4-[2-oxo-6-[2-(trifluoromethyl)-1-piperidyl]-1H-pyridin-4-yl]-2-pyridyl]acetamide; N-[4-[2-oxo-6-[2-(trifluoromethyl)-1-piperidyl]-1H-pyridin-4-yl]-2-pyridyl]cyclopropanecarboxamide; N-[4-[2-oxo-6-[3-(trifluoromethyl)morpholin-4-yl]-1H-pyridin-4-yl]-2-pyridyl]acetamide; methyl N-[4-[2-(2-chlorophenyl)-6-oxo-1H-pyridin-4-yl]-2-pyridyl]carbamate; methyl N-[4-[2-[1-ethyl-3-(trifluoromethyl)pyrazol-4-yl]-6-oxo-1H-pyridin-4-yl]-2-pyridyl]carbamate; methyl N-[4-[2-oxo-6-[2-(trifluoromethyl)-3-pyridyl]-1H-pyridin-4-yl]-2-pyridyl]carbamate; methyl N-[4-[2-oxo-6-[2-(trifluoromethyl)phenyl]-1H-pyridin-4-yl]-2-pyridyl]carbamate; N-[4-[2-oxo-6-[2-(trifluoromethyl)phenyl]-1H-pyridin-4-yl]-2-pyridyl]acetamide; N-[4-[2-(4-methyl-3-pyridyl)-6-oxo-1H-pyridin-4-yl]-2-pyridyl]acetamide; N-[4-[2-oxo-6-[2-(trifluoromethyl)-3-pyridyl]-1H-pyridin-4-yl]-2-pyridyl]acetamide; N-[4-[2-[1-ethyl-3-(trifluoromethyl)pyrazol-4-yl]-6-oxo-1H-pyridin-4-yl]-2-pyridyl]acetamide; methyl N-[4-[2-oxo-6-[3-(trifluoromethyl)morpholin-4-yl]-1H-pyridin-4-yl]-2-pyridyl]carbamate; methyl N-[4-[2-oxo-6-[2-(trifluoromethyl)-1-piperidyl]-1H-pyridin-4-yl]-2-pyridyl]carbamate; N-[4-[2-(3-cyclopropylmorpholin-4-yl)-6-oxo-1H-pyridin-4-yl]-2-pyridyl]acetamide; N-[4-[2-[4-ethylsulfonyl-2-(trifluoromethyl)piperazin-1-yl]-6-oxo-1H-pyridin-4-yl]-2-pyridyl]acetamide; and pharmaceutically acceptable salts, tautomers, and stereoisomers thereof.

21. A method of treating cancer in a patient in need thereof, comprising:
(i) administering to the patient a therapeutically effective amount of a compound represented by Formula V: 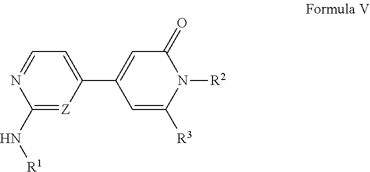
or a pharmaceutically acceptable salt, stereoisomer, or tautomer thereof, wherein:
R¹ is selected from phenyl and monocyclic 5-6 membered heteroaryl, wherein each of phenyl and monocyclic 5-6 membered heteroaryl is optionally substituted with one or more substituents independently selected from the group consisting of halogen, C₁-C₆alkyl, C₃-C₄cycloalkyl, C₁-C₆alkoxy, C₁-C₆haloalkyl, C₁-C₆haloalkoxy, amino, N—C₁-C₃alkylamino and N,N-diC₁-C₃alkylamino;
R² is selected from the group consisting of H, C₁-C₃haloalkyl, and C₁-C₃alkyl; R³ is selected from the group consisting of A, phenyl, and monocyclic heteroaryl, wherein each of phenyl and heteroaryl is optionally substituted with one or more occurrences of a substituent independently selected from the group consisting of R⁴, R⁵, R⁶, and R⁷;
each of R⁴, R⁵, R⁶, and R⁷ is independently selected from the group consisting of halogen, C₁-C₆alkyl, C₃-C₄cycloalkyl, C₁-C₆alkoxy, C₁-C₆haloalkyl, C₁-C₆haloalkoxy, azetidine, amino, N—C₁-C₃alkylamino, N,N-diC₁-C₃alkylamino, NHSO₂R⁸, SO₂R⁹, and hydroxy;
R⁸ is selected from C₁-C₃haloalkyl and C₁-C₃alkyl;
each R⁹ is independently selected from the group consisting of R¹⁰, C₁-C₆alkyl, amino, N—C₁-C₃alkylamino, N,N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen;
each R¹⁰ is independently selected from the group consisting of phenyl, benzyl, monocyclic heteroaryl, C₃-C₆cycloalkyl, and heterocyclyl, wherein each of phenyl, benzyl, monocyclic heteroaryl, C₃-C₆cycloalkyl, and heterocyclyl is optionally substituted with one or more occurrences of R¹¹;
each R¹¹ is independently selected from the group consisting of halogen, C₁-C₃haloalkyl, C₃-C₄cycloalkyl, C₁-C₃alkyl, amino, N—C₁-C₃alkylamino, N,N-diC₁-C₃alkylamino, and C₁-C₃alkoxyC₁-C₃alkyl;
A is 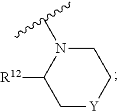
R¹² is selected from the group consisting of H, halogen, COR¹³, C₁-C₆alkyl, C₃-C₆cycloalkyl, C₁-C₃alkoxyC₁-C₃alkyl, C₁-C₆alkoxy, C₃-C₆cycloalkyl, C₁-C₃cyanoalkyl, and C₁-C₃haloalkyl;
R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹⁵, NSO₂R⁹, NCOCH₂R⁹, O, and a bond;
R¹⁴ is selected from H, C₁-C₃haloalkyl, C₁-C₃alkoxyC₁-C₃alkyl, C₁-C₃alkyl, and C₃-C₆cycloalkyl; and
R¹⁵ is selected from R¹⁰, C₁-C₆alkyl, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and
Z is selected from CH and N; and
(ii) administering to the patient a therapeutically effective amount of a STING agonist;
wherein administering the therapeutically effective amount of the STING agonist and the compound results in an increased expression level of at least one chemokine in the patient as compared to any increase in the expression level of the at least one chemokine resulting from administering the compound alone to the patient.

22. A method of upregulating at least one chemokine in a cell comprising: contacting the cell sample with:
(i) a compound represented by: 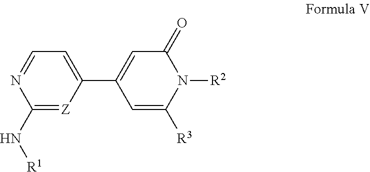
or a pharmaceutically acceptable salt, stereoisomer, or tautomer thereof, wherein:
R¹ is selected from phenyl and monocyclic 5-6 membered heteroaryl, wherein each of phenyl and monocyclic 5-6 membered heteroaryl is optionally substituted with one or more substituents independently selected from the group consisting of halogen, C₁-C₆alkyl, C₃-C₄cycloalkyl, C₁-C₆alkoxy, C₁-C₆haloalkyl, C₁-C₆haloalkoxy, amino, N—C₁-C₃alkylamino and N,N-diC₁-C₃alkylamino;
R² is selected from the group consisting of H, C₁-C₃haloalkyl, and C₁-C₃alkyl; R³ is selected from the group consisting of A, phenyl, and monocyclic heteroaryl, wherein each of phenyl and heteroaryl is optionally substituted with one or more occurrences of a substituent independently selected from the group consisting of R⁴, R⁵, R⁶, and R⁷;
each of R⁴, R⁵, R⁶, and R⁷ is independently selected from the group consisting of halogen, C₁-C₆alkyl, C₃-C₄cycloalkyl, C₁-C₆alkoxy, C₁-C₆haloalkyl, C₁-C₆haloalkoxy, azetidine, amino, N—C₁-C₃alkylamino, N,N-diC₁-C₃alkylamino, NHSO₂R⁸, SO₂R⁹, and hydroxy;
R⁸ is selected from C₁-C₃haloalkyl and C₁-C₃alkyl;
each R⁹ is independently selected from the group consisting of R¹⁰, C₁-C₆alkyl, amino, N—C₁-C₃alkylamino, N,N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen;
each R¹⁰ is independently selected from the group consisting of phenyl, benzyl, monocyclic heteroaryl, C₃-C₆cycloalkyl, and heterocyclyl, wherein each of phenyl, benzyl, monocyclic heteroaryl, C₃-C₆cycloalkyl, and heterocyclyl is optionally substituted with one or more occurrences of R¹¹;
each R¹¹ is independently selected from the group consisting of halogen, C₁-C₃haloalkyl, C₃-C₄cycloalkyl, C₁-C₃alkyl, amino, N—C₁-C₃alkylamino, N,N-diC₁-C₃alkylamino, and C₁-C₃alkoxyC₁-C₃alkyl;
A is 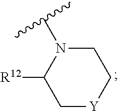
R¹² is selected from the group consisting of H, halogen, COR¹³, C₁-C₆alkyl, C₃-C₆cycloalkyl, C₁-C₃alkoxyC₁-C₃alkyl, C₁-C₆alkoxy, C₃-C₆cycloalkyl, C₁-C₃cyanoalkyl, and C₁-C₃haloalkyl;
R¹³ is selected from the group consisting of C₁-C₃alkoxy, N—C₁-C₃alkylamino, N,N-diC₁-C₃alkylamino, 1-pyrrolidinyl, 1-piperidinyl, and 1-azetidinyl; Y is selected from the group consisting of CH₂, S, SO, SO₂, NR¹⁴, NCOR⁹, NCOOR¹S, NSO₂R⁹, NCOCH₂R⁹, O, and a bond;
R¹⁴ is selected from H, C₁-C₃haloalkyl, C₁-C₃alkoxyC₁-C₃alkyl, C₁-C₃alkyl, and C₃-C₆cycloalkyl; and
R¹⁵ is selected from R¹⁰, C₁-C₆alkyl, and C₁-C₃alkoxyC₁-C₃alkyl, wherein each of C₁-C₆alkyl and C₁-C₃alkoxyC₁-C₃alkyl is optionally substituted with one occurrence of R¹⁰, and each of C₁-C₆alkyl and C₁-C₃alkoxyC₁-C₃alkyl is optionally substituted with or one or more independent occurrences of halogen; and
Z is selected from CH and N; and
in an amount sufficient to induce a Type I interferon response by the cell; and
(ii) a STING agonist in an amount sufficient to increase the expression level of the at least one chemokine in the cell.

23. The method of claim 21 or 22, wherein the compound is selected from the group consisting of: 4-(2-anilinopyrimidin-4-yl)-6-(2-chlorophenyl)-1H-pyridin-2-one; 4-(2-anilinopyrimidin-4-yl)-6-(3-pyridyl)-1H-pyridin-2-one; 4-(2-anilinopyrimidin-4-yl)-6-(4-pyridyl)-1H-pyridin-2-one; 4-(2-anilinopyrimidin-4-yl)-6-morpholino-1H-pyridin-2-one; 4-[2-[(2-Methylpyrimidin-4-yl)amino]-4-pyridyl]-6-[2-(trifluoromethyl)-1-piperidyl]-1H-pyridin-2-one; 4-(2-anilinopyrimidin-4-yl)-6-[2-(trifluoromethyl)-1-piperidyl]-1H-pyridin-2-one; 4-[2-[(2-Methylpyrimidin-4-yl)amino]-4-pyridyl]-6-[3-(trifluoromethyl)morpholin-4-yl]-1H-pyridin-2-one; 4-(2-anilinopyrimidin-4-yl)-6-[3-(trifluoromethyl)morpholin-4-yl]-1H-pyridin-2-one; 6-[4-[(4-Fluorophenyl)methylsulfonyl]-2-(trifluoromethyl)piperazin-1-yl]-4-[2-[(2-methylpyrimidin-4-yl)amino]-4-pyridyl]-1H-pyridin-2-one; 6-[4-Ethylsulfonyl-2-(trifluoromethyl)piperazin-1-yl]-4-[2-[(2-methylpyrimidin-4-yl)amino]-4-pyridyl]-1H-pyridin-2-one; 4-[2-(Oxazol-2-ylamino)-4-pyridyl]-6-[3-(trifluoromethyl)morpholin-4-yl]-1H-pyridin-2-one; 4-[2-[(2-Methylthiazol-4-yl)amino]-4-pyridyl]-6-[3-(trifluoromethyl)morpholin-4-yl]-1H-pyridin-2-one; 4-[2-[(2-methylpyrimidin-4-yl)amino]-4-pyridyl]-6-[2-(trifluoromethyl)-phenyl]-1H-pyridin-2-one; 4-[2-[(2-Methylpyrazol-3-yl)amino]-4-pyridyl]-6-[2-(trifluoromethyl)phenyl]-1H-pyridin-2-one; 4-[2-[(2-Methylthiazol-4-yl)amino]-4-pyridyl]-6-[2-(trifluoromethyl)phenyl]-1H-pyridin-2-one; 6-(4-methyl-3-pyridyl)-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-3-pyridyl]-1H-pyridin-2-one; 6-[1-ethyl-3-(trifluoromethyl)pyrazol-4-yl]-4-[2-[(2-methylpyrimidin-4-yl)am 4-pyridyl]-1H-pyridin-2-one; 4-[2-[(1-Methylimidazol-4-yl)amino]-4-pyridyl]-6-[2-(trifluoromethyl)phenyl pyridin-2-one; 6-(2-chlorophenyl)-4-[2-[(2-methylpyrimidin-4-yl)amino]-4-pyridyl]-1H-pyridin-2-one, and pharmaceutically acceptable salts, stereoisomers, and tautomers thereof.

24. The method of claim 21 or 22, wherein the compound is selected from the group consisting of: 4-(2-anilinopyrimidin-4-yl)-6-(2-chlorophenyl)-1H-pyridin-2-one; 4-(2-anilinopyrimidin-4-yl)-6-(3-pyridyl)-1H-pyridin-2-one; 4-(2-anilinopyrimidin-4-yl)-6-(4-pyridyl)-1H-pyridin-2-one; 4-(2-anilinopyrimidin-4-yl)-6-morpholino-1H-pyridin-2-one; 4-[2-[(2-Methylpyrimidin-4-yl)amino]-4-pyridyl]-6-[2-(trifluoromethyl)-1-piperidyl]-1H-pyridin-2-one; 4-(2-anilinopyrimidin-4-yl)-6-[2-(trifluoromethyl)-1-piperidyl]-1H-pyridin-2-one; 4-[2-[(2-Methylpyrimidin-4-yl)amino]-4-pyridyl]-6-[3-(trifluoromethyl)morpholin-4-yl]-1H-pyridin-2-one; 4-(2-anilinopyrimidin-4-yl)-6-[3-(trifluoromethyl)morpholin-4-yl]-1H-pyridin-2-one; 6-[4-[(4-Fluorophenyl)methylsulfonyl]-2-(trifluoromethyl)piperazin-1-yl]-4-[2-[(2-methylpyrimidin-4-yl)amino]-4-pyridyl]-1H-pyridin-2-one; 6-[4-Ethylsulfonyl-2-(trifluoromethyl)piperazin-1-yl]-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-phenyl]-1H-pyridin-2-one; 6-(4-methyl-3-pyridyl)-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-3-pyridyl]-1H-pyridin-2-one; 6-[1-ethyl-3-(trifluoromethyl)pyrazol-4-yl]-4-[2-[(2-methylpyrimidin-4-yl)am 4-pyridyl]-1H-pyridin-2-one; 6-(2-chlorophenyl)-4-[2-[(2-methylpyrimidin-4-yl)amino]-4-pyridyl]-1H-pyridin-2-one; 6-(3-cyclopropylmorpholin-4-yl)-4-[2-[(2-methylpyrimidin-4-yl)amino]-4-pyridyl]-1H-pyridin-2-one, and pharmaceutically acceptable salts, stereoisomers, and tautomers thereof.

25. The method of claim 21 or 22, wherein the compound is selected from the group consisting of: 4-(2-anilinopyrimidin-4-yl)-6-(2-chlorophenyl)-1H-pyridin-2-one; 4-(2-anilinopyrimidin-4-yl)-6-(3-pyridyl)-1H-pyridin-2-one; 4-(2-anilinopyrimidin-4-yl)-6-(4-pyridyl)-1H-pyridin-2-one 4-(2-anilinopyrimidin-4-yl)-6-morpholino-1H-pyridin-2-one; 4-[2-[(2-Methylpyrimidin-4-yl)amino]-4-pyridyl]-6-[2-(trifluoromethyl)-1-piperidyl]-1H-pyridin-2-one; 4-(2-anilinopyrimidin-4-yl)-6-[2-(trifluoromethyl)-1-piperidyl]-1H-pyridin-2-one; 4-[2-[(2-Methylpyrimidin-4-yl)amino]-4-pyridyl]-6-[3-(trifluoromethyl)morpholin-4-yl]-1H-pyridin-2-one; 4-(2-anilinopyrimidin-4-yl)-6-[3-(trifluoromethyl)morpholin-4-yl]-1H-pyridin-2-one; 6-[4-[(4-Fluorophenyl)methylsulfonyl]-2-(trifluoromethyl)piperazin-1-yl]-4-[2-[(2-methylpyrimidin-4-yl)amino]-4-pyridyl]-1H-pyridin-2-one; 6-[4-Ethylsulfonyl-2-(trifluoromethyl)piperazin-1-yl]-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-phenyl]-1H-pyridin-2-one; 6-(4-methyl-3-pyridyl)-4-[2-[(2-methylpyrimidin-4-yl)amino]-4-pyridyl]-1H-pyridin-2-one; 4-[2-[(2-methylpyrimidin-4-yl)amino]-4-pyridyl]-6-[2-(trifluoromethyl)-3-pyridyl]-1H-pyridin-2-one; 6-[1-ethyl-3-(trifluoromethyl)pyrazol-4-yl]-4-[2-[(2-methylpyrimidin-4-yl)amino]-4-pyridyl]-1H-pyridin-2-one; 6-(2-chlorophenyl)-4-[2-[(2-methylpyrimidin-4-yl)amino]-4-pyhdyl]-1H-pyridin-2-one, and pharmaceutically acceptable salts, stereoisomers, and tautomers thereof.

26. A method of treating cancer in a patient in need thereof, comprising:
(i) administering to the patient a therapeutically effective amount of a compound represented by Formula VI: 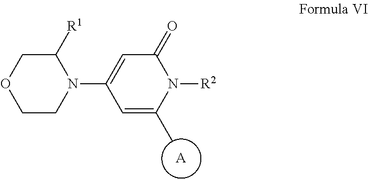
or a pharmaceutically acceptable salt, stereoisomer, or tautomer thereof, wherein:
R¹ is selected from C₁-C₃alkyl and cyclopropyl; R² is selected from the group consisting of H, C₁-C₃haloalkyl, and C₁-C₃alkyl;
A is selected from: 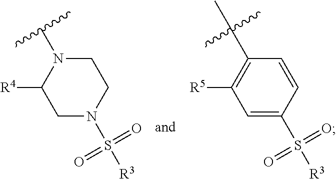
each R³ is independently selected from the group consisting of R⁶, C₁-C₆alkyl, amino N—C₁-C₃alkylamino, N, N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R⁶, and each of C₁-C₆alkyl and C₁-C₃alkoxyC₁-C₃alkyl is optionally substituted with or one or more independent occurrences of halogen;
R⁴ is selected from the group consisting of C₁-C₆alkyl, C₁-C₆alkoxy, C₁-C₆haloalkyl, C₃-C₆cycloalkyl, and phenyl, wherein phenyl is optionally substituted with one or more occurrences of a substituent independently selected from the group consisting of fluoro, chloro, methyl, methoxy, dimethylamino, trifluoromethoxy, trifluoromethyl, and cyclopropyl;
R⁵ is selected from the group consisting of halogen, C₁-C₆alkyl, C₁-C₆alkoxy, C₁-C₆haloalkyl, and C₃-C₆cycloalkyl;
each R⁶ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁷; and
each R⁷ is independently selected from the group consisting of halogen, amino, N—C₁-C₃alkylamino, N,N-diC₁-C₃alkylamino and C₁-C₃alkoxyC₁-C₃alkyl, C₁-C₃alkoxy, C₁-C₃haloalkoxy, C₃-C₆cycloalkyl, C₁-C₃haloalkyl, and C₁-C₃alkyl; and
(ii) administering to the patient a therapeutically effective amount of a STING agonist;
wherein administering the therapeutically effective amount of the STING agonist and the compound results in an increased expression level of at least one chemokine in the patient as compared to any increase in the expression level of the at least one chemokine resulting from administering the compound alone to the patient.

27. A method of upregulating at least one chemokine in a cell comprising: contacting the cell sample with:
(i) a compound represented by: 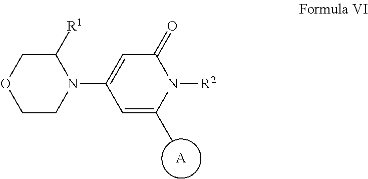
or a pharmaceutically acceptable salt, stereoisomer, or tautomer thereof, wherein:
R¹ is selected from C₁-C₃alkyl and cyclopropyl; R² is selected from the group consisting of H, C₁-C₃haloalkyl, and C₁-C₃alkyl;
A is selected from: 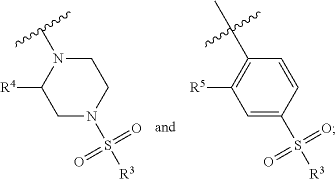
each R³ is independently selected from the group consisting of R⁶, C₁-C₆alkyl, amino N—C₁-C₃alkylamino, N, N-diC₁-C₃alkylamino, and C₁-C₃alkoxyC₁-C₃alkyl, wherein each of C₁-C₆alkyl and C₁-C₃alkoxyC₁-C₃alkyl is optionally substituted with one occurrence of R⁶, and each of C₁-C₆alkyl and C₁-C₃alkoxyC₁-C₃alkyl is optionally substituted with or one or more independent occurrences of halogen;
R⁴ is selected from the group consisting of C₁-C₆alkyl, C₁-C₆alkoxy, C₁-C₆haloalkyl, C₃-C₆cycloalkyl, and phenyl, wherein phenyl is optionally substituted with one or more occurrences of a substituent independently selected from the group consisting of fluoro, chloro, methyl, methoxy, dimethylamino, trifluoromethoxy, trifluoromethyl, and cyclopropyl;
R⁵ is selected from the group consisting of halogen, C₁-C₆alkyl, C₁-C₆alkoxy, C₁-C₆haloalkyl, and C₃-C₀cycloalkyl;
each R⁶ is independently selected from the group consisting of phenyl, monocyclic heteroaryl, C₃-C₆cycloalkyl, and heterocyclyl, wherein each of phenyl, monocyclic heteroaryl, C₃-C₆cycloalkyl, and heterocyclyl is optionally substituted with one or more occurrences of R⁷; and
each R⁷ is independently selected from the group consisting of halogen, amino, N—C₁-C₃alkylamino, N,N-diC₁-C₃alkylamino and C₁-C₃alkoxyC₁-C₃alkyl, C₁-C₃alkoxy, C₁-C₃haloalkoxy, C₃-C₆cycloalkyl, C₁-C₃haloalkyl, and C₁-C₃alkyl;
in an amount sufficient to induce a Type I interferon response by the cell; and
(ii) a STING agonist in an amount sufficient to increase the expression level of the at least one chemokine in the cell.

28. The method of claim 26 or 27, wherein the compound is selected from the group consisting of: 4-(3-methylmorpholin-4-yl)-6-[4-methylsulfonyl-2-(trifluoromethyl)piperazin-1-yl]-1H-pyridin-2-one; 6-[4-[(4-Fluorophenyl)methylsulfonyl]-2-(trifluoromethyl)piperazin-1-yl]-4-(3-methylmorpholin-4-yl)-1H-pyridin-2-one; 6-[4-[(5-Fluoro-3-pyridyl)sulfonyl]-2-(trifluoromethyl)piperazin-1-yl]-4-(3-methylmorpholin-4-yl)-1H-pyridin-2-one; 4-(3-methylmorpholin-4-yl)-6-[4-tetrahydrofuran-3-ylsulfonyl-2-(trifluoromethyl)piperazin-1-yl]-1H-pyridin-2-one; 4-(3-methylmorpholin-4-yl)-6-[4-pyrrolidin-1-ylsulfonyl-2-(trifluoromethyl)piperazin-1-yl]-1H-pyridin-2-one; N,N-dimethyl-4-[4-(3-methylmorpholin-4-yl)-6-oxo-1H-pyridin-2-yl]-3-(trifluoromethyl)piperazine-1-sulfonamide; 6-[4-(2-methoxyethylsulfonyl)-2-(trifluoromethyl)piperazin-1-yl]-4-(3-methylmorpholin-4-yl)-1H-pyridin-2-one; 6-[4-(4-fluorophenyl)sulfonyl-2-(trifluoromethyl)piperazin-1-yl]-4-(3-methylmorpholin-4-yl)-1H-pyridin-2-one; 4-(3-methylmorpholin-4-yl)-6-[4-(2-methylpyrazol-3-yl)sulfonyl-2-(trifluoromethyl)piperazin-1-yl]-1H-pyridin-2-one; 6-[4-Cyclopropylsulfonyl-2-(trifluoromethyl)piperazin-1-yl]-4-(3-methylmorpholin-4-yl)-1H-pyridin-2-one; 4-(3-methylmorpholin-4-yl)-6-[4-(1-piperidylsulfonyl)-2-(trifluoromethyl)piperazin-1-yl]-1H-pyridin-2-one; 4-(3-methylmorpholin-4-yl)-6-[4-morpholinosulfonyl-2-(trifluoromethyl)piperazin-1-yl]-1H-pyridin-2-one; 6-[4-(1,2-Dimethylimidazol-4-yl)sulfonyl-2-(trifluoromethyl)piperazin-1-yl]-4-(3-methylmorpholin-4-yl)-1H-pyridin-2-one; 6-[4-(1-methylcyclopropyl)sulfonyl-2-(trifluoromethyl)piperazin-1-yl]-4-(3-methylmorpholin-4-yl)-1H-pyridin-2-one; 4-(3-methylmorpholin-4-yl)-6-[4-methylsulfonyl-2-(trifluoromethyl)phenyl]-1H-pyridin-2-one; N,N-dimethyl-4-[4-(3-methylmorpholin-4-yl)-6-oxo-1H-pyridin-2-yl]-3-(trifluoromethyl)benzenesulfonamide; and pharmaceutically acceptable salts, stereoisomers, and tautomers thereof.

29. The method of claim 26 or 27, wherein the compound is selected from the group consisting of: 4-(3-methylmorpholin-4-yl)-6-[4-methylsulfonyl-2-(trifluoromethyl)piperazin-1-yl]-1H-pyridin-2-one; 6-[4-[(4-Fluorophenyl)methylsulfonyl]-2-(trifluoromethyl)piperazin-1-yl]-4-(3-methylmorpholin-4-yl)-1H-pyridin-2-one; 6-[4-[(5-Fluoro-3-pyridyl)sulfonyl]-2-(trifluoromethyl)piperazin-1-yl]-4-(3-methylmorpholin-4-yl)-1H-pyridin-2-one; 4-(3-methylmorpholin-4-yl)-6-[4-tetrahydrofuran-3-ylsulfonyl-2-(trifluoromethyl)piperazin-1-yl]-H-pyridin-2-one; 4-(3-methylmorpholin-4-yl)-6-[4-pyrrolidin-1-ylsulfonyl-2-(trifluoromethyl)piperazin-1-yl]-1H-pyridin-2-one; N,N-dimethyl-4-[4-(3-methylmorpholin-4-yl)-6-oxo-1H-pyridin-2-yl]-3-(trifluoromethyl)piperazine-1-sulfonamide; 6-[4-(2-methoxyethylsulfonyl)-2-(trifluoromethyl)piperazin-1-yl]-4-(3-methylmorpholin-4-yl)-1H-pyhdin-2-one; 6-[4-(4-fluorophenyl)sulfonyl-2-(trifluoromethyl)piperazin-1-yl]-4-(3-methylmorpholin-4-yl)-1H-pyridin-2-one; 4-(3-methylmorpholin-4-yl)-6-[4-(2-methylpyrazol-3-yl)sulfonyl-2-(trifluoromethyl)piperazin-1-yl]-1H-pyridin-2-one; and pharmaceutically acceptable salts, stereoisomers, and tautomers thereof.

30. The method of any one of claims 1-29, wherein the at least one chemokine is selected from the group consisting of CCL5 and CXCL10.

31. The method of any one of claims 1, 3-9, 11-12, 14-16, 18-21, 23-26, and 28-29, wherein the cancer is selected from the group consisting of gastrointestinal stromal tumors, a esophageal cancer, a gastric cancer, a melanoma, a glioma, a glioblastoma, an ovarian cancer, a bladder cancer, a head cancer, a neck cancer, a urothelial cancer, a uterine cancer, a pancreatic cancer, a prostate cancer, a lung cancer, a breast cancer, a renal cancer, a hepatic cancer, an osteosarcoma, a sarcoma, a multiple myeloma, a cervical carcinoma, a cancer that is metastatic to bone, a papillary thyroid carcinoma, a non-small cell lung cancer, a lymphoma, a leukemia, and a colorectal cancer.

32. The method of any one of claims 2-8, 10-11, 13-15, 17-20, 22-25, and 27-29, wherein the cancerous cell is of a cancer selected from the group consisting of gastrointestinal stromal tumors, a esophageal cancer, a gastric cancer, a melanoma, a glioma, a glioblastoma, an ovarian cancer, a bladder cancer, a head cancer, a neck cancer, a urothelial cancer, a uterine cancer, a pancreatic cancer, a prostate cancer, a lung cancer, a breast cancer, a renal cancer, a hepatic cancer, an osteosarcoma, a sarcoma, a multiple myeloma, a cervical carcinoma, a cancer that is metastatic to bone, a papillary thyroid carcinoma, a non-small cell lung cancer, a lymphoma, a leukemia, and a colorectal cancer.

33. The method of claim 31 or 32, wherein the cancer is selected from the group consisting of a renal cancer and a melanoma.

34. The method of any one of claims 31-33, wherein the renal cancer is renal cell carcinoma.

35. The method of claim 34, wherein the renal cell carcinoma is clear-cell renal cell carcinoma.

36. The method of any one of claims 1-35, further comprising administering an additional therapeutic agent to the patient.

37. The method of claim 36, wherein the additional therapeutic agent is selected from the group consisting of a PD-1 pathway antagonist, a TIM-3 pathway antagonist, a Vista pathway antagonist, a BTLA pathway antagonist, a LAG-3 pathway antagonist, a TIGIT pathway antagonist, and a CTLA4 pathway antagonist.

38. A method of treating cancer in a patient in need thereof, comprising:
(i) means for inducing a Type I interferon response in a cancerous cell in the patient; and
(ii) administering a therapeutically effective amount of a STING agonist to the patient;
wherein the therapeutically effective amount of the STING agonist results in an increased expression level of at least one chemokine in the patient as compared to any increase in the expression level of the at least one chemokine resulting from administering the compound to the patient.

39. The method of any one of claims 1-38, wherein the STING agonist is selected from the group consisting of 5,6-dimethylxanthenone-4-acetic acid (DMXAA), ADU-S100, MK-1454, MK-2118, BMS-986301, GSK3745417, SB-11285, B11387446 (BI-STING), E7766, TAK-676, SNX281, SYNB1891, JNJ-67544412, JNJ-′6196, GSK532, TTI-10001, ALG-031048, MSA-1, MSA-2, CRD-5500, MV-626, SR-8314, SR-8291, SR8541A, SR-717, STING antibody-drug conjugates (ADC), and IMSA-101, and pharmaceutically acceptable salts thereof.

40. The method of any one of claims 1-38, wherein the STING agonist is selected from the group consisting of ADU-S100, MK-1454, MK-2118, BMS-986301, GSK3745417, SB-11285, BI1387446 (BI-STING), E7766, TAK-676, SNX281, SYNB1891, and IMSA-101, and pharmaceutically acceptable salts thereof.

41. The method of any one of claims 1-40, wherein the STING agonist is selected from ADU-S100 and pharmaceutically acceptable salts thereof.


SEQUENCE LISTING (22 sequences)

SEQ ID NO: 1 - DNA, 23 bp, synthetic construct
  source 1..23: mol_type = other DNA
gcttggattcctacaaagaagca

SEQ ID NO: 2 - DNA, 20 bp, synthetic construct
  source 1..20: mol_type = other DNA
ctgtgcgagtgtaccggatg

SEQ ID NO: 3 - DNA, 21 bp, synthetic construct
  source 1..21: mol_type = other DNA
gctggacgtgaccatcatgta

SEQ ID NO: 4 - DNA, 22 bp, synthetic construct
  source 1..22: mol_type = other DNA
gccctacaaggtgtatcagttg

SEQ ID NO: 5 - DNA, 20 bp, synthetic construct
  source 1..20: mol_type = other DNA
gaagcagcaaccatgcgtga

SEQ ID NO: 6 - DNA, 21 bp, synthetic construct
  source 1..21: mol_type = other DNA
atagatggtcaatgcggcgtc

SEQ ID NO: 7 - DNA, 21 bp, synthetic construct
  source 1..21: mol_type = other DNA
atccccacatgacttcctctt

SEQ ID NO: 8 - DNA, 19 bp, synthetic construct
  source 1..19: mol_type = other DNA
gggccgtataggaacgtgc

SEQ ID NO: 9 - DNA, 21 bp, synthetic construct
  source 1..21: mol_type = other DNA
tgctgtcgctttgatggtact

SEQ ID NO: 10 - DNA, 20 bp, synthetic construct
  source 1..20: mol_type = other DNA
ggcattgccaatctggacac

SEQ ID NO: 11 - DNA, 20 bp, synthetic construct
  source 1..20: mol_type = other DNA
ctccccatattcctcggaca

SEQ ID NO: 12 - DNA, 22 bp, synthetic construct
  source 1..22: mol_type = other DNA
tggcattcaaggagtacctctc

SEQ ID NO: 13 - DNA, 19 bp, synthetic construct
  source 1..19: mol_type = other DNA
actccttgatgtgggcacg

SEQ ID NO: 14 - DNA, 20 bp, synthetic construct
  source 1..20: mol_type = other DNA
atgctgatgcaggtacagcg

SEQ ID NO: 15 - DNA, 20 bp, synthetic construct
  source 1..20: mol_type = other DNA
gctgctttgcctacctctcc

SEQ ID NO: 16 - DNA, 21 bp, synthetic construct
  source 1..21: mol_type = other DNA
ccaagtgctgccgtcattttc

SEQ ID NO: 17 - DNA, 21 bp, synthetic construct
  source 1..21: mol_type = other DNA
cagctccaagaaaggacgaac

SEQ ID NO: 18 - DNA, 21 bp, synthetic construct
  source 1..21: mol_type = other DNA
agttccagcacattttgcgag

SEQ ID NO: 19 - DNA, 21 bp, synthetic construct
  source 1..21: mol_type = other DNA
tcgagtgacaaacacgactgc

SEQ ID NO: 20 - DNA, 21 bp, synthetic construct
  source 1..21: mol_type = other DNA
ggctcgcagggatgatttcaa

SEQ ID NO: 21 - DNA, 21 bp, synthetic construct
  source 1..21: mol_type = other DNA
ggcagtgtaactcttctgcat

SEQ ID NO: 22 - DNA, 21 bp, synthetic construct
  source 1..21: mol_type = other DNA
tcatcctccgtgagttctcca